(19) United States
(12) Patent Application Publication
(10) Pub. No.: US 2026/0217799 A1
(43) Pub. Date: Jul. 30, 2026

(54) RNA COMPOSITIONS TARGETING HIV

(71) Applicant: BioNTech SE, Mainz (DE)

(72) Inventors: Valentin Le Douce, Mörfelden-Walldorf (DE); Felix Tolksdorf, Wiesbaden (DE); Johannes Nelke, Mainz (DE); Gábor Boros, Mainz (DE); Jonas Reinholz, Nierstein (DE); Katalin Karikó, Rydal, PA (US); Sergey Bessonov, Huerth (DE)

(21) Appl. No.: 19/144,213

(22) PCT Filed: Dec. 27, 2023
(86) PCT No.: PCT/IB2023/063293

§ 371 Date: Jun. 27, 2025

Related U.S. Application Data
(60) Provisional application No. 63/477,460, filed on Dec. 28, 2022.
(60) Provisional application No. 63/517,542, filed on Aug. 3, 2023.

(51) Int. Cl. C07K 16/114; A61K 39/00

(52) U.S. Cl. CPC C07K 16/114; A61K 2039/505; C07K 2317/52; C07K 2317/524; C07K 2317/526; C07K 2317/53; C07K 2317/622; C07K 2317/76; C07K 2317/94

(57) ABSTRACT

The present disclosure provides compositions (e.g., pharmaceutical compositions) for delivery of anti-HIV antibody agents and related technologies (e.g., components thereof and/or methods relating thereto). Among other things, the present disclosure provides polyribonucleotides encoding an immnunoglobulin chain of an anti-HIV antibody agent.

### CROSS REFERENCE TO RELATED APPLICATIONS

This application claims the benefit of U.S. Provisional Application No. 63/477,460, filed Dec. 28, 2022, and U.S. Provisional Application No. 63/517,542, filed Aug. 3, 2023, which are herein incorporated by reference in their entirety.

### BACKGROUND

Human Immunodeficiency Virus (HIV) is an infectious virus associated with Acquired Immune Deficiency Syndrome (AIDS). According to the World Health Organization, over 37 million people worldwide are currently affected by HIV. In 2020, approximately 680,000 people died from HIV-related causes and approximately 1.5 million people acquired HIV. There is currently no cure for HIV.

### SUMMARY

The present disclosure recognizes that HIV mutates rapidly. The highly mutable nature of HIV virons allows them to escape pressure from host immunity and/or treatment. To combat viral escape, combination therapies targeting HIV, including anti-HIV antibody agents, have been considered. However, the development of such combination therapies has been hindered by a number of challenges. First, development of individual HIV therapies is time consuming and expensive. For instance, anti-HIV antibody development is challenged by demanding and costly production, including purification and formulation methods associated with protein therapeutics. Second, combination therapies present regulatory challenges. In addition to ensuring that a combination therapy will be safe and efficacious, tight regulation over manufacture of the individual therapies, compounding of the multiple therapies together, and quality control during storage and administration complicate the use of combination therapies. Third, administration of antibodies to a subject can be painful and time consuming. Generally, antibodies are administered intravenously over a longer period of time. Administration of multiple antibodies can add to the complexity of antibody administration, increasing patient discomfort and taking up additional time. Finally, recombinant antibodies can have a short serum half-life.

The present disclosure provides insights that address these challenges, making it possible to not only deliver single anti-HIV antibody agents to a subject safely, reliably, and with strong potency, but also making it possible to deliver multiple anti-HIV therapeutics, including multiple anti-HIV antibody agents to a subject. For example, the present disclosure describes antibody agents or portions thereof (e.g., immunoglobulin chains) that are delivered to a subject via polyribonucleotides. Antibody agents that are delivered to a subject as one or more polyribonucleotides that encode the antibody agent are referred to herein as “RiboMabs.” After delivery of one or more polyribonucleotides that encode an antibody agent to a subject, the antibody agent, i.e., the “RiboMab” is expressed by the subject's body. Further, the term “RibobNAb” refers to a RiboMab that comprises all or part of a broadly neutralizing antibody (bNAb), e.g., a broadly neutralizing antibody targeting HIV. Utilizing polyribonucleotides as a therapeutic agent (in contrast to administering an antibody agent itself) involves simpler and less expensive manufacturing processes. The less complex production of polyribonucleotides encoding antibody agent(s) (e.g., anti-HIV antibody agent(s)) can streamline manufacture (e.g., by circumventing the need for intensive glycan production and profiling), mitigating regulatory and production challenges associated with developing and using antibody agents themselves. Additionally, polyribonucleotides are effective at producing similar effects to recombinant proteins but tend to require much lower volumes be administered to a subject. This is because polyribonucleotides encoding, e.g., anti-HIV antibody agent(s), can be administered to a subject and the subject's body produces the anti-HIV antibody agent(s) itself. Using a lower volume can provide a patient with a more pleasant experience and increase patient compliance with a treatment regimen. The present disclosure also provides technologies that address certain limitations of recombinant antibody technologies, including for example, the short serum half-life of recombinant antibodies by utilizing RNA technologies as a modality to express antibody agents directly in the patient's cells.

RiboMab technology also allows for two or more antibody agents to be administered to a subject simultaneously. Typically, an antibody produced by, e.g., humans, comprises four polypeptide chains—two “heavy” chains and two “light” chains. Each polypeptide chain (whether heavy or light) includes (1) a “variable” domain, having a sequence that varies among antibodies and a structure that determines the antigen to which an antibody binds, and (2) a “constant” domain(s), having a sequence and a structure that generally remain unchanged across antibodies of a given class, thus imparting little effect on antigen binding. In humans, specialized white blood cells, “B cells,” produce antibodies. Heavy chains and light chains are assembled to form an antibody through two key pairings: (1) the fragment crystallizable (Fc) domains of the two heavy chains pair together, and (2) the two light chains each pair with a heavy chain via disulfide linkages. During normal antibody production in a human, a single antibody is produced by a single B cell. In that situation, the correct pairing of heavy and light chains is ensured because only one species of heavy chain and one species of light chain are present in each B cell. However, the administration of a nucleic acid composition encoding more than one antibody agent to a subject requires the correct assembly of immunoglobulin chains (e.g., heavy chains and light chains) so that unwanted side products (e.g., antibody agents with unintended pairings) do not form.

Among other things, the present disclosure provides polyribonucleotides encoding an immunoglobulin chain of an antibody agent.

In one aspect, the present disclosure provides a polyribonucleotide encoding an immunoglobulin chain of an antibody agent, wherein the immunoglobulin chain comprises a single chain fragment variable (scFv), and the scFv comprises a heavy chain variable (VH) domain, a linker, and a light chain variable (VL) domain, wherein the VH domain comprises an HCDR1, an HCDR2, and an HCDR3, wherein the HCDR1, HCDR2, and HCDR3 comprise an amino acid sequence according to SEQ ID NO: 18, 21, and 24, respectively, and wherein the VH domain comprises a first mutation at residue 3 and a second mutation at residue 5 relative to the amino acid sequence according to SEQ ID NO: 1494; and wherein the VL domain comprises an LCDR1, an LCDR2, and an LCDR3, wherein the LCDR1, LCDR2, and LCDR3 comprise an amino acid sequence according to SEQ ID NO: 27, 30, and 33, respectively.

In some embodiments, the first mutation comprises a substitution mutation that results in a positively charged amino acid being present at residue 3. In some embodiments, the positively charged amino acid comprises an amino acid selected from Lys (K), Arg (R), or His (H). In some embodiments, the positively charged amino acid comprises a His (H) residue. In some embodiments, the second mutation comprises a substitution mutation that results in a polar amino acid being present at residue 5. In some embodiments, the polar amino acid comprises an amino acid selected from Ser (S), Thr (T), Tyr (Y), Asn (N), or Gln (Q). In some embodiments, the polar amino acid comprises a Thr (T) residue. In some embodiments, the VH domain comprises an amino acid sequence that is at least 85% identical to the amino acid sequence according to SEQ ID NO: 36. In some embodiments, the VH domain comprises an amino acid sequence that is at least 90% identical to the amino acid sequence according to SEQ ID NO: 36. In some embodiments, the VH domain comprises an amino acid sequence SEQ ID NO: 1494 with substitution mutations Q3H and V5T. In some embodiments, the VH domain comprises an amino acid sequence according to SEQ ID NO: 36.

In some embodiments, the VL domain comprises an amino acid sequence that is at least 85% identical to the amino acid sequence according to SEQ ID NO: 43. In some embodiments, the VL domain comprises an amino acid sequence that is at least 90% identical to the amino acid sequence according to SEQ ID NO: 43. In some embodiments, the VL domain comprises an amino acid sequence according to SEQ ID NO: 43.

In some embodiments, the scFv comprises, in order: (i) the VH domain, (ii) the linker, and (iii) the VL domain.

In some embodiments, the scFv comprises, in order: (i) the VL domain, (ii) the linker, and (iii) the VH domain.

In some embodiments, the linker comprises an amino acid sequence according to SEQ ID NO: 48. In some embodiments, the linker comprises an amino acid sequence according to SEQ ID NO: 59. In some embodiments, the immunoglobulin chain further comprises a second linker following the scFv domain. In some embodiments, the scFv and the second linker comprises or consists of an amino acid sequence according to SEQ ID NO: 64, 67, 70, or 73. In some embodiments, the immunoglobulin chain comprises a hinge domain following the second linker. In some embodiments, the hinge domain comprises or consists of an amino acid sequence according to SEQ ID NO: 167.

In some embodiments, the immunoglobulin chain comprises one or more constant domains, and wherein the scFv is operably linked to the one or more constant domains. In some embodiments, the hinge domain is between the scFv and the one or more constant domains. In some embodiments, the one or more constant domains comprise a CH3 domain. In some embodiments, the CH3 domain comprises a G1m17,1 or a G1m3 allotype. In some embodiments, the CH3 domain comprises one or more substitution mutations, wherein the one or more substitution mutations comprise or consist of M88L, N94S, or a combination thereof, and wherein the substitution mutation positions are relative to the amino acid sequence according to SEQ ID NO: 1495. In some embodiments, the CH3 domain comprises substitution mutations at residue 16 and residue 18 relative to the amino acid sequence according to SEQ ID NO: 1495. In some embodiments, the CH3 domain comprises an amino acid sequence SEQ ID NO: 1495 with substitution mutations D16E and L18M. In some embodiments, the CH3 domain comprises or consists of an amino acid sequence according to SEQ ID NO: 116.

In some embodiments, the immunoglobulin chain comprises or consists of a sequence according to SEQ ID NO: 1343. In some embodiments, the immunoglobulin chain comprises or consists of a sequence according to SEQ ID NO: 1346. In some embodiments, the immunoglobulin chain comprises or consists of a sequence according to SEQ ID NO: 1349. In some embodiments, the immunoglobulin chain comprises or consists of a sequence according to SEQ ID NO: 1352.

In some embodiments, the polyribonucleotide comprises a ribonucleic acid sequence encoding a secretion signal. In some embodiments, the secretion signal comprises a ribonucleic acid sequence that is at least 90% identical to SEQ ID NO: 4 or 8. In some embodiments, the secretion signal comprises a ribonucleic acid sequence comprises SEQ ID NO: 4 or 8.

In some embodiments, the polyribonucleotide comprises one or more non-coding sequence elements. In some embodiments, the one or more non-coding sequence elements enhances RNA stability and/or translation efficiency. In some embodiments, the one or more non-coding sequence elements comprise a 3′ untranslated region (UTR), a 5′ UTR, a 5′-cap, a polyadenine (polyA) tail, or any combination thereof. In some embodiments, the polyA tail is or comprises a modified polyA sequence, preferably an interrupted polyA tail. In some embodiments, the polyA tail comprises or consists of a sequence that is at least 90% identical to SEQ ID NO: 16. In some embodiments, the 3′ UTR comprises or consists of a nucleic acid sequence that is at least 90% identical to SEQ ID NO: 14. In some embodiments, the 5′ UTR comprises or consists of a nucleic acid sequence that is at least 90% identical to SEQ ID NO: 10. In some embodiments, the 5′-cap is (m27,3′-O)Gppp(m2′-O)ApG. In some embodiments, the polyribonucleotide comprises one or more modified ribonucleotides. In some embodiments, the one or more modified ribonucleotides comprise pseudouridine.

In some embodiments, the polyribonucleotide comprises or consists of a ribonucleic acid sequence according to SEQ ID NO: 1000. In some embodiments, the polyribonucleotide comprises or consists of a ribonucleic acid sequence according to SEQ ID NO: 1048. In some embodiments, the polyribonucleotide comprises or consists of a ribonucleic acid sequence according to SEQ ID NO: 1096. In some embodiments, the polyribonucleotide comprises or consists of a ribonucleic acid sequence according to SEQ ID NO: 1144.

In another aspect, the present disclosure provides, a polyribonucleotide comprising or consisting of a ribonucleic acid sequence according to any one of SEQ ID NOs: 1000, 1048, 1096, or 1044.

The present disclosure also provides, in one aspect, a composition comprising one or more polyribonucleotides described herein. In some embodiments, the composition further comprises lipid nanoparticles, polyplexes (PLX), lipidated polyplexes (LPLX), or liposomes, wherein the one or more polyribonucleotides are fully or partially encapsulated within the lipid nanoparticles, polyplexes (PLX), lipidated polyplexes (LPLX), or liposomes. In some embodiments, the composition further comprises lipid nanoparticles, wherein the one or more polyribonucleotides are encapsulated within the lipid nanoparticles. In some embodiments, the lipid nanoparticles are cationic lipid nanoparticles.

The present disclosure provides, in another aspect, a pharmaceutical composition comprising the composition of any one of claims 53-56 and at least one pharmaceutically acceptable excipient. In some embodiments, the pharmaceutical composition is used in the treatment and/or prevention of HIV comprising administering the pharmaceutical composition to a subject.

The present disclosure provides, in another aspect, a method comprising administering the pharmaceutical composition described herein to a subject.

In some embodiments, administering the pharmaceutical composition to the subject results in expression of the antibody agent in the subject. In some embodiments, the antibody agent is expressed in the subject at a titer of: (a) at least 1 μg/ml in plasma; or (b) at least 1 μg/ml in serum. In some embodiments, the antibody agent is expressed in the subject at a titer of: (a) at least 10 μg/ml in plasma; or (b) at least 10 μg/ml in serum. In some embodiments, the antibody agent is detectable in serum of a subject for period of time of at least 5 days, at least 10 days, at least 15 days, at least 20 days, at least 25 days, or at least 30 days. In some embodiments, the antibody agent is detectable in serum of a subject for a period of time of at least 30 days.

In some embodiments, the antibody agent is capable of neutralizing one or more HIV strains when tested in the TZM-bl cell pseudovirus neutralization assay at antibody agent concentrations up to 25 μg/ml. In some embodiments, the antibody agent delivered as a polyribonucleotide has higher neutralization activity of one or more HIV strains compared to an equivalent amount of parental control antibody delivered as a polyribonucleotide, wherein the parental control antibody is an IgG antibody comprising the same VH and VL domain as the antibody agent. In some embodiments, the one of more HIV strains comprises one or more HIV strains selected from ZM53M.PB12, Du156.12, Q769.d22, 0330.v4.c3, R2184.c04, 89-F1_2_25, CAP204_2_00_F6_6, Ce1176_A3, 6980.v0.c31, PVO.4, CAP45, CNE8, T250-4, 3103.v3.c10 and C1080_c3.

In some embodiments, the subject has or is at risk of developing an HIV infection.

In some embodiments, the method is a method of treating and/or preventing an HIV infection.

In some embodiments, the present disclosure also provides use of the composition described herein or the pharmaceutical composition described herein for the treatment and/or prevention of HIV in a subject.

In some embodiments, the present disclosure provides a method of producing an antibody agent comprising administering to cells the composition described herein, or the pharmaceutical composition described herein so that the cells express and secrete the antibody agent. In some embodiments, the cells are in a subject and the antibody agent is produced at a therapeutically relevant plasma concentration or a therapeutically relevant serum concentration. In some embodiments, the therapeutically relevant plasma concentration or the therapeutically relevant serum concentration is at least 10 μg/ml.

In one aspect, an immunoglobulin chain comprises a heavy chain variable (VH) domain. In some embodiments, a VH domain comprises a heavy chain complementarity determining region (HCDR)1 comprising an amino acid sequence according to SEQ ID NO: 18, an HCDR2 comprising an amino acid sequence according to SEQ ID NO: 21, and an HCDR3 comprising an amino acid sequence according to SEQ ID NO: 24.

In some embodiments, a VH domain comprises or consists of an amino acid sequence according to SEQ ID NO: 24. In some embodiments, a VH domain comprises an amino acid sequence having at least 85%, at least 90%, at least 95%, at least 96%, at least 97%, at least 98%, or at least 99% identity to an amino acid sequence according to SEQ ID NO: 36.

In some embodiments, a polyribonucleotide comprises a VH domain-encoding sequence. In some embodiments, a VH domain-encoding sequence comprises (a) the HCDR1-encoding sequence that comprises or consists of the ribonucleic acid sequence according to SEQ ID NO: 19, (b) the HCDR2-encoding sequence that comprises or consists of the ribonucleic acid sequence according to SEQ ID NO: 22, and (c) the HCDR3-encoding sequence that comprises or consists of the ribonucleic acid sequence according to SEQ ID NO: 25.

In some embodiments, a VH domain-encoding sequence comprises a ribonucleic acid sequence having at least 85%, at least 90%, at least 95%, at least 96%, at least 97%, at least 98%, or at least 99% identity to a ribonucleic acid sequence according to SEQ ID NO: 37. In some embodiments, a VH domain-encoding sequence comprises or consists of a ribonucleic acid sequence according to SEQ ID NO: 37. In some embodiments, a VH domain-encoding sequence comprises a ribonucleic acid sequence having at least 85%, at least 90%, at least 95%, at least 96%, at least 97%, at least 98%, or at least 99% identity to a ribonucleic acid sequence according to SEQ ID NO: 39. In some embodiments, a VH domain-encoding sequence comprises or consists of a ribonucleic acid sequence according to SEQ ID NO: 41. In some embodiments, a VH domain-encoding sequence comprises a ribonucleic acid sequence having at least 85%, at least 90%, at least 95%, at least 96%, at least 97%, at least 98%, or at least 99% identity to a ribonucleic acid sequence according to SEQ ID NO: 39. In some embodiments, a VH domain-encoding sequence comprises or consists of a ribonucleic acid sequence according to SEQ ID NO: 39.

In some embodiments, an immunoglobulin chain comprising a VH domain as described herein comprises one or more constant domains. In some embodiments, a VH domain is operably linked to one or more constant domains.

In some embodiments, an immunoglobulin chain comprising a VH domain as described herein comprises one or more constant domains. In some embodiments, a VH domain is operably linked to one or more constant domains.

In some embodiments, one or more constant domains comprise a CH2 domain. In some embodiments, a polyribonucleotide comprises a ribonucleic acid sequence that encodes the CH2 domain.

In some embodiments, one or more constant domains comprise a CH3 domain. In some embodiments, a polyribonucleotide comprises a ribonucleic acid sequence that encodes the CH3 domain.

In some embodiments, one or more constant domains comprise a hinge domain. In some embodiments, a polyribonucleotide comprises a ribonucleic acid sequence that encodes the hinge domain.

In some embodiments, one or more constant domains comprise a CH1 domain. In some embodiments, a polyribonucleotide comprises a ribonucleic acid sequence that encodes a CH1 domain.

In some embodiments, one or more constant domains comprise a CL domain. In some embodiments, a polyribonucleotide comprises a ribonucleic acid sequence that encodes a CL domain.

In some embodiments, an immunoglobulin chain comprising a VH domain as described herein comprises a CH1 domain, a hinge domain, a CH2 domain, and a CH3 domain.

In some embodiments, an immunoglobulin chain comprising a VH domain as described herein comprises a CL domain, a hinge domain, a CH2 domain, and a CH3 domain.

The present disclosure further provides a polyribonucleotide encoding an immunoglobulin chain of an antibody agent, where the immunoglobulin chain comprises a light chain variable (VL) domain.

In some embodiments, a VL domain comprises (a) a light chain complementarity determining region (LCDR)1 comprising an amino acid sequence according to SEQ ID NO: 27, (b) an LCDR2 comprising an amino acid sequence according to SEQ ID NO: 30, and (c) an LCDR3 comprising an amino acid sequence according to SEQ ID NO: 33.

In some embodiments, a polyribonucleotide comprises a VL domain-encoding sequence. In some embodiments, a VL domain-encoding sequence comprises (a) an LCDR1-encoding sequence that comprises or consists of a ribonucleic acid sequence according to SEQ ID NO: 28, an LCDR2-encoding sequence that comprises or consists of a ribonucleic acid sequence according to SEQ ID NO: 31, and an LCDR3-encoding sequence that comprises or consists of a ribonucleic acid sequence according to SEQ ID NO: 34.

In some embodiments, a VL domain comprises or consists of an amino acid sequence according to SEQ ID NO: 43. In some embodiments, a VL domain-encoding sequence comprises or consists of a ribonucleic acid sequence according to SEQ ID NO: 44. In some embodiments, a VL domain-encoding sequence comprises or consists of a ribonucleic acid sequence according to SEQ ID NO: 46.

In some embodiments, an immunoglobulin chain comprising a VL domain further comprises a constant domain. In some embodiments, a VL domain is operably linked to a constant domain. In some embodiments, a polyribonucleotide comprises a ribonucleic acid sequence that encodes the constant domain.

In some embodiments, an immunoglobulin chain comprising a VL domain further comprises a CL domain. In some embodiments, a polyribonucleotide comprises a ribonucleic acid sequence that encodes the CL domain. In some embodiments, a CL domain is a kappa constant domain. In some embodiments, a polyribonucleotide comprises a ribonucleic acid sequence that encodes the light chain constant domain.

In some embodiments, an immunoglobulin chain comprising a VL domain further comprises a CH1 domain. In some embodiments, a polyribonucleotide comprises a ribonucleic acid sequence that encodes the CH1 domain.

Among other things, the present disclosure provides a polyribonucleotide encoding an immunoglobulin chain of an antibody agent, where the immunoglobulin chain comprises a heavy chain variable (VH) domain and a light chain variable (VL) domain. In some embodiments, VH domain comprises an HCDR1 comprising an amino acid sequence according to SEQ ID NO: 18, an HCDR2 comprising an amino acid sequence according to SEQ ID NO: 21, and an HCDR3 comprising an amino acid sequence according to SEQ ID NO: 24. In some embodiments, a VL domain comprises an LCDR1 comprising an amino acid sequence according to SEQ ID NO: 27, an LCDR2 comprising an amino acid sequence according to SEQ ID NO: 30, and an LCDR3 comprising an amino acid sequence according to SEQ ID NO: 33.

In some embodiments, a polyribonucleotide comprises a VH domain-encoding sequence and a VL domain-encoding sequence. In some embodiments, a VH domain-encoding sequence comprises an HCDR1-encoding sequence that comprises or consists of the ribonucleic acid sequence according to SEQ ID NO: 19, the HCDR2-encoding sequence that comprises or consists of the ribonucleic acid sequence according to SEQ ID NO: 22, and the HCDR3-encoding sequence that comprises or consists of the ribonucleic acid sequence according to SEQ ID NO: 25. In some embodiments, a VL domain-encoding sequence comprises an LCDR1-encoding sequence that comprises or consists of a ribonucleic acid sequence according to SEQ ID NO: 28, an LCDR2-encoding sequence that comprises or consists of a ribonucleic acid sequence according to SEQ ID NO: 31, and an LCDR3-encoding sequence that comprises or consists of a ribonucleic acid sequence according to SEQ ID NO: 34.

In some embodiments, an immunoglobulin chain comprises a single chain fragment variable (scFv). In some embodiments, an scFv comprises a VH domain, a linker, and a VL domain.

In some embodiments, an scFv comprises, in order, a VH domain, a linker, and a VL domain. In some embodiments, an scFv comprises, in order, a VH domain comprising or consisting of an amino acid sequence according to SEQ ID NO: 36, a linker, and a VL domain comprising or consisting of an amino acid sequence according to SEQ ID NO: 43.

In some embodiments, an scFv comprises, in order, a VL domain, a linker, and a VH domain. In some embodiments, an scFv comprises, in order, a VL domain comprising or consisting of an amino acid sequence according to SEQ ID NO: 43, a linker, and a VH domain comprising or consisting of an amino acid sequence according to SEQ ID NO: 36.

In some embodiments, a linker comprises an amino acid sequence according to SEQ ID NO: 48. In some embodiments, a polyribonucleotide comprises a ribonucleic acid sequence that encodes the linker and comprises or consists of a sequence according to SEQ ID NO: 49, 51, 53, 55, or 57.

In some embodiments, a linker comprises an amino acid sequence according to SEQ ID NO: 59. In some embodiments, a polyribonucleotide comprises a ribonucleic acid sequence that encodes the linker and comprises or consists of a sequence according to SEQ ID NO: 60 or 62.

In some embodiments, an immunoglobulin chain comprising a VH domain and a VL domain as described herein comprises one or more constant domains. In some embodiments, a VH domain and a VL domain are operably linked to one or more constant domains.

In some embodiments, an immunoglobulin chain comprises one or more constant domains, and a hinge domain is between the scFv and the one or more constant domains.

In some embodiments, one or more constant domains comprise a CH2 domain. In some embodiments, a polyribonucleotide comprises a ribonucleic acid sequence that encodes the CH2 domain.

In some embodiments, one or more constant domains comprise a CH3 domain. In some embodiments, a polyribonucleotide comprises a ribonucleic acid sequence that encodes the CH3 domain.

In some embodiments, one or more constant domains comprise a hinge domain. In some embodiments, a polyribonucleotide comprises a ribonucleic acid sequence that encodes the hinge domain.

In some embodiments, one or more constant domains comprise a CH1 domain. In some embodiments, a polyribonucleotide comprises a ribonucleic acid sequence that encodes a CH1 domain.

According to the present disclosure, a CH2 domain of any of the embodiments above can comprise an amino acid sequence having at least 85%, at least 90%, at least 95%, at least 96%, at least 97%, at least 98%, or at least 99% identity to an amino acid sequence according to SEQ ID NO: 53. In some embodiments, a CH2 domain comprises or consists of an amino acid sequence according to SEQ ID NO: 53.

In some embodiments, a ribonucleic acid sequence that encodes a CH2 domain comprises a ribonucleic acid sequence having at least 85%, at least 90%, at least 95%, at least 96%, at least 97%, at least 98%, or at least 99% identity to a ribonucleic acid sequence according to SEQ ID NO: 96. In some embodiments, a ribonucleic acid sequence that encodes the CH2 domain comprises or consists of a sequence according to SEQ ID NO: 96.

In some embodiments, a CH2 domain comprises one or more substitution mutations. In some embodiments, one or more substitution mutations in a CH2 domain comprise or consist of G236A, A330L, I332E, or a combination thereof, and wherein the substitution mutation positions are according to EU numbering. In some embodiments, one or more substitution mutations in a CH2 domain comprise or consist of G236A, and wherein the substitution mutation positions are according to EU numbering. In some embodiments, one or more substitution mutations in a CH2 domain comprise or consist of I332E, and wherein the substitution mutation positions are according to EU numbering. In some embodiments, one or more substitution mutations in a CH2 domain comprise or consist of G236A and I332E, and wherein the substitution mutation positions are according to EU numbering. In some embodiments, one or more substitution mutations in a CH2 domain comprise or consist of G236A, A330L, and I332E, and wherein the substitution mutation positions are according to EU numbering.

In some embodiments, a CH2 domain comprises an amino acid sequence having at least 85%, at least 90%, at least 95%, at least 96%, at least 97%, at least 98%, or at least 99% identity to an amino acid sequence according to SEQ ID NO: 56. In some embodiments, a CH2 domain comprises or consists of an amino acid sequence according to SEQ ID NO: 99. In some embodiments, a CH2 domain comprises an amino acid sequence having at least 85%, at least 90%, at least 95%, at least 96%, at least 97%, at least 98%, or at least 99% identity to an amino acid sequence according to SEQ ID NO: 102. In some embodiments, a CH2 domain comprises or consists of an amino acid sequence according to SEQ ID NO: 102.

In some embodiments, a polyribonucleotide comprises a ribonucleic acid sequence that encodes a CH2 domain with a ribonucleic acid sequence having at least 85%, at least 90%, at least 95%, at least 96%, at least 97%, at least 98%, or at least 99% identity to a ribonucleic acid sequence according to SEQ ID NO: 100. In some embodiments, a polyribonucleotide comprises a ribonucleic acid sequence that encodes a CH2 domain and comprises or consists of a sequence according to SEQ ID NO: 100. In some embodiments, a polyribonucleotide comprises a ribonucleic acid sequence that encodes a CH2 domain with a ribonucleic acid sequence having at least 85%, at least 90%, at least 95%, at least 96%, at least 97%, at least 98%, or at least 99% identity to a ribonucleic acid sequence according to SEQ ID NO: 103. In some embodiments, a polyribonucleotide comprises a ribonucleic acid sequence that encodes a CH2 domain and comprises or consists of a sequence according to SEQ ID NO: 103.

In some embodiments, a CH2 domain comprises an amino acid sequence having at least 85%, at least 90%, at least 95%, at least 96%, at least 97%, at least 98%, or at least 99% identity to an amino acid sequence according to SEQ ID NO: 104. In some embodiments, a CH2 domain comprises or consists of an amino acid sequence according to SEQ ID NO: 104.

In some embodiments, a polyribonucleotide comprises a ribonucleic acid sequence that encodes a CH2 domain and has at least 85%, at least 90%, at least 95%, at least 96%, at least 97%, at least 98%, or at least 99% identity to a ribonucleic acid sequence according to SEQ ID NO: 105. In some embodiments, a polyribonucleotide comprises a ribonucleic acid sequence that encodes a CH2 domain and comprises or consists of a sequence according to SEQ ID NO: 105.

In some embodiments, a CH2 domain comprises an amino acid sequence having at least 85%, at least 90%, at least 95%, at least 96%, at least 97%, at least 98%, or at least 99% identity to an amino acid sequence according to SEQ ID NO: 107. In some embodiments, a CH2 domain comprises or consists of an amino acid sequence according to SEQ ID NO: 107.

In some embodiments, a polyribonucleotide comprises a ribonucleic acid sequence that encodes a CH2 domain and has at least 85%, at least 90%, at least 95%, at least 96%, at least 97%, at least 98%, or at least 99% identity to a ribonucleic acid sequence according to SEQ ID NO: 108. In some embodiments, a polyribonucleotide comprises a ribonucleic acid sequence that encodes a CH2 domain and comprises or consists of a sequence according to SEQ ID NO: 108.

In some embodiments, a CH2 domain comprises an amino acid sequence having at least 85%, at least 90%, at least 95%, at least 96%, at least 97%, at least 98%, or at least 99% identity to an amino acid sequence according to SEQ ID NO: 110. In some embodiments, a CH2 domain comprises or consists of an amino acid sequence according to SEQ ID NO: 110.

In some embodiments, a polyribonucleotide comprises a ribonucleic acid sequence that encodes the CH2 domain and has at least 85%, at least 90%, at least 95%, at least 96%, at least 97%, at least 98%, or at least 99% identity to a ribonucleic acid sequence according to SEQ ID NO: 111. In some embodiments, a polyribonucleotide comprises a ribonucleic acid sequence that encodes the CH2 domain and comprises or consists of a sequence according to SEQ ID NO: 111.

According to the present disclosure, a CH3 domain of any of the embodiments above can comprise an amino acid sequence having at least 85%, at least 90%, at least 95%, at least 96%, at least 97%, at least 98%, or at least 99% identity to an amino acid sequence according to SEQ ID NO: 113. In some embodiments, a CH3 domain comprises or consists of an amino acid sequence according to SEQ ID NO: 113.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 114. In some embodiments, a polyribonucleotide comprises a ribonucleic acid sequence that encodes the CH3 domain and comprises or consists of a sequence according to SEQ ID NO: 114.

In some embodiments, a CH3 domain comprises an amino acid sequence having at least 85%, at least 90%, at least 95%, at least 96%, at least 97%, at least 98%, or at least 99% identity to an amino acid sequence according to SEQ ID NO: 131. In some embodiments, a CH3 domain comprises or consists of an amino acid sequence according to SEQ ID NO: 131.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 132. In some embodiments, a polyribonucleotide comprises a ribonucleic acid sequence that encodes the CH3 domain and comprises or consists of a sequence according to SEQ ID NO: 132.

In some embodiments, a CH3 domain comprises one or more substitution mutations. In some embodiments, one or more substitution mutations in a CH3 domain comprise or consist of M428L, N434S, or a combination thereof, and wherein the substitution mutation positions are according to EU numbering.

In some embodiments, a CH3 domain comprises or consists of an amino acid sequence according to SEQ ID NO: 116. In some embodiments, a polyribonucleotide comprises a ribonucleic acid sequence that encodes the CH3 domain and comprises or consists of a sequence according to SEQ ID NO: 117.

In some embodiments, a CH3 domain comprises an amino acid sequence having at least 85%, at least 90%, at least 95%, at least 96%, at least 97%, at least 98%, or at least 99% identity to an amino acid sequence according to SEQ ID NO: 134. In some embodiments, a CH3 domain comprises or consists of an amino acid sequence according to SEQ ID NO: 134.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 135. In some embodiments, a polyribonucleotide comprises a ribonucleic acid sequence that encodes the CH3 domain and comprises or consists of a sequence according to SEQ ID NO: 135.

In some embodiments, one or more substitution mutations in a CH3 domain comprise or consist of Y349C, T366S, L368A, Y407V, or a combination thereof, and wherein the substitution mutation positions are according to EU numbering. In some embodiments, one or more substitution mutations in a CH3 domain comprise or consist of Y349C, T366S, L368A, Y407V, M428L, N434S, or a combination thereof, and wherein the substitution mutation positions are according to EU numbering. In some embodiments, one or more substitution mutations in a CH3 domain comprise or consist of Y349C, T366S, L368A, Y407V, M428L, and N434S, and wherein the substitution mutation positions are according to EU numbering.

In some embodiments, a CH3 domain comprises an amino acid sequence having at least 85%, at least 90%, at least 95%, at least 96%, at least 97%, at least 98%, or at least 99% identity to an amino acid sequence according to SEQ ID NO: 122. In some embodiments, a CH3 domain comprises or consists of an amino acid sequence according to SEQ ID NO: 122.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 123. In some embodiments, a polyribonucleotide comprises a ribonucleic acid sequence that encodes the CH3 domain and comprises or consists of a sequence according to SEQ ID NO: 123.

In some embodiments, a CH3 domain comprises an amino acid sequence having at least 85%, at least 90%, at least 95%, at least 96%, at least 97%, at least 98%, or at least 99% identity to an amino acid sequence according to SEQ ID NO: 140. In some embodiments, a CH3 domain comprises or consists of an amino acid sequence according to SEQ ID NO: 140.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 141. In some embodiments, a polyribonucleotide comprises a ribonucleic acid sequence that encodes the CH3 domain and comprises or consists of a sequence according to SEQ ID NO: 141.

In some embodiments, one or more substitution mutations in a CH3 domain comprise or consist of S354C, T366W, or a combination thereof, and wherein the substitution mutation positions are according to EU numbering. In some embodiments, one or more substitution mutations in a CH3 domain comprise or consist of S354C, T366W, M428L, N434S, or a combination thereof, and wherein the substitution mutation positions are according to EU numbering. In some embodiments, one or more substitution mutations in a CH3 domain comprise or consist of S354C, T366W, M428L, and N434S, and wherein the substitution mutation positions are according to EU numbering.

In some embodiments, a CH3 domain comprises an amino acid sequence having at least 85%, at least 90%, at least 95%, at least 96%, at least 97%, at least 98%, or at least 99% identity to an amino acid sequence according to SEQ ID NO: 128. In some embodiments, a CH3 domain comprises or consists of an amino acid sequence according to SEQ ID NO: 128.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 129. In some embodiments, a polyribonucleotide comprises a ribonucleic acid sequence that encodes the CH3 domain and comprises or consists of a sequence according to SEQ ID NO: 129.

In some embodiments, a CH3 domain comprises an amino acid sequence having at least 85%, at least 90%, at least 95%, at least 96%, at least 97%, at least 98%, or at least 99% identity to an amino acid sequence according to SEQ ID NO: 146. In some embodiments, a CH3 domain comprises or consists of an amino acid sequence according to SEQ ID NO: 146.

In some embodiments, a polyribonucleotide comprises a ribonucleic acid sequence that encodes the CH3 domain and has at least 85%, at least 90%, at least 95%, at least 96%, at least 97%, at least 98%, or at least 99% identity to a ribonucleic acid sequence according to SEQ ID NO: 147. In some embodiments, a polyribonucleotide comprises a ribonucleic acid sequence that encodes the CH3 domain and comprises or consists of a sequence according to SEQ ID NO: 147.

According to the present disclosure, a hinge domain of any of the embodiments above can comprise an amino acid sequence having at least 85%, at least 90%, at least 95%, at least 96%, at least 97%, at least 98%, or at least 99% identity to an amino acid sequence according to SEQ ID NO: 161. In some embodiments, a hinge domain comprises or consists of an amino acid sequence according to SEQ ID NO: 161.

In some embodiments, a polyribonucleotide comprises a ribonucleic acid sequence that encodes the hinge domain and has at least 85%, at least 90%, at least 95%, at least 96%, at least 97%, at least 98%, or at least 99% identity to a ribonucleic acid sequence according to SEQ ID NO: 162. In some embodiments, a polyribonucleotide comprises a ribonucleic acid sequence that encodes the hinge domain and comprises or consists of a sequence according to SEQ ID NO: 162.

In some embodiments, a hinge domain comprises an amino acid sequence having at least 85%, at least 90%, at least 95%, at least 96%, at least 97%, at least 98%, or at least 99% identity to an amino acid sequence according to SEQ ID NO: 164. In some embodiments, a hinge domain comprises or consists of an amino acid sequence according to SEQ ID NO: 164.

In some embodiments, a polyribonucleotide comprises a ribonucleic acid sequence that encodes the hinge domain and has at least 85%, at least 90%, at least 95%, at least 96%, at least 97%, at least 98%, or at least 99% identity to a ribonucleic acid sequence according to SEQ ID NO: 165. In some embodiments, a polyribonucleotide comprises a ribonucleic acid sequence that encodes the hinge domain and comprises or consists of a sequence according to SEQ ID NO: 165.

In some embodiments, a hinge domain comprises an amino acid sequence having at least 85%, at least 90%, at least 95%, at least 96%, at least 97%, at least 98%, or at least 99% identity to an amino acid sequence according to SEQ ID NO: 167. In some embodiments, a hinge domain comprises or consists of an amino acid sequence according to SEQ ID NO: 167.

In some embodiments, a polyribonucleotide comprises a ribonucleic acid sequence that encodes the hinge domain and has at least 85%, at least 90%, at least 95%, at least 96%, at least 97%, at least 98%, or at least 99% identity to a ribonucleic acid sequence according to SEQ ID NO: 168. In some embodiments, a polyribonucleotide comprises a ribonucleic acid sequence that encodes the hinge domain and comprises or consists of a sequence according to SEQ ID NO: 168.

According to the present disclosure, a CH1 domain of any of the embodiments above can comprise an amino acid sequence having at least 85%, at least 90%, at least 95%, at least 96%, at least 97%, at least 98%, or at least 99% identity to an amino acid sequence according to SEQ ID NO: 76. In some embodiments, a CH1 domain comprises or consists of an amino acid sequence according to SEQ ID NO: 76.

In some embodiments, a polyribonucleotide comprises a ribonucleic acid sequence that encodes a CH1 domain and has at least 85%, at least 90%, at least 95%, at least 96%, at least 97%, at least 98%, or at least 99% identity to a ribonucleic acid sequence according to SEQ ID NO: 77. In some embodiments, a polyribonucleotide comprises a ribonucleic acid sequence that encodes a CH1 domain and comprises or consists of a sequence according to SEQ ID NO: 77.

In some embodiments, a CH1 domain comprises an amino acid sequence having at least 85%, at least 90%, at least 95%, at least 96%, at least 97%, at least 98%, or at least 99% identity to an amino acid sequence according to SEQ ID NO: 81. In some embodiments, a CH1 domain comprises or consists of an amino acid sequence according to SEQ ID NO: 81.

In some embodiments, a polyribonucleotide comprises a ribonucleic acid sequence that encodes a CH1 domain and has at least 85%, at least 90%, at least 95%, at least 96%, at least 97%, at least 98%, or at least 99% identity to a ribonucleic acid sequence according to SEQ ID NO: 82. In some embodiments, a polyribonucleotide comprises a ribonucleic acid sequence that encodes a CH1 domain and comprises or consists of a sequence according to SEQ ID NO: 82.

In some embodiments, a CH1 domain comprises one or more substitution mutations. In some embodiments, one or more substitution mutations in a CH1 domain comprise or consist of K147E, K213D, or a combination thereof, and wherein the substitution mutation positions are according to EU numbering.

In some embodiments, a CH1 domain comprises an amino acid sequence having at least 85%, at least 90%, at least 95%, at least 96%, at least 97%, at least 98%, or at least 99% identity to an amino acid sequence according to SEQ ID NO: 84. In some embodiments, a CH1 domain comprises or consists of an amino acid sequence according to SEQ ID NO: 84.

In some embodiments, a polyribonucleotide comprises a ribonucleic acid sequence that encodes the CH1 domain and has at least 85%, at least 90%, at least 95%, at least 96%, at least 97%, at least 98%, or at least 99% identity to a ribonucleic acid sequence according to SEQ ID NO: 85. In some embodiments, a polyribonucleotide comprises a ribonucleic acid sequence that encodes the CH1 domain and comprises or consists of a sequence according to SEQ ID NO: 85.

In some embodiments, a CH1 domain comprises an amino acid sequence having at least 85%, at least 90%, at least 95%, at least 96%, at least 97%, at least 98%, or at least 99% identity to an amino acid sequence according to SEQ ID NO: 76. In some embodiments, a CH1 domain comprises or consists of an amino acid sequence according to SEQ ID NO: 76.

In some embodiments, a polyribonucleotide comprises a ribonucleic acid sequence that encodes the CH1 domain and has at least 85%, at least 90%, at least 95%, at least 96%, at least 97%, at least 98%, or at least 99% identity to a ribonucleic acid sequence according to SEQ ID NO: 77. In some embodiments, a polyribonucleotide comprises a ribonucleic acid sequence that encodes the CH1 domain and comprises or consists of a sequence according to SEQ ID NO: 77.

In some embodiments, a CH1 domain comprises an amino acid sequence having at least 85%, at least 90%, at least 95%, at least 96%, at least 97%, at least 98%, or at least 99% identity to an amino acid sequence according to SEQ ID NO: 81. In some embodiments, a CH1 domain comprises or consists of an amino acid sequence according to SEQ ID NO: 81.

In some embodiments, a polyribonucleotide comprises a ribonucleic acid sequence that encodes the CH1 domain and has at least 85%, at least 90%, at least 95%, at least 96%, at least 97%, at least 98%, or at least 99% identity to a ribonucleic acid sequence according to SEQ ID NO: 82. In some embodiments, a polyribonucleotide comprises a ribonucleic acid sequence that encodes the CH1 domain and comprises or consists of a sequence according to SEQ ID NO: 82.

According to the present disclosure, a CL domain of any of the embodiments above can comprise an amino acid sequence having at least 85%, at least 90%, at least 95%, at least 96%, at least 97%, at least 98%, or at least 99% identity to an amino acid sequence according to SEQ ID NO: 149. In some embodiments, a CL domain of any of the embodiments above can comprise or consists of an amino acid sequence according to SEQ ID NO: 149.

In some embodiments, a polyribonucleotide comprises a ribonucleic acid sequence that encodes the CL domain and has at least 85%, at least 90%, at least 95%, at least 96%, at least 97%, at least 98%, or at least 99% identity to a ribonucleic acid sequence according to SEQ ID NO: 150. In some embodiments, a polyribonucleotide comprises a ribonucleic acid sequence that encodes the CL domain and comprises or consists of a sequence according to SEQ ID NO: 150.

In some embodiments, a polyribonucleotide comprises a ribonucleic acid sequence that encodes the light chain constant domain CL domain comprises one or more substitution mutations. In some embodiments, one or more substitution mutations in a CL domain comprise or consist of Q124E, and wherein the substitution mutation positions are according to EU numbering. In some embodiments, one or more substitution mutations in a CL domain comprise or consist of R108A, T109S, or a combination thereof, and wherein the substitution mutation positions are according to EU numbering. In some embodiments, one or more substitution mutations in a CL domain comprise or consist of R108A, T109S, Q124E or a combination thereof, and wherein the substitution mutation positions are according to EU numbering.

In some embodiments, a CL domain comprises an amino acid sequence having at least 85%, at least 90%, at least 95%, at least 96%, at least 97%, at least 98%, or at least 99% identity to an amino acid sequence according to SEQ ID NO: 152. In some embodiments, a CL domain comprises or consists of an amino acid sequence according to SEQ ID NO: 152.

In some embodiments, a polyribonucleotide comprises a ribonucleic acid sequence that encodes the light chain constant domain and has at least 85%, at least 90%, at least 95%, at least 96%, at least 97%, at least 98%, or at least 99% identity to a ribonucleic acid sequence according to SEQ ID NO: 153. In some embodiments, a polyribonucleotide comprises a ribonucleic acid sequence that encodes the light chain constant domain and comprises or consists of a sequence according to SEQ ID NO: 153.

In some embodiments, a CL domain comprises one or more substitution mutations. In some embodiments, one or more substitution mutations in a CL domain comprise or consist of E123K, Q124R, or a combination thereof, and wherein the substitution mutation positions are according to EU numbering.

In some embodiments, a CL domain comprises an amino acid sequence having at least 85%, at least 90%, at least 95%, at least 96%, at least 97%, at least 98%, or at least 99% identity to an amino acid sequence according to SEQ ID NO: 155. In some embodiments, a CL domain comprises or consists of an amino acid sequence according to SEQ ID NO: 155.

In some embodiments, a polyribonucleotide comprises a ribonucleic acid sequence that encodes the light chain constant domain and has at least 85%, at least 90%, at least 95%, at least 96%, at least 97%, at least 98%, or at least 99% identity to a ribonucleic acid sequence according to SEQ ID NO: 156. In some embodiments, a polyribonucleotide comprises a ribonucleic acid sequence that encodes the light chain constant domain and comprises or consists of a sequence according to SEQ ID NO: 156.

In some embodiments, a one or more substitution mutations in a CL domain comprise or consist of E123R, Q124K, or a combination thereof, and wherein the substitution mutation positions are according to EU numbering.

In some embodiments, a CL domain comprises an amino acid sequence having at least 85%, at least 90%, at least 95%, at least 96%, at least 97%, at least 98%, or at least 99% identity to an amino acid sequence according to SEQ ID NO: 158. In some embodiments, a CL domain comprises or consists of an amino acid sequence according to SEQ ID NO: 158.

In some embodiments, a polyribonucleotide comprises a ribonucleic acid sequence that encodes the light chain constant domain and has at least 85%, at least 90%, at least 95%, at least 96%, at least 97%, at least 98%, or at least 99% identity to a ribonucleic acid sequence according to SEQ ID NO: 159. In some embodiments, a polyribonucleotide comprises a ribonucleic acid sequence that encodes the light chain constant domain and comprises or consists of a sequence according to SEQ ID NO: 159.

In some embodiments, a polyribonucleotide as provided herein encodes an immunoglobulin chain, wherein:
(i) the polyribonucleotide comprises a ribonucleic acid sequence that encodes a CH2 domain, and the CH2 domain comprises one or more substitution mutations, wherein the one or more substitution mutations comprise or consist of G236A, A330L, I332E, or a combination thereof,
(ii) the polyribonucleotide comprises a ribonucleic acid sequence that encodes a CH3 domain, and the CH3 domain comprises one or more substitution mutations, wherein the one or more substitution mutations comprise or consist of Y349C, S354C, T366S, T366W, L368A, Y407V, M428L, N434S, or a combination thereof,
(iii) the polyribonucleotide comprises a ribonucleic acid sequence that encodes a CH1 domain, and the CH1 domain comprises one or more substitution mutations, wherein the one or more substitution mutations comprise or consist of K147E, K213D, or a combination thereof,
(iv) the polyribonucleotide comprises a ribonucleic acid sequence that encodes a CL domain, and the CL domain comprises one or more substitution mutations, wherein the one or more substitution mutations comprise or consist of R108A, T109S, E123K, E123R, Q124E, Q124K, Q124R, or a combination thereof, or
(v) a combination thereof;

wherein the substitution mutation positions are according to EU numbering.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1307. In some embodiments, a polyribonucleotide as provided herein encodes an immunoglobulin chain that comprises or consists of an amino acid sequence according to SEQ ID NO: 1307.

In some embodiments, a polyribonucleotide as provided herein comprises a ribonucleic acid sequence that is at least 85%, at least 90%, at least 95%, at least 96%, at least 97%, at least 98%, or at least 99% identity to a ribonucleic acid sequence according to SEQ ID NO: 1306. In some embodiments, a polyribonucleotide comprises or consists of a ribonucleic acid sequence according to SEQ ID NO: 1306.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1310. In some embodiments, a polyribonucleotide as provided herein encodes an immunoglobulin chain that comprises or consists of an amino acid sequence according to SEQ ID NO: 1310.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1309. In some embodiments, a polyribonucleotide as provided herein comprises or consists of a ribonucleic acid sequence according to SEQ ID NO: 1309.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1316. In some embodiments, a polyribonucleotide as provided herein encodes an immunoglobulin chain that comprises or consists of an amino acid sequence according to SEQ ID NO: 1316.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1315. In some embodiments, a polyribonucleotide as provided herein comprises or consists of a ribonucleic acid sequence according to SEQ ID NO: 1315.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1325. In some embodiments, a polyribonucleotide as provided herein encodes an immunoglobulin chain that comprises or consists of an amino acid sequence according to SEQ ID NO: 1325.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1324. In some embodiments, a polyribonucleotide as provided herein comprises or consists of a ribonucleic acid sequence according to SEQ ID NO: 1324.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1319. In some embodiments, a polyribonucleotide as provided herein encodes an immunoglobulin chain that comprises or consists of an amino acid sequence according to SEQ ID NO: 1319.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1318. In some embodiments, a polyribonucleotide as provided herein comprises or consists of a ribonucleic acid sequence according to SEQ ID NO: 1318.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1331. In some embodiments, a polyribonucleotide as provided herein encodes an immunoglobulin chain that comprises or consists of an amino acid sequence according to SEQ ID NO: 1331.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1330. In some embodiments, a polyribonucleotide as provided herein comprises or consists of a ribonucleic acid sequence according to SEQ ID NO: 1330.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1334. In some embodiments, a polyribonucleotide as provided herein encodes an immunoglobulin chain that comprises or consists of an amino acid sequence according to SEQ ID NO: 1334.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1333. In some embodiments, a polyribonucleotide as provided herein comprises or consists of a ribonucleic acid sequence according to SEQ ID NO: 1333.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1337. In some embodiments, a polyribonucleotide as provided herein encodes an immunoglobulin chain that comprises or consists of an amino acid sequence according to SEQ ID NO: 1337.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1336. In some embodiments, a polyribonucleotide as provided herein comprises or consists of a ribonucleic acid sequence according to SEQ ID NO: 1336.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1340. In some embodiments, a polyribonucleotide as provided herein encodes an immunoglobulin chain that comprises or consists of an amino acid sequence according to SEQ ID NO: 1340.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1339. In some embodiments, a polyribonucleotide as provided herein comprises or consists of a ribonucleic acid sequence according to SEQ ID NO: 1339.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1313. In some embodiments, a polyribonucleotide as provided herein encodes an immunoglobulin chain that comprises or consists of an amino acid sequence according to SEQ ID NO: 1313.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1312. In some embodiments, a polyribonucleotide as provided herein comprises or consists of a ribonucleic acid sequence according to SEQ ID NO: 1312.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1328. In some embodiments, a polyribonucleotide as provided herein encodes an immunoglobulin chain that comprises or consists of an amino acid sequence according to SEQ ID NO: 1328.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1327. In some embodiments, a polyribonucleotide as provided herein comprises or consists of a ribonucleic acid sequence according to SEQ ID NO: 1327.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1355. In some embodiments, a polyribonucleotide as provided herein encodes an immunoglobulin chain that comprises or consists of an amino acid sequence according to SEQ ID NO: 1355.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1354. In some embodiments, a polyribonucleotide as provided herein polyribonucleotide comprises or consists of a ribonucleic acid sequence according to SEQ ID NO: 1354.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1322. In some embodiments, a polyribonucleotide as provided herein encodes an immunoglobulin chain that comprises or consists of an amino acid sequence according to SEQ ID NO: 1322.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1321. In some embodiments, a polyribonucleotide as provided herein comprises or consists of a ribonucleic acid sequence according to SEQ ID NO: 1321.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1343. In some embodiments, a polyribonucleotide as provided herein encodes an immunoglobulin chain that comprises or consists of a sequence according to SEQ ID NO: 1343.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1342. In some embodiments, a polyribonucleotide as provided herein comprises or consists of a ribonucleic acid sequence according to SEQ ID NO: 1342.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1346. In some embodiments, a polyribonucleotide as provided herein encodes an immunoglobulin chain that comprises or consists of a sequence according to SEQ ID NO: 1346.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1345. In some embodiments, a polyribonucleotide as provided herein comprises or consists of a ribonucleic acid sequence according to SEQ ID NO: 1345.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1349. In some embodiments, a polyribonucleotide as provided herein encodes an immunoglobulin chain that comprises or consists of a sequence according to SEQ ID NO: 1349.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1348. In some embodiments, a polyribonucleotide as provided herein comprises or consists of a ribonucleic acid sequence according to SEQ ID NO: 1348.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1352. In some embodiments, a polyribonucleotide as provided herein encodes an immunoglobulin chain that comprises or consists of a sequence according to SEQ ID NO: 1362.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1351. In some embodiments, a polyribonucleotide as provided herein comprises or consists of a ribonucleic acid sequence according to SEQ ID NO: 1351.

In some embodiments, a polyribonucleotide as provided herein encodes an immunoglobulin chain that comprises an amino acid sequence having at least 85%, at least 90%, at least 95%, at least 96%, at least 97%, at least 98%, or at least 99% identity to an amino acid sequence according to SEQ ID NO: 1358, 1361, 1364, 1367, 1370, 1373, 1376, 1379, 1382, 1385, 1388, 1391, 1394, 1397, 1400, 1403, 1406, 1409, 1412, 1415, 1418, 1421, 1424, 1427, 1430, 1433, 1436, 1439, 1442, 1445, 1448, 1451, 1454, 1457, or 1460. In some embodiments, a polyribonucleotide as provided herein encodes an immunoglobulin chain that comprises or consists of a sequence according to SEQ ID NO: 1358, 1361, 1364, 1367, 1370, 1373, 1376, 1379, 1382, 1385, 1388, 1391, 1394, 1397, 1400, 1403, 1406, 1409, 1412, 1415, 1418, 1421, 1424, 1427, 1430, 1433, 1436, 1439, 1442, 1445, 1448, 1451, 1454, 1457, or 1460.

In some embodiments, a polyribonucleotide as provided herein comprises a ribonucleic acid sequence that is at least 85%, at least 90%, at least 95%, at least 96%, at least 97%, at least 98%, or at least 99% identical to a ribonucleic acid sequence according to SEQ ID NO: 1357, 1360, 1363, 1366, 1369, 1372, 1375, 1378, 1381, 1384, 1387, 1390, 1393, 1396, 1399, 1402, 1405, 1408, 1411, 1414, 1417, 1420, 1423, 1426, 1429, 1432, 1435, 1438, 1441, 1444, 1447, 1450, 1453, 1456, or 1459. In some embodiments, a polyribonucleotide as provided herein comprises or consists of a ribonucleic acid sequence according to SEQ ID NO: 1357, 1360, 1363, 1366, 1369, 1372, 1375, 1378, 1381, 1384, 1387, 1390, 1393, 1396, 1399, 1402, 1405, 1408, 1411, 1414, 1417, 1420, 1423, 1426, 1429, 1432, 1435, 1438, 1441, 1444, 1447, 1450, 1453, 1456, or 1459.

In some embodiments, a polyribonucleotide comprises a ribonucleic acid sequence encoding a secretion signal.

In some embodiments, a secretion signal comprises a ribonucleic acid sequence according to SEQ ID NO: 2, 4, 6, or 8.

In some embodiments, a polyribonucleotide comprises one or more non-coding sequence elements.

In some embodiments, one or more non-coding sequence elements enhances RNA stability and/or translation efficiency.

In some embodiments, one or more non-coding sequence elements comprise a 3′ untranslated region (UTR), a 5′ UTR, a 5′-cap, a polyadenine (polyA) tail, or combination thereof.

In some embodiments, a polyA tail is or comprises a modified polyA sequence, preferably an interrupted polyA tail.

In some embodiments, a polyA tail comprises or consists of a sequence that is at least 90%, at least 95%, or at least 99% identical to SEQ ID NO: 416.

In some embodiments, a 3′ UTR comprises or consists of a nucleic acid sequence that is at least 90%, at least 95%, or at least 99% identical to SEQ ID NO: 14.

In some embodiments, a 5′ UTR comprises or consists of a nucleic acid sequence that is at least 90%, at least 95%, or at least 99% identical to SEQ ID NO: 10.

In some embodiments, a 5′-cap is (m27,3′-O)Gppp(m2′-O)ApG.

In some embodiments, a polyribonucleotide comprises one or more modified ribonucleotides. In some embodiments, one or more modified ribonucleotides comprise pseudouridine.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 850. In some embodiments, a polyribonucleotide comprises or consists of a ribonucleic acid sequence according to SEQ ID NO: 850.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 851. In some embodiments, a polyribonucleotide comprises or consists of a ribonucleic acid sequence according to SEQ ID NO: 851.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 853. In some embodiments, a polyribonucleotide comprises or consists of a ribonucleic acid sequence according to SEQ ID NO: 853.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 900. In some embodiments, a polyribonucleotide comprises or consists of a ribonucleic acid sequence according to SEQ ID NO: 900.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 901. In some embodiments, a polyribonucleotide comprises or consists of a ribonucleic acid sequence according to SEQ ID NO: 901.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 905. In some embodiments, a polyribonucleotide comprises or consists of a ribonucleic acid sequence according to SEQ ID NO: 905.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 906. In some embodiments, a polyribonucleotide comprises or consists of a ribonucleic acid sequence according to SEQ ID NO: 906.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 910. In some embodiments, a polyribonucleotide comprises or consists of a ribonucleic acid sequence according to SEQ ID NO: 910.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 950. In some embodiments, a polyribonucleotide comprises or consists of a ribonucleic acid sequence according to SEQ ID NO: 950.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 898. In some embodiments, a polyribonucleotide comprises or consists of a ribonucleic acid sequence according to SEQ ID NO: 898.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 947. In some embodiments, a polyribonucleotide comprises or consists of a ribonucleic acid sequence according to SEQ ID NO: 947.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 997. In some embodiments, a polyribonucleotide comprises or consists of a ribonucleic acid sequence according to SEQ ID NO: 997.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 1000. In some embodiments, a polyribonucleotide comprises or consists of a ribonucleic acid sequence according to SEQ ID NO: 1000.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 1048. In some embodiments, a polyribonucleotide comprises or consists of a ribonucleic acid sequence according to SEQ ID NO: 1048.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 1096. In some embodiments, a polyribonucleotide comprises or consists of a ribonucleic acid sequence according to SEQ ID NO: 1096.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to SEQ ID NO: 1144. In some embodiments, a polyribonucleotide comprises or consists of a ribonucleic acid sequence according to SEQ ID NO: 1144.

In some embodiments, a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NOs: 850-1190. In some embodiments, a polyribonucleotide comprises or consists of a ribonucleic acid sequence according to any one of SEQ ID NO: 850-1190.

In some embodiments, a polyribonucleotide is a non-natural polyribonucleotide.

In some embodiments, a polyribonucleotide is an engineered polyribonucleotide.

In some embodiments, a polyribonucleotide is an isolated polyribonucleotide.

Among other things, the present disclosure provides composition comprising one or more polyribonucleotides as described herein.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1307 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1313.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1306 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1312.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1310 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1313.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1309 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1312.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1316 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1313.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1315 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1312.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1319 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1322.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1318 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1321.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1331 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1322.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1330 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1321.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1334 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1322.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1333 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1321.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1337 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1322.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1336 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1321.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1340 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1322.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1339 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1321.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1325 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1328.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1324 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1327.

In some embodiments, a composition comprises or consists of a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1325 and a polyribonucleotide encoding an immunoglobulin chain that comprises an amino acid sequence having at least 85%, at least 90%, at least 95%, at least 96%, at least 97%, at least 98%, or at least 99% identity to an amino acid sequence according to SEQ ID NO: 1355.

In some embodiments, a composition comprises or consists of a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1324 and a polyribonucleotide comprises a ribonucleic acid sequence that is at least 85%, at least 90%, at least 95%, at least 96%, at least 97%, at least 98%, or at least 99% identical to a ribonucleic acid sequence according to any one of SEQ ID NO: 1354.

In some embodiments, a composition further comprises lipid nanoparticles, polyplexes (PLX), lipidated polyplexes (LPLX), or liposomes, where the one or more polyribonucleotides are fully or partially encapsulated within the lipid nanoparticles, polyplexes (PLX), lipidated polyplexes (LPLX), or liposomes.

In some embodiments, a composition further comprises lipid nanoparticles, where the one or more polyribonucleotides are encapsulated within the lipid nanoparticles.

In some embodiments, lipid nanoparticles target liver cells.

In some embodiments, lipid nanoparticles target secondary lymphoid organ cells.

In some embodiments, lipid nanoparticles target lung cells.

In some embodiments, lipid nanoparticles are cationic lipid nanoparticles.

In some embodiments, lipid nanoparticles each comprise a polymer-conjugated lipid, a cationic lipid, and one or more neutral lipids.

In some embodiments, a polymer-conjugated lipid comprises a PEG-conjugated lipid.

In some embodiments, a polymer-conjugated lipid comprises 2-[(polyethylene glycol)-2000]-N,N-ditetradecylacetamide.

In some embodiments, one or more neutral lipids comprise 1,2-Distearoyl-sn-glycero-3-phosphocholine (DPSC).

In some embodiments, one or more neutral lipids comprise cholesterol.

In some embodiments, a cationic lipid comprises ((3-hydroxypropyl)azanediyl)bis(nonane-9,1-diyl) bis(2-butyloctanoate).

In some embodiments, lipid nanoparticles each comprise 2-[(polyethylene glycol)-2000]-N,N-ditetradecylacetamide, DPSC, cholesterol, and ((3-hydroxypropyl)azanediyl)bis(nonane-9,1-diyl) bis(2-butyloctanoate).

In some embodiments, lipid nanoparticles comprise the polymer-conjugated lipid at about 1-2.5 mol % of the total lipids, the cationic lipid at 35-65 mol % of the total lipids, and the one or more neutral lipids are present in 35-65 mol % of the total lipids.

In some embodiments, lipid nanoparticles have an average diameter of about 50-150 nm.

The present disclosure also provides pharmaceutical compositions. In some embodiments, a pharmaceutical composition comprises a composition provided herein and at least one pharmaceutically acceptable excipient.

In some embodiments, a pharmaceutical comprises a cryoprotectant. In some embodiments, a pharmaceutical comprises an aqueous buffered solution.

Among other things, the present disclosure provides methods.

In some embodiments, a method comprises administering a pharmaceutical composition provided herein to a subject.

In some embodiments, a pharmaceutical composition provided herein is for use in the treatment of HIV comprising administering the pharmaceutical composition to a subject.

In some embodiments, a pharmaceutical composition provided herein is for use in the prevention of HIV comprising administering the pharmaceutical composition to a subject.

In some embodiments, a method or pharmaceutical composition for use provided herein, comprises administering the pharmaceutical composition to the subject, which results in expression in the subject of an immunoglobulin chain of antibody agent, an antibody agent, or both.

In some embodiments, an immunoglobulin chain of antibody agent, the antibody agent, or both is expressed in the subject at a titer of at least 1 μg/ml in plasma or serum.

In some embodiments, an antibody agent exhibits a geometric mean IC50 of five neutralized strains of less than 0.3 μg/ml against the neutralized strains of a global reference panel when tested in the TZM-bl cell pseudovirus neutralization assay at antibody agent concentrations up to 25 μg/ml.

In some embodiments, an antibody agent is capable of neutralizing one or more HIV strains when tested in the TZM-bl cell pseudovirus neutralization assay at antibody agent concentrations up to 25 μg/ml.

In some embodiments, an antibody agent is capable of neutralizing one or more HIV strains at a level that is within 3-fold of a level of an equivalent amount of recombinant benchmark antibody.

In some embodiments, a recombinant benchmark antibody is an unmodified wild-type IgG antibody comprising the same HCDR1, HCDR2, HCDR2, LCDR1, LCDR2, and LCDR3 as the antibody agent.

In some embodiments, administering the pharmaceutical composition to the subject comprises administering one or more doses of the pharmaceutical composition to the subject. In some embodiments, one or more doses of the pharmaceutical composition are administered to the subject weekly. In some embodiments, one or more doses of the pharmaceutical composition are administered to the subject bi-weekly.

In some embodiments, a pharmaceutical composition is administered intravenously. In some embodiments, a pharmaceutical composition is administered intramuscularly. In some embodiments, a pharmaceutical composition is administered subcutaneously.

In some embodiments, a subject has or is at risk of developing an HIV infection.

In some embodiments, a method is a method of treating an HIV infection.

In some embodiments, a method is a method of preventing an HIV infection.

Also provided herein are uses of polyribonucleotides, compositions, and pharmaceutical compositions described herein.

In some embodiments, provided are uses of a composition or pharmaceutical composition as provided herein for the treatment of HIV in a subject.

In some embodiments, provided are uses of a composition or pharmaceutical composition as provided herein for the prevention of HIV in a subject.

In some embodiments, a subject has or is at risk of developing an HIV infection.

Further, the present disclosure provides methods of producing an antibody agent. In some embodiments, a method comprises administering to cells a composition or pharmaceutical composition as provided herein so that the cells express and secrete the antibody agent.

In some embodiments, cells are liver cells.

In some embodiments, cells are in a subject.

In some embodiments, cells are ex vivo cells.

In some embodiments, an antibody agent is produced at a therapeutically relevant plasma concentration or a therapeutically relevant serum concentration. In some embodiments, therapeutically relevant plasma concentration or the therapeutically relevant serum concentration is at least 1 μg/ml.

The present disclosure further provides methods of determining one or more features of an antibody agent expressed from a polyribonucleotide, a composition or a pharmaceutical composition provided herein. In some embodiments, a polyribonucleotide, a composition or a pharmaceutical composition provided herein are introduced into cells. In some embodiments, one or more features comprises: (i) protein expression level of the antibody agent; (ii) binding specificity of the antibody agent to the Apex binding site of HIV; (iii) efficacy of the antibody agent to mediate target cell death through antibody-dependent cellular cytotoxicity (ADCC); and (iv) efficacy of the antibody agent to mediate target cell death through complement dependent cytotoxicity (CDC).

The present disclosure further provides methods comprising contacting cells with a polyribonucleotide, a composition or a pharmaceutical composition provided herein. In some embodiments, a method further comprises detecting the antibody agent produced by the cells.

In some embodiments, cells are liver cells.

In some embodiments, the step of determining comprises comparing the one or more features of the antibody agent with that of a reference antibody that specifically binds to a Apex binding site of HIV.

In some embodiments, the step of determining comprises assessing the protein expression level of the antibody agent above a threshold level.

In some embodiments, a threshold level is a level that is sufficient to induce ADCC.

In some embodiments, the step of determining comprises assessing binding of the antibody agent to a Apex (e.g., the V1V2 region) binding site of HIV.

In some embodiments, the step of determining comprises assessing the antibody agent in the TZM-bl cell pseudovirus neutralization assay at antibody agent concentrations up to 25 μg/ml.

In some embodiments, cells are present in a subject.

In some embodiments, cells are ex vivo cells.

In some embodiments, one or more features include antibody level in one or more tissues in the subject.

The present disclosure also provides methods of manufacture. In some embodiments, a method comprises (A) determining one or more features of a polyribonucleotide, a composition or a pharmaceutical composition provided herein, which one or more features comprise or consist of:
(i) length and/or sequence of the polyribonucleotide;
(ii) integrity of the polyribonucleotide;
(iii) presence and/or location of one or more chemical moieties of the polyribonucleotide;
(iv) extent of expression of the antibody agent when the polyribonucleotide is introduced into a cell;
(v) stability of the polyribonucleotide or composition thereof;
(vi) level of antibody agent in a biological sample from an organism into which the polyribonucleotide has been introduced;
(vii) binding specificity of the antibody agent expressed from the polyribonucleotide, optionally to a CD4 binding site of HIV;
(viii) efficacy of the antibody agent to mediate target cell death through ADCC;
(ix) efficacy of the antibody agent to mediate target cell death through complement dependent cytotoxicity (CDC);
(x) lipid identity and amount/concentration within the composition;
(xi) size of lipid nanoparticles within the composition;
(xii) polydispersity of lipid nanoparticles within the composition;
(xiii) amount/concentration of the polyribonucleotide within the composition;
(xiv) extent of encapsulation of the polyribonucleotide within lipid nanoparticles;
(xv) a level of double stranded RNA; and
(xvi) combinations thereof;
   (B) comparing the one or more features of the polyribonucleotide with that of an appropriate reference standard; and
   (C) (i) designating the polyribonucleotide or composition thereof for one or more further steps of manufacturing and/or distribution if the comparison demonstrates that the polyribonucleotide or composition thereof meets or exceeds the reference standard; or
   (C) (ii) taking an alternative action if the comparison demonstrates that the polyribonucleotide or composition thereof does not meet or exceed the reference standard.

In some embodiments, a polyribonucleotide is assessed and the one or more further steps of step (C)(i) are or comprise at least formulation of the polyribonucleotide.

In some embodiments, a composition or the pharmaceutical composition is assessed, and the one or more further steps of step (C)(i) are or comprise release and distribution of the composition or the pharmaceutical composition.

The present disclosure thus provides technologies that allow for multiple anti-HIV antibodies to be expressed in a subject, increasing the breadth and potency of the anti-HIV antibodies present in a subject at one time and decreasing the likelihood of viral escape.

Provide technologies, including exemplary polyribonucleotides, compositions comprising such polyribonucleotides, and methods of making and using such polyribonucleotides, are described in more detail herein.

### BRIEF DESCRIPTION OF THE DRAWING

FIG. 1 shows a schematic of the HIV genome (Panel A) and the structure of a HIV virion particle (Panel B). Drawings modified from Musumeci et al., 2015 Molecules, which is incorporated herein by reference in its entirety.

FIG. 2 shows potential epitope regions on an HIV virion particle to which an antibody agent (e.g., a broadly neutralizing antibody (bNAb) or variant thereof) can bind. Drawings modified from McCoy and Burton, 2017 Immunol Rev., which is incorporated herein by reference in its entirety.

FIG. 3 shows an exemplary therapeutic strategy utilizing RiboMab technology as described herein for delivery and expression of anti-HIV RibobNabs.

FIG. 4 shows exemplary formats of PGDM1400 RibobNabs as described herein. Exemplary formats may include IgG (Panel A), CrossMab^{CH1-CLx}, (Panel B), CrossMab^{CH1-CLv}, (Panel D), or various orientations/linkers of scFv-Fc RibobNAbs (Panel C and Panel E).

FIG. 5 shows exemplary Fc modifications of PGDM1400 RibobNabs as described herein. Exemplary RiboMab formats may include an unmodified Fc domain (Panel A), or modifications shown in Panels B-D, including GAALIE/GAIE/GA/IE (Panel B), L/S, (Panel D), and/or knob-into-holes RibobNAbs (Panel C).

FIG. 6 shows schematics of exemplary polyribonucleotides which encode a heavy chain (Panel A) and a light chain (Panel B) of an exemplary PGDM1400 IgG1 antibody agent.

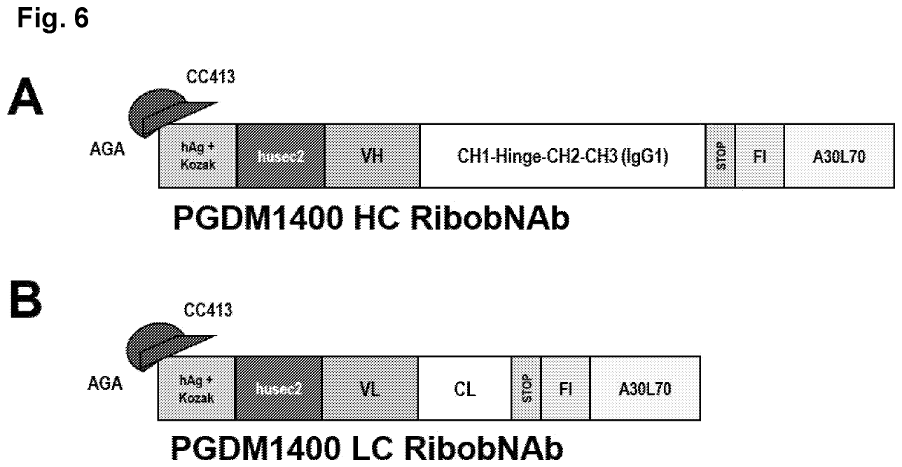

FIG. 7 shows % breadth and potency of various broadly neutralizing antibodies to HIV, including PGDM1400, as tested against a panel of 109 pseudoviruses.

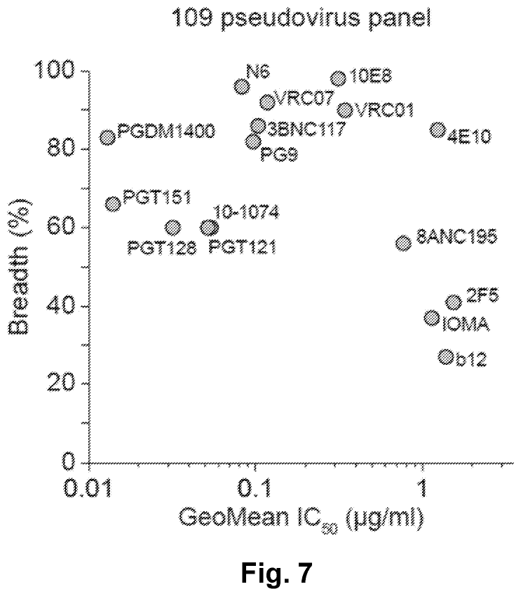

FIG. 8 shows schematics of exemplary polyribonucleotides which encode scFv-Fc PGDM1400 antibody agents: PGDM1400 scFV-Fc VH-LL4/5-VL (Panel A) and PGDM1400 scFV-Fc VL-LL4/5-VH (Panel B).

FIG. 9 shows schematics of exemplary polyribonucleotides which encode heavy chain (Panel A) and light chain (Panel B) of an exemplary PGDM1400 CrossMab^{CH1-CLx} antibody agent.

FIG. 10 shows schematics of exemplary polyribonucleotides which encode heavy chain (Panel A) and light chain (Panel B) of an exemplary PGDM1400 CrossMab^{CH1-CLcv} antibody agent.

FIG. 11 shows exemplary concentrations of PGDM1400 and PGDM1400 L/S RibobNAbs compared to a control RiboMab as determined by Gyros ELISA from Example 5.

FIG. 12 shows exemplary Western Blot analysis of PGDM1400 and PGDM1400 L/S RibobNAbs compared to a control RiboMab under nonreducing conditions from Example 5.

FIG. 13 shows exemplary concentrations of scFv-Fc PGDM1400 L/S RibobNAbs (e.g., VH-LL4-VL, VL-LL5-VH, VH-LL5-VL, and VL-LL4-VH) compared to an IgG control RiboMab and a parental IgG as determined by Gyros ELISA from Example 6.

FIG. 14 shows exemplary Western Blot analysis of scFv-Fc PGDM1400 L/S RibobNAbs (e.g., VH-LL4-VL, VL-LL5-VH, VH-LL5-VL, and VL-LL4-VH) compared to a control RiboMab and a parental IgG under nonreducing conditions from Example 6.

FIG. 15 shows exemplary concentrations of CrossMab PGDM1400 L/S RibobNAbs (e.g., CrossMab^{CH1-CLcv} and CrossMab^{CH1-CLx}) compared to a control RiboMab and a parental Ab as determined by Gyros ELISA from Example 7.

FIG. 16 shows exemplary Western Blot analysis of CrossMab PGDM1400 L/S RibobNAbs (e.g., CrossMab^{CH1-CLcv} and CrossMab^{CH1-CLx}) compared to a control RiboMab and a parental Ab from Example 7.

FIG. 17 shows exemplary pharmacokinetic (PK) profiles of of PGDM1400 IgG and PGDM1400 IgG L/S RibobNAbs compared to a control RiboMab as determined by Gyros ELISA from Example 8 in NSG mice (panel A) and NSG Tg32 mice (panel B). Quantification of PGDM1400 RibobNAbs was performed using sera obtained from NSG mice and hFcRn NSG Tg32 mice. The in vivo concentrations in μg/mL are shown in log scale on the y-axis. The x-axis shows the time in days of the respective blood sampling.

FIG. 18 shows exemplary pharmacokinetic (PK) profiles of PGDM1400 IgG, IgG L/S and scFv-Fc L/S RibobNAbs. Quantification of PGDM1400 RibobNAbs was performed using sera obtained from hFcRn NSG Tg32 mice. The in vivo concentrations in μg/mL are shown in log scale on the y-axis. The x-axis shows the time in days of the respective blood sampling. Results of PGDM1400 VL-LL5-VH L/S scFv-Fc are depicted in comparison to PGDM1400 IgG and PGDM1400 L/S IgG RibobNAbs as determined by Gyros ELISA from Example 12. Two different doses of the PGDM1400 VL-LL5-VH L/S scFv-Fc (30 μg and 19.56 μg) and PGDM1400 L/S IgG (30 μg and 10 μg) were analyzed.

FIG. 19 shows exemplary results from a pseudovirus viral neutralization test (pVNT) where TZM.b1 cells were exposed to PGDM1400 scFv-Fc L/S (VH-LL4-VL, VL-LL5-VH, VH-LL5-VL, and VL-LL4-VH configurations) and pseudoviruses ZM53M.PB12, Du156.12, Q769.d22, 0330.v4.c3, R2184.c04, and 89-F1_2_25. A Murine Leukemia Virus (MuLV) pseudovirus was used as a negative control. Results are expressed as IC50 and IC80 values or antibody/IgG concentrations resulting in a 50% and 80% reduction in relative luminescence units (RLUs) compared to untreated virus control wells.

FIG. 20 shows exemplary results from pVNT where TZM.b1 cells were exposed to PGDM1400 scFv-Fc L/S (VH-LL4-VL, VH-LL5-VL, VL-LL4-VH, and VL-LL5-VH configurations) and pseudoviruses CAP304_2_00_F6_6, Ce1176_A3, 6980.v0.c31, 1012_11_TC21_3257, and PVO.4. A Murine Leukemia Virus (MuLV) pseudovirus was used as a negative control. Results are expressed as IC50 and IC80 values or antibody/IgG concentrations resulting in a 50% and 80% reduction in relative luminescence units (RLUs) compared to untreated virus control wells.

FIG. 21 shows exemplary results from pVNT where TZM.b1 cells were exposed to PGDM1400 CrossMab^{CH1-CLx} L/S and PGDM1400 CrossMab^{CH1-CHcv} L/S RibobNAbs and pseudoviruses ZM53M.PB12, Du156.12, Q769.d22, 0330.v4.c3, R2184.c04, and 89-F1_2_25. A Murine Leukemia Virus (MuLV) pseudovirus was used as a negative control. Results are expressed as IC50 and IC80 values or antibody/IgG concentrations resulting in a 50% and 80% reduction in relative luminescence units (RLUs) compared to untreated virus control wells.

FIG. 22 shows exemplary results from pVNT where TZM.b1 cells were exposed to PGDM1400 scFv-Fc (VL-LL5-VH) L/S RibobNAbs and pseudoviruses CAP45, X2088_c9, CNE8, T250-4, QH0692.42, and 3103.v3.c10 (panel A) and C1080_c3, T278-50, ZM109F.PB4, and Du156.12 (panel B). A Murine Leukemia Virus (MuLV) pseudovirus was used as a negative control. Results are expressed as IC50 and IC80 values or antibody/IgG concentrations resulting in a 50% and 80% reduction in relative luminescence units (RLUs) compared to untreated virus control wells.

### DEFINITIONS

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, provided compounds show one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by 13C- or 14C-enriched carbon are within the scope of this disclosure.

About. The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent. As used herein, the term “agent,” may refer to a physical entity. In some embodiments, an agent may be characterized by a particular feature and/or effect. For example, as used herein, the term “therapeutic agent” refers to a physical entity has a therapeutic effect and/or elicits a desired biological and/or pharmacological effect. In some embodiments, an agent may be a compound, molecule, or entity of any chemical class including, for example, a small molecule, polypeptide, nucleic acid, saccharide, lipid, metal, or a combination or complex thereof.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl,” used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene. The term “alkylene” is refers to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” or “-enyl” when appended to certain groups herein are intended to refer to a bifunctional moiety of said group. For example, “-ene” or “-enyl”, when appended to “cyclopropyl” becomes “cyclopropylene” or “cyclopropylenyl” and is intended to refer to a bifunctional cyclopropyl group, e.g.,

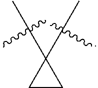 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody agent: As used herein, the term “antibody agent” refers to any polypeptide or polypeptide complex that includes immunoglobulin structural elements sufficient to confer specific binding to a particular antigen. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In some embodiments, the term “antibody agent” is used to refer to one or more of the art-known or developed constructs or formats for utilizing antibody structural and functional features in alternative presentation. For example, in some embodiments, an antibody agent utilized in accordance with the present disclosure is in a format selected from, but not limited to, intact IgA, IgG, IgE or IgM antibodies; bi- or multi-specific antibodies (e.g., Zybodies®, etc.); CrossMabs (e.g., CrossMab^{CH1-CL}; CrossMab^{CH1-CLcv}; bispecific CrossMab^{CH1-CL} with knob-in-hole); antibody fragments such as Fab fragments, Fab′ fragments, F(ab′)2 fragments, Fd′ fragments, Fd fragments, and isolated complementarity determining regions (CDRs) or sets thereof; single chain Fvs (scFvs); scFv-Fc fusions; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, chains and/or fragments of such antibodies and fragments may be used in combination, e.g., a scFv-Fc arm with a conventional antibody arm. In some embodiments, an antibody agent is a broadly neutralizing antibody agent (e.g., a broadly neutralizing antibody (bNab)). A “broadly neutralizing antibody agent” is an antibody agent that is capable of neutralizing two or more genetic variants (e.g., strains) of a virus (e.g., HIV). In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)). In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Aryl: The term “aryl” refers to monocyclic and bicyclic ring systems having a total of six to fourteen ring members (e.g., C6-C14), wherein at least one ring in the system is aromatic and wherein each ring in the system contains three to seven ring members. In some embodiments, an “aryl” group contains between six and twelve total ring members (e.g., C6-C12). The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

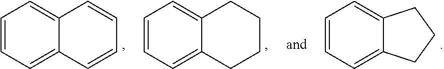

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Co-administration: As used herein, the term “co-administration” refers to use of a composition (e.g., a pharmaceutical composition) described herein and one or more additional therapeutic agents. In some embodiments, one or more additional therapeutic agents comprises at least one polyribonucleotide encoding another antibody agent (e.g., an anti-HIV antigen antibody agent). The combined use of a composition (e.g., a pharmaceutical composition) described herein and an additional therapeutic agent may be performed concurrently or separately (e.g., sequentially in any order). In some embodiments, a composition (e.g., a pharmaceutical composition) described herein and an additional therapeutic agent may be combined in one pharmaceutically-acceptable excipient, or they may be placed in separate excipient and delivered to a target cell or administered to a subject at different times. Each of these situations is contemplated as falling within the meaning of “co-administration” or “combination,” provided that a composition (e.g., a pharmaceutical composition) described herein and an additional therapeutic agent are delivered or administered sufficiently close in time that there is at least some temporal overlap in biological effect(s) generated by each on a target cell or a subject being treated.

Combination therapy: As used herein, the term “combination therapy” refers to those situations in which a subject is simultaneously exposed to two or more therapeutic regimens (e.g., two or more therapeutic agents (e.g., two or more antibody agents)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, administration of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition. In some embodiments, a combination therapy comprises polyribonucleotides encoding two or more antibody agents (e.g., anti-HIV antibody agents).

Comparable. As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Cycloaliphatic: As used herein, the term “cycloaliphatic” refers to a monocyclic C₃₋₈ hydrocarbon or a bicyclic C₆₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms. Exemplary monocyclic cycloalkyl rings include cyclopropyl, cyclobutyl, cyclopentyl, cyclohexyl, and cycloheptyl.

Derived. In the context of an amino acid sequence (peptide or polypeptide) “derived from” a designated amino acid sequence (peptide or polypeptide), it refers to a structural analogue of a designated amino acid sequence. In some embodiments, an amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, antibody agents utilized according to the present disclosure may include amino acid sequences (e.g., CDRs, variable domains, constant domains, etc.) derived from other antibodies, e.g., naturally produced antibodies.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Dosing regimen: Those skilled in the art will appreciate that the term “dosing regimen” (or “therapeutic regimen”) may be used to refer to a set of unit doses (typically more than one) that are administered individually to a subject, typically separated by periods of time. In some embodiments, a given therapeutic agent has a recommended dosing regimen, which may involve one or more doses.

Encode: As used herein, the term “encode” or “encoding” refers to sequence information of a first molecule that guides production of a second molecule having a defined sequence of nucleotides (e.g., a polyribonucleotide) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule encodes a polypeptide if transcription and translation of RNA corresponding to that gene produces the polypeptide in a cell or other biological system. In some embodiments, a coding region of a polyribonucleotide encoding a target antigen refers to a coding strand, the nucleotide sequence of which is identical to the polyribonucleotide sequence of such a target antigen. In some embodiments, a coding region of a polyribonucleotide encoding a target antigen refers to a non-coding strand of such a target antigen, which may be used as a template for transcription of a gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: As used herein, the term “epitope” refers to a moiety that is specifically recognized by an immunoglobulin (e.g., antibody or receptor) binding component. For example, an epitope may be recognized by a T cell, a B cell, or an antibody. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Accordingly, in some embodiments, an epitope of an antigen may include a continuous or discontinuous portion of the antigen. In some embodiments, an epitope is or comprises a T cell epitope. In some embodiments, an epitope may have a length of about 5 to about 30 amino acids, or about 10 to about 25 amino acids, or about 5 to about 15 amino acids, or about 5 to 12 amino acids, or about 6 to about 9 amino acids.

Expression: As used herein, the term “expression” of a nucleic acid sequence refers to the generation of a gene product from the nucleic acid sequence. In some embodiments, a gene product can be a transcript, e.g., a polyribonucleotide as provided herein. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Heteroaliphatic: The term “heteroaliphatic” or “heteroaliphatic group,” as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 n-electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatonm: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 6- to 10-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided composition (e.g., a pharmaceutical composition) may be “increased” relative to that obtained with a comparable reference composition. Alternatively or additionally, in some embodiments, an assessed value achieved in a subject may be “increased” relative to that obtained in the same subject under different conditions (e.g., prior to or after an event; or presence or absence of an event such as administration of a composition (e.g., a pharmaceutical composition) as described herein, or in a different, comparable subject (e.g., in a comparable subject that differs from the subject of interest in prior exposure to a condition, e.g., absence of administration of a composition (e.g., a pharmaceutical composition) as described herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

In order: As used herein with reference to a polynucleotide or polyribonucleotide, “in order” refers to the order of features from 5′ to 3′ along the polynucleotide or polyribonucleotide. As used herein with reference to a polypeptide, “in order” refers to the order of features moving from the N-terminal-most of the features to the C-terminal-most of the features along the polypeptide. “In order” does not mean that no additional features can be present among the listed features. For example, if Features A, B, and C of a polynucleotide are described herein as being “in order, Feature A, Feature B, and Feature C,” this description does not exclude, e.g., Feature D being located between Features A and B.

Ionizable: The term “ionizable” refers to a compound or group or atom that is charged at a certain pH. In the context of an ionizable amino lipid, such a lipid or a function group or atom thereof bears a positive charge at a certain pH. In some embodiments, an ionizable amino lipid is positively charged at an acidic pH. In some embodiments, an ionizable amino lipid is predominately neutral at physiological pH values, e.g., in some embodiments about 7.0-7.4, but becomes positively charged at lower pH values. In some embodiments, an ionizable amino lipid may have a pKa within a range of about 5 to about 7.

Isolated. The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

RNA lipid nanoparticle: As used herein, the term “RNA lipid nanoparticle” refers to a nanoparticle comprising at least one lipid and RNA molecule(s), e.g., one or more polyribonucleotides as provided herein. In some embodiments, an RNA lipid nanoparticle comprises at least one cationic amino lipid. In some embodiments, an RNA lipid nanoparticle comprises at least one cationic amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the average particle diameter. In some embodiments, RNA lipid nanoparticles may be prepared by mixing lipids with RNA molecules described herein.

Neutralization: As used herein, the term “neutralization” refers to an event in which binding agents such as antibodies bind to a biological active site of a virus such as a receptor binding protein, thereby inhibiting the parasitic infection of cells. In some embodiments, the term “neutralization” refers to an event in which binding agents eliminate or significantly reduce ability of infecting cells.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutically effective amount: The term “pharmaceutically effective amount” or “therapeutically effective amount” refers to the amount which achieves a desired reaction or a desired effect alone or together with further doses. In the case of the treatment of a particular disease (e.g., HIV), a desired reaction in some embodiments relates to inhibition of the course of the disease (e.g., HIV). In some embodiments, such inhibition may comprise slowing down the progress of a disease (e.g., HIV) and/or interrupting or reversing the progress of the disease (e.g., HIV). In some embodiments, a desired reaction in a treatment of a disease (e.g., HIV) may be or comprise delay or prevention of the onset of a disease (e.g., HIV) or a condition (e.g., an HIV associated condition). An effective amount of a composition (e.g., a pharmaceutical composition) described herein will depend, for example, on disease (e.g., HIV) or a condition (e.g., an HIV associated condition) to be treated, the severity of such a disease (e.g., HIV) or a condition (e.g., an HIV associated condition), individual parameters of the patient, including, e.g., age, physiological condition, size and weight, the duration of treatment, the type of an accompanying therapy (if present), the specific route of administration and similar factors. Accordingly, doses of a composition (e.g., a pharmaceutical composition) described herein may depend on various of such parameters. In the case that a reaction in a patient is insufficient with an initial dose, higher doses (or effectively higher doses achieved by a different, more localized route of administration) may be used.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA) or Polyribonucleotide: As used herein, the term “ribonucleic acid,” “RNA,” or “polyribonucleotide” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is a mRNA. In some embodiments, where an RNA is a mRNA, a RNA typically comprises at its 3′ end a poly(A) region. In some embodiments, where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is expressed as a risk relative to a risk associated with a reference sample or group of reference samples. In some embodiments, a reference sample or group of reference samples have a known risk of a disease, disorder, condition and/or event. In some embodiments, a reference sample or group of reference samples are from individuals comparable to a particular individual. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

Selective or specific: The term “selective” or “specific,” when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

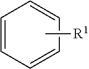

refers to at least

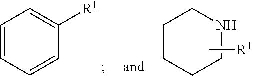

and refers to at least

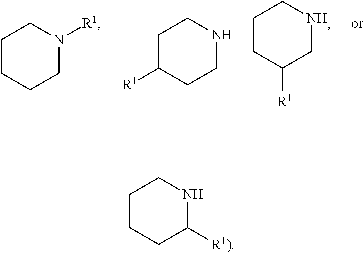

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄₀R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄₀C(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂Rt; wherein each R^{†} is independently hydrogen, C₁₋₆ aliphatic which may be substituted as defined below, unsubstituted—OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., HIV, an HIV-associated condition, etc.). In some embodiments, a subject is susceptible to a disease, disorder, or condition (e.g., HIV, an HIV-associated condition, etc.). In some embodiments, a subject displays one or more symptoms or characteristics of a disease, disorder, or condition (e.g., HIV, an HIV-associated condition, etc.). In some embodiments, a subject displays one or more non-specific symptoms of a disease, disorder, or condition (e.g., HIV, an HIV-associated condition, etc.). In some embodiments, a subject does not display any symptom or characteristic of a disease, disorder, or condition (e.g., HIV, an HIV-associated condition, etc.). In some embodiments, a subject is someone with one or more features characteristic of susceptibility to or risk of a disease, disorder, or condition (e.g., HIV, an HIV-associated condition, etc.). In some embodiments, a subject is a patient. In some embodiments, a subject is an individual to whom diagnosis and/or therapy is and/or has been administered.

Suffering from: An individual who is “suffering from” a disease, disorder, and/or condition (e.g., HIV, an HIV-associated condition, etc.) has been diagnosed with and/or displays one or more symptoms of a disease, disorder, and/or condition.

Susceptible to: An individual who is “susceptible to” a disease, disorder, and/or condition (e.g., HIV, an HIV-associated condition, etc.) is one who has a higher risk of developing the disease, disorder, and/or condition (e.g., HIV, an HIV-associated condition, etc.) than does a member of the general public. In some embodiments, an individual who is susceptible to a disease, disorder and/or condition (e.g., HIV, an HIV-associated condition, etc.) may not have been diagnosed with the disease, disorder, and/or condition (e.g., HIV, an HIV-associated condition, etc.). In some embodiments, an individual who is susceptible to a disease, disorder, and/or condition (e.g., HIV, an HIV-associated condition, etc.) may exhibit symptoms of the disease, disorder, and/or condition (e.g., HIV, an HIV-associated condition, etc.). In some embodiments, an individual who is susceptible to a disease, disorder, and/or condition (e.g., HIV, an HIV-associated condition, etc.) may not exhibit symptoms of the disease, disorder, and/or condition (e.g., HIV, an HIV-associated condition, etc.). In some embodiments, an individual who is susceptible to a disease, disorder, and/or condition (e.g., HIV, an HIV-associated condition, etc.) will develop the disease, disorder, and/or condition (e.g., HIV, an HIV-associated condition, etc.). In some embodiments, an individual who is susceptible to a disease, disorder, and/or condition (e.g., HIV, an HIV-associated condition, etc.) will not develop the disease, disorder, and/or condition (e.g., HIV, an HIV-associated condition, etc.).

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition (e.g., HIV, an HIV-associated condition, etc.). In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition (e.g., HIV, an HIV-associated condition, etc.). Treatment may be administered to a subject who does not exhibit signs of a disease, disorder, and/or condition (e.g., HIV, an HIV-associated condition, etc.). In some embodiments, treatment may be administered to a subject who exhibits only early signs of the disease, disorder, and/or condition (e.g., HIV, an HIV-associated condition, etc.), for example for the purpose of decreasing the risk of developing pathology associated with the disease, disorder, and/or condition. In some embodiments, treatment may be administered to a subject at a later-stage of disease, disorder, and/or condition (e.g., HIV, an HIV-associated condition, etc.).

### DETAILED DESCRIPTION OF CERTAIN EMBODIMENTS

### I. Human Immunodeficiency Virus (HIV)

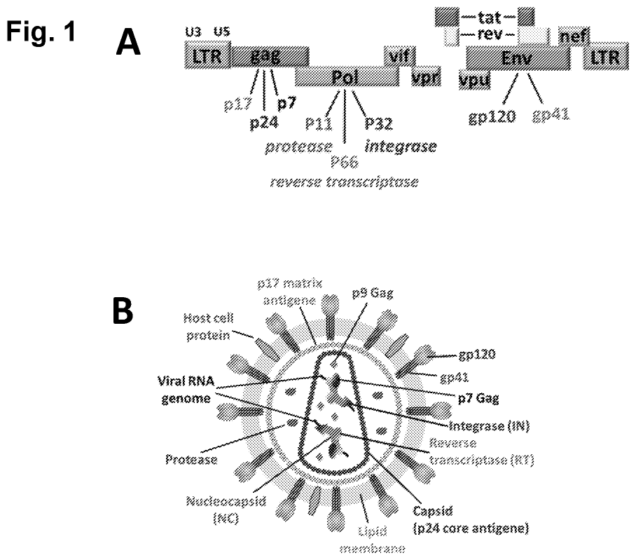

Human Immunodeficiency Virus (HIV) is a lentivirus within the family Retroviridae. A mature HIV particle is generally round in shape and approximately 100 nm in diameter. It is composed of (from innermost to outermost) a core comprising of two identical single-stranded RNA molecules, a capsid, and an envelope (FIG. 1, Panel B) (Musumeci et al., Molecules 20.9 (2015): 17511-17532, which is herein incorporated by reference). The envelope is composed of a lipid bilayer and Env proteins. These Env proteins exist as trimers of gp120 surface protein anchored in the envelope membrane through a gp41 transmembrane protein. The viral capsid, surrounded by the envelope, comprises a symmetrical outer capsid membrane, which is made up of matrix protein p17. Within the outer capsid membrane is the conical capsid comprised of inner capsid protein p24. The inner capsid is attached to the outer capsid membrane at its tapered cone. The inner capsid contains the viral RNA (two identical copies) and viral enzymes: reverse transcriptase, integrase, and protease. Also contained with the viral particle are oligopeptides generated by proteolytic processing of Gag and Gag/Pol precursor proteins p55 and p160 that occurs during the maturation of the viral particle (GAC, Transfusion Medicine Hemotherapy, 43:203-222, 2016, which is herein incorporated by reference).

There are two main types of HIV, HIV-1 and HIV-2. HIV-1 is the most common type of HIV and accounts for 95% of all infections worldwide. HIV-2 is relatively uncommon and less infectious. HIV-2 is mainly concentrated in West Africa and the surrounding countries.

HIV-1 and HIV-2 have many similarities including their intracellular replication pathways, transmission modes and clinical effects leading to acquired immune deficiency syndrome (AIDS). However, HIV-2 is less likely to progress into AIDS because of its lower transmissibility. Thus, individuals infected by HIV-2 generally do not see disease progression for a long period of time, while patients infected by HIV-1 progress faster and tend to contract AIDS.

Once progression begins, however, the pathological process for both viruses is largely similar. One difference is that HIV-2 is found to progress at higher CD4 counts. Additionally, HIV-2 infections are characterized by lower viral loads of over 10,000 copies/mL compared to millions of copies/mL of HIV-1. A subject's immune response tends to be more protective in the case of HIV-2 infection, thus slowing down disease progression.

HIV-1 and HIV-2 are, in turn, further divided into groups and subtypes. HIV-1 is divided into main or M group, outlier or O group and non-M/O or N group. The most common group is group M, which is mostly responsible for the HIV epidemic worldwide. The other groups are relatively uncommon and are seen in select geographies including Gabon, Cameroon and Equatorial Guinea.

Group M is still further divided into genetically distinct subtypes: A, B, C, D, F, G, H, J and K. Some of these subtypes combine to form a hybrid virus called “circulating recombinant form.” Globally, subtype B accounts for 12% of HIV infections. Subtype B is the dominant HIV-1 subtype found in the Americas, Australasia, and Western Europe. As a result, most of the clinical research on HIV to date is focused on these populations.

Although subtype C represents almost 50% of all HIV affected individuals, less research has focused on this subtype. Subtype C is commonly found in countries in Southern Africa, where the incidence of HIV is very high. Cameroon and the Democratic Republic of Congo, the region of origin of HIV-1, have great diversity of HIV-1 subtypes. However, the pattern of subtype distribution across the globe is changing now, due to population mixing and migration.

There are about eight HIV-2 subtypes identified to date. The two main subtypes of HIV-2 that are considered epidemic are A and B. HIV-2 group A infections are mostly seen in West Africa, though a few cases have been reported in Brazil, Europe, US and India. HIV-2 group B infections have only been seen in West Africa.

Because HIV subtypes can be geographically diverse, ideal therapeutics are able to target and neutralize more than one subtype, and even more preferably, multiple strains of HIV. As discussed further below, anti-HIV antibodies capable of binding to and at least temporarily neutralizing HIV virions have been developed. Nonetheless, issues persist with such anti-HIV antibodies, including challenges with administration, antibody persistence in vivo, and viral escape. Polyribonucleotides and compositions of the present disclosure address these challenges, as described herein.

### A. HIV Genome

HIV contains two identical copies of single-stranded DNA encoding its genome. When the virus integrates into a host cell, reverse transcription of the viral RNA into double stranded DNA occurs, which leads to degradation of the RNA and integration of the double stranded DNA, or proviral DNA, into the host genome. The HIV genome is flanked at both ends by an LTR (long terminal repeat) region, including a 5′ LTR encoding a transcriptional promotor. The RNA genome is 9749 nucleotides and comprises a 5′ cap, a 3′ poly(A) tail, and several open reading frames ORFs (Wain-Hobson et al., Cell 40(1):9-17, 1985, which is herein incorporated by reference).

The HIV genome includes the following genes: gag, pol, vif, vpr, tat, rev, vpu, env, and nef (see FIG. 1, Panel A). The proteins encoded by gag, pol, and env are viral structural proteins. The proteins encoded by tat and rev are essential regulatory proteins. The proteins encoded by nef, vpr, vif, and vpu are accessory regulatory proteins. The gag gene encodes a P555Gag precursor protein of proteins of the outer core membrane (p17), the capsid protein (p24), the nucleoprotein (p7), Pr55Gag, and p6 protein. Protein p24 forms the conical capsid and protein p17 forms the inner membrane layer. Protein p6 is involved in viral particle release.

The pol gene encodes Pr160GagPol precursor protein, protease enzyme p10, reverse transcriptase (p51), and RNase H (p15) or both together as p66 protein, and integrase p32. Pr160GagPol is the precursor of the viral enzymes p10, p51, and p15. Proteolytic cleavage of Gag (Pr55) and Gag-Pol (Pr160GagPol) results in protease p10. Protein p51 reverse transcriptase is responsible for transcription of HIV RNA into proviral DNA. Protein p55 (RNAse H) functions to degrade viral RNA in the viral RNA/DNA complex when proviral DNA is generated. Protein p32 integrase functions in integrating proviral DNA into a host cell genome.

The env gene encodes precursor protein PrGp160 of two envelope glycoproteins gp120 (surface protein) and gp41 (transmembrane protein). Proteins gp120 and gp41 are generated by protease cleavage of precursor protein PrGp160. Protein gp120 functions in the attachment of the virus to a target host cell. Protein gp41 anchors gp120 into the viral membrane and functions to fuse the viral and target cell membrane.

The gene tat encodes Tat protein p14 (transactivator protein), which activates transcription of viral genes. The rev gene encodes Rev protein p19 (RNA splicing-regulator), which regulates export of mRNA (both non-spliced and partially spliced). The nef gene encodes Nef protein p27 (negative regulating factor), which functions in HIV replication and enhances the infectivity of the virus in a host cell. Protein p27 also functions to downregulate CD4 and HLA on target cells. The vif gene encodes Vif protein p23 (viral infectivity factor), which functions in the production of the virus in a host cell. The gene vpr encodes Vpr protein p15 (virus protein r). This protein interacts with p6 protein and facilitates infectivity of the virus in a host cell. The gene vpu encodes Vpu protein p16 (virus protein unique), which allows for efficient release of the viral particle and controls CD4 degradation on a target cell. Protein p16 also controls intracellular signaling. The gene vpx encodes Vpx protein p15 (virus protein x), which functions to interact with p6 protein and is important in the early phases of viral replication. The gene tev encodes Tat/Rev protein p26, which is a fusion protein that regulates Tat and Rev proteins (GAC, Transfusion Medicine Hemotherapy, 43:203-222, 2016, which is herein incorporated by reference).

### B. Lifecycle

The lifecycle of HIV involves HIV virion entry into a target host cell, reverse transcription of the viral genome, integration into the host genome, and protein maturation. To initiate infection, an HIV particle comes into contact with a target host cell. Surface glycoprotein env gp120 of a mature HIV particle binds to a CD4 receptor on the target host cell, which initiates the additional binding of gp120 to a co-receptor, i.e., chemokine receptor 5 (CCR5) or chemokine receptor 4 (CXCR4 of fusin). Binding of gp120 to CD4 and the co-receptor triggers a conformational change of gp120 so that gp41 is presented on the viral membrane and can fuse with the plasma membrane of the target host cell. The viral capsid then enters the cytoplasm of the host cell. The capsid is taken up by an endosome releases its contents, i.e., the viral RNA. Upon entry and release of the virus into the target host cell, the virus undergoes reverse transcription, where the viral RNA is reverse transcribed into single stranded cDNA. The RNA strand is then degraded by RNase H, and the single stranded cDNA is converted to double stranded DNA by DNA-dependent DNA polymerase activity by the reverse transcriptase enzyme.

The double-stranded DNA, or proviral DNA, forms a complex with integrase and is transported into the nucleus of the host cell, and inserts itself at random into the host cell genome. Once integrated into the genome, the proviral genome is replicated. The proviral genome can be replicated with the host cell genome as part of cell division or can replicate using its own machinery. For example, the LTR promotor creates an attachment site for cellular DNA-dependent RNA polymerases and transcription factors to initiate transcription. Transcription of proviral DNA is accelerated by the Tat protein.

The process of entry into a target host cell, reverse transcription, integration and protein maturation can be completed in less than 24 hours, and progeny viral particles have been detected within 12 hours of infection. The first progeny viral particles after infection may be released from the infected cells about 24 hours after infection. Infected T cells are typically eliminated at a rate of 2-4 days by the immune system (e.g., by cytotoxic T cells). As HIV infected T cells are destroyed and production of T cells is limited, there is a decline in T helper cells. The proteins nef and tat also inhibit maturation and replacement of helper T cells. As a result, an HIV infection over time will result in immunodeficiency (GAC, Transfusion Medicine Hemotherapy, 43:203-222, 2016, which is herein incorporated by reference).

### C. Transmission and Pathology

HIV enters the body through intact mucous membranes, injured skin or by parenteral inoculation. HIV is most commonly transmitted sexually. Upon infection, HIV can be detected throughout the body by about 10-14 days, and transmission via blood or transplanted organs is possible after about 5-6 days post infection. Clinical symptoms typically manifest after 3-6 weeks post infection and can include fever, lymph node enlargement, fatigue, rash, gastrointestinal symptoms, acute neuropathy, myalgias and/or malaise. However, during this acute phase some individuals are asymptomatic. These symptoms of the acute or primary infection can persist for 2-6 weeks. This initial symptomatic phase is then typically followed by an asymptomatic phase or one with occasional symptoms, which can last several years.

Left untreated, HIV infection causes progressive CD4+ T cell loss, which can lead to a suite of immunological abnormalities and an increased risk of infectious and oncological complications. Additionally, HIV infection also implicates cardiovascular disease, bone disease, renal and hepatic dysfunction, and several other common morbidities.

Although antiviral therapies (ART) have been developed to treat HIV infection, ART can only prevent new cells from becoming infected, i.e., ART cannot eliminate infection if a cell already contains viral DNA integrated into its genome. Additionally, HIV establishes a latent infection in CD4+ T cells that can be maintained indefinitely, some having the capability of self-renewal. HIV may continue to reinitiate replication in a cell once it is integrated into its genome. (Deeks et al. Nature reviews 1.1 2015 and GAC, Transfusion Medicine Hemotherapy, 43:203-222, 2016 which are herein incorporated by reference).

### D. Therapeutic Strategies

Development of therapeutics targeting HIV face many challenges. One challenging factor is the heterogeneity of the virus. HIV can be divided into at least two major types (HIV-1, found worldwide, and HIV-2, found largely in west Africa), while HIV-1 has been further subdivided into three subgroups (M, N, O and P), and M has been still further subdivided into subtypes A-L. Subtypes are also able to recombine upon co-infection, resulting in yet further recombinant subtypes.

Another challenging factor is that the high mutation rate of HIV in vivo. A recent study quantified the HIV-1 genome-wide rate of spontaneous mutation in DNA sequences from peripheral blood mononuclear cells and revealed an extremely high mutation rate of (4.1±1.7)×10⁻³ per base per cell, which is the highest rate reported for any biological entity (Cuevas et al, PloS Biol 2015, which is herein incorporated by reference). The ability to identify and develop a therapeutic targeting a conserved epitope across the multiple groups and subtypes of a continuously mutating HIV sequence is therefore extremely challenging, and the virus has a unique ability to evade the immune system.

Aside from the high mutational frequency, HIV presents other challenges for the immune system that make it uniquely challenging to treat. Therapeutic targets on HIV include the HIV envelop protein (HIV Env), however, the HIV Env is heavily glycosylated and the Env sites are therefore shielded from a therapeutic by the glycans present. Additionally, Env glycans are derived from the host and can be extremely heterogeneous.

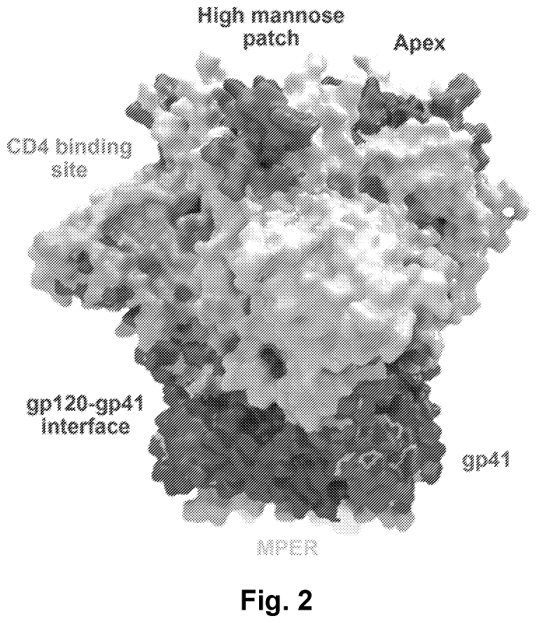

A recent therapeutic strategy involves use of broadly neutralizing antibodies (bNAbs), which are antibodies that can neutralize a diversity of global HIV isolates. Such antibodies have been identified from individuals infected by HIV considered to be “Elite Neutralizers,” making up<10% of HIV patients (Burton and Hangartner, Ann. Rev. Immunol. 2016, which is herein incorporated by reference). Such antibodies provide insights for potential target epitopes and structures of therapeutics. Other advances to aid the advance of therapeutics include the generation of a stable HIV Env spike trimer (Sanders and Moore, Immunol. Rev. 2017, which is herein incorporated by reference) and characterization of its structure at high resolution (Ward and Wilson, Immunol. Rev. 2017, which is herein incorporated by reference). Examples of Env sites that are potential targets include the apex site, the high-mannose patch of the gp120 region, the gp120-gp41 interface region, the gp41 membrane proximal region (MPER), and the CD4 binding site (see FIG. 2, from McCoy and Burton, Immunol Rev. 275.1 11-20 2017, which is herein incorporated by reference). These sites each face unique challenges as a therapeutic target for bnAbs. For example, bnAbs that target the gp41-gp120 interface must be able bind to complex heterogeneous glycans. BnAbs that target the CD4 binding site of the Env protein have been found to show high levels of somatic hypermutation.

Nonetheless, among these sites, the apex site is of particular interest because it is a conserved region identified in the HIV envelop trimer (Env).

As discussed herein, the HIV-1 envelope (Env) glycoprotein trimer, which is a trimer of gp120-gp41 heterodimers, is the only viral protein on the surface of HIV-1 virons and are therefore an important target of bnAbs. The apex of Env trimer includes the variable 1 and 2 (V1V2) loops and the variable 3 (V3) loop. The V1V2 loops contain four β-strands. Additionally, the V1V2 epitope is of particular interest as a target because it is a region of gp120 that upon binding with CD4, undergoes large conformational changes. For example, CD4 and gp120 interaction stabilizes an open Env conformation that is able to interact with a chemokine receptor to cause additional conformation changes that ultimately results in insertion of the gp41 fusion protein into a target cell membrane. In the closed state, V1V2 regions from the three gp120s shield the V3 and coreceptor binding site. As such, certain apex-targeting bNAbs (e.g., targeting V1V2 epitopes) are characterized by the ability to prevent the opening of the Env trimer to expose the V3 lop and coreceptor binding site, thereby blocking conformational changes that lead to fusion of the viral and target host cell membranes (see Wang, Haoqing, et al., “Asymmetric recognition of HIV-1 Envelope trimer by V1V2 loop-targeting antibodies.” Elife 6 (2017): e27389, which is herein incorporated by reference).

The present disclosure provides, among other things, polyribonucleotides that encode antibody agents, e.g., bNABs, that target a broader group of HIV variants, and as such and are able to treat a greater number of HIV patients. Additionally, the present disclosure provides compositions for the delivery of polyribonucleotides that encode antibody agents, e.g., bNABs, targeting various HIV sequences.

### 1. Anti-Viral Treatments for HIV

HIV infection is currently primarily treated with antiretroviral therapy (ART). ART is a type of drug that can reduce HIV multiplication, increase CD4 cell count, and reduce transmission risk in an infected individual. The World Health Organization (WHO) recommends that ART be initiated in all adults infected with HIV regardless of clinical stage or CD4 cell count (Consolidated guidelines on HIV prevention, testing, treatment, service delivery and monitoring: recommendations for a public health approach. Geneva: World Health Organization; 2021, which is herein incorporated by reference). However, ART is not a curative therapy, and viremia (e.g., viral load) will quickly rebound if an infected individual stops taking ART. The high mutation rate of HIV also constrains patient in having a strict observance with their therapy to avoid the emergence of escape mutants and treatment failure. Thus, ART is intended to be taken every day for the entirety of an infected subject's life.

There are several classes of FDA-approved ART to treat HIV that act via different mechanisms. Effective management of HIV infection often involves combinations of at least 3 ARTs to treat the complex pathogenicity of the disease. The most effective combination of ARTs is often different between infected individuals (see, for example, Bhatti et al., Cureus 2016, which is herein incorporated by reference). Cihlar et al., Current opinion in virology, 2016, hereby incorporated by reference in its entirety, reviews the classes of ART drugs for the treatment of HIV.

TABLE 1
Exemplary classes of ART used to treat HIV, mechanisms
of action, and exemplary compounds in each class

| ART Class | Mechanism of Action | Examples |
|---|---|---|
| CCR5 Antagonist | Inhibits HIV entry into cell by | Maraviroc |
| | blocking CCR5 and viral interaction | |
| Fusion Inhibitors | Inhibits HIV envelope from | Enfuvirtide |
| | merging with host cell membrane | |
| Integrase Strand Transfer Inhibitor | Inhibits integration of viral DNA | Bictegravir, Dolutegravir, |
| (INSTI) | into host genome by blocking | Elvitegravir, Raltegravir |
| | integrase enzyme | |
| Non-Nucleoside Reverse | Inhibits HIV replication by blocking | Delavirdine, Doravirine, Efavirenz, |
| Transcriptase Inhibitors (NNRTI) | reverse transcriptase enzyme to | Etravirine, Nevirapine, Rilpivirine, |
| | suppress conversion of RNA into | |
| | DNA | |
| Nucleoside Reverse Transcriptase | Inhibits HIV replication by blocking | Abacavir, Emtricitabine, |
| Inhibitors (NRTI) | reverse transcriptase enzyme to | Lamivudine, Tenofovir, Zidovudine |
| | suppress conversion of RNA into | |
| | DNA | |
| Post-Attachment Inhibitor | Inhibits HIV entry into cell after | Ibalizumab |
| | binding has occurred | |
| Protease Inhibitors | Inhibits HIV particle development | Atazanavir, Darunavir, |
| | and maturity by blocking protease | Fosamprenavir, Lopinavir, |
| | enzyme | Nelfinavir, Ritonavir, Tipranavir |

### 2. HIV Antibody Agents

In addition to ART, anti-HIV antibodies have been developed. Using anti-HIV antibodies for treating HIV generally requires antibodies having specific characteristics, including safety, a favorable pharmacokinetic profile, highly potent neutralizing activity, and broad neutralizing activity to effectively target the diversity present in HIV virions. As with other HIV therapeutics (including, e.g., ART), viral escape from anti-HIV antibodies present as significant challenge.

For example, Barouch, et al. infected rhesus macaques with SHIV-SF162P3 (Barouch, et al., Nature 503: 7475 224-228, 2013), which is incorporated herein by reference in its entirety. The rhesus macaques were then treated with 3 monoclonal antibodies (mAbs): N332 glycan-dependent mAb PGT121 and CD4 binding site-specific mAbs 3BNC117 and b12. mAbs were administered as a cocktail on days 0 and 7 at 10 mg/kg each, as a cocktail on day 0 alone at 10 mg/kg each or as a combination of just PGT121 and 3BNC117 at 10 mg/kg each. Transient viral suppression was observed until bNAbs level dropped below 10 μg/mL. mAbs were also singly administered to macaques, and PGT121 alone resulted in rapid virologic control which rebounded after 6-8 weeks in most animals. The macaques that received a combination of PGT121 and 3BNC117 were given a second dose on day 105, after viral levels had rebounded. Viral re-suppression was observed, although the control was less durable than the previous administration.

Shingai, et al. describes rhesus macaques were infected with SHIV_{AD8EO} (Shingai, et al., Nature 503: 7475 277-280, 2013, which is incorporated herein by reference in its entirety). The rhesus macaques were then treated with 10-1074 and 3BNC117 mAbs singly or in combination. When administered singly at 12 weeks post inoculation at 10 mg/kg, both antibodies caused rapid viral suppression, but virus levels quickly rebounded. Administration of both antibodies in combination to chronically infected animals resulted in longer periods of suppression and increased CD4+ T cell levels, although viral levels did rebound later. In other studies, both antibodies were singly pre-treated to macaques and were found to prevent virus acquisition. Single genome analysis of rebounded virus in 10-074 treated macaques revealed mutations that eliminated the gp120 N332 glycan, rendering resistance to the mAb. However, SGA analysis of rebounded virus in macaques treated with both 10-074 and 3BNC117 revealed that not all of the macaques contained virus with changes that imparted mAb resistance.

Caskey, et al. describe a first-in-human dose escalation phase 1 clinical trial of 3BNC117 (CD4 binding site antibody) (Caskey, et al., Nature 522.7557: 487-491, 2015, which is incorporated herein by reference in its entirety). Uninfected and HIV-1-infected individuals were enrolled in the trial. 1, 3, 10, or 30 mg/kg doses of 3BNC117 were found to be generally safe and well tolerated; no grade 3, 4, or serious adverse events were observed. HIV-1-infected individuals were observed to have a quicker clearance rate of the antibody than uninfected control subjects. The effect of the treatment on viral load was dose-dependent; 10 and 30 mg/kg doses decreased viral load by up to 2.5 log. Viral resistance was observed to develop in some individuals regardless of mAb dose, but not in other individuals. Viruses were cloned and sequenced, G459D was a commonly observed mutation in the 10 mg/kg group, others showed a longer V5 loop (other mutations described). Both mutations can alter sensitivity to anti-CD4bs.

Caskey, et al. also assessed 10-074, which is a highly potent mAb that targets the V3 loop of the HIV-1 envelope spike (Caskey et al., Nature Medicine 23.2: 185-191, 2017), which is incorporated herein by reference in its entirety. An open label phase 1 first-in-human clinical trial was performed with 14 uninfected and 19 HIV-1-infected individuals. A single intravenous infusion was administered at 3, 10, or 30 mg/kg. The mAb was found to be generally safe and well-tolerated; no grade 3, 4, or serious adverse events were observed. HIV-1-infected individuals were observed to have a quicker clearance rate of the antibody than uninfected controls. Treatment suppressed viral load in individuals with 10-074 sensitive strains, followed by rebound. Single genome sequencing (SGS) of rebounded virus revealed all patients that responded to treatment displayed a PNGS at position N332 and an intact ³²⁴G(D/N)IR³²⁷ motif. Four weeks after infusion, 91% of envelope sequences contained amino acid mutations, 97% of which eliminated the PNGS at position 332 by mutating either N332 or S334. 3% of mutated sequences showed changes at D/N325 in the ³²⁴G(D/N)IR³²⁷ motif. Majority of mutations at nucleic acid level were transitions, consistent with reverse transcriptase errors. Neutralization assay testing showed that mutated HIV-1 resistant to 10-074 was not resistant to 3BNC117, VRC01, or PGDM1400 (mAbs targeting other regions of HIV-1). SGS performed 1 week after infusion revealed that resistant variants are preexisting or rapidly generated.

Bar, et al. performed two open-lab trials, which were conducted on the safety, side-effect profile, pharmacokinetic properties, and antiviral activity of VRC01 (bNAb targeting CD4-binding site of HIV) in patients undergoing interruption of antiretroviral therapy (ART) (Bar et al., New England Journal of Medicine 375.21: 2037-2050, 2016, which is incorporated herein by reference in its entirety). In one trial, 40 mg/kg was infused 3 times over a 6 week period, and in the other trial, 40 mg/kg was infused 8 times over a 6 month period. Treatment was well tolerated, no grade 3 or higher adverse events were observed. Neither trial produced durable suppression of plasma viremia, although a slight increase in time to rebound was found relative to historical controls. Regardless of time to rebound, resistance to VRC01 increased almost universally in participants in one trial. Viral isolates showed greater resistance to VRC01 neutralization in pre-treated samples versus post-treated. Treatment with VRC01 did not impact the susceptibility to neutralization with other bNAbs.

Mendoza, et al. performed a phase 1b clinical trial assessing the combination of 3BNC117 and 10-1074, which were infused at 30 mg/kg dose on weeks 0, 3, and 6 (Mendoza, et al., Nature 561.7724: 479-484, 2018, which is incorporated herein by reference in its entirety). These two bNAbs target independent sites on HIV-1 envelope spike. Infusions were generally found to be safe and well tolerated with no reported serious adverse events. Median time to rebound was significantly extended with combination bNAb treatment. Two earliest rebounder individuals were found to previously harbor strains resistant to one or the other bNAb. Rebounded virus clustered within low diversity lineages consistent with expansion of 1-2 recrudescent viruses (escape). Most rebounded virus was found to contain 10-1074 mutations as compared to 3BNC117 mutations. However, combination bNAb therapy proved more effective at containing viral escape than single bNAb treatment.

Gautam, et al. assayed rhesus macaques that were infected with SHIV_{AD8EO} and treated with 3BNC117-LS and 10-074-LS mAbs (Gautam, Rajeev, et al., Nature Medicine 24.5: 610-616, 2018, which is incorporated herein by reference in its entirety). M428L and N343S (collectively referred to as LS) are mutations in the fragment domains of the mAbs to increase half-life. The LS mutations had no effect on virus neutralization in in vitro assays. LS mAbs were administered singly at 20 mg/kg, and were well tolerated in all monkeys. 10-1074-LS recipients demonstrated increased protection from virus acquisition than 3BNC117-LS recipients, but LS mutations in both antibodies were more effective than WT. 10-1074-LS decayed at a slower rate than 3BNC117-LS in serum. mAb concentration/neutralization activity were determined to be predictive of the probability of infection. Only experiments in which antibody pre-treatment followed by virus challenge were performed.

Schommers, et al. characterized an anti-HIV antibody, referred to as “1-18,” in in vitro assays, as well as HIV-1 infected humanized mice. 1-18 was reported to bind to the CD4 binding site of HIV and have strong potency and breadth against HIV strains. Schommers reported that 1-18 had certain characteristics previously found in other anti-HIV antibodies, which seemed to contribute to 1-18's potency and breadth: (1) 1-18 has an aromatic residue that mimics residue Phe43 of CD4 to target the “Phe43gp120 pocket,” a characteristic previously reported for the anti-HIV antibody, N6; (2) 1-18 contacts with the adjacent gp120 protomer, as previously observed with the anti-HIV antibody, 3BNC117, but with increased buried surface area (via its six-residue insertion in CDRH1); and (3) a larger buried surface area on gp120 than other the anti-HIV antibodies. In addition, 1-18 was reported to contact conserved residues on HIV gp120 that other anti-HIV antibodies did not contact. Schommers hypothesized that these contacts may allow 1-18 to rely less on classical CD4 binding site contacts, which may make viral escape more difficult. Nonetheless, Schommers observed that a small number of HIV strains was found to be 1-18 resistant.

Sok et al., reported that PGDM1400 antibodies showed exceptional potency (a median IC50 of 0.003 μg/ml) against a 106-virus panel, as compared with other bNAbs (e.g., PGT121, PGT128, and PGT151). Additionally, PGDM1400 antibodies were shown to also have a high neutralization breadth (83% coverage) (see Sok, Devin, et al., “Recombinant HIV envelope trimer selects for quaternary-dependent antibodies targeting the trimer apex.” Proceedings of the National Academy of Sciences 111.49 (2014): 17624-17629; van der Velden, Yme U., et al. “Diverse HIV-1 escape pathways from broadly neutralizing antibody PGDM1400 in humanized mice.” Mabs. 12.1 (2020) e1845908, which are incorporated herein by reference in their entirety).

Together, the above data suggest that administration of antibodies can be effective for treating or preventing HIV. However, the difficulties with targeting such a mutable virus are evident from the studies above, as well as studies showing that (1) when a single broadly neutralizing antibody (bNAb) was used for therapy, HIV resistance to the therapy developed within a few weeks (Bar et al, Effect of HIV Antibody VRC01 on Viral Rebound after Treatment Interruption, N. Engl. J. Med. 375, 2037-2050 (2016); Caskey et al, Viraemia suppressed in HIV-1-infected humans by broadly neutralizing antibody 3BNC117. Nature 522, 487-491 (2015); Caskey et al, Antibody 10-1074 suppresses viremia in HIV-1-infected individuals. Nat. Med. 23, 185-191 (2017); Klein et al, HIV therapy by a combination of broadly neutralizing antibodies in humanized mice, Nature 492, 118-122 (2012); Lynch et al, Virologic effects of broadly neutralizing antibody VRC01 administration during chronic HIV-1 infection, Sci. Transl. Med. 7, 319ra206 (2015); Scheid et al, HIV-1 antibody 3BNC117 suppresses viral rebound in humans during treatment interruption, Nature 535, 556-560 (2016), each of which is herein incorporated by reference in its entirety), and (2) certain antibody combinations resulted in improved viral control by preventing early development of resistance (Bar-On et al, Safety and antiviral activity of combination HIV-1 broadly neutralizing antibodies in viremic individuals, Nat. Med. 24, 1701-1707 (2018); Klein et al, 2012; Mendoza et al, Combination therapy with anti-HIV-1 antibodies maintains viral suppression, Nature 561, 479-484 (2018), each of which is herein incorporated by reference in its entirety). The viral rebound observed with some of these antibodies suggests that the antibodies may only be effective for a limited time, e.g., prior to HIV escape mutations developing.

Accordingly, treatments and prophylactic therapeutics that can avoid viral escape and remain effective for HIV neutralization are still needed. As discussed herein, the present disclosure provides technologies useful for administering polyribonucleotides encoding one or more antibody agents, e.g., anti-HIV antibody agents, to a subject. Use of the technologies and approaches described herein allows for, e.g., simultaneous production of different antibody agents from polyribonucleotides. The formats of antibody agents have been designed to minimize or eliminate the risk of immunoglobulin chain mispairing. Being able to combine multiple antibody agent formulations as described herein (e.g., including a PGDM1400 antibody agent) allows for the development of a composition (e.g., a pharmaceutical composition) that delivers multiple antibody agents together so that they can bind different epitopes of the HIV virus, thereby minimizing viral escape through mutations and increasing overall efficacy.

### II. Polyribonucleotides for Delivery of Antibody Agents

The present disclosure, among other things, utilizes RNA technologies as a modality to express antibody agents directly in a subject as a novel class of antibody-based therapeutics. In some embodiments, a polyribonucleotide as described herein encodes an immunoglobulin chain of an antibody agent.

In some embodiments, an antibody agent targets HIV. In some embodiments, an antibody agent targeting HIV specifically binds to particular epitope of an HIV polypeptide. For example, in some embodiments, an antibody agent specifically binds to an epitope encompassing the Env trimer apex or a portion thereof. See FIG. 2, from McCoy and Burton, Immunol Rev. 275.1 11-20, 2017, which is herein incorporated by reference.

In some embodiments, an antibody agent may have a binding affinity (e.g., as measured by a dissociation constant) for an HIV epitope (e.g., an epitope of the Env trimer apex) of at least about 10⁻⁴M, at least about 10⁻⁵M, at least about 10⁻⁶M, at least about 10⁻⁷M, at least about 10⁻⁸M, at least about 10⁻⁹M, or lower. In some embodiments, an HIV antibody agent selectively binds a target epitope of HIV such that binding between the HIV antibody agent and the target epitope is greater than 2-fold, greater than 5-fold, greater than 10-fold, or greater than 100-fold as compared with binding of the HIV antibody agent to a non-target epitope. In some embodiments, an HIV antibody agent may have binding affinity for an HIV epitope and also variants of said HIV epitope. Those skilled in the art will appreciate that, in some cases, binding affinity (e.g., as measured by a dissociation constant) may be influenced by non-covalent intermolecular interactions such as hydrogen bonding, electrostatic interactions, hydrophobic and Van der Waals forces between the two molecules. Alternatively or additionally, binding affinity between a ligand and its target molecule may be affected by the presence of other molecules. Those skilled in the art will be familiar with a variety of technologies for measuring binding affinity and/or dissociation constants in accordance with the present disclosure, including, e.g., but not limited to ELISAs, gel-shift assays, pull-down assays, equilibrium dialysis, analytical ultracentrifugation, surface plasmon resonance (SPR), bio-layer interferometry, grating-coupled interferometry, and spectroscopic assays.

In some embodiments, an antibody agent targeting HIV may comprise or be derived from a broadly neutralizing antibody (bNAb). In some embodiments, an antibody agent targeting HIV may be any one of the HIV-targeting antibodies described in Sok, et al., PNAS 111.49: 17624-17629, 2014; van der Velden, Yme U., et al. Mabs. 12.1: e1845908, 2020, Barouch, et al., Nature 503: 7475 224-228, 2013, Shingai, et al., Nature 503: 7475 277-280, 2013, Caskey, et al., Nature 522.7557: 487-491, 2015, Caskey et al., Nature Medicine 23.2: 185-191, 2017, Bar et al., New England Journal of Medicine 375.21: 2037-2050, 2016, Mendoza, et al., Nature 561.7724: 479-484, 2018, Gautam, Rajeev, et al., Nature Medicine 24.5: 610-616, 2018, the contents of each of which are incorporated herein by reference in their entirety for the purposes described herein.

In some embodiments, an antibody agent targeting HIV may be e.g., 1-18, PGT121, 3BNC117, b12, 10-1074, 10E8, 10E8v4, VRC01, VRC07-523-L/S, PGDM1400, fragments thereof, or combinations thereof. Exemplary anti-HIV antibodies that can be used in compositions described herein include, but are not limited to, 1-18, PGT121, 3BNC117, b12, 10-1074, 10E8, 10E8v4, VRC01, VRC07-523-L/S, PGDM1400, fragments thereof, or combinations thereof. For example, in some embodiments, a polyribonucleotide as described herein can comprise one or more heavy chain complementarity determining regions (HCDRs) (e.g., HCDR1, HCDR2, and/or HCDR3) from 1-18, PGT121, 3BNC117, b12, 10-1074, 10E8, 10E8v4, VRC01, VRC07-523-L/S, or PGDM1400. In some embodiments, a polyribonucleotide as described herein can comprise HCDR1, HCDR2, and HCDR3 from 1-18, PGT121, 3BNC117, b12, 10-1074, 10E8, 10E8v4, VRC01, VRC07-523-L/S, or PGDM1400. In some embodiments, a polyribonucleotide as described herein can comprise a heavy chain variable domain from 1-18, PGT121, 3BNC117, b12, 10-1074, 10E8, 10E8v4, VRC01, VRC07-523-L/S, or PGDM1400. In some embodiments, a polyribonucleotide as described herein can comprise one or more light chain complementarity determining regions (LCDRs) (e.g., LCDR1, LCDR2, and/or LCDR3) from 1-18, PGT121, 3BNC117, b12, 10-1074, 10E8, 10E8v4, VRC01, VRC07-523-L/S, or PGDM1400. In some embodiments, a polyribonucleotide as described herein can comprise LCDR1, LCDR2, and LCDR3 from 1-18, PGT121, 3BNC117, b12, 10-1074, 10E8, 10E8v4, VRC01, VRC07-523-L/S, or PGDM1400. In some embodiments, a polyribonucleotide as described herein can comprise a light chain variable domain from 1-18, PGT121, 3BNC117, b12, 10-1074, 10E8, 10E8v4, VRC01, VRC07-523-L/S, or PGDM1400.

In some embodiments, a plurality of polyribonucleotides that each encode an immunoglobulin chain of an antibody agent can be used to deliver (e.g., by administration to a subject) two or more antibody agents (e.g., 1-18, PGT121, 3BNC117, b12, 10-1074, 10E8, 10E8v4, VRC01, VRC07-523-L/S, or PGDM1400, or fragments or variants thereof). In some embodiments, a plurality of polyribonucleotides that each encode an immunoglobulin chain of an antibody agent can be used to deliver (e.g., by administration to a subject) three or more antibody agents (e.g., 1-18, PGT121, 3BNC117, b12, 10-1074, 10E8, 10E8v4, VRC01, VRC07-523-L/S, or PGDM1400, or fragments or variants thereof). In some embodiments, a plurality of polyribonucleotides that each encode an immunoglobulin chain of an antibody agent can be used to deliver (e.g., by administration to a subject) four or more antibody agents (e.g., 1-18, PGT121, 3BNC117, b12, 10-1074, 10E8, 10E8v4, VRC01, VRC07-523-L/S, or PGDM1400, or fragments or variants thereof). In some embodiments, a plurality of polyribonucleotides that each encode an immunoglobulin chain of an antibody agent can be used to deliver (e.g., by administration to a subject) two, three, four, five or six antibody agents (e.g., 1-18, PGT121, 3BNC117, b12, 10-1074, 10E8, 10E8v4, VRC01, VRC07-523-L/S, or PGDM1400, or fragments or variants thereof). In some embodiments, an antibody agent encoded by one or more polyribonucleotides described herein includes all or part of a PGDM1400 antibody.

Antibody agents (e.g., PGDM1400 antibody agents), as described herein, may be characterized by the amino acid sequence of one of more domains within their antibody structure. For example, an antibody agent can comprise at least one heavy (H) chain and at least one light (L) chain interconnected, e.g., by disulfide bonds. Each H chain comprises a heavy chain variable region (abbreviated herein as VH) and a heavy chain constant region. Each light chain comprises a light chain variable region (abbreviated herein as VL) and a light chain constant region. The variable regions of each light/heavy chain (VL/VH) pair form the antigen-binding domain.

Within each light or heavy chain variable domain, there are three short segments called the complementarity determining regions (“CDRs”). The six CDRs in an antibody variable domain (three in the light chain variable domain and three in the heavy chain variable domain) fold up together in 3-dimensional space to form the actual antibody binding site. The terms “LCDR1,” “LCDR2” and “LCDR3” as provided herein refer to the complementarity determining regions (CDR) 1, 2, and 3 of the variable light (L) chain of an antibody agent. In some embodiments, the light chain variable domain provided herein includes in N-terminal to C-terminal direction a LCDR1, a LCDR2 and a LCDR3. Likewise, the terms “HCDR1,” “HCDR2” and “HCDR3” as provided herein refer to the complementarity determining regions (CDR) 1, 2, and 3 of the variable heavy (H) chain of an antibody agent. In certain embodiments, the heavy chain variable domain provided herein includes in N-terminal to C-terminal direction a HCDR1, a HCDR2 and a HCDR3.

Also within the variable region, but not contained within the CDRs, are regions called the framework regions (“FR”). Thus, the complementarity determining regions (CDRs) are interspersed with the framework regions. Accordingly, each VH and VL comprises three CDRs and four FRs, arranged from amino-terminus to carboxy-terminus in the following order: FR1, CDR1, FR2, CDR2, FR3, CDR3, and FR4. Generally, framework regions are more conserved than variable regions across naturally produced antibodies.

PGDM1400 antibodies, including their nucleotide and amino acid sequences, are disclosed in WO2021/087015 and U.S. Pat. No. 10,093,720, which are herein incorporated by reference in their entirety.

In some embodiments, a PGDM1400 antibody agent comprises an HCDR1, an HCDR2, and an HCDR3 of the heavy chain variable domain having an amino acid sequence SEQ ID NO: 36. In some embodiments, an PGDM1400 antibody agent comprises an LCDR1, and LCDR2, and an LCDR3 of the light chain variable domain having an amino acid sequence SEQ ID NO: 43.

The positions of the CDRs and framework regions within the VH and VL domains of an antibody agent described herein (e.g., a PGDM1400 antibody agent) can be determined using various numbering systems known in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); AI-Lazikani et al., J. Mol. Biol., 273:927-748 (1997) ImMunoGenTics (IMGT) numbering; Lefranc, M.-P., The Immunologist, 7, 132-136 (1999); Lefranc, M. P. et al., Dev. Comp. Immunol., 27, 55-77 (2003), each of which is incorporated herein by reference). Accordingly, CDRs within PGDM1400 antibody agents within the same VH or VL domain can be determined by different numbering systems.

In some embodiments, an antibody agent (e.g., a PGDM1400 antibody agent) comprises HCDR1, HCDR2, and HCDR3 amino acid sequences of the heavy chain variable domain represented by amino acid sequence SEQ ID NO: 36 according to Kabat numbering. In some embodiments, an antibody agent (e.g., a PGDM1400 antibody agent) comprises HCDR1, HCDR2, and HCDR3 amino acid sequences of the heavy chain variable domain represented by amino acid sequence SEQ ID NO: 36 according to Chothia numbering. In some embodiments, an antibody agent (e.g., a PGDM1400 antibody agent) comprises HCDR1, HCDR2, and HCDR3 amino acid sequences of the heavy chain variable domain represented by amino acid sequence SEQ ID NO: 36 according to IMGT numbering. In some embodiments, an antibody agent (e.g., a PGDM1400 antibody agent) comprises HCDR1, HCDR2, and HCDR3 amino acid sequences of the heavy chain variable domain represented by amino acid sequence SEQ ID NO: 36 according to AbM numbering.

In some embodiments, an antibody agent (e.g., a PGDM1400 antibody agent) comprises LCDR1, LCDR2, and LCDR3 amino acid sequences of of the light chain variable domain represented by amino acid sequence SEQ ID NO: 43 according to Kabat numbering. In some embodiments, an antibody agent (e.g., a PGDM1400 antibody agent) comprises LCDR1, LCDR2, and LCDR3 amino acid sequences of of the light chain variable domain represented by amino acid sequence SEQ ID NO: 43 according to Chothia numbering. In some embodiments, an antibody agent (e.g., a PGDM1400 antibody agent) comprises LCDR1, LCDR2, and LCDR3 amino acid sequences of of the light chain variable domain represented by amino acid sequence SEQ ID NO: 43 according to IMGT numbering. In some embodiments, an antibody agent (e.g., a PGDM1400 antibody agent) comprises LCDR1, LCDR2, and LCDR3 amino acid sequences of of the light chain variable domain represented by amino acid sequence SEQ ID NO: 43 according to AbM numbering.

In some embodiments, an HCDR1 of an antibody agent (e.g., a PGDM1400 antibody agent) comprises an amino acid sequence as set out in SEQ ID NO: 18, 1484, or 1490.

In some embodiments, an HCDR2 of an antibody agent (e.g., a PGDM1400 antibody agent) comprises an amino acid sequence as set out in SEQ ID NO: 21, 1485, 1491, or 1492.

In some embodiments, an HCDR3 of an antibody agent (e.g., a PGDM1400 antibody agent) comprises an amino acid sequence as set out in SEQ ID NO: 24, 1486, or 1493.

In some embodiments, an LCDR1 of an antibody agent (e.g., a PGDM1400 antibody agent) comprises an amino acid sequence as set out in SEQ ID NO: 27 or 1487.

In some embodiments, an LCDR2 of an antibody agent (e.g., a PGDM1400 antibody agent) comprises an amino acid sequence as set out in SEQ ID NO: 30 or 1488.

In some embodiments, an LCDR3 of an antibody agent (e.g., a PGDM1400 antibody agent) comprises an amino acid sequence as set out in SEQ ID NO: 33 or 1489.

In some embodiments, an antibody agent (e.g., a PGDM1400 antibody agent) comprises one or more CDRs or any set of CDRs as shown in Table 2 below.

TABLE 2
Exemplary PGDM1400 Antibody Agent CDR Sequences

| | | |
|---|---|---|
| SEQ ID | | |
| NO | Description | Sequence |

Exemplary PGDM1400-1

| | | | |
|---|---|---|---|
| 18 | PGDM1400 HCDR1 | AA | GNTLKTYD |
| 21 | PGDM1400 HCDR2 | AA | ISHEGDKK |
| 24 | PGDM1400 HCDR3 | AA | CAKGSKHRLRDYALYDDDGALNWAVDVDYLSNLEFW |
| 27 | PGDM1400 LCDR1 | AA | HSLIHGDRNNY |
| 30 | PGDM1400 LCDR2 | AA | LAS |
| 33 | PGDM1400 LCDR3 | AA | CMQGRESPWTF |

Exemplary PGDM1400-2

| | | | |
|---|---|---|---|
| 1484 | PGDM1400 HCDR1 | AA | TYDLH |
| 1485 | PGDM1400 HCDR2 | AA | WISHEGDK |
| 1486 | PGDM1400 HCDR3 | AA | GSKHRLRDYALYDDDGALNWAVDVDYLSNLEF |
| 1487 | PGDM1400 LCDR1 | AA | KSSHSLIHGDRNNYLA |
| 1488 | PGDM1400 LCDR2 | AA | LASSRAS |
| 1489 | PGDM1400 LCDR3 | AA | MQGRESPWT |

Exemplary PGDM1400-3

| | | | |
|---|---|---|---|
| 1490 | PGDM1400 HCDR1 | AA | GNTLKTY |
| 1491 | PGDM1400 HCDR2 | AA | SHEGDK |
| 1486 | PGDM1400 HCDR3 | AA | GSKHRLRDYALYDDDGALNWAVDVDYLSNLEF |
| 1487 | PGDM1400 LCDR1 | AA | KSSHSLIHGDRNNYLA |
| 1488 | PGDM1400 LCDR2 | AA | LASSRAS |
| 1489 | PGDM1400 LCDR3 | AA | MQGRESPWT |

Exemplary PGDM1400-4

| | | | |
|---|---|---|---|
| 1484 | PGDM1400 HCDR1 | AA | TYDLH |
| 1492 | PGDM1400 HCDR2 | AA | WISHEGDKKVIVERFKA |
| 1486 | PGDM1400 HCDR3 | AA | GSKHRLRDYALYDDDGALNWAVDVDYLSNLEF |
| 1487 | PGDM1400 LCDR1 | AA | KSSHSLIHGDRNNYLA |
| 1488 | PGDM1400 LCDR2 | AA | LASSRAS |
| 1489 | PGDM1400 LCDR3 | AA | MQGRESPWT |

Exemplary PGDM1400-5

| | | | |
|---|---|---|---|
| 1490 | PGDM1400 HCDR1 | AA | GNTLKTY |
| 1491 | PGDM1400 HCDR2 | AA | SHEGDK |
| 1486 | PGDM1400 HCDR3 | AA | GSKHRLRDYALYDDDGALNWAVDVDYLSNLEF |
| 1487 | PGDM1400 LCDR1 | AA | KSSHSLIHGDRNNYLA |
| 1488 | PGDM1400 LCDR2 | AA | LASSRAS |
| 1489 | PGDM1400 LCDR3 | AA | MQGRESPWT |

Exemplary PGDM1400-6

| | | | |
|---|---|---|---|
| 18 | PGDM1400 HCDR1 | AA | GNTLKTYD |
| 21 | PGDM1400 HCDR2 | AA | ISHEGDKK |
| 1493 | PGDM1400 HCDR3 | AA | AKGSKHRLRDYALYDDDGALNWAVDVDYLSNLEF |
| 27 | PGDM1400 LCDR1 | AA | HSLIHGDRNNY |
| 30 | PGDM1400 LCDR2 | AA | LAS |
| 1489 | PGDM1400 LCDR3 | AA | MQGRESPWT |

In some embodiments, an antibody agent (e.g., a PGDM1400 antibody agent) comprises HCDR1, HCDR2, and HCDR3 amino acid sequences as set out in SEQ ID NOs: 1484, 1492, and 1486, respectively. In some embodiments, an antibody agent (e.g., a PGDM1400 antibody agent) comprises HCDR1, HCDR2, and HCDR3 amino acid sequences as set out in SEQ ID NOs: 1490, 1491, and 1486, respectively. In some embodiments, an antibody agent (e.g., a PGDM1400 antibody agent) comprises HCDR1, HCDR2, and HCDR3 amino acid sequences as set out in SEQ ID NOs: 18, 21, and 1493, respectively.

In some embodiments, an antibody agent (e.g., a PGDM1400 antibody agent) comprises HCDR1, HCDR2, HCDR3 amino acid sequences as set out in SEQ ID NOs: 18, 21, and 24, respectively. In some embodiments, an antibody agent (e.g., a PGDM1400 antibody agent) comprises HCDR1, HCDR2, and HCDR3 amino acid sequences as set out in SEQ ID NOs: 1484, 1485, and 1486, respectively.

In some embodiments, an antibody agent (e.g., a PGDM1400 antibody agent) comprises LCDR1, LCDR2, and LCDR3 amino acid sequences as set out in SEQ ID NOs: 1487, 1488, and 1489, respectively. In some embodiments, an antibody agent (e.g., a PGDM1400 antibody agent) comprises LCDR1, LCDR2, and LCDR3 amino acid sequences as set out in SEQ ID NOs: 27, 30 and 1489, respectively.

In some embodiments, an antibody agent (e.g., a PGDM1400 antibody agent) comprises LCDR1, LCDR2, LCDR3 amino acid sequences as set out in SEQ ID NOs: 27, 30, and 33, respectively.

In some embodiments, an antibody agent (e.g., a PGDM1400 antibody agent) comprises (i) HCDR1, HCDR2, and HCDR3 amino acid sequences as set out in SEQ ID NOs: 18, 21, and 24, respectively; and (ii) LCDR1, LCDR2, and LCDR3 amino acid sequences as set out SEQ ID NOs: 27, 30, and 33, respectively.

In some embodiments, an antibody agent (e.g., a PGDM1400 antibody agent) comprises (i) HCDR1, HCDR2, and HCDR3 amino acid sequences as set out in SEQ ID NOs: 1484, 1485, and 1486, respectively; and (ii) LCDR1, LCDR2, and LCDR3 amino acid sequences as set out SEQ ID NOs: 1487, 1488, and 1489, respectively.

In some embodiments, an antibody agent (e.g., a PGDM1400 antibody agent) comprises (i) HCDR1, HCDR2, and HCDR3 amino acid sequences as set out in SEQ ID NOs: 1490, 1491, and 1486, respectively; and (ii) LCDR1, LCDR2, and LCDR3 amino acid sequences as set out SEQ ID NOs: 1487, 1488, and 1489, respectively.

In some embodiments, an antibody agent (e.g., a PGDM1400 antibody agent) comprises (i) HCDR1, HCDR2, and HCDR3 amino acid sequences as set out in SEQ ID NOs: 1484, 1492, and 1486, respectively; and (ii) LCDR1, LCDR2, and LCDR3 amino acid sequences as set out SEQ ID NOs: 1487, 1488, and 1489, respectively.

In some embodiments, an antibody agent (e.g., a PGDM1400 antibody agent) comprises (i) HCDR1, HCDR2, and HCDR3 amino acid sequences as set out in SEQ ID NOs: 1490, 1491, and 1486, respectively; and (ii) LCDR1, LCDR2, and LCDR3 amino acid sequences as set out SEQ ID NOs: 1487, 1488, and 1489, respectively.

In some embodiments, an antibody agent (e.g., a PGDM1400 antibody agent) comprises (i) HCDR1, HCDR2, and HCDR3 amino acid sequences as set out in SEQ ID NOs: 18, 21, and 1493, respectively; and (ii) LCDR1, LCDR2, and LCDR3 amino acid sequences as set out SEQ ID NOs: 27, 30, and 1489, respectively.

In some embodiments, a PGDM1400 antibody agent comprises a FR1, FR2, FR3, and/or a FR4 domain of the heavy chain variable domain represented by amino acid sequence SEQ ID NO: 36. In some embodiments, an PGDM1400 antibody agent comprises a FR1, FR2, FR3, and/or a FR4 domain of the light chain variable domain represented by amino acid sequence SEQ ID NO: 43.

In some embodiments, an antibody agent (e.g., a PGDM1400 antibody agent) comprises a heavy chain FR1 comprising amino acid sequence as shown in SEQ ID NO: 1496. In some embodiments, an antibody agent (e.g., a PGDM1400 antibody agent) comprises a heavy chain FR2 comprising amino acid sequence as shown in SEQ ID NO: 1497. In some embodiments, an antibody agent (e.g., a PGDM1400 antibody agent) comprises a heavy chain FR3 comprising amino acid sequence as shown in SEQ ID NO: 1498. In some embodiments, an antibody agent (e.g., a PGDM1400 antibody agent) comprises a heavy chain FR4 comprising amino acid sequence as shown in SEQ ID NO: 1499.

In some embodiments, an antibody agent (e.g., a PGDM1400 antibody agent) comprises a light chain FR1 comprising amino acid sequence as shown in SEQ ID NO: 1500. In some embodiments, an antibody agent (e.g., a PGDM1400 antibody agent) comprises a light chain FR2 comprising amino acid sequence as shown in SEQ ID NO: 1501. In some embodiments, an antibody agent (e.g., a PGDM1400 antibody agent) comprises a light chain FR3 comprising amino acid sequence as shown in SEQ ID NO: 1502. In some embodiments, an antibody agent (e.g., a PGDM1400 antibody agent) comprises a light chain FR4 comprising amino acid sequence as shown in SEQ ID NO: 1503.

In some embodiments, an antibody agent (e.g., a PGDM1400 antibody agent) heavy chain FR1, FR2, FR3, and/or a FR4 domains comprise amino acid sequences as set out in SEQ ID NOs: 1496, 1497, 1498, and 1499 respectively. In some embodiments, an antibody agent (e.g., a PGDM1400 antibody agent) light chain FR1, FR2, FR3, and/or a FR4 domains comprise amino acid sequences as set out in SEQ ID NOs: 1500, 1501, 1502, and 1503 respectively.

In some embodiments, an antibody agent (e.g., a PGDM1400 antibody agent) comprises a VH domain that comprises HCDR1, FR1, HCDR2, and HCDR3 amino acid sequences as set out in SEQ ID NOs: 18, 1496, 21, and 24, respectively.

In some embodiments, an antibody agent (e.g., a PGDM1400 antibody agent) comprises (i) a VH domain that comprises HCDR1, FR1, HCDR2, and HCDR3 amino acid sequences as set out in SEQ ID NOs: 18, 1496, 21, and 24, respectively; and (ii) a VL domain that comprises LCDR1, LCDR2, and LCDR3 amino acid sequences as set out SEQ ID NOs: 27, 30, and 33, respectively.

In some embodiments, an antibody agent encoded by one or more polyribonucleotides provided herein includes all or part of a PGDM1400 antibody. In some embodiments, an antibody agent comprises a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof. In some embodiments, an antibody agent comprises a heavy chain variable domain comprising: (i) HCDR1 (SEQ ID NO: 18); (ii) HCDR2 (SEQ ID NO: 21); and (iii) HCDR3 (SEQ ID NO: 24). In some embodiments, an antibody agent comprises a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, an antibody agent comprises a light chain variable domain comprising: (i) LCDR1 (SEQ ID NO: 27); (ii) LCDR2 (LAS; SEQ ID NO: 30); and (iii) LCDR3 (SEQ ID NO: 33). In some embodiments, an antibody agent comprises (a) a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof; and (b) a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, an antibody agent comprises (a) a heavy chain variable domain comprising: (i) HCDR1 (SEQ ID NO: 18); (ii) HCDR2 (SEQ ID NO: 21); and (iii) HCDR3 (SEQ ID NO: 24); and (b) a light chain variable domain comprising: (i) LCDR1 (SEQ ID NO: 27); (ii) LCDR2 (LAS; SEQ ID NO: 30); and (iii) LCDR3 (SEQ ID NO: 33).

In some embodiments, a polyribonucleotide described herein encodes all or part of a PGDM1400 antibody. In some embodiments, a polyribonucleotide described herein encodes an immunoglobulin chain comprising a heavy chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof. In some embodiments, a polyribonucleotide described herein encodes an immunoglobulin chain comprising a heavy chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24). In some embodiments, a polyribonucleotide described herein encodes an immunoglobulin chain comprising a light chain variable domain, wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain comprising a light chain variable domain, wherein the light chain variable domain comprises: (i) LCDR1 (SEQ ID NO: 27); (ii) LCDR2 (LAS; SEQ ID NO: 30); and (iii) LCDR3 (SEQ ID NO: 33). In some embodiments, a polyribonucleotide described herein encodes an immunoglobulin chain comprising a heavy chain variable domain and a light chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof; and the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain comprising a heavy chain variable domain and a light chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24); and the light chain variable domain comprises: (i) LCDR1 (SEQ ID NO: 27); (ii) LCDR2 (LAS; SEQ ID NO: 30); and (iii) LCDR3 (SEQ ID NO: 33). In some embodiments, a polyribonucleotide described herein encodes two immunoglobulin chains: a first immunoglobulin chain comprising a heavy chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof; and a second immunoglobulin chain comprising a light chain variable domain, wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes two immunoglobulin chains: a first immunoglobulin chain comprising a heavy chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24); and a second immunoglobulin chain comprising a light chain variable domain, wherein the light chain variable domain comprises: (i) LCDR1 (SEQ ID NO: 27); (ii) LCDR2 (LAS; SEQ ID NO: 30); and (iii) LCDR3 (SEQ ID NO: 33).

In some embodiments, an antibody agent encoded by one or more polyribonucleotides provided herein comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, an antibody agent encoded by one or more polyribonucleotides provided herein comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, an antibody agent comprises a heavy chain variable domain represented by SEQ ID NO: 36. In some embodiments, an antibody agent comprises a light chain variable domain represented by SEQ ID NO: 43.

In some embodiments, an antibody agent (e.g., a PGDM1400 antibody agent) encoded by one or more polyribonucleotides provided herein comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 1494. In some embodiments, an antibody agent (e.g., a PGDM1400 antibody agent) encoded by one or more polyribonucleotides provided herein comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, an antibody agent comprises a heavy chain variable domain represented by SEQ ID NO: 1494. In some embodiments, an antibody agent comprises a light chain variable domain represented by SEQ ID NO: 43.

In some embodiments, an antibody agent (e.g., a PGDM1400 antibody agent) encoded by one or more polyribonucleotides provided herein comprises a heavy chain variable domain that comprises one or more mutations relative to an amino acid sequence represented by SEQ ID NO: 1494. In some embodiments, substitution mutations are present at residues 3 and/or 5 relative to SEQ ID NO: 1494. In some embodiments, a substitution mutation at residue 3 results in a positively charged amino acid being present at residue 3 relative to SEQ ID NO: 1494. Positively charged amino acids include Lys (K), Arg (R), and His (H). In some embodiments, a substitution mutation at residue 3 relative to SEQ ID NO: 1494 comprises Q3H. In some embodiments, a substitution mutation at residue 5 results in a polar amino acid being present at residue 5 relative to SEQ ID NO: 1494. Polar amino acids include Ser (S), Thr (T), Tyr (Y), Asn (N), and Gln (Q). In some embodiments, a substitution mutation at residue 5 relative to SEQ ID NO: 1494 comprises V5T.

In some embodiments, an antibody agent (e.g., a PGDM1400 antibody agent) encoded by one or more polyribonucleotides provided herein comprises a heavy chain variable domain that comprises SEQ ID NO: 1494 with substitution mutations Q3H and V5T.

In some embodiments, a polyribonucleotide described herein encodes an immunoglobulin chain comprising a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a polyribonucleotide described herein encodes an immunoglobulin chain comprising a light chain variable domain having at least 80%, 85%, 90%, 91%, 92%, 93%, 94%, 95%, 96%, 97%, 98%, or 99% sequence identity to an amino acid sequence represented by SEQ ID NO: 43. In some embodiments, a polyribonucleotide described herein encodes an immunoglobulin chain comprising a heavy chain variable domain and a light chain variable domain, wherein the heavy chain variable domain has at least 80%, 85%, 90%, 91%, 92%, 93%, 94%, 95%, 96%, 97%, 98%, or 99% sequence identity to an amino acid sequence represented by SEQ ID NO: 36, and wherein the light chain variable domain has at least 80%, 85%, 90%, 91%, 92%, 93%, 94%, 95%, 96%, 97%, 98%, or 99% sequence identity to an amino acid sequence represented by SEQ ID NO: 43.

In some embodiments, a polyribonucleotide as described herein encodes an immunoglobulin chain of an antibody agent, where the immunoglobulin chain comprises a heavy chain variable (VH) domain. In some embodiments, a VH domain comprises a VH domain of a PGDM1400 antibody. In some embodiments, a polyribonucleotide encodes a VH domain of an antibody selected from: PGT121, 3BNC117, b12, 10-1074, 10-1074-LS, 10E8, VRC01, VRC07-523, or 1-18 (e.g., as described herein).

In some embodiments, a polyribonucleotide comprises a VH domain encoding sequence that comprises (a) an HCDR1-encoding sequence that comprises a ribonucleic acid sequence according to SEQ ID NO: 19; (b) an HCDR2-encoding sequence that comprises a ribonucleic acid sequence according to SEQ ID NO: 22; (c) an HCDR3-encoding sequence that comprises a ribonucleic acid sequence according to SEQ ID NO: 25, or (d) a combination thereof. In some embodiments, a polyribonucleotide comprises a VH domain encoding sequence that comprises (a) an HCDR1-encoding sequence that comprises a ribonucleic acid sequence according to SEQ ID NO: 19; (b) an HCDR2-encoding sequence that comprises a ribonucleic acid sequence according to SEQ ID NO: 22; and (c) an HCDR3-encoding sequence that comprises a ribonucleic acid sequence according to SEQ ID NO: 25. In some embodiments, a polyribonucleotide encodes a VH domain, and comprises a VH-encoding sequence that is at least 90%, 91%, 92%, 93%, 94%, 95%, 96%, 97%, 98%, or 99% to identical to SEQ ID NO: 37, 39, or 41. In some embodiments, a polyribonucleotide encodes a VH domain, and comprises a VH-encoding sequence according to SEQ ID NO: 37, 39, or 41.

In some embodiments, a polyribonucleotide as described herein comprises an immunoglobulin chain of an antibody agent, where the immunoglobulin chain comprises a light chain variable (VL) domain. In some embodiments, a VL domain comprises the VL domain of a PGDM1400 antibody. In some embodiments, a polyribonucleotide encodes a VL domain of an antibody selected from: PGT121, 3BNC117, b12, 10-1074, 10-1074-LS, 10E8, VRC01, VRC07-523, or 1-18 (e.g., as described herein).

In some embodiments, a polyribonucleotide comprises one or more coding regions that encode an immunoglobulin chain of an antibody agent, where the immunoglobulin chain comprises a light chain variable (VL) domain. In some embodiments, a polyribonucleotide comprises a VL domain encoding sequence that comprises (a) an LCDR1-encoding sequence that comprises a ribonucleic acid sequence according to SEQ ID NO: 28; (b) an LCDR2-encoding sequence that comprises a ribonucleic acid sequence according to SEQ ID NO: 31; (c) an LCDR3-encoding sequence that comprises a ribonucleic acid sequence according to SEQ ID NO: 34; or (d) a combination thereof. In some embodiments, a polyribonucleotide comprises a VL domain encoding sequence that comprises (a) an LCDR1-encoding sequence that comprises a ribonucleic acid sequence according to SEQ ID NO: 28; (b) an LCDR2-encoding sequence that comprises a ribonucleic acid sequence according to SEQ ID NO: 31; and (c) an LCDR3-encoding sequence that comprises a ribonucleic acid sequence according to SEQ ID NO: 34. In some embodiments, a polyribonucleotide encodes a VL domain, and comprises a VL-encoding sequence that is at least 90%, 91%, 92%, 93%, 94%, 95%, 96%, 97%, 98%, or 99% identical to SEQ ID NO: 44 or 46. In some embodiments, a polyribonucleotide encodes a VL domain, and comprises a VL-encoding sequence according to SEQ ID NO: 44 or 46.

In some embodiments, an antibody agent is formed by one, two, three, or four immunoglobulin chains.

In some embodiments, a polyribonucleotide as described herein encodes a single immunoglobulin chain. In some embodiments, a first polyribonucleotide encodes a first immunoglobulin chain of an antibody agent. In some embodiments, a first polyribonucleotide encodes a first immunoglobulin chain of an antibody agent and a second polyribonucleotide encodes a second immunoglobulin chain of the antibody agent. In some embodiments, a first polyribonucleotide encodes a first immunoglobulin chain of an antibody agent, a second polyribonucleotide encodes a second immunoglobulin chain of the antibody agent, and a third polyribonucleotide encodes a third immunoglobulin chain of the antibody agent. In some embodiments, a first polyribonucleotide encodes a first immunoglobulin chain of an antibody agent, a second polyribonucleotide encodes a second immunoglobulin chain of the antibody agent, a third polyribonucleotide encodes a third immunoglobulin chain of the antibody agent, and a fourth polyribonucleotide encodes a fourth immunoglobulin chain of the antibody agent.

In some embodiments, a polyribonucleotide as described herein encodes two immunoglobulin chains. In some embodiments, a single polyribonucleotide can include a first coding region that encodes a first immunoglobulin chain of an antibody and a second coding region that encodes a second immunoglobulin chain of the antibody. In some embodiments, the first coding region and the second coding region are separated by an internal ribosome entry sides (IRES), an internal promoter, or a peptide sequence, such as “self-cleaving” 2A or 2A-like sequences (see, e.g., Szymczak et al. Nat Biotechnol 22:589, May 2004; ePub Apr. 4 2004, which is herein incorporated by reference) to yield the first immunoglobulin chain and the second immunoglobulin chain from the single polyribonucleotide.

Antibody agents encoded by one or more polyribonucleotides described herein may be in various formats described herein. Exemplary types of antibody agents include, but are not limited to, monoclonal antibodies or polyclonal antibodies. In some embodiments, an antibody agent may include one or more sequence elements that are humanized, chimeric, etc., as is known in the art. An antibody agent utilized in accordance with the present disclosure, in some embodiments, is in a format selected from, but not limited to, intact IgG, IgA, IgG, IgE or IgM antibodies; bi- or multi-specific antibodies (e.g., Zybodies®, etc.); CrossMabs (e.g., CrossMab^{CH1-CLx}; CrossMab^{CH1-CLcv}; bispecific CrossMab^{CH1-CLx} with knob-in-holes); antibody fragments such as Fab fragments, Fab′ fragments, F(ab′)2 fragments, Fd′ fragments, Fd fragments, and isolated complementarity determining regions (CDRs) or sets thereof; single chain Fvs (scFvs); scFv-Fc fusions; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, immunoglobulin chains and/or fragments of such antibodies may be used in combination, e.g., a scFv-Fc arm with a conventional antibody arm.

Exemplary formats that may be used in accordance with the present disclosure are described further below.

### A. Conventional Antibody

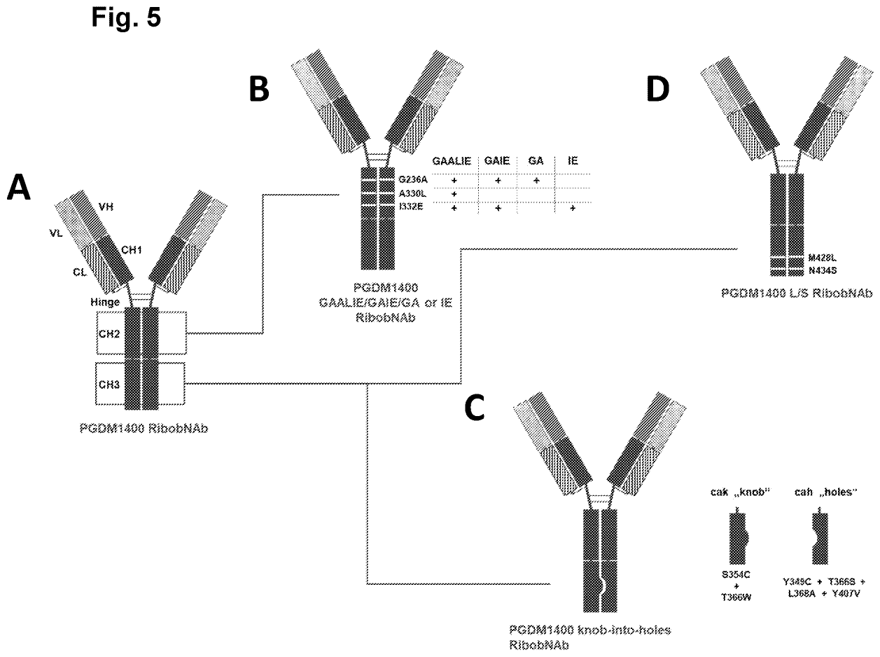

In some embodiments, polyribonucleotides described herein can be used to express a conventional antibody. As used herein, a “conventional antibody” refers to an antibody agent that includes two heavy chains and two light chains (see e.g., FIG. 5, Panel A). Each heavy chain includes a heavy chain variable domain operably linked to one or more heavy chain constant domains. In some embodiments, one or more heavy chain constant domains comprise a CH1 domain, a hinge domain, a CH₂ domain, a CH₃ domain, or a combination thereof. In some instances, one or more heavy chain constant domains comprise a CH1 domain, a hinge domain, a CH₂ domain, a CH₃ domain, a CH₄ domain, or a combination thereof. Each light chain includes a light chain variable domain operably linked to a light chain constant domain.

Typically, a heavy chain variable domain and a light chain variable domain can be further subdivided into regions of variability, termed complementarity determining regions (CDRs), interspersed with regions that are more conserved, termed framework regions (FR). Such heavy chain variable domains and light chain variable domains can each include, e.g., three CDRs and four framework regions, arranged from amino-terminus to carboxyl-terminus in the following order: FR1, CDR1, FR2, CDR2, FR3, CDR3, FR4, one or more of which can be engineered as described herein. The CDRs in a heavy chain are designated “HCDR1”, “HCDR2”, and “HCDR3”, respectively, and the CDRs in a light chain are designated “LCDR1”, “LCDR2”, and “LCDR3”.

A conventional antibody as described herein may comprise any one of the five major classes of antibodies: IgA, IgD, IgE, IgG, and IgM. In some embodiments, a conventional antibody comprises an IgG or IgA antibody. In some embodiments, a conventional antibody described herein comprises a particular isotype selected from the group of IgA and IgG isotypes: IgG1, IgG2, IgG3, IgG4, IgA1, and IgA2. Additionally, in some embodiments, a conventional antibody may include any particular heavy chain constant domains that correspond to the different classes of immunoglobulins, which include a, b, E, y, and p, respectively. In some embodiments, a conventional antibody is an intact IgG1 antibody or other antibody class or isotype as described herein. (see, e.g., Hudson et al., Nat. Med., 9:129-134 (2003); Pluckthun, The Pharmacology of Monoclonal Antibodies, vol. 113, pp. 269-315 (1994); Hollinger et al., Proc. Natl. Acad. Sci. USA, 90: 6444-6448 (1993); WO93/01161; and U.S. Pat. Nos. 5,571,894, 5,869,046, 6,248,516, and 5,587,458, each of which are herein incorporated by reference). In addition to the various isotypes, allelic variation is present among the IgG subclasses, giving rise to allotypic variants or allotypes. An IgG antibody agent as described herein may comprises a particular allotype, including but not limited to G1m3, G1m17, G1m17,1 or G1m17,1,2 or G1m3,1 (see Vidarsson et al., Front. Immunol, 5(520): 1-17, 2014, which is incorporated herein by reference in its entirety).

The Fc region of conventional antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, conventional antibodies produced and/or utilized in accordance with the present invention include glycosylated Fc domains, including Fc domains with modified or engineered such glycosylation. In some embodiments, conventional antibodies are naturally produced (e.g., generated by an organism reacting to an antigen), or produced by recombinant engineering, chemical synthesis, or other artificial system or methodology. In some embodiments, a conventional antibody is polyclonal; in some embodiments, a conventional antibody is monoclonal. In some embodiments, a conventional antibody has constant region sequences that are characteristic of mouse, rabbit, primate, or human antibodies. In some embodiments, a conventional antibody sequence elements are humanized, primatized, chimeric, etc., as is known in the art.

A conventional antibody as described herein is an antibody having a structure substantially similar to a native antibody structure or having heavy chains that contain an Fc region as defined herein.

In some embodiments, a conventional antibody encoded by one or more polyribonucleotides provided herein includes all or part of a PGDM1400 antibody. In some embodiments, a conventional antibody comprises a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof. In some embodiments, a conventional antibody comprises a heavy chain variable domain comprising (i) HCDR1 (SEQ ID NO: 18); (ii) HCDR2 (SEQ ID NO: 21); and (iii) HCDR3 (SEQ ID NO: 24). In some embodiments, a conventional antibody comprises a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a conventional antibody comprises a light chain variable domain comprising: (i) LCDR1 (SEQ ID NO: 27); (ii) LCDR2 (LAS; SEQ ID NO: 30); and (iii) LCDR3 (SEQ ID NO: 33). In some embodiments, a conventional antibody comprises (a) a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof; and (b) a light chain variable domain comprising: ((i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a conventional antibody comprises (a) a heavy chain variable domain comprising: (i) HCDR1 (SEQ ID NO: 18); (ii) HCDR2 (SEQ ID NO: 21); and (iii) HCDR3 (SEQ ID NO: 24); and (b) a light chain variable domain comprising: (i) LCDR1 (SEQ ID NO: 27); (ii) LCDR2 (LAS; SEQ ID NO: 30); and (iii) LCDR3 (SEQ ID NO: 33).

In some embodiments, a polyribonucleotide described herein encodes an immunoglobulin chain of a conventional antibody, wherein the immunoglobulin chain comprises a heavy chain variable domain, and wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof. In some embodiments, a polyribonucleotide described herein encodes an immunoglobulin chain of a conventional antibody, wherein the immunoglobulin chain comprises a heavy chain variable domain, and wherein the heavy chain variable domain comprises: (i) HCDR1 (SEQ ID NO: 18); (ii) HCDR2 (SEQ ID NO: 21); and (iii) HCDR3 (SEQ ID NO: 24). In some embodiments, a polyribonucleotide described herein encodes an immunoglobulin chain of a conventional antibody, wherein the immunoglobulin chain comprises a light chain variable domain, and wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain of a conventional antibody, wherein the immunoglobulin chain comprises a light chain variable domain, and wherein the light chain variable domain comprises: (i) LCDR1 (SEQ ID NO: 27); (ii) LCDR2 (LAS; SEQ ID NO: 30); and (iii) LCDR3 (SEQ ID NO: 33). In some embodiments, a polyribonucleotide described herein encodes two immunoglobulin chains of a conventional antibody: a first immunoglobulin chain comprising a heavy chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof; and a second immunoglobulin chain comprising a light chain variable domain, wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes two immunoglobulin chains of a conventional antibody: a first immunoglobulin chain comprising a heavy chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24); and a second immunoglobulin chain comprising a light chain variable domain, wherein the light chain variable domain comprises: (i) LCDR1 (SEQ ID NO: 27); (ii) LCDR2 (LAS; SEQ ID NO: 30); and (iii) LCDR3 (SEQ ID NO: 33).

In some embodiments, a conventional antibody encoded by one or more polyribonucleotides provided herein comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a conventional antibody encoded by one or more polyribonucleotides provided herein comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a conventional antibody comprises a heavy chain variable domain represented by SEQ ID NO: 36. In some embodiments, conventional antibody comprises a light chain variable domain represented by SEQ ID NO: 43.

In some embodiments, a conventional antibody encoded by one or more polyribonucleotides provided herein comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 1494. In some embodiments, a conventional antibody encoded by one or more polyribonucleotides provided herein comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a conventional antibody comprises a heavy chain variable domain represented by SEQ ID NO: 1494. In some embodiments, conventional antibody comprises a light chain variable domain represented by SEQ ID NO: 43.

In some embodiments, an antibody agent (e.g., a PGDM1400 antibody agent) encoded by one or more polyribonucleotides provided herein comprises a heavy chain variable domain that comprises one or more mutations relative to an amino acid sequence represented by SEQ ID NO: 1494. In some embodiments, substitution mutations are present at residues 3 and/or 5 relative to SEQ ID NO: 1494. In some embodiments, a substitution mutation at residue 3 results in a positively charged amino acid being present at residue 3 relative to SEQ ID NO: 1494. Positively charged amino acids include Lys (K), Arg (R), and His (H). In some embodiments, a substitution mutation at residue 3 relative to SEQ ID NO: 1494 comprises Q3H. In some embodiments, a substitution mutation at residue 5 results in a polar amino acid being present at residue 5 relative to SEQ ID NO: 1494. Polar amino acids include Ser (S), Thr (T), Tyr (Y), Asn (N), and Gln (Q). In some embodiments, a substitution mutation at residue 5 relative to SEQ ID NO: 1494 comprises V5T.

In some embodiments, an antibody agent (e.g., a PGDM1400 antibody agent) encoded by one or more polyribonucleotides provided herein comprises a heavy chain variable domain that comprises SEQ ID NO: 1494 with substitution mutations Q3H and V5T.

In some embodiments, a polyribonucleotide described herein encodes an immunoglobulin chain of a conventional antibody, wherein the immunoglobulin chain comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a polyribonucleotide described herein encodes an immunoglobulin chain of a conventional antibody, wherein the immunoglobulin chain comprises a light chain variable domain having at least 80%, 85%, 90%, 91%, 92%, 93%, 94%, 95%, 96%, 97%, 98%, or 99% sequence identity to an amino acid sequence represented by SEQ ID NO: 43.

Conventional antibodies encoded by one or more polyribonucleotides as described herein may comprise one or more heavy chain constant domains. In some embodiments, one or more heavy chain constant domains comprise a CH₃ domain. In some embodiments, a conventional antibody encoded by one or more polyribonucleotides as described herein comprises a CH₃ domain that comprises a G1m3, G1m17, or a G1m17,1 allotype. In some embodiments, a conventional antibody encoded by one or more polyribonucleotides as described herein comprises a CH₃ domain having an amino acid sequence at least 90%, 91%, 92%, 93%, 94%, 95%, 96%, 97%, 98%, or 99% identical to a sequence represented in any one of SEQ ID NOs: 113, 116, 119, 122, 125, 128, 131, 134, 137, 140, 143, 146, or 1495. In some embodiments, a conventional antibody encoded by one or more polyribonucleotides as described herein comprises a CH₃ domain having an amino acid sequence represented in any one of SEQ ID NOs: 113, 116, 119, 122, 125, 128, 131, 134, 137, 140, 143, 146, or 1495. In some embodiments, a CH₃ domain comprises amino acid sequence SEQ ID NO: 1495 with substitution mutations D16E and L18M.

Conventional antibodies encoded by one or more polyribonucleotides as described herein may comprise one or more heavy chain constant domains comprising an amino acid modification (e.g., a substitution or deletion) at one or more amino acid positions. For example, a conventional antibody encoded by one or more polyribonucleotides as described herein may include an L/S mutation within a CH₃ region (for enhanced FcRn binding) (see Zalevsky J et al. Nat Biotechnol. 2010, which is herein incorporated by reference). Such mutations are noted as M428L and N434S according to EU numbering (i.e., M88L and N94S within the CH₃ domain, e.g., SEQ ID NO: 113, 116, or 1495), and referred to herein as “LS” or “L/S” (see e.g., FIG. 5, Panel D). In some embodiments, a conventional antibody encoded by one or more polyribonucleotides as described herein comprises an E294 deletion (for Fc hypersialylation) (see Bas M et al. J Immunol 2019, which is herein incorporated by reference.

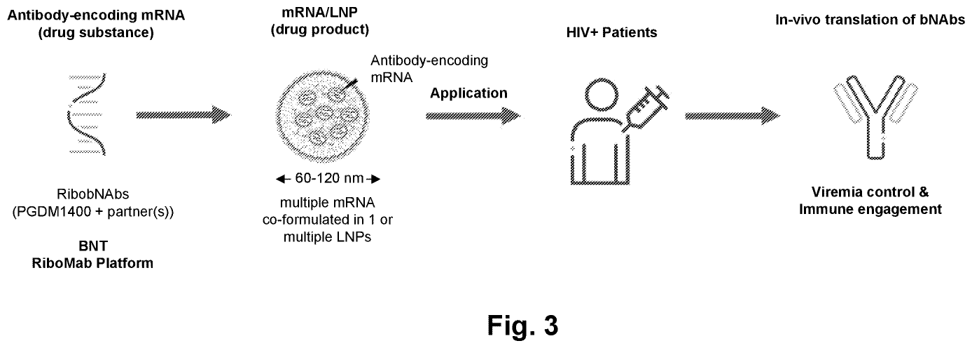

The present disclosure also provides technologies that can be used to express an antibody agent, e.g., as illustrated in FIG. 3 or described in Stadler et al. (2016) Oncoimmunology 5(3): e1091555; and/or in Stadler et al. (2017) Nature Medicine 23(7): 815-817. Challenges exist in producing multiple antibody agents from a single composition (e.g., a composition comprising polyribonucleotides sufficient to encode multiple antibody agents), particularly because the random pairing of different antibody heavy and light chains can yield undesired antibody species. Due to the presence of mispaired byproducts, and significantly reduced production yields, sophisticated purification procedures are required to isolate the desired antibody agent in those situations (see e.g., Morrison, S. L., Nature Biotech. 25, 1233-1234, 2007, which is herein incorporated by reference). In general, the same problem of mispaired byproducts remains if recombinant expression techniques are used. One approach to solve the problem of mispaired byproducts is known as “knob-into-holes technology” (KIH), which aims to force the pairing of two different antibody heavy chains by introducing mutations into the CH₃ domains to modify the contact interface. On one chain bulky amino acids were replaced by amino acids with short side chains to create a “hole” and amino acids with large side chains were introduced into the other CH₃ domain, to create a “knob”. By co-expressing these two heavy chains with two light chains, high yields of heterodimer formation versus homodimer was observed (see Ridgway, J. B., et al, Protein Eng. 9, 617-621, 1996; and WO 96/027011, which are herein incorporated by reference). In some embodiments, antibody agents described herein utilize KIH technology as described in, e.g., WO 1998/050431, which is herein incorporated by reference in its entirety. As described herein, an antibody agent may comprise certain mutations that utilize KIH technology that include, but are not limited to, a CH₃ modification. In some embodiments, an antibody agent comprises a CH₃ domain comprising one or more of the following mutations: Y349C, T366S, L368A, and Y407V (according to EU numbering). In some embodiments, an antibody agent comprises a CH₃ domain, wherein the CH₃ domain comprises each of the following mutations: Y349C, T366S, L368A, and Y407V (according to EU numbering). Such a combination of mutations is referred to herein as “cah”. In some embodiments, an antibody agent comprises a CH₃ domain comprising one or more mutations selected from: S354C and T366W (according to EU numbering). In some embodiments, an antibody agent comprises a CH₃ domain comprising each of the following mutations: S354C and T366W (according to EU numbering). Such a combination of CH₃ mutations is referred to herein as “cak”.

Accordingly, in some embodiments, a conventional antibody encoded by one or more polyribonucleotides as described herein comprises a CH₃ domain comprising one or more of the following mutations: Y349C, T366S, L368A, and Y407V (according to EU numbering). In some embodiments, a conventional antibody encoded by one or more polyribonucleotides as described herein comprises a CH₃ domain comprising one or more mutations selected from: S354C and T366W (according to EU numbering).

In some embodiments, a polyribonucleotide encodes a CH₃ domain that comprises one of the following substitution mutations: M428L, N434S, or a combination thereof (e.g., an “L/S” mutation). In some embodiments, a polyribonucleotide comprises a CH₃ ribonucleic acid sequence that comprises any one of SEQ ID NOs: 117 and 135. In some embodiments, a polyribonucleotide encodes a CH₃ domain that comprises one or more of the following substitution mutations: Y349C, T366S, L368A, and Y407V (according to EU numbering). In some embodiments, a polyribonucleotide comprises a ribonucleic acid sequence according to SEQ ID NO: 120 and 138. In some embodiments, a polyribonucleotide encodes a CH₃ domain that comprises one or both of the following substitution mutations: S354C and T366W (according to EU numbering). In some embodiments, a polyribonucleotide comprises a ribonucleic acid sequence according to SEQ ID NO: 126 and 144.

In some embodiments, a polyribonucleotide encodes an immunoglobulin chain that comprises a VH domain operably linked to one or more constant domains, where the one or more constant domains comprise a CH₃ domain. In some embodiments, a polyribonucleotide comprises a ribonucleic acid sequence according to SEQ ID NO: 114. In some embodiments, polyribonucleotide comprises a CH₃ ribonucleic acid sequence that encodes the CH₃ domain that comprises a G1m3, G1m17, or a G1m17,1 allotype. In some embodiments, a polyribonucleotide comprises a CH₃ ribonucleic acid sequence that comprises any one of SEQ ID NOs: 114 and 132.

In some embodiments, a conventional antibody encoded by one or more polyribonucleotides as described herein comprises a CH₁ domain. In some embodiments, a conventional antibody encoded by one or more polyribonucleotides as described herein comprises a CH₁ domain that comprises a G1m3 allotype. In some embodiments, a conventional antibody encoded by one or more polyribonucleotides as described herein comprises a CH₁ domain that comprises a G1m17 allotype. In some embodiments, a conventional antibody encoded by one or more polyribonucleotides as described herein comprises a CH₁ domain that comprises an amino acid sequence represented in SEQ ID NO: 76 or 81.

In some embodiments, a polyribonucleotide encodes an immunoglobulin chain that comprises a VH domain operably linked to one or more constant domains, where the one or more constant domains comprise a CH₁ domain. In some embodiments, a polyribonucleotide comprises a CH₁ ribonucleic acid sequence according to SEQ ID NO: 77 or 79. In some embodiments, a polyribonucleotide encodes a CH₁ domain that comprises a G1m3 allotype. In some embodiments, a polyribonucleotide encodes a CH₁ domain that comprises a G1m17 allotype. In some embodiments, a polyribonucleotide encodes a CH₁ ribonucleic acid sequence according to SEQ ID NO: 77, 79, or 82.

In some embodiments, a polyribonucleotide encodes a CH₁ domain that comprises one or more mutations. In some embodiments, a polyribonucleotide encodes a CH₁ domain that comprises the addition of one or more serine residues. In some embodiments, a polyribonucleotide encodes a CH₁ domain that comprises addition of two additional serine residues (referred to herein as “SS”). In some embodiments, a polyribonucleotide encodes a CH₁ ribonucleic acid sequence that is at least 90%, 91%, 92%, 93%, 94%, 95%, 96%, 97%, 98%, or 99% identical to SEQ ID NO: 91 or 94. In some embodiments, a polyribonucleotide encodes a CH₁ ribonucleic acid sequence represented in SEQ ID NO: 91 or 94. In some embodiments, a polyribonucleotide encodes a CH₁ domain that comprises one or more charge variant mutations. In some embodiments, a polyribonucleotide encodes a CH₁ domain comprising one or more substitution mutations selected from: K147E, K213D, or a combination thereof. In some embodiments, a polyribonucleotide encodes a CH₁ ribonucleic acid sequence that has at least 90%, 91%, 92%, 93%, 94%, 95%, 96%, 97%, 98%, or 99% sequence identity to SEQ ID NO: 85 or 88. In some embodiments, a polyribonucleotide encodes a CH₁ ribonucleic acid sequence according to SEQ ID NO: 85 or 88.

In some embodiments, a conventional antibody encoded by one or more polyribonucleotides as described herein comprises a hinge domain. In some embodiments, a conventional antibody encoded by one or more polyribonucleotides as described herein comprises a hinge domain that comprises an amino acid sequence represented in SEQ ID NO: 161 (herein referred to as “hinge” in Tables 2 and 4).

In some embodiments, a polyribonucleotide encodes a hinge domain. In some embodiments, a polyribonucleotide encodes a hinge ribonucleic acid sequence that represented in SEQ ID NO: 162. In some embodiments, a polyribonucleotide encodes a hinge domain that comprises an amino acid modification that comprises a deletion of one or more amino acid residues. In some embodiments, a polyribonucleotide encodes a hinge domain an amino acid modification that comprises a deletion of amino acid residues EPKSC in a conventional Ig hinge domain (represented in SEQ ID NO: 161). Such a modification is referred to herein as “Hinge_del” or “ΔEPKSC”. In some embodiments, a polyribonucleotide encodes a hinge ribonucleic acid sequence represented in SEQ ID NO: 168. In some embodiments, a polyribonucleotide encodes a hinge domain that comprises an amino acid modification that comprises a C220S mutation (according to EU numbering). Such a mutated hinge domain is referred to herein as “Hinge_S” or “C/S”. In some embodiments, a polyribonucleotide encodes a hinge ribonucleic acid sequence represented in SEQ ID NO: 165.

In some embodiments, a conventional antibody encoded by one or more polyribonucleotides as described herein comprises a CH₂ domain. In some embodiments, a conventional antibody encoded by one or more polyribonucleotides as described herein comprises a CH₂ domain having an amino acid sequence at least 90%, 91%, 92%, 93%, 94%, 95%, 96%, 97%, 98%, or 99% identical to represented in SEQ ID NO: 96. In some embodiments, a conventional antibody encoded by one or more polyribonucleotides as described herein comprises a CH₂ domain having an amino acid sequence represented in SEQ ID NO: 96.

In some embodiments, a conventional antibody encoded by one or more polyribonucleotides as described herein comprises a CH₂ domain having one or more mutations (e.g., with respect to SEQ ID NO: 96). For example, in some embodiments, a conventional antibody encoded by one or more polyribonucleotides as described herein comprises one or more of the following mutations: G236A, A330L, and I332E (according to EU numbering). In some embodiments, a conventional antibody encoded by one or more polyribonucleotides as described herein comprises the following mutations: G236A, A330L, and I332E (according to EU numbering), referred to herein as “GAALIE”. Such mutations in the CH₂ domain have been associated with increased affinity to Fc receptors FcgRIIA and FcgRIII for enhanced antibody effector function.

In some embodiments, a conventional antibody encoded by one or more polyribonucleotides as described herein comprises a CH₂ domain having an amino acid sequence that is at least 90%, 91%, 92%, 93%, 94%, 95%, 96%, 97%, 98%, or 99% identical to SEQ ID NO: 99 or 102. In some embodiments, a conventional antibody encoded by one or more polyribonucleotides as described herein comprises a CH₂ domain having an amino acid sequence represented in SEQ ID NO: 99 or 102.

In some embodiments, a conventional antibody encoded by one or more polyribonucleotides as described herein comprises one or more mutations selected from: G236A and I332E (according to EU numbering). In some embodiments, a conventional antibody encoded by one or more polyribonucleotides as described herein comprises the mutations selected from: G236A and I332E (according to EU numbering), referred to herein as “GAIE”. In some embodiments, a conventional antibody encoded by one or more polyribonucleotides as described herein comprises a CH₂ domain having an amino acid sequence at least 90%, 91%, 92%, 93%, 94%, 95%, 96%, 97%, 98%, or 99% identical to SEQ ID NO: 104. In some embodiments, a conventional antibody encoded by one or more polyribonucleotides as described herein comprises a CH₂ domain having an amino acid sequence represented in SEQ ID NO: 104.

In some embodiments, a conventional antibody encoded by one or more polyribonucleotides as described herein comprises a mutation: G236A (according to EU numbering), referred to herein as “GA”. In some embodiments, a conventional antibody encoded by one or more polyribonucleotides as described herein comprises a CH₂ domain having an amino acid sequence at least 90%, 91%, 92%, 93%, 94%, 95%, 96%, 97%, 98%, or 99% identical to SEQ ID NO: 107. In some embodiments, a conventional antibody encoded by one or more polyribonucleotides as described herein comprises a CH₂ domain having an amino acid sequence represented in SEQ ID NO: 107.

In some embodiments, a conventional antibody encoded by one or more polyribonucleotides as described herein comprises a mutation: I332E (according to EU numbering), referred to herein as “IE”. In some embodiments, a conventional antibody encoded by one or more polyribonucleotides as described herein comprises a CH₂ domain having an amino acid sequence at least 90%, 91%, 92%, 93%, 94%, 95%, 96%, 97%, 98%, or 99% identical to SEQ ID NO: 110. In some embodiments, a conventional antibody encoded by one or more polyribonucleotides as described herein comprises a CH₂ domain having an amino acid sequence represented in SEQ ID NO: 110.

In some embodiments, a polyribonucleotide encodes an immunoglobulin chain that comprises a VH domain operably linked to one or more constant domains, wherein the one or more constant domains comprise a CH₂ domain. In some embodiments, a polyribonucleotide comprises a ribonucleic acid sequence according to SEQ ID NO: 97. In some embodiments, a CH₂ ribonucleic acid encodes a CH₂ domain with one or more amino acid substitution mutations. For example, in some embodiments, a CH₂ ribonucleic acid sequence encodes one or more of the following mutations: G236A, A330L, and I332E (according to EU numbering), referred to herein as “GAALIE”. Such mutations in the CH₂ domain have been associated with increased affinity to Fc receptors FcgRIIA and FcgRIII for enhanced antibody effector function. In some embodiments, a CH₂ ribonucleic sequence comprises or consists of a sequence according to SEQ ID NO: 100 or 103. In some embodiments, a CH₂ ribonucleic acid sequence encodes the one or more of the following mutation: G236A and I332E (according to EU numbering), referred to herein as “GAIE”. In some embodiments, a CH₂ ribonucleic sequence comprises of a sequence according to SEQ ID NO: 105. In some embodiments, a CH₂ ribonucleic acid sequence encodes the mutation G236A (according to EU numbering), referred to herein as “GA”. In some embodiments, a CH₂ ribonucleic sequence comprises of a sequence according to SEQ ID NO: 108. In some embodiments, a CH₂ ribonucleic acid sequence encodes the mutation: I332E (according to EU numbering), referred to herein as “IE”. In some embodiments, a CH₂ ribonucleic sequence comprises of a sequence according to SEQ ID NO: 111. In some embodiments, a CH₂ ribonucleic acid sequence encodes a CH₂ domain that comprises an E294 deletion (according to EU numbering).

In some embodiments, a conventional antibody encoded by one or more polyribonucleotides as described herein comprises a signal peptide comprising a human signal peptide. In some embodiments, a conventional antibody encoded by one or more polyribonucleotides as described herein comprises a signal peptide comprising SEQ ID NO: 1.

In some embodiments, a conventional antibody encoded by one or more polyribonucleotides as described herein comprises a light chain constant domain, where the light chain constant domain comprises a kappa light chain constant domain. In some embodiments, a conventional antibody encoded by one or more polyribonucleotides as described herein comprises a kappa light chain constant domain having an amino acid sequence at least 80, 85, 90%, 91%, 92%, 93%, 94%, 95%, 96%, 97%, 98%, or 99% identical to SEQ ID NO: 149. In some embodiments, a conventional antibody encoded by one or more polyribonucleotides as described herein comprises a kappa light chain constant domain having an amino acid sequence represented in SEQ ID NO: 149. In some embodiments, a conventional antibody encoded by one or more polyribonucleotides as described herein comprises a lambda chain variable domain.

In some embodiments, a conventional antibody encoded by one or more polyribonucleotides as described herein comprises an immunoglobulin chain (e.g., an immunoglobulin heavy chain) encoded by a nucleic acid sequence that has at least 80%, 85%, 90%, 91%, 92%, 93%, 94%, 95%, 96%, 97%, 98%, or 99% sequence identity to a sequence represented by SEQ ID NOs: 170-265, 1305-1306, 1308-1309, 1314-1315. In some embodiments, a conventional antibody encoded by one or more polyribonucleotides as described herein comprises an immunoglobulin chain (e.g., an immunoglobulin heavy chain) encoded by a nucleic acid sequence represented by any one of SEQ ID NOs: 170-265, 1305-1306, 1308-1309, and 1314-1315. In some embodiments, a conventional antibody encoded by one or more polyribonucleotides as described herein comprises an immunoglobulin chain (e.g., an immunoglobulin light chain) encoded by a nucleic acid sequence that has at least at least 80%, 85%, 90%, 91%, 92%, 93%, 94%, 95%, 96%, 97%, 98%, or 99% sequence identity to SEQ ID NO: 842,843, and 1311-1323. In some embodiments, a conventional antibody encoded by one or more polyribonucleotides as described herein comprises an immunoglobulin chain (e.g., an immunoglobulin light chain) encoded by a nucleic acid sequence represented by SEQ ID NO: 842-843 and 1311-1312.

In some embodiments, a conventional antibody encoded by one or more polyribonucleotides as described herein comprises an immunoglobulin chain (e.g., an immunoglobulin heavy chain) that comprises an amino acid sequence that has at least 80%, 85%, 90%, 91%, 92%, 93%, 94%, 95%, 96%, 97%, 98%, or 99% sequence identity to a sequence represented by any one of SEQ ID NOs: 1307, 1310, or 1316. In some embodiments, a conventional antibody encoded by one or more polyribonucleotides as described herein comprises an immunoglobulin chain (e.g., an immunoglobulin heavy chain) that comprises an amino acid sequence represented by any one of SEQ ID NOs: 1307, 1310 and 1316.

In some embodiments, a conventional antibody encoded by one or more polyribonucleotides as described herein comprises an immunoglobulin chain (e.g., an immunoglobulin light chain) that comprises an amino acid sequence that has at least at least 80%, 85%, 90%, 91%, 92%, 93%, 94%, 95%, 96%, 97%, 98%, or 99% sequence identity to SEQ ID NO: 1313. In some embodiments, a conventional antibody encoded by one or more polyribonucleotides as described herein comprises an immunoglobulin chain (e.g., an immunoglobulin light chain) that comprises an amino acid sequence represented by SEQ ID NO: 1313.

Exemplary immunoglobulin chain (e.g., immunoglobulin heavy chain or light chain) configurations of a conventional antibody, as described herein, are shown in Table 3 below.

TABLE 3
Exemplary Immunoglobulin Chain Configurations

| IgG Format - | | | | | | |
|---|---|---|---|---|---|---|
| Heavy Chain | Allotype | VH | CH1 | Hinge | CH2 | CH3 |
| PGDM1400 VH | G1m3 | PGDM1400 | CH1_G1m3 | Hinge | CH2 | CH3_G1m3/17 |
| G1m3 | | | | | | |
| PGDM1400 VH | G1m3 | PGDM1400 | CH1_G1m3 | Hinge | CH2 | CH3_LS_G1m3/17 |
| G1m3 LS | | | | | | |
| PGDM1400 VH | G1m3 | PGDM1400 | CH1_G1m3 | Hinge | CH2 | CH3_cah_LS_G1m3/17 |
| G1m3 cah LS | | | | | | |
| PGDM1400 VH | G1m3 | PGDM1400 | CH1_G1m3 | Hinge | CH2 | CH3_cak_LS_G1m3/17 |
| G1m3 cak LS | | | | | | |
| PGDM1400 VH | G1m3 | PGDM1400 | CH1_G1m3 | Hinge | CH2_GAALIE | CH3_LS_G1m3/17 |
| G1m3 GAALIE | | | | | | |
| LS | | | | | | |
| PGDM1400 VH | G1m3 | PGDM1400 | CH1_G1m3 | Hinge | CH2_GAIE | CH3_LS_G1m3/17 |
| G1m3 GAIE | | | | | | |
| LS | | | | | | |
| PGDM1400 VH | G1m3 | PGDM1400 | CH1_G1m3 | Hinge | CH2_GA | CH3_LS_G1m3/17 |
| G1m3 GA LS | | | | | | |
| PGDM1400 VH | G1m3 | PGDM1400 | CH1_G1m3 | Hinge | CH2_IE | CH3_LS_G1m3/17 |
| G1m3 IE LS | | | | | | |
| PGDM1400 VH | G1m3 | PGDM1400 | CH1_G1m3 | Hinge | CH2_GAALIE | CH3_cah_LS_G1m3/17 |
| G1m3 GAALIE | | | | | | |
| cah LS | | | | | | |
| PGDM1400 VH | G1m3 | PGDM1400 | CH1_G1m3 | Hinge | CH2_GAIE | CH3_cah_LS_G1m3/17 |
| G1m3 GAIE | | | | | | |
| cah LS | | | | | | |
| PGDM1400 VH | G1m3 | PGDM1400 | CH1_G1m3 | Hinge | CH2_GA | CH3_cah_LS_G1m3/17 |
| G1m3 GA cah | | | | | | |
| LS | | | | | | |
| PGDM1400 VH | G1m3 | PGDM1400 | CH1_G1m3 | Hinge | CH2_IE | CH3_cah_LS_G1m3/17 |
| G1m3 IE cah | | | | | | |
| LS | | | | | | |
| PGDM1400 VH | G1m3 | PGDM1400 | CH1_G1m3 | Hinge | CH2_GAALIE | CH3_cak_LS_G1m3/17 |
| G1m3 GAALIE | | | | | | |
| cak LS | | | | | | |
| PGDM1400 VH | G1m3 | PGDM1400 | CH1_G1m3 | Hinge | CH2_GAIE | CH3_cak_LS_G1m3/17 |
| G1m3 GAIE | | | | | | |
| cak LS | | | | | | |
| PGDM1400 VH | G1m3 | PGDM1400 | CH1_G1m3 | Hinge | CH2_GA | CH3_cak_LS_G1m3/17 |
| G1m3 GA cak | | | | | | |
| LS | | | | | | |
| PGDM1400 VH | G1m3 | PGDM1400 | CH1_G1m3 | Hinge | CH2_IE | CH3_cak_LS_G1m3/17 |
| G1m3 IE cak | | | | | | |
| LS | | | | | | |
| PGDM1400 VH | G1m17 | PGDM1400 | CH1_G1m17 | Hinge | CH2 | CH3_G1m3/17 |
| G1m17 | | | | | | |
| PGDM1400 VH | G1m17 | PGDM1400 | CH1_G1m17 | Hinge | CH2 | CH3_LS_G1m3/17 |
| G1m17 LS | | | | | | |
| PGDM1400 VH | G1m17 | PGDM1400 | CH1_G1m17 | Hinge | CH2 | CH3_cah_LS_G1m3/17 |
| G1m17 cah | | | | | | |
| LS | | | | | | |
| PGDM1400 VH | G1m17 | PGDM1400 | CH1_G1m17 | Hinge | CH2 | CH3_cak_LS_G1m3/17 |
| G1m17 cak | | | | | | |
| LS | | | | | | |
| PGDM1400 VH | G1m17 | PGDM1400 | CH1_G1m17 | Hinge | CH2_GAALIE | CH3_LS_G1m3/17 |
| G1m17 | | | | | | |
| GAALIE LS | | | | | | |
| PGDM1400 VH | G1m17 | PGDM1400 | CH1_G1m17 | Hinge | CH2_GAIE | CH3_LS_G1m3/17 |
| G1m17 GAIE | | | | | | |
| LS | | | | | | |
| PGDM1400 VH | G1m17 | PGDM1400 | CH1_G1m17 | Hinge | CH2_GA | CH3_LS_G1m3/17 |
| G1m17 GA LS | | | | | | |
| PGDM1400 VH | G1m17 | PGDM1400 | CH1_G1m17 | Hinge | CH2_IE | CH3_LS_G1m3/17 |
| G1m17 IE LS | | | | | | |
| PGDM1400 VH | G1m17 | PGDM1400 | CH1_G1m17 | Hinge | CH2_GAALIE | CH3_cah_LS_G1m3/17 |
| G1m17 | | | | | | |
| GAALIE cah LS | | | | | | |
| PGDM1400 VH | G1m17 | PGDM1400 | CH1_G1m17 | Hinge | CH2_GAIE | CH3_cah_LS_G1m3/17 |
| G1m17 GAIE | | | | | | |
| cah LS | | | | | | |
| PGDM1400 VH | G1m17 | PGDM1400 | CH1_G1m17 | Hinge | CH2_GA | CH3_cah_LS_G1m3/17 |
| G1m17 GA | | | | | | |
| cah LS | | | | | | |
| PGDM1400 VH | G1m17 | PGDM1400 | CH1_G1m17 | Hinge | CH2_IE | CH3_cah_LS_G1m3/17 |
| G1m17 IE cah | | | | | | |
| LS | | | | | | |
| PGDM1400 VH | G1m17 | PGDM1400 | CH1_G1m17 | Hinge | CH2_GAALIE | CH3_cak_LS_G1m3/17 |
| G1m17 | | | | | | |
| GAALIE cak LS | | | | | | |
| PGDM1400 VH | G1m17 | PGDM1400 | CH1_G1m17 | Hinge | CH2_GAIE | CH3_cak_LS_G1m3/17 |
| G1m17 GAIE | | | | | | |
| cak LS | | | | | | |
| PGDM1400 VH | G1m17 | PGDM1400 | CH1_G1m17 | Hinge | CH2_GA | CH3_cak_LS_G1m3/17 |
| G1m17 GA | | | | | | |
| cak LS | | | | | | |
| PGDM1400 VH | G1m17 | PGDM1400 | CH1_G1m17 | Hinge | CH2_IE | CH3_cak_LS_G1m3/17 |
| G1m17 IE cak | | | | | | |
| LS | | | | | | |
| PGDM1400 VH | G1m17,1 | PGDM1400 | CH1_G1m17 | Hinge | CH2 | CH3_G1m17,1 |
| G1m17,1 | | | | | | |
| PGDM1400 VH | G1m17,1 | PGDM1400 | CH1_G1m17 | Hinge | CH2 | CH3_LS_G1m17,1 |
| G1m17,1 LS | | | | | | |
| PGDM1400 VH | G1m17,1 | PGDM1400 | CH1_G1m17 | Hinge | CH2 | CH3_cah_LS_G1m17,1 |
| G1m17,1 cah | | | | | | |
| LS | | | | | | |
| PGDM1400 VH | G1m17,1 | PGDM1400 | CH1_G1m17 | Hinge | CH2 | CH3_cak_LS_G1m17,1 |
| G1m17,1 cak | | | | | | |
| LS | | | | | | |
| PGDM1400 VH | G1m17,1 | PGDM1400 | CH1_G1m17 | Hinge | CH2_GAALIE | CH3_LS_G1m17,1 |
| G1m17,1 | | | | | | |
| GAALIE LS | | | | | | |
| PGDM1400 VH | G1m17,1 | PGDM1400 | CH1_G1m17 | Hinge | CH2_GAIE | CH3_LS_G1m17,1 |
| G1m17,1 | | | | | | |
| GAIE LS | | | | | | |
| PGDM1400 VH | G1m17,1 | PGDM1400 | CH1_G1m17 | Hinge | CH2_GA | CH3_LS_G1m17,1 |
| G1m17,1 GA | | | | | | |
| LS | | | | | | |
| PGDM1400 VH | G1m17,1 | PGDM1400 | CH1_G1m17 | Hinge | CH2_IE | CH3_LS_G1m17,1 |
| G1m17,1 IE | | | | | | |
| LS | | | | | | |
| PGDM1400 VH | G1m17,1 | PGDM1400 | CH1_G1m17 | Hinge | CH2_GAALIE | CH3_cah_LS_G1m17,1 |
| G1m17,1 | | | | | | |
| GAALIE cah LS | | | | | | |
| PGDM1400 VH | G1m17,1 | PGDM1400 | CH1_G1m17 | Hinge | CH2_GAIE | CH3_cah_LS_G1m17,1 |
| G1m17,1 | | | | | | |
| GAIE cah LS | | | | | | |
| PGDM1400 VH | G1m17,1 | PGDM1400 | CH1_G1m17 | Hinge | CH2_GA | CH3_cah_LS_G1m17,1 |
| G1m17,1 GA | | | | | | |
| cah LS | | | | | | |
| PGDM1400 VH | G1m17,1 | PGDM1400 | CH1_G1m17 | Hinge | CH2_IE | CH3_cah_LS_G1m17,1 |
| G1m17,1 IE | | | | | | |
| cah LS | | | | | | |
| PGDM1400 VH | G1m17,1 | PGDM1400 | CH1_G1m17 | Hinge | CH2_GAALIE | CH3_cak_LS_G1m17,1 |
| G1m17,1 | | | | | | |
| GAALIE cak LS | | | | | | |
| PGDM1400 VH | G1m17,1 | PGDM1400 | CH1_G1m17 | Hinge | CH2_GAIE | CH3_cak_LS_G1m17,1 |
| G1m17,1 | | | | | | |
| GAIE cak LS | | | | | | |
| PGDM1400 VH | G1m17,1 | PGDM1400 | CH1_G1m17 | Hinge | CH2_GA | CH3_cak_LS_G1m17,1 |
| G1m17,1 GA | | | | | | |
| cak LS | | | | | | |
| PGDM1400 VH | G1m17,1 | PGDM1400 | CH1_G1m17 | Hinge | CH2_IE | CH3_cak_LS_G1m17,1 |
| G1m17,1 IE | | | | | | |
| cak LS | | | | | | |

| IgG Format - | | | | | | |
|---|---|---|---|---|---|---|
| Light Chain | — | VL | CL | — | — | — |
| PGDM1400 VL | — | PGDM1400 | Ck | — | — | — |
| Ck | | | | | | |

### B. CrossMab^{CH1-CLx}

The present disclosure also provides technologies that can be used to deliver and express an antibody agent as described herein in “CrossMab” format (see e.g., WO2015/101588 A1, WO 2009/080253A1, and Schaefer, W. et al., PNAS, 108, 11187-1191, 2011, which are herein incorporated by reference in their entirety). In some embodiments, antibody agents in CrossMab format contain a CL-CH₁ crossover in one or both binding arms (referred to herein as “CrossMab^{CH1-CLx}” or “CH₁-CLx”). Such a modification reduces the byproduct formation caused by a mismatch of a light chain of a first antibody that specifically binds to a first antigen with the wrong heavy chain of a second antibody that specifically binds to a second antigen (when compared to approaches without such domain exchanges).

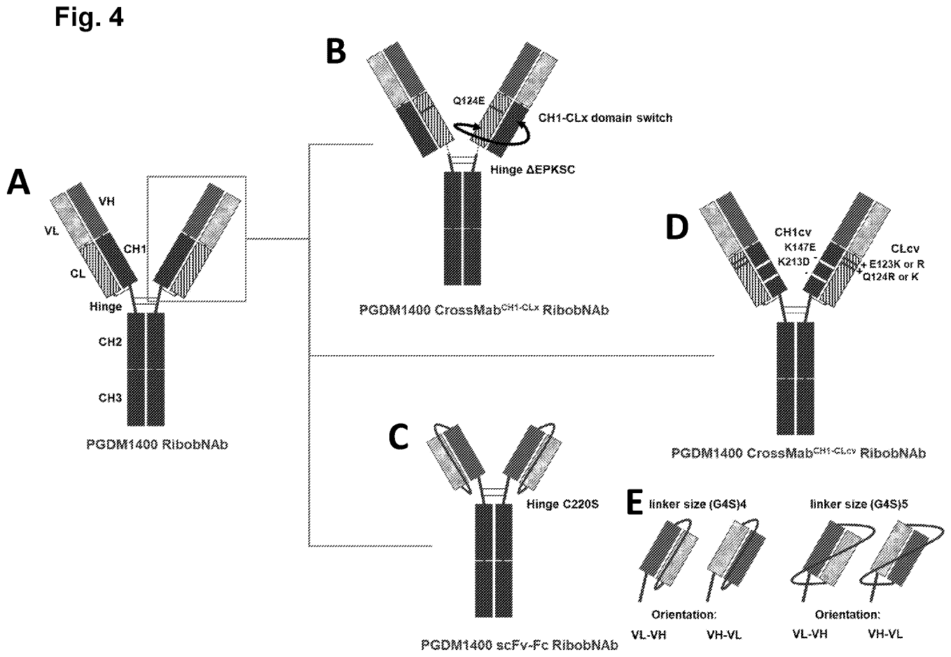

In some embodiments, an antibody agent encoded by one or more polyribonucleotides provided herein comprises a first immunoglobulin chain and a second immunoglobulin chain. In some embodiments, a polyribonucleotide may encode a first immunoglobulin chain and a second immunoglobulin chain of a CrossMab^{CH1-CLx} antibody agent as described herein. In some embodiments, a polyribonucleotide encoding a first immunoglobulin chain comprises a ribonucleic acid sequence encoding a VH domain, a CL domain, a hinge domain, a CH₂ domain, and a CH₃ domain. In some embodiments, a polyribonucleotide encoding a second immunoglobulin chain comprises a ribonucleic acid sequence encoding a light chain variable (VL) domain and a CH₁ domain (see e.g., FIG. 4, Panel B). In some embodiments, a polyribonucleotide encoding a CrossMab^{CH1-CLx} antibody agent comprises a ribonucleic acid sequence that encodes any one of the immunoglobulin chain configurations in Table 4, corresponding to SEQ ID NOs: 266-361, 1317-1318, 1329-1330, 1332-1333, 1335-1336, and 1338-1339. In some embodiments, a polyribonucleotide encoding a CrossMab^{CH1-CLx} agent antibody comprises a ribonucleic acid sequence that encodes any one of the immunoglobulin chain configurations in Table 4, corresponding to SEQ ID NOs: 844-847 and 1230-1321.

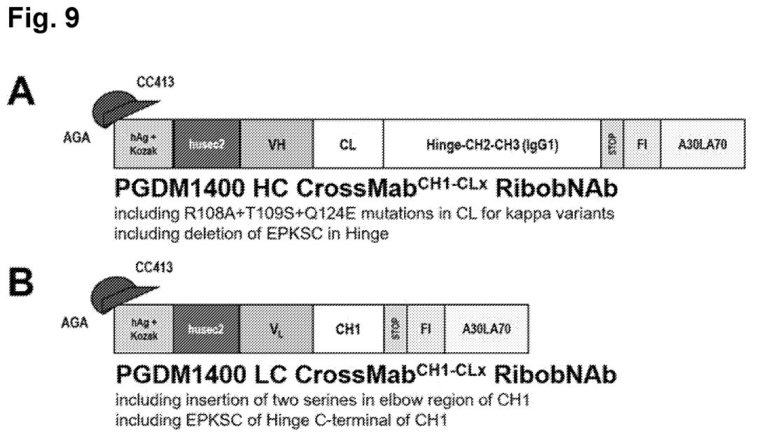

In some embodiments, a CrossMab^{CH1-CLx} antibody agent may be encoded by two separate polyribonucleotide: a first polyribonucleotide comprising a coding region that encodes (in 5′ to 3′ order): a heavy chain variable domain (VH), a light chain constant region (CL), a hinge region, a CH₂ domain, and a CH₃ domain (see e.g., FIG. 9, Panel A); and a second polyribonucleotide comprising a coding region that encodes (in 5′ to 3′ order): a light chain variable domain (VL) and a CH₁ domain (see e.g., FIG. 9, Panel B).

In some embodiments, a CrossMab^{CH1-CLx} antibody agent encoded by one or more polyribonucleotides provided herein includes all or part of a PGDM1400 antibody. In some embodiments, a CrossMab^{CH1-CLx} antibody agent comprises a heavy chain variable domain comprising: (i) HCDR1 (SEQ ID NO: 18); (ii) HCDR2 SEQ ID NO: 21); (iii) HCDR3 (SEQ ID NO: 24); or (iv) a combination thereof. In some embodiments, a CrossMab^{CH1-CLx} antibody agent comprises a heavy chain variable domain comprising: (i) HCDR1 (SEQ ID NO: 18); (ii) HCDR2 (SEQ ID NO: 21); and (iii) HCDR3 (SEQ ID NO: 24). In some embodiments, a CrossMab^{CH1-CLx} antibody agent comprises a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a CrossMab^{CH1-CLx} antibody agent comprises a light chain variable domain comprising: (i) LCDR1 (SEQ ID NO: 27); (ii) LCDR2 (LAS; SEQ ID NO: 30); and (iii) LCDR3 (SEQ ID NO: 33). In some embodiments, a CrossMab^{CH1-CLx} antibody agent comprises (a) a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof; and (b) a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a CrossMab^{CH1-CLx} antibody agent comprises (a) a heavy chain variable domain comprising: (i) HCDR1 (SEQ ID NO: 18); (ii) HCDR2 (SEQ ID NO: 21); and (iii) HCDR3 (SEQ ID NO: 24); and (b) a light chain variable domain comprising: (i) LCDR1 (SEQ ID NO: 27); (ii) LCDR2 (LAS; SEQ ID NO: 30); and (iii) LCDR3 (SEQ ID NO: 33).

In some embodiments, a polyribonucleotide described herein encodes an immunoglobulin chain of a CrossMab^{CH1-CLx} antibody agent, wherein the immunoglobulin chain comprises a heavy chain variable domain, and wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof. In some embodiments, a polyribonucleotide described herein encodes an immunoglobulin chain of a CrossMab^{CH1-CLx} antibody agent, wherein the immunoglobulin chain comprises a heavy chain variable domain, and wherein the heavy chain variable domain comprises: (i) HCDR1 (SEQ ID NO: 18); (ii) HCDR2 (SEQ ID NO: 21); and (iii) HCDR3 (SEQ ID NO: 24). In some embodiments, a polyribonucleotide described herein encodes an immunoglobulin chain of a CrossMab^{CH1-CLx} antibody agent, wherein the immunoglobulin chain comprises a light chain variable domain, and wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain of a CrossMab^{CH1-CLx} antibody agent, wherein the immunoglobulin chain comprises a light chain variable domain, and wherein the light chain variable domain comprises: (i) LCDR1 (SEQ ID NO: 27); (ii) LCDR2 (LAS; SEQ ID NO: 30); and (iii) LCDR3 (SEQ ID NO: 33). In some embodiments, a polyribonucleotide described herein encodes two immunoglobulin chains of a CrossMab^{CH1-CLx} antibody agent: a first immunoglobulin chain comprising a heavy chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof; and a second immunoglobulin chain comprising a light chain variable domain, wherein the light chain variable domain comprises (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes two immunoglobulin chains of a CrossMab^{CH1-CLx} antibody agent: a first immunoglobulin chain comprising a heavy chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24); and a second immunoglobulin chain comprising a light chain variable domain, wherein the light chain variable domain comprises: (i) LCDR1 (SEQ ID NO: 27); (ii) LCDR2 (LAS; SEQ ID NO: 30); and (iii) LCDR3 (SEQ ID NO: 33).

In some embodiments, a CrossMab^{CH1-CLx} antibody agent encoded by one or more polyribonucleotides provided herein comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a CrossMab^{CH1-CLx} antibody agent encoded by one or more polyribonucleotides provided herein comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a CrossMab^{CH1-CLx} antibody agent encoded by one or more polyribonucleotides provided herein comprises a heavy chain variable domain represented by SEQ ID NO: 36. In some embodiments, a CrossMab^{CH1-CLx} antibody agent encoded by one or more polyribonucleotides provided herein comprises a light chain variable domain represented by SEQ ID NO: 43.

In some embodiments, a CrossMab^{CH1-CLx} antibody agent encoded by one or more polyribonucleotides provided herein comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 1494. In some embodiments, a CrossMab^{CH1-CLx} antibody agent encoded by one or more polyribonucleotides provided herein comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a CrossMab^{CH1-CLx} antibody agent encoded by one or more polyribonucleotides provided herein comprises a heavy chain variable domain represented by SEQ ID NO: 1494. In some embodiments, a CrossMab^{CH1-CLx} antibody agent encoded by one or more polyribonucleotides provided herein comprises a light chain variable domain represented by SEQ ID NO: 43.

In some embodiments, an antibody agent (e.g., a PGDM1400 antibody agent) encoded by one or more polyribonucleotides provided herein comprises a heavy chain variable domain that comprises one or more mutations relative to an amino acid sequence represented by SEQ ID NO: 1494. In some embodiments, substitution mutations are present at residues 3 and/or 5 relative to SEQ ID NO: 1494. In some embodiments, a substitution mutation at residue 3 results in a positively charged amino acid being present at residue 3 relative to SEQ ID NO: 1494. Positively charged amino acids include Lys (K), Arg (R), and His (H). In some embodiments, a substitution mutation at residue 3 relative to SEQ ID NO: 1494 comprises Q3H. In some embodiments, a substitution mutation at residue 5 results in a polar amino acid being present at residue 5 relative to SEQ ID NO: 1494. Polar amino acids include Ser (S), Thr (T), Tyr (Y), Asn (N), and Gln (Q). In some embodiments, a substitution mutation at residue 5 relative to SEQ ID NO: 1494 comprises V5T.

In some embodiments, an antibody agent (e.g., a PGDM1400 antibody agent) encoded by one or more polyribonucleotides provided herein comprises a heavy chain variable domain that comprises SEQ ID NO: 1494 with substitution mutations Q3H and V5T.

In some embodiments, a polyribonucleotide described herein encodes an immunoglobulin chain of a CrossMab^{CH1-CLx} antibody agent, wherein the immunoglobulin chain comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a polyribonucleotide described herein encodes an immunoglobulin chain of a CrossMab^{CH1-CLx} antibody agent, wherein the immunoglobulin chain comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a polyribonucleotide described herein encodes an immunoglobulin chain of a CrossMab^{CH1-CLx} antibody agent, wherein the immunoglobulin chain comprises a heavy chain variable domain represented by SEQ ID NO: 36. In some embodiments, a polyribonucleotide described herein encodes an immunoglobulin chain of a CrossMab^{CH1-CLx} antibody agent, wherein the immunoglobulin chain comprises a light chain variable domain represented by SEQ ID NO: 43.

As described above, CrossMab^{CH1-CLx} antibody agents encoded by one or more polyribonucleotides as described herein may comprise one or more heavy chain constant domains. In some embodiments, one or more heavy chain constant domains comprise a CH₃ domain. In some embodiments, a CrossMab^{CH1-CLx} antibody agent encoded by one or more polyribonucleotides as described herein comprises a CH₃ domain that comprises a G1m3, G1m17, or a G1m17,1 allotype. In some embodiments, a CrossMab^{CH1-CLx} antibody agent encoded by one or more polyribonucleotides as described herein comprises a CH₃ domain having an amino acid sequence at least 90%, 91%, 92%, 93%, 94%, 95%, 96%, 97%, 98%, or 99% identical to a sequence represented in any one of SEQ ID NOs: 113, 116, 119, 122, 125, 128, 131, 134, 137, 140, 143, 146, or 1494. In some embodiments, a CrossMab^{CH1-CLx} antibody agent encoded by one or more polyribonucleotides as described herein comprises a CH₃ domain having an amino acid sequence represented in any one of SEQ ID NOs: 113, 116, 119, 122, 125, 128, 131, 134, 137, 140, 143, 146, or 1495. In some embodiments, a CH₃ domain comprises amino acid sequence SEQ ID NO: 1495 with substitution mutations D16E and L18M.

CrossMab^{CH1-CLx} antibody agents encoded by one or more polyribonucleotides as described herein may comprise one or more heavy chain constant domains comprising an amino acid modification (e.g., a substitution or deletion) at one or more amino acid positions. For example, a CrossMab^{CH1-CLx} antibody agent encoded by one or more polyribonucleotides as described herein may include an L/S mutation within a CH₃ region (for enhanced FcRn binding) (see Zalevsky J et al., Nat Biotechnol. 2010, which is herein incorporated by reference). Such mutations are noted as M428L and N434S according to EU numbering (i.e., M88L and N94S within the CH₃ domain, e.g., SEQ ID NO: 113, 116, or 1495), and referred to herein as “LS” or “L/S” (see e.g., FIG. 5, Panel D). In some embodiments, a CrossMab^{CH1-CLx} antibody agent encoded by one or more polyribonucleotides as described herein comprise an E294 deletion (for Fc hypersialylation) (see Bas M et al., J Immunol 2019, which is herein incorporated by reference).

In some embodiments, a CrossMab^{CH1-CLx} antibody agent encoded by one or more polyribonucleotides as described herein comprises a CH₃ domain comprising one or more of the following mutations: Y349C, T366S, L368A, and Y407V (according to EU numbering). This combination of mutations is referred to herein as “cah”. In some embodiments, a CrossMab^{CH1-CLx} antibody agent encoded by one or more polyribonucleotides as described herein comprises a CH₃ domain comprising one or more mutations selected from: S354C and T366W (according to EU numbering). This combination of CH₃ mutations is referred to herein as “cak”.

In some embodiments, a polyribonucleotide encodes a CH₃ domain that comprises one of the following substitution mutations: M428L, N434S, or a combination thereof (e.g., an “L/S” mutation). In some embodiments, a polyribonucleotide comprises a CH₃ ribonucleic acid sequence that comprises any one of SEQ ID NOs: 117 and 135. In some embodiments, a polyribonucleotide encodes a CH₃ domain that comprises one of the following substitution mutations: Y349C, T366S, L368A, and Y407V (according to EU numbering). In some embodiments, a polyribonucleotide comprises a ribonucleic acid sequence according to SEQ ID NO: 120 and 138. In some embodiments, a polyribonucleotide encodes a CH₃ domain that comprises one of the following substitution mutations: S354C and T366W (according to EU numbering). In some embodiments, a polyribonucleotide comprises a ribonucleic acid sequence according to SEQ ID NO: 126 and 144.

In some embodiments, a polyribonucleotide encodes an immunoglobulin chain that comprises a VH domain operably linked to one or more constant domains, where the one or more constant domains comprise a CH₃ domain. In some embodiments, a polyribonucleotide comprises a ribonucleic acid sequence according to SEQ ID NO: 114. In some embodiments, polyribonucleotide comprises a CH₃ ribonucleic acid sequence that encodes the CH₃ domain that comprises a G1m3, G1m17, or a G1m17,1 allotype. In some embodiments, a polyribonucleotide comprises a CH₃ ribonucleic acid sequence that comprises any one of SEQ ID NOs: 114 and 132.

In some embodiments, a CrossMab^{CH1-CLx} antibody agent encoded by one or more polyribonucleotides as described herein comprises a light chain constant domain. In some embodiments, a light chain constant domain comprises a kappa light chain constant domain. In some embodiments, a CrossMab^{CH1-CLx} antibody agent encoded by one or more polyribonucleotides as described herein comprises a kappa light chain constant domain having an amino acid sequence at least 80, 85, 90%, 91%, 92%, 93%, 94%, 95%, 96%, 97%, 98%, or 99% identical to SEQ ID NO: 153. In some embodiments, a CrossMab^{CH1-CLx} antibody agent encoded by one or more polyribonucleotides as described herein comprises a kappa light chain constant domain having an amino acid sequence represented in SEQ ID NO: 153. In some embodiments, a CrossMab^{CH1-CLx} antibody agent encoded by one or more polyribonucleotides as described herein comprises a lambda chain variable domain.

In some embodiments, a CrossMab^{CH1-CLx} antibody agent encoded by one or more polyribonucleotides as described herein comprises a hinge domain. In some embodiments, a CrossMab^{CH1-CLx} antibody agent encoded by one or more polyribonucleotides as described herein comprises a hinge domain that comprises an amino acid sequence represented in SEQ ID NO: 161 (herein referred to as “hinge” in Tables 2 and 4).

In some embodiments, a polyribonucleotide encodes a hinge domain. In some embodiments, a polyribonucleotide encodes a hinge ribonucleic acid sequence that represented in SEQ ID NO: 162. In some embodiments, a polyribonucleotide encodes a hinge domain that comprises an amino acid modification that comprises a deletion of one or more amino acid residues. In some embodiments, a polyribonucleotide encodes a hinge domain an amino acid modification that comprises a deletion of amino acid residues EPKSC in a conventional Ig hinge domain (represented in SEQ ID NO: 161). Such a modification is referred to herein as “Hinge_del” or “ΔEPKSC”. In some embodiments, a polyribonucleotide encodes a hinge ribonucleic acid sequence represented in SEQ ID NO: 168. In some embodiments, a polyribonucleotide encodes a hinge domain that comprises an amino acid modification that comprises a C220S mutation (according to EU numbering). Such a mutated hinge domain is referred to herein as “Hinge_S” or “C/S”. In some embodiments, a polyribonucleotide encodes a hinge ribonucleic acid sequence represented in SEQ ID NO: 165.

In some embodiments, a CrossMab^{CH1-CLx} antibody agent encoded by one or more polyribonucleotides as described herein comprises a CH₂ domain. In some embodiments, a CrossMab^{CH1-CLx} antibody agent encoded by one or more polyribonucleotides as described herein comprises a CH₂ domain having an amino acid sequence at least 90%, 91%, 92%, 93%, 94%, 95%, 96%, 97%, 98%, or 99% identical to represented in SEQ ID NO: 96. In some embodiments, a CrossMab^{CH1-CLx} antibody agent encoded by one or more polyribonucleotides as described herein comprises a CH₂ domain having an amino acid sequence represented in SEQ ID NO: 96.

In some embodiments, a CrossMab^{CH1-CLx} antibody agent encoded by one or more polyribonucleotides as described herein comprises a CH₂ domain having one or more mutations (e.g., with respect to SEQ ID NO: 96). For example, in some embodiments, a CrossMab^{CH1-CLx} antibody agent encoded by one or more polyribonucleotides as described herein comprises one or more of the following mutations: G236A, A330L, and I332E (according to EU numbering). Such a combination of mutations is referred to herein as “GAALIE”. Such mutations in the CH₂ domain have been associated with increased affinity to Fc receptors FcgRIIA and FcgRIII for enhanced antibody effector function.

In some embodiments, a CrossMab^{CH1-CLx} antibody agent encoded by one or more polyribonucleotides as described herein comprises a CH₂ domain having an amino acid sequence that is at least 90%, 91%, 92%, 93%, 94%, 95%, 96%, 97%, 98%, or 99% identical to SEQ ID NO: 99 or 102. In some embodiments, a CrossMab^{CH1-CLx} antibody agent encoded by one or more polyribonucleotides as described herein comprises a CH₂ domain having an amino acid sequence represented in SEQ ID NO: 99 or 102.

In some embodiments, a CrossMab^{CH1-CLx} antibody agent encoded by one or more polyribonucleotides as described herein comprises one or more mutations selected from: G236A and I332E (according to EU numbering). Such a combination of CH₂ mutations is referred to herein as “GAIE”. In some embodiments, a CrossMab^{CH1-CLx} antibody agent encoded by one or more polyribonucleotides as described herein comprises a CH₂ domain having an amino acid sequence at least 90%, 91%, 92%, 93%, 94%, 95%, 96%, 97%, 98%, or 99% identical to SEQ ID NO: 104. In some embodiments, a CrossMab^{CH1-CLx} antibody agent encoded by one or more polyribonucleotides as described herein comprises a CH₂ domain having an amino acid sequence represented in SEQ ID NO: 104.

In some embodiments, a CrossMab^{CH1-CLx} antibody agent encoded by one or more polyribonucleotides as described herein comprises a mutation: G236A (according to EU numbering), referred to herein as “GA”. In some embodiments, a CrossMab^{CH1-CLx} antibody agent encoded by one or more polyribonucleotides as described herein comprises a CH₂ domain having an amino acid sequence at least 90%, 91%, 92%, 93%, 94%, 95%, 96%, 97%, 98%, or 99% identical to SEQ ID NO: 107. In some embodiments, a CrossMab^{CH1-CLx} antibody agent encoded by one or more polyribonucleotides as described herein comprises a CH₂ domain having an amino acid sequence represented in SEQ ID NO: 107.

In some embodiments, a CrossMab^{CH1-CLx} antibody agent encoded by one or more polyribonucleotides as described herein comprises a mutation: I332E (according to EU numbering), referred to herein as “IE”. In some embodiments, a CrossMab^{CH1-CLx} antibody agent encoded by one or more polyribonucleotides as described herein comprises a CH₂ domain having an amino acid sequence at least 90%, 91%, 92%, 93%, 94%, 95%, 96%, 97%, 98%, or 99% identical to SEQ ID NO: 110. In some embodiments, a CrossMab^{CH1-CLx} antibody agent encoded by one or more polyribonucleotides as described herein comprises a CH₂ domain having an amino acid sequence represented in SEQ ID NO: 110.

In some embodiments, a polyribonucleotide encodes an immunoglobulin chain that comprises a VH domain operably linked to one or more constant domains, wherein the one or more constant domains comprise a CH₂ domain. In some embodiments, a polyribonucleotide comprises a ribonucleic acid sequence that according to SEQ ID NO: 97. In some embodiments, a CH₂ ribonucleic acid encodes a CH₂ domain with one or more amino acid substitution mutations. For example, in some embodiments, a CH₂ ribonucleic acid sequence encodes one or more of the following mutations: G236A, A330L, and I332E (according to EU numbering), referred to herein as “GAALIE”. Such mutations in the CH₂ domain have been associated with increased affinity to Fc receptors FcgRIIA and FcgRIII for enhanced antibody effector function. In some embodiments, a CH₂ ribonucleic sequence comprises or consists of a sequence according to SEQ ID NO: 100 or 103. In some embodiments, a CH₂ ribonucleic acid sequence encodes the one or more of the following mutations: G236A and I332E (according to EU numbering), referred to herein as “GAIE”. In some embodiments, a CH₂ ribonucleic sequence comprises of a sequence according to SEQ ID NO: 105. In some embodiments, a CH₂ ribonucleic acid sequence encodes the mutation G236A (according to EU numbering), referred to herein as “GA”. In some embodiments, a CH₂ ribonucleic sequence comprises of a sequence according to SEQ ID NO: 108. In some embodiments, a CH₂ ribonucleic acid sequence encodes the mutation: I332E (according to EU numbering), referred to herein as “IE”. In some embodiments, a CH₂ ribonucleic sequence comprises of a sequence according to SEQ ID NO: 111. In some embodiments, a CH₂ ribonucleic acid sequence encodes a CH₂ domain that comprises a E294 deletion (according to EU numbering), referred to herein as “E294del”.

In some embodiments, a CrossMab^{CH1-CLx} antibody agent encoded by one or more polyribonucleotides as described herein comprises a CH₁ domain. In some embodiments, a CrossMab^{CH1-CLx} antibody agent encoded by one or more polyribonucleotides as described herein comprises a CH₁ domain that comprises a G1m3 allotype. In some embodiments, a CrossMab^{CH1-CLx} antibody agent encoded by one or more polyribonucleotides as described herein comprises a CH₁ domain that comprises a G1m17 allotype. In some embodiments, a CrossMab^{CH1-CLx} antibody agent encoded by one or more polyribonucleotides as described herein comprises a CH₁ domain that comprises an amino acid sequence represented in SEQ ID NO: 76 or 81.

In some embodiments, a polyribonucleotide encodes an immunoglobulin chain that comprises a VH domain operably linked to one or more constant domains, where the one or more constant domains comprise a CH₁ domain. In some embodiments, a polyribonucleotide comprises a CH₁ ribonucleic acid sequence according to SEQ ID NO: 77 or 79. In some embodiments, a polyribonucleotide encodes a CH₁ domain that comprises a G1m3 allotype. In some embodiments, a polyribonucleotide encodes a CH₁ domain that comprises a G1m17 allotype. In some embodiments, a polyribonucleotide encodes a CH₁ ribonucleic acid sequence according to SEQ ID NO: 77, 79, or 82.

In some embodiments, a polyribonucleotide encodes a CH₁ domain that comprises one or more mutations. In some embodiments, a polyribonucleotide encodes a CH₁ domain that comprises the addition of one or more serine residues. In some embodiments, a polyribonucleotide encodes a CH₁ domain that comprises addition of two additional serine residues (referred to herein as “SS”). In some embodiments, a polyribonucleotide encodes a CH₁ ribonucleic acid sequence that is at least 90%, 91%, 92%, 93%, 94%, 95%, 96%, 97%, 98%, or 99% identical to SEQ ID NO: 91 or 94. In some embodiments, a polyribonucleotide encodes a CH₁ ribonucleic acid sequence represented in SEQ ID NO: 91 or 94. In some embodiments, a polyribonucleotide encodes a CH₁ domain that comprises one or more charge variant mutations. In some embodiments, a polyribonucleotide encodes a CH₁ domain comprising one or more substitution mutations selected from: K147E, K213D, or a combination thereof. In some embodiments, a polyribonucleotide encodes a CH₁ ribonucleic acid sequence that has at least 90%, 91%, 92%, 93%, 94%, 95%, 96%, 97%, 98%, or 99% sequence identity to SEQ ID NO: 85 or 88. In some embodiments, a polyribonucleotide encodes a CH₁ ribonucleic acid sequence according to SEQ ID NO: 85 or 88.

In some embodiments, a CrossMab^{CH1-CLx} antibody agent encoded by one or more polyribonucleotides as described herein comprises a signal peptide comprising a husec2 signal peptide. In some embodiments, a CrossMab^{CH1-CLx} antibody agent encoded by one or more polyribonucleotides as described herein comprises a signal peptide comprising SEQ ID NO: 1.

In some embodiments, a CrossMab^{CH1-CLx} antibody agent encoded by one or more polyribonucleotides as described herein comprises an immunoglobulin chain encoded by a nucleic acid sequence that has at least 80%, 85%, 90%, 91%, 92%, 93%, 94%, 95%, 96%, 97%, 98%, or 99% sequence identity to a sequence represented by SEQ ID NOs: 266-361, 1317-1318, 1329-1330, 1332-1333, 1335-1336, 1338-1339 (heavy chain) and SEQ ID NOs: 844-847, 1320-1321 (light chain). In some embodiments, a CrossMab^{CH1-CLx} antibody agent encoded by one or more polyribonucleotides as described herein comprises an immunoglobulin chain encoded by a nucleic acid sequence represented by any one of SEQ ID NOs: 266-361, 1317-1318, 1329-1330, 1332-1333, 1335-1336, 1338-1339 (heavy chain) and SEQ ID NOs: 844-847, 1320-1321 (light chain).

In some embodiments, a CrossMab^{CH1-CLx} antibody agent encoded by one or more polyribonucleotides as described herein comprises an immunoglobulin chain comprising an amino acid sequence that has at least 80%, 85%, 90%, 91%, 92%, 93%, 94%, 95%, 96%, 97%, 98%, or 99% sequence identity to a sequence represented by SEQ ID NOs: 1319, 1331, 1334, 1337, 1340 (heavy chain) and SEQ ID NOs: 1322 (light chain). In some embodiments, a CrossMab^{CH1-CLx} antibody agent encoded by one or more polyribonucleotides as described herein comprises an immunoglobulin chain comprising an amino acid sequence represented by any one of SEQ ID NOs: 1319, 1331, 1334, 1337, 1340 (heavy chain) 1322 (heavy chain) and SEQ ID NO: 1322 (light chain).

Exemplary heavy chain and light chain configurations of a CrossMab^{CH1-CLx} antibody agent, as described herein, are shown in Table 4 below.

TABLE 4
Exemplary CrossMab^{CH1-CLx} Chain Configurations

| CrossMab^{CH1-CLx} | | | | | | |
|---|---|---|---|---|---|---|
| format Heavy Chain | Allotype | VH | CL | Hinge | CH2 | CH3 |
| PGDM1400 VH G1m3 | G1m3 | PGDM1400 | Ck_ASE | Hinge_del | CH2 | CH3_G1m3/17 |
| Ck(AS/Q124E) del216- | | | | | | |
| 220 | | | | | | |
| PGDM1400 VH G1m3 | G1m3 | PGDM1400 | Ck_ASE | Hinge_del | CH2 | CH3_LS_G1m3/17 |
| Ck(AS/Q124E) del216- | | | | | | |
| 220 LS | | | | | | |
| PGDM1400 VH G1m3 | G1m3 | PGDM1400 | Ck_ASE | Hinge_del | CH2_GAALIE | CH3_LS_G1m3/17 |
| Ck(AS/Q124E) del216- | | | | | | |
| 220 GAALIE LS | | | | | | |
| PGDM1400 VH G1m3 | G1m3 | PGDM1400 | Ck_ASE | Hinge_del | CH2_GAIE | CH3_LS_G1m3/17 |
| Ck(AS/Q124E) del216- | | | | | | |
| 220 GAIE LS | | | | | | |
| PGDM1400 VH G1m3 | G1m3 | PGDM1400 | Ck_ASE | Hinge_del | CH2_GA | CH3_LS_G1m3/17 |
| Ck(AS/Q124E) del216- | | | | | | |
| 220 GA LS | | | | | | |
| PGDM1400 VH G1m3 | G1m3 | PGDM1400 | Ck_ASE | Hinge_del | CH2_IE | CH3_LS_G1m3/17 |
| Ck(AS/Q124E) del216- | | | | | | |
| 220 IE LS | | | | | | |
| PGDM1400 VH G1m3 | G1m3 | PGDM1400 | Ck_ASE | Hinge_del | CH2 | CH3_cah_LS_G1m3/17 |
| Ck(AS/Q124E) del216- | | | | | | |
| 220 cah LS | | | | | | |
| PGDM1400 VH G1m3 | G1m3 | PGDM1400 | Ck_ASE | Hinge_del | CH2_GAALIE | CH3_cah_LS_G1m3/17 |
| Ck(AS/Q124E) del216- | | | | | | |
| 220 GAALIE cah LS | | | | | | |
| PGDM1400 VH G1m3 | G1m3 | PGDM1400 | Ck_ASE | Hinge_del | CH2_GAIE | CH3_cah_LS_G1m3/17 |
| Ck(AS/Q124E) del216- | | | | | | |
| 220 GAIE cah LS | | | | | | |
| PGDM1400 VH G1m3 | G1m3 | PGDM1400 | Ck_ASE | Hinge_del | CH2_GA | CH3_cah_LS_G1m3/17 |
| Ck(AS/Q124E) del216- | | | | | | |
| 220 GA cah LS | | | | | | |
| PGDM1400 VH G1m3 | G1m3 | PGDM1400 | Ck_ASE | Hinge_del | CH2_IE | CH3_cah_LS_G1m3/17 |
| Ck(AS/Q124E) del216- | | | | | | |
| 220 IE cah LS | | | | | | |
| PGDM1400 VH G1m3 | G1m3 | PGDM1400 | Ck_ASE | Hinge_del | CH2 | CH3_cak_LS_G1m3/17 |
| Ck(AS/Q124E) del216- | | | | | | |
| 220 cak LS | | | | | | |
| PGDM1400 VH G1m3 | G1m3 | PGDM1400 | Ck_ASE | Hinge_del | CH2_GAALIE | CH3_cak_LS_G1m3/17 |
| Ck(AS/Q124E) del216- | | | | | | |
| 220 GAALIE cak LS | | | | | | |
| PGDM1400 VH G1m3 | G1m3 | PGDM1400 | Ck_ASE | Hinge_del | CH2_GAIE | CH3_cak_LS_G1m3/17 |
| Ck(AS/Q124E) del216- | | | | | | |
| 220 GAIE cak LS | | | | | | |
| PGDM1400 VH G1m3 | G1m3 | PGDM1400 | Ck_ASE | Hinge_del | CH2_GA | CH3_cak_LS_G1m3/17 |
| Ck(AS/Q124E) del216- | | | | | | |
| 220 GA cak LS | | | | | | |
| PGDM1400 VH G1m3 | G1m3 | PGDM1400 | Ck_ASE | Hinge_del | CH2_IE | CH3_cak_LS_G1m3/17 |
| Ck(AS/Q124E) del216- | | | | | | |
| 220 IE cak LS | | | | | | |
| PGDM1400 VH G1m17 | G1m17 | PGDM1400 | Ck_ASE | Hinge_del | CH2 | CH3_G1m3/17 |
| Ck(AS/Q124E) del216- | | | | | | |
| 220 | | | | | | |
| PGDM1400 VH G1m17 | G1m17 | PGDM1400 | Ck_ASE | Hinge_del | CH2 | CH3_LS_G1m3/17 |
| Ck(AS/Q124E) del216- | | | | | | |
| 220 LS | | | | | | |
| PGDM1400 VH G1m17 | G1m17 | PGDM1400 | Ck_ASE | Hinge_del | CH2_GAALIE | CH3_LS_G1m3/17 |
| Ck(AS/Q124E) del216- | | | | | | |
| 220 GAALIE LS | | | | | | |
| PGDM1400 VH G1m17 | G1m17 | PGDM1400 | Ck_ASE | Hinge_del | CH2_GAIE | CH3_LS_G1m3/17 |
| Ck(AS/Q124E) del216- | | | | | | |
| 220 GAIE LS | | | | | | |
| PGDM1400 VH G1m17 | G1m17 | PGDM1400 | Ck_ASE | Hinge_del | CH2_GA | CH3_LS_G1m3/17 |
| Ck(AS/Q124E) del216- | | | | | | |
| 220 GA LS | | | | | | |
| PGDM1400 VH G1m17 | G1m17 | PGDM1400 | Ck_ASE | Hinge_del | CH2_IE | CH3_LS_G1m3/17 |
| Ck(AS/Q124E) del216- | | | | | | |
| 220 IE LS | | | | | | |
| PGDM1400 VH G1m17 | G1m17 | PGDM1400 | Ck_ASE | Hinge_del | CH2 | CH3_cah_LS_G1m3/17 |
| Ck(AS/Q124E) del216- | | | | | | |
| 220 cah LS | | | | | | |
| PGDM1400 VH G1m17 | G1m17 | PGDM1400 | Ck_ASE | Hinge_del | CH2_GAALIE | CH3_cah_LS_G1m3/17 |
| Ck(AS/Q124E) del216- | | | | | | |
| 220 GAALIE cah LS | | | | | | |
| PGDM1400 VH G1m17 | G1m17 | PGDM1400 | Ck_ASE | Hinge_del | CH2_GAIE | CH3_cah_LS_G1m3/17 |
| Ck(AS/Q124E) del216- | | | | | | |
| 220 GAIE cah LS | | | | | | |
| PGDM1400 VH G1m17 | G1m17 | PGDM1400 | Ck_ASE | Hinge_del | CH2_GA | CH3_cah_LS_G1m3/17 |
| Ck(AS/Q124E) del216- | | | | | | |
| 220 GA cah LS | | | | | | |
| PGDM1400 VH G1m17 | G1m17 | PGDM1400 | Ck_ASE | Hinge_del | CH2_IE | CH3_cah_LS_G1m3/17 |
| Ck(AS/Q124E) del216- | | | | | | |
| 220 IE cah LS | | | | | | |
| PGDM1400 VH G1m17 | G1m17 | PGDM1400 | Ck_ASE | Hinge_del | CH2 | CH3_cak_LS_G1m3/17 |
| Ck(AS/Q124E) del216- | | | | | | |
| 220 cak LS | | | | | | |
| PGDM1400 VH G1m17 | G1m17 | PGDM1400 | Ck_ASE | Hinge_del | CH2_GAALIE | CH3_cak_LS_G1m3/17 |
| Ck(AS/Q124E) del216- | | | | | | |
| 220 GAALIE cak LS | | | | | | |
| PGDM1400 VH G1m17 | G1m17 | PGDM1400 | Ck_ASE | Hinge_del | CH2_GAIE | CH3_cak_LS_G1m3/17 |
| Ck(AS/Q124E) del216- | | | | | | |
| 220 GAIE cak LS | | | | | | |
| PGDM1400 VH G1m17 | G1m17 | PGDM1400 | Ck_ASE | Hinge_del | CH2_GA | CH3_cak_LS_G1m3/17 |
| Ck(AS/Q124E) del216- | | | | | | |
| 220 GA cak LS | | | | | | |
| PGDM1400 VH G1m17 | G1m17 | PGDM1400 | Ck_ASE | Hinge_del | CH2_IE | CH3_cak_LS_G1m3/17 |
| Ck(AS/Q124E) del216- | | | | | | |
| 220 IE cak LS | | | | | | |
| PGDM1400 VH G1m17,1 | G1m17,1 | PGDM1400 | Ck_ASE | Hinge_del | CH2 | CH3_G1m17,1 |
| Ck(AS/Q124E) del216- | | | | | | |
| 220 | | | | | | |
| PGDM1400 VH G1m17,1 | G1m17,1 | PGDM1400 | Ck_ASE | Hinge_del | CH2 | CH3_LS_G1m17,1 |
| Ck(AS/Q124E) del216- | | | | | | |
| 220 LS | | | | | | |
| PGDM1400 VH G1m17,1 | G1m17,1 | PGDM1400 | Ck_ASE | Hinge_del | CH2_GAALIE | CH3_LS_G1m17,1 |
| Ck(AS/Q124E) del216- | | | | | | |
| 220 GAALIE LS | | | | | | |
| PGDM1400 VH G1m17,1 | G1m17,1 | PGDM1400 | Ck_ASE | Hinge_del | CH2_GAIE | CH3_LS_G1m17,1 |
| Ck(AS/Q124E) del216- | | | | | | |
| 220 GAIE LS | | | | | | |
| PGDM1400 VH G1m17,1 | G1m17,1 | PGDM1400 | Ck_ASE | Hinge_del | CH2_GA | CH3_LS_G1m17,1 |
| Ck(AS/Q124E) del216- | | | | | | |
| 220 GA LS | | | | | | |
| PGDM1400 VH G1m17,1 | G1m17,1 | PGDM1400 | Ck_ASE | Hinge_del | CH2_IE | CH3_LS_G1m17,1 |
| Ck(AS/Q124E) del216- | | | | | | |
| 220 IE LS | | | | | | |
| PGDM1400 VH G1m17,1 | G1m17,1 | PGDM1400 | Ck_ASE | Hinge_del | CH2 | CH3_cah_LS_G1m17,1 |
| Ck(AS/Q124E) del216- | | | | | | |
| 220 cah LS | | | | | | |
| PGDM1400 VH G1m17,1 | G1m17,1 | PGDM1400 | Ck_ASE | Hinge_del | CH2_GAALIE | CH3_cah_LS_G1m17,1 |
| Ck(AS/Q124E) del216- | | | | | | |
| 220 GAALIE cah LS | | | | | | |
| PGDM1400 VH G1m17,1 | G1m17,1 | PGDM1400 | Ck_ASE | Hinge_del | CH2_GAIE | CH3_cah_LS_G1m17,1 |
| Ck(AS/Q124E) del216- | | | | | | |
| 220 GAIE cah LS | | | | | | |
| PGDM1400 VH G1m17,1 | G1m17,1 | PGDM1400 | Ck_ASE | Hinge_del | CH2_GA | CH3_cah_LS_G1m17,1 |
| Ck(AS/Q124E) del216- | | | | | | |
| 220 GA cah LS | | | | | | |
| PGDM1400 VH G1m17,1 | G1m17,1 | PGDM1400 | Ck_ASE | Hinge_del | CH2_IE | CH3_cah_LS_G1m17,1 |
| Ck(AS/Q124E) del216- | | | | | | |
| 220 IE cah LS | | | | | | |
| PGDM1400 VH G1m17,1 | G1m17,1 | PGDM1400 | Ck_ASE | Hinge_del | CH2 | CH3_cak_LS_G1m17,1 |
| Ck(AS/Q124E) del216- | | | | | | |
| 220 cak LS | | | | | | |
| PGDM1400 VH G1m17,1 | G1m17,1 | PGDM1400 | Ck_ASE | Hinge_del | CH2_GAALIE | CH3_cak_LS_G1m17,1 |
| Ck(AS/Q124E) del216- | | | | | | |
| 220 GAALIE cak LS | | | | | | |
| PGDM1400 VH G1m17,1 | G1m17,1 | PGDM1400 | Ck_ASE | Hinge_del | CH2_GAIE | CH3_cak_LS_G1m17,1 |
| Ck(AS/Q124E) del216- | | | | | | |
| 220 GAIE cak LS | | | | | | |
| PGDM1400 VH G1m17,1 | G1m17,1 | PGDM1400 | Ck_ASE | Hinge_del | CH2_GA | CH3_cak_LS_G1m17,1 |
| Ck(AS/Q124E) del216- | | | | | | |
| 220 GA cak LS | | | | | | |
| PGDM1400 VH G1m17,1 | G1m17,1 | PGDM1400 | Ck_ASE | Hinge_del | CH2_IE | CH3_cak_LS_G1m17,1 |
| Ck(AS/Q124E) del216- | | | | | | |
| 220 IE cak LS | | | | | | |

| CrossMabCH1-CLx | | | | | | |
|---|---|---|---|---|---|---|
| format Light Chain | Allotype | VL | CH1 | — | — | — |
| PGDM1400 VL CH1(SS) (G1m3) | G1m3 | PGDM1400 | CH1(SS) | — | — | — |
| | | | (G1m3) | | | |
| PGDM1400 VL CH1(SS) (G1m17±,1) | G1m17±,1 | PGDM1400 | CH1(SS) | — | — | — |
| | | | (G1m17±,1) | | | |

### C. CrossMab^{CH1-CLcv}

In some embodiments, antibody agents as described herein are in a format where one or more charge variants (cv) are introduced into a domain (e.g., a constant domain, e.g., a CH₁ domain, a CL domain, or a combination thereof). Such formats are referred to herein as “CrossMab^{CH1-CLcv}” or “CH₁-cv”. In some embodiments, such antibody agents include charge variants in both arms of the antibody (see e.g., FIG. 4, Panel D). Exemplary charge variants are described e.g., in WO2017055539 A1, which is herein incorporated by reference in its entirety. In some embodiments, in contrast to the formats described in WO2017055539 A1, CH₁-cv antibody agents described do not include a swap of the antibody CH₁ and CL domains in either arm of the antibody.

In some embodiments, a charge variant (cv) is introduced into a CH₁ and/or CL domain of an antibody agent in order to prevent mispairing of immunoglobulin chains of an antibody agent. Such charge variants may include, for example, introducing one or more positively charged amino acid residues in a CH₁ domain and one or more negatively charged amino acid residues in the CL, or vice versa, at specific positions in the CH₁ and CL interface.

In some embodiments, a polyribonucleotide may encode a heavy chain and/or light chain of a CrossMab^{CH1-CLcv} antibody agent as described herein.

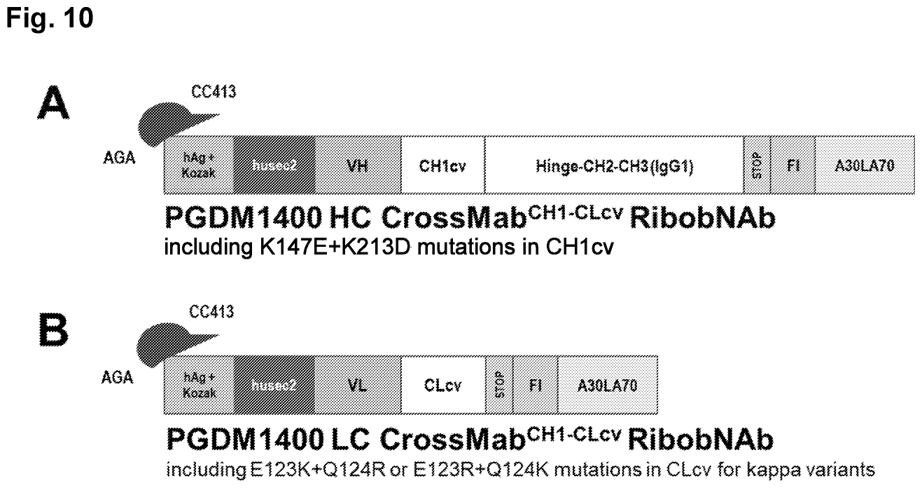

In some embodiments, a CrossMab^{CH1-CLcv} antibody agent may be encoded by two separate polyribonucleotide: a first polyribonucleotide comprising a coding region that encodes (in 5′ to 3′ order): a heavy chain variable domain (VH), a CH₁ domain that includes one or more charge variants as described herein, a hinge region, a CH₂ domain, and a CH₃ domain (see e.g., FIG. 10, Panel A); and a second polyribonucleotide comprising a coding region that encodes (in 5′ to 3′ order): a light chain variable domain (VL) and a light chain constant domain (CL) that includes one or more charge variants as described herein (see e.g., FIG. 10, Panel B).

In some embodiments, a CrossMab^{CH1-CLcv} antibody agent comprises a first immunoglobulin chain, wherein the first immunoglobulin chain comprises a CL domain and the CL domain an amino acid at position 123 (EU numbering) that is substituted by an amino acid selected from K, R and H. In some embodiments, a CrossMab^{CH1-CLcv} antibody agent comprises a first immunoglobulin chain, wherein the first immunoglobulin chain comprises a CL domain and the CL domain an amino acid at position 124 (EU numbering) that is substituted by an amino acid selected from K, R and H. In some embodiments, a CrossMab^{CH1-CLcv} antibody agent comprises a second immunoglobulin chain, wherein the second immunoglobulin chain comprises a CH₁ domain having an amino acid at position 147 (EU numbering) that is substituted by an amino acid selected from E or D (see WO2017055539A1, which is herein incorporated by reference in its entirety).

In some embodiments, a CrossMab^{CH1-CLcv} antibody agent encoded by one or more polyribonucleotides as described herein comprises a CH₁ domain that comprises one or more charge variant mutations. In some embodiments, a CrossMab^{CH1-CLcv} antibody agent comprises a CH₁ domain, wherein the CH₁ domain comprises one or more substitutions including K147E, K213D, or a combination thereof. In some embodiments, a CrossMab^{CH1-CLcv} antibody agent comprises a CH₁ domain that has at least 90%, 91%, 92%, 93%, 94%, 95%, 96%, 97%, 98%, or 99% sequence identity to SEQ ID NO: 84 or 87. In some embodiments, a CrossMab^{CH1-CLcv} antibody agent comprises a CH₁ domain having an amino acid sequence according to SEQ ID NO: 84 or 87. In some embodiments, a CrossMab^{CH1-CLcv}, antibody agent comprises a CH₁ domain that comprises a G1m3 allotype. In some embodiments, a CrossMab^{CH1-CLcv} antibody agent comprises a CH₁ domain that comprises a G1m17 allotype.

In some embodiments, a CrossMab^{CH1-CLcv} antibody agent encoded by one or more polyribonucleotides as described herein comprises a heavy chain encoded by a nucleic acid sequence that has at least 80%, 85%, 90%, 91%, 92%, 93%, 94%, 95%, 96%, 97%, 98%, or 99% sequence identity to a sequence represented by SEQ ID NO: 362-457, or 1323-1324. In some embodiments, a CrossMab^{CH1-CLcv} antibody agent comprises a heavy chain encoded by a nucleic acid sequence represented by any one of SEQ ID NOs: 362-457, 1323-1324. In some embodiments, a CrossMab^{CH1-CLcv} antibody agent comprises a light chain encoded by a nucleic acid sequence that has at least at least 80%, 85%, 90%, 91%, 92%, 93%, 94%, 95%, 96%, 97%, 98%, or 99% sequence identity to SEQ ID NO: 848-849, 1326-1327, or 1353-1354. In some embodiments, a CrossMab^{CH1-CLcv} antibody agent comprises a light chain encoded by a nucleic acid sequence represented in SEQ ID NO: 848-849, 1326-1327, or 1353-1354.

In some embodiments, a CrossMab^{CH1-CLcv} antibody agent encoded by one or more polyribonucleotides as described herein comprises a heavy chain comprising an amino acid sequence that has at least 80%, 85%, 90%, 91%, 92%, 93%, 94%, 95%, 96%, 97%, 98%, or 99% sequence identity to a sequence represented by SEQ ID NO: 1325. In some embodiments, a CrossMab^{CH1-CLcv} antibody agent comprises a heavy chain comprising an amino acid sequence represented by SEQ ID NOs: 1325. In some embodiments, a CrossMab^{CH1-CLcv} antibody agent comprises a light chain comprising an amino acid sequence that has at least at least 80%, 85%, 90%, 91%, 92%, 93%, 94%, 95%, 96%, 97%, 98%, or 99% sequence identity to SEQ ID NO: 1328 or 1355. In some embodiments, a CrossMab^{CH1-CLcv} antibody agent comprises a light chain encoded by a nucleic acid sequence represented in SEQ ID NO: 1328 or 1355.

In some embodiments, a CrossMab^{CH1-CLcv} antibody agent encoded by one or more polyribonucleotides provided herein includes all or part of a PGDM1400 antibody. In some embodiments, a CrossMab^{CH1-CLcv} antibody agent comprises a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof. In some embodiments, a CrossMab^{CH1-CLcv} antibody agent comprises a heavy chain variable domain comprising: (i) HCDR1 (SEQ ID NO: 18); (ii) HCDR2 (SEQ ID NO: 21); and (iii) HCDR3 (SEQ ID NO: 24). In some embodiments, a CrossMab^{CH1-CLcv} antibody agent comprises a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a CrossMab^{CH1-CLcv} antibody agent comprises a light chain variable domain comprising: (i) LCDR1 (SEQ ID NO: 27); (ii) LCDR2 (LAS; SEQ ID NO: 30); and (iii) LCDR3 (SEQ ID NO: 33). In some embodiments, a CrossMab^{CH1-CLcv} antibody agent comprises (a) a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof; and (b) a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a CrossMab^{CH1-CLcv} antibody agent comprises (a) a heavy chain variable domain comprising: (i) HCDR1 (SEQ ID NO: 18); (ii) HCDR2 (SEQ ID NO: 21); and (iii) HCDR3 (SEQ ID NO: 24) and (b) a light chain variable domain comprising: (i) LCDR1 (SEQ ID NO: 27); (ii) LCDR2 (LAS; SEQ ID NO: 30); and (iii) LCDR3 (SEQ ID NO: 33).

In some embodiments, a polyribonucleotide described herein encodes an immunoglobulin chain of a CrossMab^{CH1-CLcv} antibody agent, wherein the immunoglobulin chain comprises a heavy chain variable domain, and wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof. In some embodiments, a polyribonucleotide described herein encodes an immunoglobulin chain of a CrossMab^{CH1-CLcv} antibody agent, wherein the immunoglobulin chain comprises a heavy chain variable domain, and wherein the heavy chain variable domain comprises: (i) HCDR1 (SEQ ID NO: 18); (ii) HCDR2 (SEQ ID NO: 21); and (iii) HCDR3 (SEQ ID NO: 24). In some embodiments, a polyribonucleotide described herein encodes an immunoglobulin chain of a CrossMab^{CH1-CLcv} antibody agent, wherein the immunoglobulin chain comprises a light chain variable domain, and wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain of a CrossMab^{CH1-CLcv} antibody agent, wherein the immunoglobulin chain comprises a light chain variable domain, and wherein the light chain variable domain comprises: (i) LCDR1 (SEQ ID NO: 27); (ii) LCDR2 (LAS; SEQ ID NO: 30); and (iii) LCDR3 (SEQ ID NO: 33). In some embodiments, a polyribonucleotide described herein encodes two immunoglobulin chains of a CrossMab^{CH1-CLcv} antibody agent: a first immunoglobulin chain comprising a heavy chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof; and a second immunoglobulin chain comprising a light chain variable domain, wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes two immunoglobulin chains of a CrossMab^{CH1-CLcv} antibody agent: a first immunoglobulin chain comprising a heavy chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24); and a second immunoglobulin chain comprising a light chain variable domain, wherein the light chain variable domain comprises: (i) LCDR1 (SEQ ID NO: 27); (ii) LCDR2 (LAS; SEQ ID NO: 30); and (iii) LCDR3 (SEQ ID NO: 33).

In some embodiments, a CrossMab^{CH1-CLcv} antibody agent comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a CrossMab^{CH1-CLcv} antibody agent comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a CrossMab^{CH1-CLcv} antibody agent comprises a heavy chain variable domain represented by SEQ ID NO: 36. In some embodiments, a CrossMab^{CH1-CLcv} antibody agent comprises a light chain variable domain represented by SEQ ID NO: 43.

In some embodiments, a CrossMab^{CH1-CLcv} antibody agent comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 1494. In some embodiments, a CrossMab^{CH1-CLcv} antibody agent comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a CrossMab^{CH1-CLcv} antibody agent comprises a heavy chain variable domain represented by SEQ ID NO: 1494. In some embodiments, a CrossMab^{CH1-CLcv} antibody agent comprises a light chain variable domain represented by SEQ ID NO: 43.

In some embodiments, an antibody agent (e.g., a PGDM1400 antibody agent) encoded by one or more polyribonucleotides provided herein comprises a heavy chain variable domain that comprises one or more mutations relative to an amino acid sequence represented by SEQ ID NO: 1494. In some embodiments, substitution mutations are present at residues 3 and/or 5 relative to SEQ ID NO: 1494. In some embodiments, a substitution mutation at residue 3 results in a positively charged amino acid being present at residue 3 relative to SEQ ID NO: 1494. Positively charged amino acids include Lys (K), Arg (R), and His (H). In some embodiments, a substitution mutation at residue 3 relative to SEQ ID NO: 1494 comprises Q3H. In some embodiments, a substitution mutation at residue 5 results in a polar amino acid being present at residue 5 relative to SEQ ID NO: 1494. Polar amino acids include Ser (S), Thr (T), Tyr (Y), Asn (N), and Gln (Q). In some embodiments, a substitution mutation at residue 5 relative to SEQ ID NO: 1494 comprises V5T.

In some embodiments, an antibody agent (e.g., a PGDM1400 antibody agent) encoded by one or more polyribonucleotides provided herein comprises a heavy chain variable domain that comprises SEQ ID NO: 1494 with substitution mutations Q3H and V5T.

In some embodiments, a polyribonucleotide described herein encodes an immunoglobulin chain of a CrossMab^{CH1-CLcv} antibody agent, wherein the immunoglobulin chain comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a polyribonucleotide described herein encodes an immunoglobulin chain of a CrossMab^{CH1-CLcv} antibody agent, wherein the immunoglobulin chain comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a polyribonucleotide described herein encodes an immunoglobulin chain of a CrossMab^{CH1-CLcv} antibody agent, wherein the immunoglobulin chain comprises a heavy chain variable domain represented by SEQ ID NO: 36. In some embodiments, a polyribonucleotide described herein encodes an immunoglobulin chain of a CrossMab^{CH1-CLcv} antibody agent, wherein the immunoglobulin chain comprises a light chain variable domain represented by SEQ ID NO: 43.

As described above, CrossMab^{CH1-CLcv} antibody agents encoded by one or more polyribonucleotides as described herein may comprise one or more heavy chain constant domains. In some embodiments, one or more heavy chain constant domains comprise a CH₃ domain. In some embodiments, a CrossMab^{CH1-CLcv} antibody agent comprises a CH₃ domain that comprises a G1m3, G1m17, or a G1m17,1 allotype. In some embodiments, a CrossMab^{CH1-CLcv} antibody agent comprises a CH₃ domain that comprises an amino acid sequence at least 90%, 91%, 92%, 93%, 94%, 95%, 96%, 97%, 98%, or 99% identical to a sequence represented by represented in any one of SEQ ID NOs: 113, 116, 119, 122, 125, 128, 131, 134, 137, 140, 143, 146, or 1495. In some embodiments, a CrossMab^{CH1-CLcv} antibody agent comprises a CH₃ domain that comprises an amino acid sequence represented in any one of SEQ ID NOs: 113, 116, 119, 122, 125, 128, 131, 134, 137, 140, 143, 146, 1495. In some embodiments, a CH₃ domain comprises amino acid sequence SEQ ID NO: 1495 with substitution mutations D16E and L18M.

CrossMab^{CH1-CLcv} antibody agents encoded by one or more polyribonucleotides as described herein may comprise one or more heavy chain constant domains comprising an amino acid modification (e.g., a substitution or deletion) at one or more amino acid positions. In some embodiments, a CrossMab^{CH1-CLcv} antibody agent as described herein may include the L/S mutation within the CH₃ region (for enhanced FcRn binding) (see Zalevsky J et al., Nat Biotechnol. 2010, which is herein incorporated by reference). Such mutations are noted as M428L and N434S according to EU numbering (i.e., M88L and N94S within the CH₃ domain, e.g., SEQ ID NO: 113, 116, or 1495), and referred to herein as “LS” or “L/S”. In some embodiments, a CrossMab^{CH1-CLcv} antibody agent comprises an E294 deletion (for Fc hypersialylation) (see Bas M et al., J Immunol 2019, which is herein incorporated by reference).

In some embodiments, a CrossMab^{CH1-CLcv} antibody agent encoded by one or more polyribonucleotides comprises a CH₃ domain comprising one or more of the following mutations: Y349C, T366S, L368A, and Y407V (according to EU numbering). Such a combination of mutations is referred to herein as “cah”. In some embodiments, a CH₁-cv antibody agent comprises a CH₃ domain comprising one or more mutations selected from: S354C and T366W (according to EU numbering). Such a combination of CH₃ mutations is referred to herein as “cak”.

In some embodiments, a CrossMab^{CH1-CLcv} antibody agent encoded by one or more polyribonucleotides comprises a hinge domain that comprises an amino acid sequence represented in SEQ ID NO: 161.

In some embodiments, a CrossMab^{CH1-CLcv} antibody agent encoded by one or more polyribonucleotides comprises a CH₂ domain that comprises an amino acid sequence at least 90%, 91%, 92%, 93%, 94%, 95%, 96%, 97%, 98%, or 99% identical to represented in SEQ ID NO: 96. In some embodiments, a CH₁-cv antibody agent comprises a CH₂ domain that comprises an amino acid sequence represented in SEQ ID NO: 53. In some embodiments, a CrossMab^{CH1-CLcv} antibody agent comprises a CH₂ domain that comprises one or more mutations (e.g., with respect to SEQ ID NO: 96). For example, in some embodiments, a CrossMab^{CH1-CLcv} antibody agent comprises one or more of the following mutations: G236A, A330L, and I332E (according to EU numbering). Such a combination of mutations is referred to herein as “GAALIE”. Such mutations in the CH₂ domain have been associated with increased affinity to Fc receptors FcgRIIA and FcgRIII for enhanced antibody effector function. In some embodiments, a CrossMab^{CH1-CLcv} antibody agent comprises a CH₂ domain that comprises an amino acid sequence that is at least 90%, 91%, 92%, 93%, 94%, 95%, 96%, 97%, 98%, or 99% identical to SEQ ID NO: 99 or 102. In some embodiments, a CrossMab^{CH1-CLcv} antibody agent comprises a CH₂ domain that comprises an amino acid sequence represented in SEQ ID NO: 99 or 102. In some embodiments, a CrossMab^{CH1-CLcv} antibody agent comprises a CH₂ domain with one or more mutations selected from: G236A and I332E (according to EU numbering). Such a combination of CH₂ mutations is referred to herein as “GAIE”. In some embodiments, a CrossMab^{CH1-CLcv} antibody agent comprises a CH₂ domain that comprises an amino acid sequence at least 90%, 91%, 92%, 93%, 94%, 95%, 96%, 97%, 98%, or 99% identical to SEQ ID NO: 104. In some embodiments, a CrossMab^{CH1-CLcv} antibody agent comprises a CH₂ domain that comprises an amino acid sequence represented in SEQ ID NO: 104. In some embodiments, a CrossMab^{CH1-CLcv} antibody agent comprises a mutation: G236A (according to EU numbering), referred to herein as “GA”. In some embodiments, a CrossMab^{CH1-CLcv} antibody agent comprises a CH₂ domain that comprises an amino acid sequence at least 90%, 91%, 92%, 93%, 94%, 95%, 96%, 97%, 98%, or 99% identical to SEQ ID NO: 107. In some embodiments, a CrossMab^{CH1-CLcv} antibody agent comprises a CH₂ domain that comprises an amino acid sequence represented in SEQ ID NO: 107. In some embodiments, a CrossMab^{CH1-CLcv} antibody agent comprises a mutation: I332E (according to EU numbering), referred to herein as “IE”. In some embodiments, a CH₁-cv antibody agent comprises a CH₂ domain that comprises an amino acid sequence at least 90%, 91%, 92%, 93%, 94%, 95%, 96%, 97%, 98%, or 99% identical to SEQ ID NO: 110. In some embodiments, a CrossMab^{CH1-CLcv} antibody agent comprises a CH₂ domain that comprises an amino acid sequence represented in SEQ ID NO: 110.

In some embodiments, a CrossMab^{CH1-CLcv} antibody agent encoded by one or more polyribonucleotides comprises a signal peptide comprising a husec2 signal peptide. In some embodiments, a CrossMab^{CH1-CLcv} antibody agent comprises a signal peptide comprising SEQ ID NO: 1.

In some embodiments, a CrossMab^{CH1-CLcv} antibody agent encoded by one or more polyribonucleotides comprises a light chain constant domain that comprises a kappa light chain constant domain. In some embodiments, a CrossMab^{CH1-CLcv} antibody agent comprises a kappa light chain constant domain comprising one or more charge variant mutations. In some embodiments, a CrossMab^{CH1-CLcv} antibody agent comprises one or more charge variants selected from the mutations: a E123K and Q124R (i.e., mutations that impart a positive charge). In some embodiments, a CrossMab^{CH1-CLcv} antibody agent comprises one or more charge variants selected from the mutations: a E123R and Q124K (i.e., mutations that impart a positive charge). Such mutations, when paired with a CH₁ domain with mutations that impart a negative charge (e.g., K147E and/or K213D) in the CH₁ and CL interface, prevent mispairing between the antibody variable domains of an antibody agent. In some embodiments, a CrossMab^{CH1-CLcv} antibody comprises a kappa light chain constant domain comprising an amino acid sequence at least 80, 85, 90%, 91%, 92%, 93%, 94%, 95%, 96%, 97%, 98%, or 99% identical to SEQ ID NO: 155 or 158. In some embodiments, a CrossMab^{CH1-CLcv} antibody comprises a kappa light chain constant domain comprising an amino acid sequence represented in SEQ ID NO: 155 or 158. In some embodiments, a CrossMab^{CH1-CLcv} antibody agent comprises a lambda light chain constant domain.

In some embodiments, an antibody agent described herein may be a combination of a CrossMab^{CH1-CLx} and CrossMab^{CH1-CLcv} antibody agent. For example, an antibody agent described herein can include one arm of the antibody that is in a CrossMab^{CH1-CLx} format and a second arm of the antibody in a CrossMab^{CH1-CLv} format.

In some embodiments, antibody agents described herein can be designed to include one or more mutations to utilize KIH technology. For example, in some embodiments, a CrossMab^{CH1-CLx} antibody agent as described herein can be designed to further include mutations to utilize KIH technology. In some embodiments, a CrossMab^{CH1-CLx} antibody agent as described herein can be designed to further include mutations to utilize KIH technology. In some embodiments, KIH technology may be applied to promote pairing of two different heavy chains, for example, to produce a bispecific and/or bivalent antibody agent. In some embodiments, KIH technology may be applied to promote pairing of two different heavy chains, for example, to produce a bispecific and/or bivalent antibody comprising one arm of the antibody that comprises an immunoglobulin chain of a CrossMab^{CH1-CLx} antibody agent and a second arm that comprises an immunoglobulin chain of a CrossMab^{CH1-cv} antibody agent.

In some embodiments, a polyribonucleotide encoding a CrossMab^{CH1-CLcv} antibody agent comprises a ribonucleic acid sequence that encodes any one of the immunoglobulin heavy chain configurations in Table 5, corresponding to SEQ ID NOs: 362-457, and 1323-1324. In some embodiments, a polyribonucleotide encoding a CrossMab^{CH1-CLcv} antibody comprises a ribonucleic acid sequence that encodes any one of the immunoglobulin light chain configurations in Table 5, corresponding to SEQ ID NOs: 848-849, 1326-1327, and 1353-1354.

Exemplary heavy chain and light chain configurations of a CrossMab^{CH1-CLcv} antibody agent, as described herein, are shown in Table 5 below.

TABLE 5
CrossMab^{CH1-CLcv} Exemplary Chain Configurations

| CrossMab^{CH1-CLcv} | | | | | | |
|---|---|---|---|---|---|---|
| Heavy Chain Formats | Allotype | VH | CH1 | Hinge | CH2 | CH3 |
| PGDM1400 VH G1m3 | G1m3 | PGDM1400 | CH1_ED_G1m3 | Hinge | CH2 | CH3_G1m3/17 |
| K147E + K213D | | | | | | |
| PGDM1400 VH G1m3 | G1m3 | PGDM1400 | CH1_ED_G1m3 | Hinge | CH2 | CH3_LS_G1m3/17 |
| K147E + K213D LS | | | | | | |
| PGDM1400 VH G1m3 | G1m3 | PGDM1400 | CH1_ED_G1m3 | Hinge | CH2_GAALIE | CH3_LS_G1m3/17 |
| K147E + K213D | | | | | | |
| GAALIE LS | | | | | | |
| PGDM1400 VH G1m3 | G1m3 | PGDM1400 | CH1_ED_G1m3 | Hinge | CH2_GAIE | CH3_LS_G1m3/17 |
| K147E + K213D GAIE | | | | | | |
| LS | | | | | | |
| PGDM1400 VH G1m3 | G1m3 | PGDM1400 | CH1_ED_G1m3 | Hinge | CH2_GA | CH3_LS_G1m3/17 |
| K147E + K213D GA LS | | | | | | |
| PGDM1400 VH G1m3 | G1m3 | PGDM1400 | CH1_ED_G1m3 | Hinge | CH2_IE | CH3_LS_G1m3/17 |
| K147E + K213D IE LS | | | | | | |
| PGDM1400 VH G1m3 | G1m3 | PGDM1400 | CH1_ED_G1m3 | Hinge | CH2 | CH3_cah_LS_G1m3/17 |
| K147E + K213D cah | | | | | | |
| LS | | | | | | |
| PGDM1400 VH G1m3 | G1m3 | PGDM1400 | CH1_ED_G1m3 | Hinge | CH2_GAALIE | CH3_cah_LS_G1m3/17 |
| K147E + K213D | | | | | | |
| GAALIE cah LS | | | | | | |
| PGDM1400 VH G1m3 | G1m3 | PGDM1400 | CH1_ED_G1m3 | Hinge | CH2_GAIE | CH3_cah_LS_G1m3/17 |
| K147E + K213D GAIE | | | | | | |
| cah LS | | | | | | |
| PGDM1400 VH G1m3 | G1m3 | PGDM1400 | CH1_ED_G1m3 | Hinge | CH2_GA | CH3_cah_LS_G1m3/17 |
| K147E + K213D GA cah | | | | | | |
| LS | | | | | | |
| PGDM1400 VH G1m3 | G1m3 | PGDM1400 | CH1_ED_G1m3 | Hinge | CH2_IE | CH3_cah_LS_G1m3/17 |
| K147E + K213D IE cah | | | | | | |
| LS | | | | | | |
| PGDM1400 VH G1m3 | G1m3 | PGDM1400 | CH1_ED_G1m3 | Hinge | CH2 | CH3_cak_LS_G1m3/17 |
| K147E + K213D cak LS | | | | | | |
| PGDM1400 VH G1m3 | G1m3 | PGDM1400 | CH1_ED_G1m3 | Hinge | CH2_GAALIE | CH3_cak_LS_G1m3/17 |
| K147E + K213D | | | | | | |
| GAALIE cak LS | | | | | | |
| PGDM1400 VH G1m3 | G1m3 | PGDM1400 | CH1_ED_G1m3 | Hinge | CH2_GAIE | CH3_cak_LS_G1m3/17 |
| K147E + K213D GAIE | | | | | | |
| cak LS | | | | | | |
| PGDM1400 VH G1m3 | G1m3 | PGDM1400 | CH1_ED_G1m3 | Hinge | CH2_GA | CH3_cak_LS_G1m3/17 |
| K147E + K213D GA cak | | | | | | |
| LS | | | | | | |
| PGDM1400 VH G1m3 | G1m3 | PGDM1400 | CH1_ED_G1m3 | Hinge | CH2_IE | CH3_cak_LS_G1m3/17 |
| K147E + K213D IE cak | | | | | | |
| LS | | | | | | |
| PGDM1400 VH G1m17 | G1m17 | PGDM1400 | CH1_ED_G1m17 | Hinge | CH2 | CH3_G1m3/17 |
| K147E + K213D | | | | | | |
| PGDM1400 VH G1m17 | G1m17 | PGDM1400 | CH1_ED_G1m17 | Hinge | CH2 | CH3_LS_G1m3/17 |
| K147E + K213D LS | | | | | | |
| PGDM1400 VH G1m17 | G1m17 | PGDM1400 | CH1_ED_G1m17 | Hinge | CH2_GAALIE | CH3_LS_G1m3/17 |
| K147E + K213D | | | | | | |
| GAALIE LS | | | | | | |
| PGDM1400 VH G1m17 | G1m17 | PGDM1400 | CH1_ED_G1m17 | Hinge | CH2_GAIE | CH3_LS_G1m3/17 |
| K147E + K213D GAIE | | | | | | |
| LS | | | | | | |
| PGDM1400 VH G1m17 | G1m17 | PGDM1400 | CH1_ED_G1m17 | Hinge | CH2_GA | CH3_LS_G1m3/17 |
| K147E + K213D GA LS | | | | | | |
| PGDM1400 VH G1m17 | G1m17 | PGDM1400 | CH1_ED_G1m17 | Hinge | CH2_IE | CH3_LS_G1m3/17 |
| K147E + K213D IE LS | | | | | | |
| PGDM1400 VH G1m17 | G1m17 | PGDM1400 | CH1_ED_G1m17 | Hinge | CH2 | CH3_cah_LS_G1m3/17 |
| K147E + K213D cah | | | | | | |
| LS | | | | | | |
| PGDM1400 VH G1m17 | G1m17 | PGDM1400 | CH1_ED_G1m17 | Hinge | CH2_GAALIE | CH3_cah_LS_G1m3/17 |
| K147E + K213D | | | | | | |
| GAALIE cah LS | | | | | | |
| PGDM1400 VH G1m17 | G1m17 | PGDM1400 | CH1_ED_G1m17 | Hinge | CH2_GAIE | CH3_cah_LS_G1m3/17 |
| K147E + K213D GAIE | | | | | | |
| cah LS | | | | | | |
| PGDM1400 VH G1m17 | G1m17 | PGDM1400 | CH1_ED_G1m17 | Hinge | CH2_GA | CH3_cah_LS_G1m3/17 |
| K147E + K213D GA cah | | | | | | |
| LS | | | | | | |
| PGDM1400 VH G1m17 | G1m17 | PGDM1400 | CH1_ED_G1m17 | Hinge | CH2_IE | CH3_cah_LS_G1m3/17 |
| K147E + K213D IE cah | | | | | | |
| LS | | | | | | |
| PGDM1400 VH G1m17 | G1m17 | PGDM1400 | CH1_ED_G1m17 | Hinge | CH2 | CH3_cak_LS_G1m3/17 |
| K147E + K213D cak LS | | | | | | |
| PGDM1400 VH G1m17 | G1m17 | PGDM1400 | CH1_ED_G1m17 | Hinge | CH2_GAALIE | CH3_cak_LS_G1m3/17 |
| K147E + K213D | | | | | | |
| GAALIE cak LS | | | | | | |
| PGDM1400 VH G1m17 | G1m17 | PGDM1400 | CH1_ED_G1m17 | Hinge | CH2_GAIE | CH3_cak_LS_G1m3/17 |
| K147E + K213D GAIE | | | | | | |
| cak LS | | | | | | |
| PGDM1400 VH G1m17 | G1m17 | PGDM1400 | CH1_ED_G1m17 | Hinge | CH2_GA | CH3_cak_LS_G1m3/17 |
| K147E + K213D GA cak | | | | | | |
| LS | | | | | | |
| PGDM1400 VH G1m17 | G1m17 | PGDM1400 | CH1_ED_G1m17 | Hinge | CH2_IE | CH3_cak_LS_G1m3/17 |
| K147E + K213D IE cak | | | | | | |
| LS | | | | | | |
| PGDM1400 VH G1m17,1 | G1m17,1 | PGDM1400 | CH1_ED_G1m17 | Hinge | CH2 | CH3_G1m17,1 |
| K147E + K213D | | | | | | |
| PGDM1400 VH G1m17,1 | G1m17,1 | PGDM1400 | CH1_ED_G1m17 | Hinge | CH2 | CH3_LS_G1m17,1 |
| K147E + K213D LS | | | | | | |
| PGDM1400 VH G1m17,1 | G1m17,1 | PGDM1400 | CH1_ED_G1m17 | Hinge | CH2_GAALIE | CH3_LS_G1m17,1 |
| K147E + K213D | | | | | | |
| GAALIE LS | | | | | | |
| PGDM1400 VH G1m17,1 | G1m17,1 | PGDM1400 | CH1_ED_G1m17 | Hinge | CH2_GAIE | CH3_LS_G1m17,1 |
| K147E + K213D GAIE | | | | | | |
| LS | | | | | | |
| PGDM1400 VH G1m17,1 | G1m17,1 | PGDM1400 | CH1_ED_G1m17 | Hinge | CH2_GA | CH3_LS_G1m17,1 |
| K147E + K213D GA LS | | | | | | |
| PGDM1400 VH G1m17,1 | G1m17,1 | PGDM1400 | CH1_ED_G1m17 | Hinge | CH2_IE | CH3_LS_G1m17,1 |
| K147E + K213D IE LS | | | | | | |
| PGDM1400 VH G1m17,1 | G1m17,1 | PGDM1400 | CH1_ED_G1m17 | Hinge | CH2 | CH3_cah_LS_G1m17,1 |
| K147E + K213D cah | | | | | | |
| LS | | | | | | |
| PGDM1400 VH G1m17,1 | G1m17,1 | PGDM1400 | CH1_ED_G1m17 | Hinge | CH2_GAALIE | CH3_cah_LS_G1m17,1 |
| K147E + K213D | | | | | | |
| GAALIE cah LS | | | | | | |
| PGDM1400 VH G1m17,1 | G1m17,1 | PGDM1400 | CH1_ED_G1m17 | Hinge | CH2_GAIE | CH3_cah_LS_G1m17,1 |
| K147E + K213D GAIE | | | | | | |
| cah LS | | | | | | |
| PGDM1400 VH G1m17,1 | G1m17,1 | PGDM1400 | CH1_ED_G1m17 | Hinge | CH2_GA | CH3_cah_LS_G1m17,1 |
| K147E + K213D GA cah | | | | | | |
| LS | | | | | | |
| PGDM1400 VH G1m17,1 | G1m17,1 | PGDM1400 | CH1_ED_G1m17 | Hinge | CH2_IE | CH3_cah_LS_G1m17,1 |
| K147E + K213D IE cah | | | | | | |
| LS | | | | | | |
| PGDM1400 VH G1m17,1 | G1m17,1 | PGDM1400 | CH1_ED_G1m17 | Hinge | CH2 | CH3_cak_LS_G1m17,1 |
| K147E + K213D cak LS | | | | | | |
| PGDM1400 VH G1m17,1 | G1m17,1 | PGDM1400 | CH1_ED_G1m17 | Hinge | CH2_GAALIE | CH3_cak_LS_G1m17,1 |
| K147E + K213D | | | | | | |
| GAALIE cak LS | | | | | | |
| PGDM1400 VH G1m17,1 | G1m17,1 | PGDM1400 | CH1_ED_G1m17 | Hinge | CH2_GAIE | CH3_cak_LS_G1m17,1 |
| K147E + K213D GAIE | | | | | | |
| cak LS | | | | | | |
| PGDM1400 VH G1m17,1 | G1m17,1 | PGDM1400 | CH1_ED_G1m17 | Hinge | CH2_GA | CH3_cak_LS_G1m17,1 |
| K147E + K213D GA cak | | | | | | |
| LS | | | | | | |
| PGDM1400 VH G1m17,1 | G1m17,1 | PGDM1400 | CH1_ED_G1m17 | Hinge | CH2_IE | CH3_cak_LS_G1m17,1 |
| K147E + K213D IE cak | | | | | | |
| LS | | | | | | |

| CrossMab^{CH1-cv} | | | | | | |
|---|---|---|---|---|---|---|
| format Light Chain | Allotype | VL | CL | — | — | — |
| PGDM1400 VL E123K + Q124R | — | PGDM1400 | E123K + Q124R | — | — | — |
| PGDM1400 VL E123R + Q124K | — | PGDM1400 | E123K + Q124R | — | — | — |

D. scFv-Fc

In some embodiments, an antibody agent described herein (e.g., an HIV antibody agent) is in a single-chain Fv (sFv or scFv) format. A scFv is an antibody fragment that comprises the V_{H} and V_{L} antibody domains connected into a single polypeptide chain. An scFv polypeptide can further comprise a polypeptide linker that connects the V_{H} and V_{L} domains that enable the scFv to form the desired structure for antigen binding (see, e.g., FIG. 4, Panel E, and Pluckthun, The Pharmacology of Monoclonal Antibodies, vol. 113; Rosenburg and Moore eds., Springer-Verlag, New York, pp. 269-315 (1994); each of which is herein incorporated by reference).

Antibody agents as described herein in scFv format can be part of a fusion that includes a scFv domain fused to an Fc domain of an antibody (see e.g., FIG. 4, Panel C, herein referred to as “scFv-Fc” or “scFv-Fc fusion”). Such a format presents certain advantages over other antibody formats in that mispairing of the heavy chain and light chain of the antibody is avoided.

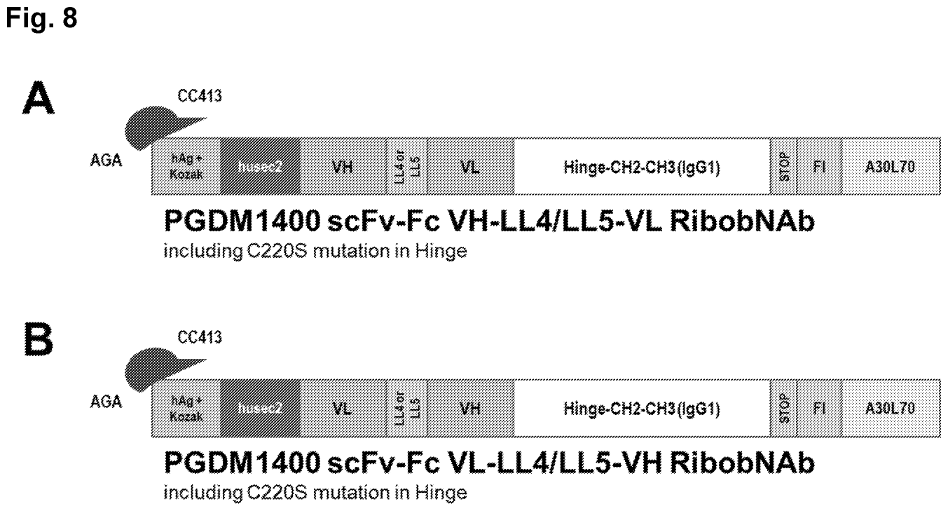

An scFv-Fc fusion antibody agent may be encoded by a single polyribonucleotide comprising a first coding region that encodes a single-chain variable fragment (scFv) that preferentially binds to an epitope of HIV and a Fc domain (e.g., hIgG1). In some embodiments, a polyribonucleotide encodes a scFv in 5′ to 3′ order: heavy chain variable region (VH), linker (e.g., (G4S)₄ (“LL4”) or (G4S)₅ (“LL5”)) and a light chain variable region (VL) (see e.g., FIG. 8, Panel A). In some embodiments, a polyribonucleotide encodes a scFv in 5′ to 3′ order: VL, linker (e.g., (GGGGS)₄ (“LL4”) or (GGGGS)₅(“LL5”)), VH (see e.g., FIG. 8, Panel B).

In some embodiments, a polyribonucleotide may encode a scFv-Fc antibody agent as described herein. In some embodiments, a polyribonucleotide encoding a scFv-Fc antibody heavy chain comprises a ribonucleic acid sequence encoding a VH domain, a hinge domain, a CH₂ domain, and a CH₃ domain.

Linkers included in scFv formats described herein may include a flexible linker. In some embodiments, a flexible linker contains at least 1 flexible amino acid (e.g., Gly). Exemplary flexible linkers include glycine polymers (G)ₙ, glycine-serine polymers (including, for example, (GS)ₙ, (GSGGS)ₙ (SEQ ID NO: 1481) and (GGGGS)ₙ (SEQ ID NO: 1482), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11:173-142 (1992), which is herein incorporated by reference). Exemplary flexible linkers to be used in scFv formats described herein include, but are not limited to:

(SEQ ID NO: 48)
GGGGSGGGGSGGGGSGGGGS
(SEQ ID NO: 59)
GGGGSGGGGSGGGGSGGGGSGGGGS

Linkers utilized in scFv formats as described herein can be readily selected and can be of various lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids (e.g., at least 1, 2, 3, 4, 5, 6, 7, 8, 9, 10, 11, 12, 13, 14, 15, 16, 17, 18, or 19 amino acids).

In some embodiments, an scFv-Fc encoded by one or more polyribonucleotides described herein includes all or part of a PGDM1400 antibody. In some embodiments, an scFv-Fc encoded by one or more polyribonucleotides provided herein includes all or part of a PGDM1400 antibody. In some embodiments, an scFv-Fc comprises a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof. In some embodiments, an scFv-Fc comprises a heavy chain variable domain comprising: (i) HCDR1 (SEQ ID NO: 18); (ii) HCDR2 (SEQ ID NO: 21); and (iii) HCDR3 (SEQ ID NO: 24). In some embodiments, an scFv-Fc comprises a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, an scFv-Fc comprises a light chain variable domain comprising: (i) LCDR1 (SEQ ID NO: 27); (ii) LCDR2 (LAS; SEQ ID NO: 30); and (iii) LCDR3 (SEQ ID NO: 33). In some embodiments, an scFv-Fc comprises (a) a heavy chain variable domain comprising: (i) HCDR1 (SEQ ID NO: 18); (ii) HCDR2 (SEQ ID NO: 21); (iii) HCDR3 (SEQ ID NO: 24); or (iv) a combination thereof; and (b) a light chain variable domain comprising: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, an scFv-Fc comprises (a) a heavy chain variable domain comprising: (i) HCDR1 (SEQ ID NO: 18); (ii) HCDR2 (SEQ ID NO: 21); and (iii) HCDR3 (SEQ ID NO: 24); and (b) a light chain variable domain comprising: (i) LCDR1 (SEQ ID NO: 27); (ii) LCDR2 (LAS; SEQ ID NO: 30); and (iii) LCDR3 (SEQ ID NO: 33).

In some embodiments, a polyribonucleotide described herein encodes all or part of a PGDM1400 antibody. In some embodiments, a polyribonucleotide described herein encodes an immunoglobulin chain comprising a heavy chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof. In some embodiments, a polyribonucleotide described herein encodes an immunoglobulin chain comprising a heavy chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24). In some embodiments, a polyribonucleotide described herein encodes an immunoglobulin chain comprising a light chain variable domain, wherein the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain comprising a light chain variable domain, wherein the light chain variable domain comprises: (i) LCDR1 (SEQ ID NO: 27); (ii) LCDR2 (LAS; SEQ ID NO: 30); and (iii) LCDR3 (SEQ ID NO: 33). In some embodiments, a polyribonucleotide described herein encodes an immunoglobulin chain comprising a heavy chain variable domain and a light chain variable domain, wherein the heavy chain variable domain comprises: (i) HCDR1 (SEQ ID NO: 18); (ii) HCDR2 (SEQ ID NO: 21); (iii) HCDR3 (SEQ ID NO: 24); or (iv) a combination thereof; and the light chain variable domain comprises: (i) LCDR1 (SEQ ID NO: 27); (ii) LCDR2 (LAS; SEQ ID NO: 30); (iii) LCDR3 (SEQ ID NO: 33); or (iv) a combination thereof. In some embodiments, a polyribonucleotide described herein encodes an immunoglobulin chain comprising a heavy chain variable domain and a light chain variable domain, wherein the heavy chain variable domain comprises: (i) HCDR1 (SEQ ID NO: 18); (ii) HCDR2 (SEQ ID NO: 21); and (iii) HCDR3 (SEQ ID NO: 24); and the light chain variable domain comprises: (i) LCDR1 (SEQ ID NO: 27); (ii) LCDR2 (LAS; SEQ ID NO: 30); and (iii) LCDR3 (SEQ ID NO: 33).

In some embodiments, a scFv-Fc comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a scFv-Fc comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a scFv-Fc comprises a heavy chain variable domain represented by SEQ ID NO: 36. In some embodiments, scFv-Fc comprises a light chain variable domain represented by SEQ ID NO: 43.

In some embodiments, a scFv-Fc comprises a heavy chain variable domain having at least 80%, 85%, 90%, 91%, 92%, 93%, 94%, 95%, 96%, 97%, 98%, or 99% sequence identity to an amino acid sequence represented by SEQ ID NO: 1494. In some embodiments, a scFv-Fc comprises a light chain variable domain having at least 80%, 85%, 90%, 91%, 92%, 93%, 94%, 95%, 96%, 97%, 98%, or 99% identity to an amino acid sequence represented by SEQ ID NO: 43. In some embodiments, a scFv-Fc comprises a heavy chain variable domain represented by SEQ ID NO: 1494. In some embodiments, scFv-Fc comprises a light chain variable domain represented by SEQ ID NO: 43.

In some embodiments, an antibody agent (e.g., a PGDM1400 antibody agent) encoded by one or more polyribonucleotides provided herein comprises a heavy chain variable domain that comprises one or more mutations relative to an amino acid sequence represented by SEQ ID NO: 1494. In some embodiments, substitution mutations are present at residues 3 and/or 5 relative to SEQ ID NO: 1494. In some embodiments, a substitution mutation at residue 3 results in a positively charged amino acid being present at residue 3 relative to SEQ ID NO: 1494. Positively charged amino acids include Lys (K), Arg (R), and His (H). In some embodiments, a substitution mutation at residue 3 relative to SEQ ID NO: 1494 comprises Q3H. In some embodiments, a substitution mutation at residue 5 results in a polar amino acid being present at residue 5 relative to SEQ ID NO: 1494. Polar amino acids include Ser (S), Thr (T), Tyr (Y), Asn (N), and Gln (Q). In some embodiments, a substitution mutation at residue 5 relative to SEQ ID NO: 1494 comprises V5T.

In some embodiments, an antibody agent (e.g., a PGDM1400 antibody agent) encoded by one or more polyribonucleotides provided herein comprises a heavy chain variable domain that comprises SEQ ID NO: 1494 with substitution mutations Q3H and V5T.

In some embodiments, a polyribonucleotide described herein encodes an immunoglobulin chain comprising a heavy chain variable domain having at least 80%, 85%, 90%, 91%, 92%, 93%, 94%, 95%, 96%, 97%, 98%, or 99% sequence identity to an amino acid sequence represented by SEQ ID NO: 36. In some embodiments, a polyribonucleotide described herein encodes an immunoglobulin chain comprising a light chain variable domain having at least 80%, 85%, 90%, 91%, 92%, 93%, 94%, 95%, 96%, 97%, 98%, or 99% sequence identity to an amino acid sequence represented by SEQ ID NO: 43. In some embodiments, a polyribonucleotide described herein encodes an immunoglobulin chain comprising a heavy chain variable domain and a light chain variable domain, wherein the heavy chain variable domain has at least 80%, 85%, 90%, 91%, 92%, 93%, 94%, 95%, 96%, 97%, 98%, or 99% sequence identity to an amino acid sequence represented by SEQ ID NO: 36, and wherein the light chain variable domain has at least 80%, 85%, 90%, 91%, 92%, 93%, 94%, 95%, 96%, 97%, 98%, or 99% sequence identity to an amino acid sequence represented by SEQ ID NO: 43.

An Fc region of scFv-Fc described herein may include an Fc region comprising any particular heavy chain constant domain that correspond to the different classes of immunoglobulins which include a, b, E, y, and p, respectively. An Fc region of the scFv-Fc described herein may comprise a human Fc region sequence (e.g., a human IgG1, IgG2, IgG3 or IgG4 Fc region) comprising an amino acid modification (e.g., a substitution) at one or more amino acid positions. In some embodiments, a conventional antibody encoded by one or more polyribonucleotides as described herein comprises a Fc region that comprises a G1m3, G1m17, or a G1m17,1 allotype.

In some embodiments, a scFv-Fc encoded by one or more polyribonucleotides as described herein may comprise one or more heavy chain constant domains. In some embodiments, one or more heavy chain constant domains comprise a CH₃ domain. In some embodiments, a scFv-Fc as described herein may include the L/S mutation within the CH₃ region (for enhanced FcRn binding) (Zalevsky J et al., Nat Biotechnol. 2010, which is herein incorporated by reference). Such mutations are noted as M428L and N434S according to EU numbering (i.e., M88L and N94S within the CH₃ domain, e.g., SEQ ID NO: 113, 116, or 1495), and referred to herein as “LS” or “L/S”.” (see e.g., FIG. 5, Panel D). In some embodiments, a scFv-Fc comprises an E294 deletion (for Fc hypersialylation) (Bas M et al., J Immunol 2019, which is herein incorporated by reference). In some embodiments, a CH₃ domain comprises an amino acid sequence at least 90%, 91%, 92%, 93%, 94%, 95%, 96%, 97%, 98%, or 99% identical to a sequence represented by represented in SEQ ID NO: 116. In some embodiments, a scFv-Fc comprises a CH₃ domain that comprises an amino acid sequence represented in SEQ ID NO: 116.

In some embodiments, a scFv-Fc comprises a CH₃ domain comprising one or more of the following mutations: Y349C, T366S, L368A, and Y407V (according to EU numbering). Such a combination of mutations is referred to herein as “cah”. In some embodiments, a scFv-Fc comprises a CH₃ domain comprising one or more mutations selected from: S354C and T366W (according to EU numbering). Such a combination of CH₃ mutations is referred to herein as “cak”. In some embodiments, a scFv-Fc comprises a CH₃ domain that comprises a G1m3, G1m17, or a G1m17,1 allotype. In some embodiments, a scFv-Fc comprises a CH₃ domain that comprises an amino acid sequence at least 90%, 91%, 92%, 93%, 94%, 95%, 96%, 97%, 98%, or 99% identical to a sequence represented by represented in any one of SEQ ID NOs: 113, 116, 119, 122, 125, 128, 131, 134, 137, 140, 143, 146, or 1495. In some embodiments, a scFv-Fc comprises a CH₃ domain that comprises an amino acid sequence represented in any one of SEQ ID NOs: 113, 116, 119, 122, 125, 128, 131, 134, 137, 140, 143, 146, or 1495.

In some embodiments, a scFv-Fc antibody agent encoded by one or more polyribonucleotides as described herein comprises a CH₃ domain that comprises a G1m3, G1m17, or a G1m17,1 allotype. In some embodiments, a scFv-Fc antibody agent encoded by one or more polyribonucleotides as described herein comprises a CH₃ domain having an amino acid sequence at least 90%, 91%, 92%, 93%, 94%, 95%, 96%, 97%, 98%, or 99% identical to a sequence represented in any one of SEQ ID NOs: 113, 116, 119, 122, 125, 128, 131, 134, 137, 140, 143, 146, or 1495. In some embodiments, a scFv-Fc antibody agent encoded by one or more polyribonucleotides as described herein comprises a CH₃ domain having an amino acid sequence represented in any one of SEQ ID NOs: 113, 116, 119, 122, 125, 128, 131, 134, 137, 140, 143, 146, or 1495. In some embodiments, a CH₃ domain comprises amino acid sequence SEQ ID NO: 1495 with substitution mutations D16E and L18M.

In some embodiments, a CH₃ domain comprises one or more substitution mutations, M88L, N94S, or a combination thereof, relative to the amino acid sequence according to SEQ ID NO: 1495. In some embodiments, a CH₃ domain comprises substitution mutations at residue 16 and residue 18 relative to the amino acid sequence according to SEQ ID NO: 1495. In some embodiments, a CH₃ domain comprises an amino acid sequence SEQ ID NO: 1495 with substitution mutations D16E and L18M. In some embodiments, a CH₃ domain comprises or consists of an amino acid sequence according to SEQ ID NO: 116.

As described herein, a scFv-Fc comprises a scFv fused to an Fc domain of an antibody. Accordingly, such formats do not comprise a CH₁ domain.

An scFv of scFv-Fc may be encoded by a sequence that includes in 5′ to 3′ direction, the following domains: a heavy chain variable domain (VH)-linker-a light chain variable domain (VL)-Fc domain. In some embodiments, scFv-Fc fusion may be encoded by a sequence that includes, in 5′ to 3′ direction, the following domains: a VL-linker-VH-Fc domain.

In some embodiments, a scFv-Fc comprises a hinge domain that comprises an amino acid modification that comprises a C220S mutation (according to EU numbering). Such a mutated hinge domain is referred to herein as “Hinge_S” or “C/S”. In some embodiments, a scFv-Fc comprises a hinge domain that comprises an amino acid sequence that is at least 90%, 91%, 92%, 93%, 94%, 95%, 96%, 97%, 98%, or 99% identical to a sequence represented in SEQ ID NO: 164. In some embodiments, a scFv-Fc comprises a hinge domain that comprises an amino acid sequence represented in SEQ ID NO: 164.

In some embodiments, a scFv-Fc comprises a CH₂ domain that comprises an amino acid sequence at least 90%, 91%, 92%, 93%, 94%, 95%, 96%, 97%, 98%, or 99% identical to represented in SEQ ID NO: 96. In some embodiments, a scFv-Fc comprises a CH₂ domain that comprises an amino acid sequence represented in SEQ ID NO: 96. In some embodiments, a scFv-Fc comprises a CH₂ domain that comprises one or more mutations (e.g., with respect to SEQ ID NO: 96). For example, in some embodiments, a scFv-Fc comprises one or more of the following mutations: G236A, A330L, and I332E (according to EU numbering). Such a combination of mutations is referred to herein as “GAALIE”. Such mutations in the CH₂ domain have been associated with increased affinity to Fc receptors FcgRIIA and FcgRIII for enhanced antibody effector function. In some embodiments, a scFv-Fc comprises a CH₂ domain that comprises an amino acid sequence that is at least 90%, 91%, 92%, 93%, 94%, 95%, 96%, 97%, 98%, or 99% identical to SEQ ID NO: 99 or 102. In some embodiments, a scFv-Fc comprises a CH₂ domain that comprises an amino acid sequence represented in SEQ ID NO: 99 or 102. In some embodiments, a scFv-Fc comprises one or more mutations selected from: G236A and I332E (according to EU numbering). Such a combination of CH₂ mutations is referred to herein as “GAIE”. In some embodiments, a scFv-Fc comprises a CH₂ domain that comprises an amino acid sequence at least 90%, 91%, 92%, 93%, 94%, 95%, 96%, 97%, 98%, or 99% identical to SEQ ID NO: 104. In some embodiments, a scFv-Fc comprises a CH₂ domain that comprises an amino acid sequence represented in SEQ ID NO: 104. In some embodiments, a scFv-Fc comprises a mutation: G236A (according to EU numbering), referred to herein as “GA”. In some embodiments, a scFv-Fc comprises a CH₂ domain that comprises an amino acid sequence at least 90%, 91%, 92%, 93%, 94%, 95%, 96%, 97%, 98%, or 99% identical to SEQ ID NO: 107. In some embodiments, a scFv-Fc comprises a CH₂ domain that comprises an amino acid sequence represented in SEQ ID NO: 107. In some embodiments, a scFv-Fc comprises a mutation: I332E (according to EU numbering), referred to herein as “IE”. In some embodiments, a scFv-Fc comprises a CH₂ domain that comprises an amino acid sequence at least 90%, 91%, 92%, 93%, 94%, 95%, 96%, 97%, 98%, or 99% identical to SEQ ID NO: 110. In some embodiments, a scFv-Fc comprises a CH₂ domain that comprises an amino acid sequence represented in SEQ ID NO: 110.

In some embodiments, a scFv-Fc encoded by one or more polyribonucleotides as described herein comprises a signal peptide comprising a husec2 signal peptide. In some embodiments, a scFv-Fc comprises a signal peptide comprising SEQ ID NO: 1.

In some embodiments, a scFv-Fc encoded by one or more polyribonucleotides as described herein comprises an immunoglobulin chain encoded by a nucleic acid sequence that has at least 80%, 85%, 90%, 91%, 92%, 93%, 94%, 95%, 96%, 97%, 98%, or 99% sequence identity to a sequence represented by SEQ ID NO: 458-841, 1341-1342, 1344-1345, 1347-1348, 1350-1351, 1356-1357, 1359-1360, 1362-1363, 1365-1366, 1368-1369, 1371-1372, 1374-1375, 1377-1378, 1380-1381, 1383-1384, 1386-1387, 1389-1390, 1392-1393, 1395-1396, 1398-1399, 1401-1402, 1404-1405, 1407-1408, 1410-1411, 1413-1414, 1416-1417, 1419-1420, 1422-1423, 1425-1426, 1428-1429, 1431-1432, 1434-1435, 1437-1438, 1440-1441, 1443-1444, 1446-1447, 1449-1450, 1552-1553, 1455-1456, or 1458-1459. In some embodiments, a scFv-Fc encoded by one or more polyribonucleotides as described herein comprises an immunoglobulin chain encoded by a nucleic acid sequence represented by any one of SEQ ID NOs: 458-841, 1341-1342, 1344-1345, 1347-1348, 1350-1351, 1356-1357, 1359-1360, 1362-1363, 1365-1366, 1368-1369, 1371-1372, 1374-1375, 1377-1378, 1380-1381, 1383-1384, 1386-1387, 1389-1390, 1392-1393, 1395-1396, 1398-1399, 1401-1402, 1404-1405, 1407-1408, 1410-1411, 1413-1414, 1416-1417, 1419-1420, 1422-1423, 1425-1426, 1428-1429, 1431-1432, 1434-1435, 1437-1438, 1440-1441, 1443-1444, 1446-1447, 1449-1450, 1552-1553, 1455-1456, or 1458-1459.

In some embodiments, a scFv-Fc encoded by one or more polyribonucleotides as described herein comprises an immunoglobulin chain comprising an amino acid sequence that has at least 80%, 85%, 90%, 91%, 92%, 93%, 94%, 95%, 96%, 97%, 98%, or 99% sequence identity to a sequence represented by SEQ ID NO: 1343, 1346, 1349, 1352, 1358, 1361, 1364, 1367, 1370, 1373, 1376, 1379, 1382, 1385, 1388, 1391, 1394, 1397, 1400, 1403, 1406, 1409, 1412, 1415, 1418, 1421, 1424, 1427, 1430, 1433, 1436, 1439, 1442, 1445, 1448, 1451, 1454, 1457, 1460. In some embodiments, a scFv-Fc encoded by one or more polyribonucleotides as described herein comprises an immunoglobulin chain comprising an amino acid sequence represented by any one of SEQ ID NOs: 1343, 1346, 1349, 1352, 1358, 1361, 1364, 1367, 1370, 1373, 1376, 1379, 1382, 1385, 1388, 1391, 1394, 1397, 1400, 1403, 1406, 1409, 1412, 1415, 1418, 1421, 1424, 1427, 1430, 1433, 1436, 1439, 1442, 1445, 1448, 1451, 1454, 1457, or 1460.

Exemplary configurations of scFv-Fc, as described herein, is shown in Table 6 below.

TABLE 6
Exemplary scFv-Fc Configurations

| scFv-Fc formats | Allotype | VH | VL | Hinge | CH2 | CH3 |
|---|---|---|---|---|---|---|
| PGDM1400 VH- | G1m3 | PGDM1400 | PGDM1400 | Hinge_S | CH2 | CH3_G1m3/17 |
| (G4S)4-VL G1m3 | | | | | | |
| CH1del C220S | | | | | | |
| PGDM1400 VH- | G1m3 | PGDM1400 | PGDM1400 | Hinge_S | CH2 | CH3_LS_G1m3/17 |
| (G4S)4-VL G1m3 | | | | | | |
| CH1del C220S LS | | | | | | |
| PGDM1400 VH- | G1m3 | PGDM1400 | PGDM1400 | Hinge_S | CH2_GAALIE | CH3_LS_G1m3/17 |
| (G4S)4-VL G1m3 | | | | | | |
| CH1del C220S GAALIE | | | | | | |
| LS | | | | | | |
| PGDM1400 VH- | G1m3 | PGDM1400 | PGDM1400 | Hinge_S | CH2_GAIE | CH3_LS_G1m3/17 |
| (G4S)4-VL G1m3 | | | | | | |
| CH1del C220S GAIE | | | | | | |
| LS | | | | | | |
| PGDM1400 VH- | G1m3 | PGDM1400 | PGDM1400 | Hinge_S | CH2_GA | CH3_LS_G1m3/17 |
| (G4S)4-VL G1m3 | | | | | | |
| CH1del C220S GA LS | | | | | | |
| PGDM1400 VH- | G1m3 | PGDM1400 | PGDM1400 | Hinge_S | CH2_IE | CH3_LS_G1m3/17 |
| (G4S)4-VL G1m3 | | | | | | |
| CH1del C220S IE LS | | | | | | |
| PGDM1400 VH- | G1m3 | PGDM1400 | PGDM1400 | Hinge_S | CH2 | CH3_cah_LS_G1m3/17 |
| (G4S)4-VL G1m3 | | | | | | |
| CH1del C220S cah LS | | | | | | |
| PGDM1400 VH- | G1m3 | PGDM1400 | PGDM1400 | Hinge_S | CH2_GAALIE | CH3_cah_LS_G1m3/17 |
| (G4S)4-VL G1m3 | | | | | | |
| CH1del C220S GAALIE | | | | | | |
| cah LS | | | | | | |
| PGDM1400 VH- | G1m3 | PGDM1400 | PGDM1400 | Hinge_S | CH2_GAIE | CH3_cah_LS_G1m3/17 |
| (G4S)4-VL G1m3 | | | | | | |
| CH1del C220S GAIE | | | | | | |
| cah LS | | | | | | |
| PGDM1400 VH- | G1m3 | PGDM1400 | PGDM1400 | Hinge_S | CH2_GA | CH3_cah_LS_G1m3/17 |
| (G4S)4-VL G1m3 | | | | | | |
| CH1del C220S GA cah | | | | | | |
| LS | | | | | | |
| PGDM1400 VH- | G1m3 | PGDM1400 | PGDM1400 | Hinge_S | CH2_IE | CH3_cah_LS_G1m3/17 |
| (G4S)4-VL G1m3 | | | | | | |
| CH1del C220S IE cah | | | | | | |
| LS | | | | | | |
| PGDM1400 VH- | G1m3 | PGDM1400 | PGDM1400 | Hinge_S | CH2 | CH3_cak_LS_G1m3/17 |
| (G4S)4-VL G1m3 | | | | | | |
| CH1del C220S cak LS | | | | | | |
| PGDM1400 VH- | G1m3 | PGDM1400 | PGDM1400 | Hinge_S | CH2_GAALIE | CH3_cak_LS_G1m3/17 |
| (G4S)4-VL G1m3 | | | | | | |
| CH1del C220S GAALIE | | | | | | |
| cak LS | | | | | | |
| PGDM1400 VH- | G1m3 | PGDM1400 | PGDM1400 | Hinge_S | CH2_GAIE | CH3_cak_LS_G1m3/17 |
| (G4S)4-VL G1m3 | | | | | | |
| CH1del C220S GAIE | | | | | | |
| cak LS | | | | | | |
| PGDM1400 VH- | G1m3 | PGDM1400 | PGDM1400 | Hinge_S | CH2_GA | CH3_cak_LS_G1m3/17 |
| (G4S)4-VL G1m3 | | | | | | |
| CH1del C220S GA cak | | | | | | |
| LS | | | | | | |
| PGDM1400 VH- | G1m3 | PGDM1400 | PGDM1400 | Hinge_S | CH2_IE | CH3_cak_LS_G1m3/17 |
| (G4S)4-VL G1m3 | | | | | | |
| CH1del C220S IE cak | | | | | | |
| LS | | | | | | |
| PGDM1400 VH- | G1m17 | PGDM1400 | PGDM1400 | Hinge_S | CH2 | CH3_G1m3/17 |
| (G4S)4-VL G1m17 | | | | | | |
| CH1del C220S | | | | | | |
| PGDM1400 VH- | G1m17 | PGDM1400 | PGDM1400 | Hinge_S | CH2 | CH3_LS_G1m3/17 |
| (G4S)4-VL G1m17 | | | | | | |
| CH1del C220S LS | | | | | | |
| PGDM1400 VH- | G1m17 | PGDM1400 | PGDM1400 | Hinge_S | CH2_GAALIE | CH3_LS_G1m3/17 |
| (G4S)4-VL G1m17 | | | | | | |
| CH1del C220S GAALIE | | | | | | |
| LS | | | | | | |
| PGDM1400 VH- | G1m17 | PGDM1400 | PGDM1400 | Hinge_S | CH2_GAIE | CH3_LS_G1m3/17 |
| (G4S)4-VL G1m17 | | | | | | |
| CH1del C220S GAIE | | | | | | |
| LS | | | | | | |
| PGDM1400 VH- | G1m17 | PGDM1400 | PGDM1400 | Hinge_S | CH2_GA | CH3_LS_G1m3/17 |
| (G4S)4-VL G1m17 | | | | | | |
| CH1del C220S GA LS | | | | | | |
| PGDM1400 VH- | G1m17 | PGDM1400 | PGDM1400 | Hinge_S | CH2_IE | CH3_LS_G1m3/17 |
| (G4S)4-VL G1m17 | | | | | | |
| CH1del C220S IE LS | | | | | | |
| PGDM1400 VH- | G1m17 | PGDM1400 | PGDM1400 | Hinge_S | CH2 | CH3_cah_LS_G1m3/17 |
| (G4S)4-VL G1m17 | | | | | | |
| CH1del C220S cah LS | | | | | | |
| PGDM1400 VH- | G1m17 | PGDM1400 | PGDM1400 | Hinge_S | CH2_GAALIE | CH3_cah_LS_G1m3/17 |
| (G4S)4-VL G1m17 | | | | | | |
| CH1del C220S GAALIE | | | | | | |
| cah LS | | | | | | |
| PGDM1400 VH- | G1m17 | PGDM1400 | PGDM1400 | Hinge_S | CH2_GAIE | CH3_cah_LS_G1m3/17 |
| (G4S)4-VL G1m17 | | | | | | |
| CH1del C220S GAIE | | | | | | |
| cah LS | | | | | | |
| PGDM1400 VH- | G1m17 | PGDM1400 | PGDM1400 | Hinge_S | CH2_GA | CH3_cah_LS_G1m3/17 |
| (G4S)4-VL G1m17 | | | | | | |
| CH1del C220S GA cah | | | | | | |
| LS | | | | | | |
| PGDM1400 VH- | G1m17 | PGDM1400 | PGDM1400 | Hinge_S | CH2_IE | CH3_cah_LS_G1m3/17 |
| (G4S)4-VL G1m17 | | | | | | |
| CH1del C220S IE cah | | | | | | |
| LS | | | | | | |
| PGDM1400 VH- | G1m17 | PGDM1400 | PGDM1400 | Hinge_S | CH2 | CH3_cak_LS_G1m3/17 |
| (G4S)4-VL G1m17 | | | | | | |
| CH1del C220S cak LS | | | | | | |
| PGDM1400 VH- | G1m17 | PGDM1400 | PGDM1400 | Hinge_S | CH2_GAALIE | CH3_cak_LS_G1m3/17 |
| (G4S)4-VL G1m17 | | | | | | |
| CH1del C220S GAALIE | | | | | | |
| cak LS | | | | | | |
| PGDM1400 VH- | G1m17 | PGDM1400 | PGDM1400 | Hinge_S | CH2_GAIE | CH3_cak_LS_G1m3/17 |
| (G4S)4-VL G1m17 | | | | | | |
| CH1del C220S GAIE | | | | | | |
| cak LS | | | | | | |
| PGDM1400 VH- | G1m17 | PGDM1400 | PGDM1400 | Hinge_S | CH2_GA | CH3_cak_LS_G1m3/17 |
| (G4S)4-VL G1m17 | | | | | | |
| CH1del C220S GA cak | | | | | | |
| LS | | | | | | |
| PGDM1400 VH- | G1m17 | PGDM1400 | PGDM1400 | Hinge_S | CH2_IE | CH3_cak_LS_G1m3/17 |
| (G4S)4-VL G1m17 | | | | | | |
| CH1del C220S IE cak | | | | | | |
| LS | | | | | | |
| PGDM1400 VH- | G1m17,1 | PGDM1400 | PGDM1400 | Hinge_S | CH2 | CH3_G1m17,1 |
| (G4S)4-VL G1m17,1 | | | | | | |
| CH1del C220S | | | | | | |
| PGDM1400 VH- | G1m17,1 | PGDM1400 | PGDM1400 | Hinge_S | CH2 | CH3_LS_G1m17,1 |
| (G4S)4-VL G1m17,1 | | | | | | |
| CH1del C220S LS | | | | | | |
| PGDM1400 VH- | G1m17,1 | PGDM1400 | PGDM1400 | Hinge_S | CH2_GAALIE | CH3_LS_G1m17,1 |
| (G4S)4-VL G1m17,1 | | | | | | |
| CH1del C220S GAALIE | | | | | | |
| LS | | | | | | |
| PGDM1400 VH- | G1m17,1 | PGDM1400 | PGDM1400 | Hinge_S | CH2_GAIE | CH3_LS_G1m17,1 |
| (G4S)4-VL G1m17,1 | | | | | | |
| CH1del C220S GAIE | | | | | | |
| LS | | | | | | |
| PGDM1400 VH- | G1m17,1 | PGDM1400 | PGDM1400 | Hinge_S | CH2_GA | CH3_LS_G1m17,1 |
| (G4S)4-VL G1m17,1 | | | | | | |
| CH1del C220S GA LS | | | | | | |
| PGDM1400 VH- | G1m17,1 | PGDM1400 | PGDM1400 | Hinge_S | CH2_IE | CH3_LS_G1m17,1 |
| (G4S)4-VL G1m17,1 | | | | | | |
| CH1del C220S IE LS | | | | | | |
| PGDM1400 VH- | G1m17,1 | PGDM1400 | PGDM1400 | Hinge_S | CH2 | CH3_cah_LS_G1m17,1 |
| (G4S)4-VL G1m17,1 | | | | | | |
| CH1del C220S cah LS | | | | | | |
| PGDM1400 VH- | G1m17,1 | PGDM1400 | PGDM1400 | Hinge_S | CH2_GAALIE | CH3_cah_LS_G1m17,1 |
| (G4S)4-VL G1m17,1 | | | | | | |
| CH1del C220S GAALIE | | | | | | |
| cah LS | | | | | | |
| PGDM1400 VH- | G1m17,1 | PGDM1400 | PGDM1400 | Hinge_S | CH2_GAIE | CH3_cah_LS_G1m17,1 |
| (G4S)4-VL G1m17,1 | | | | | | |
| CH1del C220S GAIE | | | | | | |
| cah LS | | | | | | |
| PGDM1400 VH- | G1m17,1 | PGDM1400 | PGDM1400 | Hinge_S | CH2_GA | CH3_cah_LS_G1m17,1 |
| (G4S)4-VL G1m17,1 | | | | | | |
| CH1del C220S GA cah | | | | | | |
| LS | | | | | | |
| PGDM1400 VH- | G1m17,1 | PGDM1400 | PGDM1400 | Hinge_S | CH2_IE | CH3_cah_LS_G1m17,1 |
| (G4S)4-VL G1m17,1 | | | | | | |
| CH1del C220S IE cah | | | | | | |
| LS | | | | | | |
| PGDM1400 VH- | G1m17,1 | PGDM1400 | PGDM1400 | Hinge_S | CH2 | CH3_cak_LS_G1m17,1 |
| (G4S)4-VL G1m17,1 | | | | | | |
| CH1del C220S cak LS | | | | | | |
| PGDM1400 VH- | G1m17,1 | PGDM1400 | PGDM1400 | Hinge_S | CH2_GAALIE | CH3_cak_LS_G1m17,1 |
| (G4S)4-VL G1m17,1 | | | | | | |
| CH1del C220S GAALIE | | | | | | |
| cak LS | | | | | | |
| PGDM1400 VH- | G1m17,1 | PGDM1400 | PGDM1400 | Hinge_S | CH2_GAIE | CH3_cak_LS_G1m17,1 |
| (G4S)4-VL G1m17,1 | | | | | | |
| CH1del C220S GAIE | | | | | | |
| cak LS | | | | | | |
| PGDM1400 VH- | G1m17,1 | PGDM1400 | PGDM1400 | Hinge_S | CH2_GA | CH3_cak_LS_G1m17,1 |
| (G4S)4-VL G1m17,1 | | | | | | |
| CH1del C220S GA cak | | | | | | |
| LS | | | | | | |
| PGDM1400 VH- | G1m17,1 | PGDM1400 | PGDM1400 | Hinge_S | CH2_IE | CH3_cak_LS_G1m17,1 |
| (G4S)4-VL G1m17,1 | | | | | | |
| CH1del C220S IE cak | | | | | | |
| LS | | | | | | |
| PGDM1400 VH- | G1m3 | PGDM1400 | PGDM1400 | Hinge_S | CH2 | CH3_G1m3/17 |
| (G4S)5-VL G1m3 | | | | | | |
| CH1del C220S | | | | | | |
| PGDM1400 VH- | G1m3 | PGDM1400 | PGDM1400 | Hinge_S | CH2 | CH3_LS_G1m3/17 |
| (G4S)5-VL G1m3 | | | | | | |
| CH1del C220SLS | | | | | | |
| PGDM1400 VH- | G1m3 | PGDM1400 | PGDM1400 | Hinge_S | CH2_GAALIE | CH3_LS_G1m3/17 |
| (G4S)5-VL G1m3 | | | | | | |
| CH1del C220S GAALIE | | | | | | |
| LS | | | | | | |
| PGDM1400 VH- | G1m3 | PGDM1400 | PGDM1400 | Hinge_S | CH2_GAIE | CH3_LS_G1m3/17 |
| (G4S)5-VL | | | | | | |
| G1m3CH1del C220S | | | | | | |
| GAIE LS | | | | | | |
| PGDM1400 VH- | G1m3 | PGDM1400 | PGDM1400 | Hinge_S | CH2_GA | CH3_LS_G1m3/17 |
| (G4S)5-VL | | | | | | |
| G1m3CH1del C220S | | | | | | |
| GA LS | | | | | | |
| PGDM1400 VH- | G1m3 | PGDM1400 | PGDM1400 | Hinge_S | CH2_IE | CH3_LS_G1m3/17 |
| (G4S)5-VL | | | | | | |
| G1m3CH1del C220S IE | | | | | | |
| LS | | | | | | |
| PGDM1400 VH- | G1m3 | PGDM1400 | PGDM1400 | Hinge_S | CH2 | CH3_cah_LS_G1m3/17 |
| (G4S)5-VL | | | | | | |
| G1m3CH1del C220S | | | | | | |
| cah LS | | | | | | |
| PGDM1400 VH- | G1m3 | PGDM1400 | PGDM1400 | Hinge_S | CH2_GAALIE | CH3_cah_LS_G1m3/17 |
| (G4S)5-VL | | | | | | |
| G1m3CH1del C220S | | | | | | |
| GAALIE cah LS | | | | | | |
| PGDM1400 VH- | G1m3 | PGDM1400 | PGDM1400 | Hinge_S | CH2_GAIE | CH3_cah_LS_G1m3/17 |
| (G4S)5-VL | | | | | | |
| G1m3CH1del C220S | | | | | | |
| GAIE cah LS | | | | | | |
| PGDM1400 VH- | G1m3 | PGDM1400 | PGDM1400 | Hinge_S | CH2_GA | CH3_cah_LS_G1m3/17 |
| (G4S)5-VL | | | | | | |
| G1m3CH1del C220S | | | | | | |
| GA cah LS | | | | | | |
| PGDM1400 VH- | G1m3 | PGDM1400 | PGDM1400 | Hinge_S | CH2_IE | CH3_cah_LS_G1m3/17 |
| (G4S)5-VL | | | | | | |
| G1m3CH1del C220S IE | | | | | | |
| cah LS | | | | | | |
| PGDM1400 VH- | G1m3 | PGDM1400 | PGDM1400 | Hinge_S | CH2 | CH3_cak_LS_G1m3/17 |
| (G4S)5-VL | | | | | | |
| G1m3CH1del C220S | | | | | | |
| cak LS | | | | | | |
| PGDM1400 VH- | G1m3 | PGDM1400 | PGDM1400 | Hinge_S | CH2_GAALIE | CH3_cak_LS_G1m3/17 |
| (G4S)5-VL | | | | | | |
| G1m3CH1del C220S | | | | | | |
| GAALIE cak LS | | | | | | |
| PGDM1400 VH- | G1m3 | PGDM1400 | PGDM1400 | Hinge_S | CH2_GAIE | CH3_cak_LS_G1m3/17 |
| (G4S)5-VL | | | | | | |
| G1m3CH1del C220S | | | | | | |
| GAIE cak LS | | | | | | |
| PGDM1400 VH- | G1m3 | PGDM1400 | PGDM1400 | Hinge_S | CH2_GA | CH3_cak_LS_G1m3/17 |
| (G4S)5-VL | | | | | | |
| G1m3CH1del C220S | | | | | | |
| GA cak LS | | | | | | |
| PGDM1400 VH- | G1m3 | PGDM1400 | PGDM1400 | Hinge_S | CH2_IE | CH3_cak_LS_G1m3/17 |
| (G4S)5-VL | | | | | | |
| G1m3CH1del C220S IE | | | | | | |
| cak LS | | | | | | |
| PGDM1400 VH- | G1m17 | PGDM1400 | PGDM1400 | Hinge_S | CH2 | CH3_G1m3/17 |
| (G4S)5-VL G1m17 | | | | | | |
| CH1del C220S | | | | | | |
| PGDM1400 VH- | G1m17 | PGDM1400 | PGDM1400 | Hinge_S | CH2 | CH3_LS_G1m3/17 |
| (G4S)5-VL G1m17 | | | | | | |
| CH1del C220S LS | | | | | | |
| PGDM1400 VH- | G1m17 | PGDM1400 | PGDM1400 | Hinge_S | CH2_GAALIE | CH3_LS_G1m3/17 |
| (G4S)5-VL G1m17 | | | | | | |
| CH1del C220S GAALIE | | | | | | |
| cak LS | | | | | | |
| PGDM1400 VH- | G1m17 | PGDM1400 | PGDM1400 | Hinge_S | CH2_GAIE | CH3_LS_G1m3/17 |
| (G4S)5-VL G1m17 | | | | | | |
| CH1del C220S GAIE LS | | | | | | |
| PGDM1400 VH- | G1m17 | PGDM1400 | PGDM1400 | Hinge_S | CH2_GA | CH3_LS_G1m3/17 |
| (G4S)5-VL G1m17 | | | | | | |
| CH1del C220S GA LS | | | | | | |
| PGDM1400 VH- | G1m17 | PGDM1400 | PGDM1400 | Hinge_S | CH2_IE | CH3_LS_G1m3/17 |
| (G4S)5-VL G1m17 | | | | | | |
| CH1del C220S IE LS | | | | | | |
| PGDM1400 VH- | G1m17 | PGDM1400 | PGDM1400 | Hinge_S | CH2 | CH3_cah_LS_G1m3/17 |
| (G4S)5-VL G1m17 | | | | | | |
| CH1del C220S cah LS | | | | | | |
| PGDM1400 VH- | G1m17 | PGDM1400 | PGDM1400 | Hinge_S | CH2_GAALIE | CH3_cah_LS_G1m3/17 |
| (G4S)5-VL G1m17 | | | | | | |
| CH1del C220S GAALIE | | | | | | |
| cah LS | | | | | | |
| PGDM1400 VH- | G1m17 | PGDM1400 | PGDM1400 | Hinge_S | CH2_GAIE | CH3_cah_LS_G1m3/17 |
| (G4S)5-VL G1m17 | | | | | | |
| CH1del C220S GAIE | | | | | | |
| cah LS | | | | | | |
| PGDM1400 VH- | G1m17 | PGDM1400 | PGDM1400 | Hinge_S | CH2_GA | CH3_cah_LS_G1m3/17 |
| (G4S)5-VL G1m17 | | | | | | |
| CH1del C220S GA cah | | | | | | |
| LS | | | | | | |
| PGDM1400 VH- | G1m17 | PGDM1400 | PGDM1400 | Hinge_S | CH2_IE | CH3_cah_LS_G1m3/17 |
| (G4S)5-VL G1m17 | | | | | | |
| CH1del C220S IE cah | | | | | | |
| LS | | | | | | |
| PGDM1400 VH- | G1m17 | PGDM1400 | PGDM1400 | Hinge_S | CH2 | CH3_cak_LS_G1m3/17 |
| (G4S)5-VL G1m17 | | | | | | |
| CH1del C220S cak LS | | | | | | |
| PGDM1400 VH- | G1m17 | PGDM1400 | PGDM1400 | Hinge_S | CH2_GAALIE | CH3_cak_LS_G1m3/17 |
| (G4S)5-VL G1m17 | | | | | | |
| CH1del C220S GAALIE | | | | | | |
| cak LS | | | | | | |
| PGDM1400 VH- | G1m17 | PGDM1400 | PGDM1400 | Hinge_S | CH2_GAIE | CH3_cak_LS_G1m3/17 |
| (G4S)5-VL G1m17 | | | | | | |
| CH1del C220S GAIE | | | | | | |
| cak LS | | | | | | |
| PGDM1400 VH- | G1m17 | PGDM1400 | PGDM1400 | Hinge_S | CH2_GA | CH3_cak_LS_G1m3/17 |
| (G4S)5-VL G1m17 | | | | | | |
| CH1del C220S GA cak | | | | | | |
| LS | | | | | | |
| PGDM1400 VH- | G1m17 | PGDM1400 | PGDM1400 | Hinge_S | CH2_IE | CH3_cak_LS_G1m3/17 |
| (G4S)5-VL G1m17 | | | | | | |
| CH1del C220S IE cak | | | | | | |
| LS | | | | | | |
| PGDM1400 VH- | G1m17,1 | PGDM1400 | PGDM1400 | Hinge_S | CH2 | CH3_G1m17,1 |
| (G4S)5-VL G1m17,1 | | | | | | |
| CH1del C220S | | | | | | |
| PGDM1400 VH- | G1m17,1 | PGDM1400 | PGDM1400 | Hinge_S | CH2 | CH3_LS_G1m17,1 |
| (G4S)5-VL G1m17,1 | | | | | | |
| CH1del C220S LS | | | | | | |
| PGDM1400 VH- | G1m17,1 | PGDM1400 | PGDM1400 | Hinge_S | CH2_GAALIE | CH3_LS_G1m17,1 |
| (G4S)5-VL G1m17,1 | | | | | | |
| CH1del C220S GAALIE | | | | | | |
| LS | | | | | | |
| PGDM1400 VH- | G1m17,1 | PGDM1400 | PGDM1400 | Hinge_S | CH2_GAIE | CH3_LS_G1m17,1 |
| (G4S)5-VL G1m17,1 | | | | | | |
| CH1del C220S GAIE LS | | | | | | |
| PGDM1400 VH- | G1m17,1 | PGDM1400 | PGDM1400 | Hinge_S | CH2_GA | CH3_LS_G1m17,1 |
| (G4S)5-VL G1m17,1 | | | | | | |
| CH1del C220S GA LS | | | | | | |
| PGDM1400 VH- | G1m17,1 | PGDM1400 | PGDM1400 | Hinge_S | CH2_IE | CH3_LS_G1m17,1 |
| (G4S)5-VL G1m17,1 | | | | | | |
| CH1del C220S IE LS | | | | | | |
| PGDM1400 VH- | G1m17,1 | PGDM1400 | PGDM1400 | Hinge_S | CH2 | CH3_cah_LS_G1m17,1 |
| (G4S)5-VL G1m17,1 | | | | | | |
| CH1del C220S cah LS | | | | | | |
| PGDM1400 VH- | G1m17,1 | PGDM1400 | PGDM1400 | Hinge_S | CH2_GAALIE | CH3_cah_LS_G1m17,1 |
| (G4S)5-VL G1m17,1 | | | | | | |
| CH1del C220S GAALIE | | | | | | |
| cah LS | | | | | | |
| PGDM1400 VH- | G1m17,1 | PGDM1400 | PGDM1400 | Hinge_S | CH2_GAIE | CH3_cah_LS_G1m17,1 |
| (G4S)5-VL G1m17,1 | | | | | | |
| CH1del C220S GAIE | | | | | | |
| cah LS | | | | | | |
| PGDM1400 VH- | G1m17,1 | PGDM1400 | PGDM1400 | Hinge_S | CH2_GA | CH3_cah_LS_G1m17,1 |
| (G4S)5-VL G1m17,1 | | | | | | |
| CH1del C220S GA cah | | | | | | |
| LS | | | | | | |
| PGDM1400 VH- | G1m17,1 | PGDM1400 | PGDM1400 | Hinge_S | CH2_IE | CH3_cah_LS_G1m17,1 |
| (G4S)5-VL G1m17,1 | | | | | | |
| CH1del C220S IE cah | | | | | | |
| LS | | | | | | |
| PGDM1400 VH- | G1m17,1 | PGDM1400 | PGDM1400 | Hinge_S | CH2 | CH3_cak_LS_G1m17,1 |
| (G4S)5-VL G1m17,1 | | | | | | |
| CH1del C220S cak LS | | | | | | |
| PGDM1400 VH- | G1m17,1 | PGDM1400 | PGDM1400 | Hinge_S | CH2_GAALIE | CH3_cak_LS_G1m17,1 |
| (G4S)5-VL G1m17,1 | | | | | | |
| CH1del C220S GAALIE | | | | | | |
| cak LS | | | | | | |
| PGDM1400 VH- | G1m17,1 | PGDM1400 | PGDM1400 | Hinge_S | CH2_GAIE | CH3_cak_LS_G1m17,1 |
| (G4S)5-VL G1m17,1 | | | | | | |
| CH1del C220S GAIE | | | | | | |
| cak LS | | | | | | |
| PGDM1400 VH- | G1m17,1 | PGDM1400 | PGDM1400 | Hinge_S | CH2_GA | CH3_cak_LS_G1m17,1 |
| (G4S)5-VL G1m17,1 | | | | | | |
| CH1del C220S GA cak | | | | | | |
| LS | | | | | | |
| PGDM1400 VH- | G1m17,1 | PGDM1400 | PGDM1400 | Hinge_S | CH2_IE | CH3_cak_LS_G1m17,1 |
| (G4S)5-VL G1m17,1 | | | | | | |
| CH1del C220S IE cak | | | | | | |
| LS | | | | | | |
| PGDM1400 VL-(G4S)4- | G1m3 | PGDM1400 | PGDM1400 | Hinge_S | CH2 | CH3_G1m3/17 |
| VH G1m3 CH1del | | | | | | |
| C220S | | | | | | |
| PGDM1400 VL-(G4S)4- | G1m3 | PGDM1400 | PGDM1400 | Hinge_S | CH2 | CH3_LS_G1m3/17 |
| VH G1m3 CH1del | | | | | | |
| C220S LS | | | | | | |
| PGDM1400 VL-(G4S)4- | G1m3 | PGDM1400 | PGDM1400 | Hinge_S | CH2_GAALIE | CH3_LS_G1m3/17 |
| VH G1m3 CH1del | | | | | | |
| C220S GAALIE LS | | | | | | |
| PGDM1400 VL-(G4S)4- | G1m3 | PGDM1400 | PGDM1400 | Hinge_S | CH2_GAIE | CH3_LS_G1m3/17 |
| VH G1m3 CH1del | | | | | | |
| C220S GAIE LS | | | | | | |
| PGDM1400 VL-(G4S)4- | G1m3 | PGDM1400 | PGDM1400 | Hinge_S | CH2_GA | CH3_LS_G1m3/17 |
| VH G1m3 CH1del | | | | | | |
| C220S GA LS | | | | | | |
| PGDM1400 VL-(G4S)4- | G1m3 | PGDM1400 | PGDM1400 | Hinge_S | CH2_IE | CH3_LS_G1m3/17 |
| VH G1m3 CH1del | | | | | | |
| C220S IE LS | | | | | | |
| PGDM1400 VL-(G4S)4- | G1m3 | PGDM1400 | PGDM1400 | Hinge_S | CH2 | CH3_cah_LS_G1m3/17 |
| VH G1m3 CH1del | | | | | | |
| C220S cah LS | | | | | | |
| PGDM1400 VL-(G4S)4- | G1m3 | PGDM1400 | PGDM1400 | Hinge_S | CH2_GAALIE | CH3_cah_LS_G1m3/17 |
| VH G1m3 CH1del | | | | | | |
| C220S GAALIE cah LS | | | | | | |
| PGDM1400 VL-(G4S)4- | G1m3 | PGDM1400 | PGDM1400 | Hinge_S | CH2_GAIE | CH3_cah_LS_G1m3/17 |
| VH G1m3 CH1del | | | | | | |
| C220S GAIE cah LS | | | | | | |
| PGDM1400 VL-(G4S)4- | G1m3 | PGDM1400 | PGDM1400 | Hinge_S | CH2_GA | CH3_cah_LS_G1m3/17 |
| VH G1m3 CH1del | | | | | | |
| C220S GA cah LS | | | | | | |
| PGDM1400 VL-(G4S)4- | G1m3 | PGDM1400 | PGDM1400 | Hinge_S | CH2_IE | CH3_cah_LS_G1m3/17 |
| VH G1m3 CH1del | | | | | | |
| C220S IE cah LS | | | | | | |
| PGDM1400 VL-(G4S)4- | G1m3 | PGDM1400 | PGDM1400 | Hinge_S | CH2 | CH3_cak_LS_G1m3/17 |
| VH G1m3 CH1del | | | | | | |
| C220S cak LS | | | | | | |
| PGDM1400 VL-(G4S)4- | G1m3 | PGDM1400 | PGDM1400 | Hinge_S | CH2_GAALIE | CH3_cak_LS_G1m3/17 |
| VH G1m3 CH1del | | | | | | |
| C220S GAALIE cak LS | | | | | | |
| PGDM1400 VL-(G4S)4- | G1m3 | PGDM1400 | PGDM1400 | Hinge_S | CH2_GAIE | CH3_cak_LS_G1m3/17 |
| VH G1m3 CH1del | | | | | | |
| C220S GAIE cak LS | | | | | | |
| PGDM1400 VL-(G4S)4- | G1m3 | PGDM1400 | PGDM1400 | Hinge_S | CH2_GA | CH3_cak_LS_G1m3/17 |
| VH G1m3 CH1del | | | | | | |
| C220S GA cak LS | | | | | | |
| PGDM1400 VL-(G4S)4- | G1m3 | PGDM1400 | PGDM1400 | Hinge_S | CH2_IE | CH3_cak_LS_G1m3/17 |
| VH G1m3 CH1del | | | | | | |
| C220S IE cak LS | | | | | | |
| PGDM1400 VL-(G4S)4- | G1m17 | PGDM1400 | PGDM1400 | Hinge_S | CH2 | CH3_G1m3/17 |
| VH G1m17 CH1del | | | | | | |
| C220S | | | | | | |
| PGDM1400 VL-(G4S)4- | G1m17 | PGDM1400 | PGDM1400 | Hinge_S | CH2 | CH3_LS_G1m3/17 |
| VH G1m17 CH1del | | | | | | |
| C220S LS | | | | | | |
| PGDM1400 VL-(G4S)4- | G1m17 | PGDM1400 | PGDM1400 | Hinge_S | CH2_GAALIE | CH3_LS_G1m3/17 |
| VH G1m17 CH1del | | | | | | |
| C220S GAALIE LS | | | | | | |
| PGDM1400 VL-(G4S)4- | G1m17 | PGDM1400 | PGDM1400 | Hinge_S | CH2_GAIE | CH3_LS_G1m3/17 |
| VH G1m17 CH1del | | | | | | |
| C220S GAIE LS | | | | | | |
| PGDM1400 VL-(G4S)4- | G1m17 | PGDM1400 | PGDM1400 | Hinge_S | CH2_GA | CH3_LS_G1m3/17 |
| VH G1m17 CH1del | | | | | | |
| C220S GA LS | | | | | | |
| PGDM1400 VL-(G4S)4- | G1m17 | PGDM1400 | PGDM1400 | Hinge_S | CH2_IE | CH3_LS_G1m3/17 |
| VH G1m17 CH1del | | | | | | |
| C220S IE LS | | | | | | |
| PGDM1400 VL-(G4S)4- | G1m17 | PGDM1400 | PGDM1400 | Hinge_S | CH2 | CH3_cah_LS_G1m3/17 |
| VH G1m17 CH1del | | | | | | |
| C220S cah LS | | | | | | |
| PGDM1400 VL-(G4S)4- | G1m17 | PGDM1400 | PGDM1400 | Hinge_S | CH2_GAALIE | CH3_cah_LS_G1m3/17 |
| VH G1m17 CH1del | | | | | | |
| C220S GAALIE cah LS | | | | | | |
| PGDM1400 VL-(G4S)4- | G1m17 | PGDM1400 | PGDM1400 | Hinge_S | CH2_GAIE | CH3_cah_LS_G1m3/17 |
| VH G1m17 CH1del | | | | | | |
| C220S GAIE cah LS | | | | | | |
| PGDM1400 VL-(G4S)4- | G1m17 | PGDM1400 | PGDM1400 | Hinge_S | CH2_GA | CH3_cah_LS_G1m3/17 |
| VH G1m17 CH1del | | | | | | |
| C220S GA cah LS | | | | | | |
| PGDM1400 VL-(G4S)4- | G1m17 | PGDM1400 | PGDM1400 | Hinge_S | CH2_IE | CH3_cah_LS_G1m3/17 |
| VH G1m17 CH1del | | | | | | |
| C220S IE cah LS | | | | | | |
| PGDM1400 VL-(G4S)4- | G1m17 | PGDM1400 | PGDM1400 | Hinge_S | CH2 | CH3_cak_LS_G1m3/17 |
| VH G1m17 CH1del | | | | | | |
| C220S cak LS | | | | | | |
| PGDM1400 VL-(G4S)4- | G1m17 | PGDM1400 | PGDM1400 | Hinge_S | CH2_GAALIE | CH3_cak_LS_G1m3/17 |
| VH G1m17 CH1del | | | | | | |
| C220S GAALIE cak LS | | | | | | |
| PGDM1400 VL-(G4S)4- | G1m17 | PGDM1400 | PGDM1400 | Hinge_S | CH2_GAIE | CH3_cak_LS_G1m3/17 |
| VH G1m17 CH1del | | | | | | |
| C220S GAIE cak LS | | | | | | |
| PGDM1400 VL-(G4S)4- | G1m17 | PGDM1400 | PGDM1400 | Hinge_S | CH2_GA | CH3_cak_LS_G1m3/17 |
| VH G1m17 CH1del | | | | | | |
| C220S GA cak LS | | | | | | |
| PGDM1400 VL-(G4S)4- | G1m17 | PGDM1400 | PGDM1400 | Hinge_S | CH2_IE | CH3_cak_LS_G1m3/17 |
| VH G1m17 CH1del | | | | | | |
| C220S IE cak LS | | | | | | |
| PGDM1400 VL-(G4S)4- | G1m17,1 | PGDM1400 | PGDM1400 | Hinge_S | CH2 | CH3_G1m17,1 |
| VH G1m17,1 CH1del | | | | | | |
| C220S | | | | | | |
| PGDM1400 VL-(G4S)4- | G1m17,1 | PGDM1400 | PGDM1400 | Hinge_S | CH2 | CH3_LS_G1m17,1 |
| VH G1m17,1 CH1del | | | | | | |
| C220S LS | | | | | | |
| PGDM1400 VL-(G4S)4- | G1m17,1 | PGDM1400 | PGDM1400 | Hinge_S | CH2_GAALIE | CH3_LS_G1m17,1 |
| VH G1m17,1 CH1del | | | | | | |
| C220S GAALIE LS | | | | | | |
| PGDM1400 VL-(G4S)4- | G1m17,1 | PGDM1400 | PGDM1400 | Hinge_S | CH2_GAIE | CH3_LS_G1m17,1 |
| VH G1m17,1 CH1del | | | | | | |
| C220S GAIE LS | | | | | | |
| PGDM1400 VL-(G4S)4- | G1m17,1 | PGDM1400 | PGDM1400 | Hinge_S | CH2_GA | CH3_LS_G1m17,1 |
| VH G1m17,1 CH1del | | | | | | |
| C220S GA LS | | | | | | |
| PGDM1400 VL-(G4S)4- | G1m17,1 | PGDM1400 | PGDM1400 | Hinge_S | CH2_IE | CH3_LS_G1m17,1 |
| VH G1m17,1 CH1del | | | | | | |
| C220S IE LS | | | | | | |
| PGDM1400 VL-(G4S)4- | G1m17,1 | PGDM1400 | PGDM1400 | Hinge_S | CH2 | CH3_cah_LS_G1m17,1 |
| VH G1m17,1 CH1del | | | | | | |
| C220S cah LS | | | | | | |
| PGDM1400 VL-(G4S)4- | G1m17,1 | PGDM1400 | PGDM1400 | Hinge_S | CH2_GAALIE | CH3_cah_LS_G1m17,1 |
| VH G1m17,1 CH1del | | | | | | |
| C220S GAALIE cah LS | | | | | | |
| PGDM1400 VL-(G4S)4- | G1m17,1 | PGDM1400 | PGDM1400 | Hinge_S | CH2_GAIE | CH3_cah_LS_G1m17,1 |
| VH G1m17,1 CH1del | | | | | | |
| C220S GAIE cah LS | | | | | | |
| PGDM1400 VL-(G4S)4- | G1m17,1 | PGDM1400 | PGDM1400 | Hinge_S | CH2_GA | CH3_cah_LS_G1m17,1 |
| VH G1m17,1 CH1del | | | | | | |
| C220S GA cah LS | | | | | | |
| PGDM1400 VL-(G4S)4- | G1m17,1 | PGDM1400 | PGDM1400 | Hinge_S | CH2_IE | CH3_cah_LS_G1m17,1 |
| VH G1m17,1 CH1del | | | | | | |
| C220S IE cah LS | | | | | | |
| PGDM1400 VL-(G4S)4- | G1m17,1 | PGDM1400 | PGDM1400 | Hinge_S | CH2 | CH3_cak_LS_G1m17,1 |
| VH G1m17,1 CH1del | | | | | | |
| C220S cak LS | | | | | | |
| PGDM1400 VL-(G4S)4- | G1m17,1 | PGDM1400 | PGDM1400 | Hinge_S | CH2_GAALIE | CH3_cak_LS_G1m17,1 |
| VH G1m17,1 CH1del | | | | | | |
| C220S GAALIE cak LS | | | | | | |
| PGDM1400 VL-(G4S)4- | G1m17,1 | PGDM1400 | PGDM1400 | Hinge_S | CH2_GAIE | CH3_cak_LS_G1m17,1 |
| VH G1m17,1 CH1del | | | | | | |
| C220S GAIE cak LS | | | | | | |
| PGDM1400 VL-(G4S)4- | G1m17,1 | PGDM1400 | PGDM1400 | Hinge_S | CH2_GA | CH3_cak_LS_G1m17,1 |
| VH G1m17,1 CH1del | | | | | | |
| C220S GA cak LS | | | | | | |
| PGDM1400 VL-(G4S)4- | G1m17,1 | PGDM1400 | PGDM1400 | Hinge_S | CH2_IE | CH3_cak_LS_G1m17,1 |
| VH G1m17,1 CH1del | | | | | | |
| C220S IE cak LS | | | | | | |
| PGDM1400 VL-(G4S)5- | G1m3 | PGDM1400 | PGDM1400 | Hinge_S | CH2 | CH3_G1m3/17 |
| VH G1m3 CH1del | | | | | | |
| C220S | | | | | | |
| PGDM1400 VL-(G4S)5- | G1m3 | PGDM1400 | PGDM1400 | Hinge_S | CH2 | CH3_LS_G1m3/17 |
| VH G1m3 CH1del | | | | | | |
| C220S LS | | | | | | |
| PGDM1400 VL-(G4S)5- | G1m3 | PGDM1400 | PGDM1400 | Hinge_S | CH2_GAALIE | CH3_LS_G1m3/17 |
| VH G1m3 CH1del | | | | | | |
| C220S GAALIE LS | | | | | | |
| PGDM1400 VL-(G4S)5- | G1m3 | PGDM1400 | PGDM1400 | Hinge_S | CH2_GAIE | CH3_LS_G1m3/17 |
| VH G1m3 CH1del | | | | | | |
| C220S GAIE LS | | | | | | |
| PGDM1400 VL-(G4S)5- | G1m3 | PGDM1400 | PGDM1400 | Hinge_S | CH2_GA | CH3_LS_G1m3/17 |
| VH G1m3 CH1del | | | | | | |
| C220S GA LS | | | | | | |
| PGDM1400 VL-(G4S)5- | G1m3 | PGDM1400 | PGDM1400 | Hinge_S | CH2_IE | CH3_LS_G1m3/17 |
| VH G1m3 CH1del | | | | | | |
| C220S IE LS | | | | | | |
| PGDM1400 VL-(G4S)5- | G1m3 | PGDM1400 | PGDM1400 | Hinge_S | CH2 | CH3_cah_LS_G1m3/17 |
| VH G1m3 CH1del | | | | | | |
| C220S cah LS | | | | | | |
| PGDM1400 VL-(G4S)5- | G1m3 | PGDM1400 | PGDM1400 | Hinge_S | CH2_GAALIE | CH3_cah_LS_G1m3/17 |
| VH G1m3 CH1del | | | | | | |
| C220S GAALIE cah LS | | | | | | |
| PGDM1400 VL-(G4S)5- | G1m3 | PGDM1400 | PGDM1400 | Hinge_S | CH2_GAIE | CH3_cah_LS_G1m3/17 |
| VH G1m3 CH1del | | | | | | |
| C220S GAIE cah LS | | | | | | |
| PGDM1400 VL-(G4S)5- | G1m3 | PGDM1400 | PGDM1400 | Hinge_S | CH2_GA | CH3_cah_LS_G1m3/17 |
| VH G1m3 CH1del | | | | | | |
| C220S GA cah LS | | | | | | |
| PGDM1400 VL-(G4S)5- | G1m3 | PGDM1400 | PGDM1400 | Hinge_S | CH2_IE | CH3_cah_LS_G1m3/17 |
| VH G1m3 CH1del | | | | | | |
| C220S IE cah LS | | | | | | |
| PGDM1400 VL-(G4S)5- | G1m3 | PGDM1400 | PGDM1400 | Hinge_S | CH2 | CH3_cak_LS_G1m3/17 |
| VH G1m3 CH1del | | | | | | |
| C220S cak LS | | | | | | |
| PGDM1400 VL-(G4S)5- | G1m3 | PGDM1400 | PGDM1400 | Hinge_S | CH2_GAALIE | CH3_cak_LS_G1m3/17 |
| VH G1m3 CH1del | | | | | | |
| C220S GAALIE cak LS | | | | | | |
| PGDM1400 VL-(G4S)5- | G1m3 | PGDM1400 | PGDM1400 | Hinge_S | CH2_GAIE | CH3_cak_LS_G1m3/17 |
| VH G1m3 CH1del | | | | | | |
| C220S GAIE cak LS | | | | | | |
| PGDM1400 VL-(G4S)5- | G1m3 | PGDM1400 | PGDM1400 | Hinge_S | CH2_GA | CH3_cak_LS_G1m3/17 |
| VH G1m3 CH1del | | | | | | |
| C220S GA cak LS | | | | | | |
| PGDM1400 VL-(G4S)5- | G1m3 | PGDM1400 | PGDM1400 | Hinge_S | CH2_IE | CH3_cak_LS_G1m3/17 |
| VH G1m3 CH1del | | | | | | |
| C220S IE cak LS | | | | | | |
| PGDM1400 VL-(G4S)5- | G1m17 | PGDM1400 | PGDM1400 | Hinge_S | CH2 | CH3_G1m3/17 |
| VH G1m17 CH1del | | | | | | |
| C220S | | | | | | |
| PGDM1400 VL-(G4S)5- | G1m17 | PGDM1400 | PGDM1400 | Hinge_S | CH2 | CH3_LS_G1m3/17 |
| VH G1m17 CH1del | | | | | | |
| C220S LS | | | | | | |
| PGDM1400 VL-(G4S)5- | G1m17 | PGDM1400 | PGDM1400 | Hinge_S | CH2_GAALIE | CH3_LS_G1m3/17 |
| VH G1m17 CH1del | | | | | | |
| C220S GAALIE LS | | | | | | |
| PGDM1400 VL-(G4S)5- | G1m17 | PGDM1400 | PGDM1400 | Hinge_S | CH2_GAIE | CH3_LS_G1m3/17 |
| VH G1m17 CH1del | | | | | | |
| C220S GAIE LS | | | | | | |
| PGDM1400 VL-(G4S)5- | G1m17 | PGDM1400 | PGDM1400 | Hinge_S | CH2_GA | CH3_LS_G1m3/17 |
| VH G1m17 CH1del | | | | | | |
| C220S GA LS | | | | | | |
| PGDM1400 VL-(G4S)5- | G1m17 | PGDM1400 | PGDM1400 | Hinge_S | CH2_IE | CH3_LS_G1m3/17 |
| VH G1m17 CH1del | | | | | | |
| C220S IE LS | | | | | | |
| PGDM1400 VL-(G4S)5- | G1m17 | PGDM1400 | PGDM1400 | Hinge_S | CH2 | CH3_cah_LS_G1m3/17 |
| VH G1m17 CH1del | | | | | | |
| C220S cah LS | | | | | | |
| PGDM1400 VL-(G4S)5- | G1m17 | PGDM1400 | PGDM1400 | Hinge_S | CH2_GAALIE | CH3_cah_LS_G1m3/17 |
| VH G1m17 CH1del | | | | | | |
| C220S GAALIE cah LS | | | | | | |
| PGDM1400 VL-(G4S)5- | G1m17 | PGDM1400 | PGDM1400 | Hinge_S | CH2_GAIE | CH3_cah_LS_G1m3/17 |
| VH G1m17 CH1del | | | | | | |
| C220S GAIE cah LS | | | | | | |
| PGDM1400 VL-(G4S)5- | G1m17 | PGDM1400 | PGDM1400 | Hinge_S | CH2_GA | CH3_cah_LS_G1m3/17 |
| VH G1m17 CH1del | | | | | | |
| C220S GA cah LS | | | | | | |
| PGDM1400 VL-(G4S)5- | G1m17 | PGDM1400 | PGDM1400 | Hinge_S | CH2_IE | CH3_cah_LS_G1m3/17 |
| VH G1m17 CH1del | | | | | | |
| C220S IE cah LS | | | | | | |
| PGDM1400 VL-(G4S)5- | G1m17 | PGDM1400 | PGDM1400 | Hinge_S | CH2 | CH3_cak_LS_G1m3/17 |
| VH G1m17 CH1del | | | | | | |
| C220S cak LS | | | | | | |
| PGDM1400 VL-(G4S)5- | G1m17 | PGDM1400 | PGDM1400 | Hinge_S | CH2_GAALIE | CH3_cak_LS_G1m3/17 |
| VH G1m17 CH1del | | | | | | |
| C220S GAALIE cak LS | | | | | | |
| PGDM1400 VL-(G4S)5- | G1m17 | PGDM1400 | PGDM1400 | Hinge_S | CH2_GAIE | CH3_cak_LS_G1m3/17 |
| VH G1m17 CH1del | | | | | | |
| C220S GAIE cak LS | | | | | | |
| PGDM1400 VL-(G4S)5- | G1m17 | PGDM1400 | PGDM1400 | Hinge_S | CH2_GA | CH3_cak_LS_G1m3/17 |
| VH G1m17 CH1del | | | | | | |
| C220S GA cak LS | | | | | | |
| PGDM1400 VL-(G4S)5- | G1m17 | PGDM1400 | PGDM1400 | Hinge_S | CH2_IE | CH3_cak_LS_G1m3/17 |
| VH G1m17 CH1del | | | | | | |
| C220S IE cak LS | | | | | | |
| PGDM1400 VL-(G4S)5- | G1m17,1 | PGDM1400 | PGDM1400 | Hinge_S | CH2 | CH3_G1m17,1 |
| VH G1m17,1 CH1del | | | | | | |
| C220S | | | | | | |
| PGDM1400 VL-(G4S)5- | G1m17,1 | PGDM1400 | PGDM1400 | Hinge_S | CH2 | CH3_LS_G1m17,1 |
| VH G1m17,1 CH1del | | | | | | |
| C220S LS | | | | | | |
| PGDM1400 VL-(G4S)5- | G1m17,1 | PGDM1400 | PGDM1400 | Hinge_S | CH2_GAALIE | CH3_LS_G1m17,1 |
| VH G1m17,1 CH1del | | | | | | |
| C220S GAALIE LS | | | | | | |
| PGDM1400 VL-(G4S)5- | G1m17,1 | PGDM1400 | PGDM1400 | Hinge_S | CH2_GAIE | CH3_LS_G1m17,1 |
| VH G1m17,1 CH1del | | | | | | |
| C220S GAIE LS | | | | | | |
| PGDM1400 VL-(G4S)5- | G1m17,1 | PGDM1400 | PGDM1400 | Hinge_S | CH2_GA | CH3_LS_G1m17,1 |
| VH G1m17,1 CH1del | | | | | | |
| C220S GA LS | | | | | | |
| PGDM1400 VL-(G4S)5- | G1m17,1 | PGDM1400 | PGDM1400 | Hinge_S | CH2_IE | CH3_LS_G1m17,1 |
| VH G1m17,1 CH1del | | | | | | |
| C220S IE LS | | | | | | |
| PGDM1400 VL-(G4S)5- | G1m17,1 | PGDM1400 | PGDM1400 | Hinge_S | CH2 | CH3_cah_LS_G1m17,1 |
| VH G1m17,1 CH1del | | | | | | |
| C220S cah LS | | | | | | |
| PGDM1400 VL-(G4S)5- | G1m17,1 | PGDM1400 | PGDM1400 | Hinge_S | CH2_GAALIE | CH3_cah_LS_G1m17,1 |
| VH G1m17,1 CH1del | | | | | | |
| C220S GAALIE cah LS | | | | | | |
| PGDM1400 VL-(G4S)5- | G1m17,1 | PGDM1400 | PGDM1400 | Hinge_S | CH2_GAIE | CH3_cah_LS_G1m17,1 |
| VH G1m17,1 CH1del | | | | | | |
| C220S GAIE cah LS | | | | | | |
| PGDM1400 VL-(G4S)5- | G1m17,1 | PGDM1400 | PGDM1400 | Hinge_S | CH2_GA | CH3_cah_LS_G1m17,1 |
| VH G1m17,1 CH1del | | | | | | |
| C220S GA cah LS | | | | | | |
| PGDM1400 VL-(G4S)5- | G1m17,1 | PGDM1400 | PGDM1400 | Hinge_S | CH2_IE | CH3_cah_LS_G1m17,1 |
| VH G1m17,1 CH1del | | | | | | |
| C220S IE cah LS | | | | | | |
| PGDM1400 VL-(G4S)5- | G1m17,1 | PGDM1400 | PGDM1400 | Hinge_S | CH2 | CH3_cak_LS_G1m17,1 |
| VH G1m17,1 CH1del | | | | | | |
| C220S cak LS | | | | | | |
| PGDM1400 VL-(G4S)5- | G1m17,1 | PGDM1400 | PGDM1400 | Hinge_S | CH2_GAALIE | CH3_cak_LS_G1m17,1 |
| VH G1m17,1 CH1del | | | | | | |
| C220S GAALIE cak LS | | | | | | |
| PGDM1400 VL-(G4S)5- | G1m17,1 | PGDM1400 | PGDM1400 | Hinge_S | CH2_GAIE | CH3_cak_LS_G1m17,1 |
| VH G1m17,1 CH1del | | | | | | |
| C220S GAIE cak LS | | | | | | |
| PGDM1400 VL-(G4S)5- | G1m17,1 | PGDM1400 | PGDM1400 | Hinge_S | CH2_GA | CH3_cak_LS_G1m17,1 |
| VH G1m17,1 CH1del | | | | | | |
| C220S GA cak LS | | | | | | |
| PGDM1400 VL-(G4S)5- | G1m17,1 | PGDM1400 | PGDM1400 | Hinge_S | CH2_IE | CH3_cak_LS_G1m17,1 |
| VH G1m17,1 CH1del | | | | | | |
| C220S IE cak LS | | | | | | |

### III. Polyribonucleotides

### A. Exemplary Polyribonucleotides Features

Polyribonucleotides described herein encode an immunoglobulin chain of an antibody agent as described herein (e.g., HIV antibody agents). Additionally, polyribonucleotides described herein, in some embodiments, include other elements such as a secretion signal-encoding region. In some embodiments, polyribonucleotides described herein can comprise a nucleotide sequence that encodes a 5′UTR of interest and/or a 3′ UTR of interest. In some embodiments, polynucleotides described herein can comprise a nucleotide sequence that encodes a polyA tail. In some embodiments, polyribonucleotides described herein may comprise a 5′ cap, which may be incorporated during transcription, or joined to a polyribonucleotide post-transcription.

### 1. Secretion Signal-Encoding Region

According to certain embodiments, a signal peptide (or signal sequence) is fused, either directly or through a linker, to an encoded immunoglobulin chain of an antibody agent described herein.

In some embodiments, an open reading frame of the RNA described herein encodes an immunoglobulin chain of an antibody agent described herein with a signal sequence, e.g., that is functional in mammalian cells. In some embodiments, a utilized signal sequence is “intrinsic” in that it is, in nature, associated with (e.g., linked to) an immunoglobulin chain of an antibody agent or portion thereof.

In some embodiments, a utilized signal sequence is heterologous to an immunoglobulin chain of an antibody agent or portion thereof—e.g., is not naturally part of an immunoglobulin chain of an antibody agent or portion thereof.

In some embodiments, signal peptides are sequences, which are typically characterized by a length of about 15 to 30 amino acids.

In many embodiments, signal peptides are positioned at the N-terminus of an immunoglobulin chain of an antibody agent or portion thereof, without being limited thereto. In some embodiments, signal peptides preferably allow the transport of an immunoglobulin chain of an antibody agent or portion thereof encoded by RNAs of the present disclosure with which they are associated into a defined cellular compartment, preferably the cell surface, the endoplasmic reticulum (ER) or the endosomal-lysosomal compartment.

In some embodiments, a polyribonucleotide, as provided herein, that encodes an immunoglobulin chain of an antibody agent (e.g., an HIV antibody agent) may comprise a ribonucleic acid sequence encoding a secretion signal. In some embodiments, a ribonucleic acid sequence encoding a secretion signal allows an immunoglobulin chain of an antibody agent encoded by the polyribonucleotide to be secreted upon translation by cells, e.g., present in a subject, thus yielding a plasma concentration of a biologically active HIV antibody agent.

In some embodiments, a ribonucleic acid sequence encoding a secretion signal included in a polyribonucleotide consists of or comprises a nucleotide sequence that encodes a human secretion signal. For example, in some embodiments, such a human secretion signal may be or comprises the amino acid sequence of SEQ ID NO: 1 (husec2). In some embodiments, a ribonucleic acid sequence encoding a secretion signal included in a polyribonucleotide consists of or comprises a nucleotide sequence that encodes a non-human secretion signal. In some embodiments, a ribonucleic acid sequence encoding a secretion signal included in a polyribonucleotide encoding a heavy chain domain of an antibody agent may comprise a ribonucleic acid sequence that encodes a human secretion signal amino acid sequence. In some embodiments, a polyribonucleotide encoding a human secretion signal comprises SEQ ID NO: 2, 4, 6, or 8. In some embodiments, a ribonucleic acid sequence encoding a secretion signal included in a polyribonucleotide encoding a light chain domain of a HIV antibody agent may comprise a nucleotide sequence that encodes a human secretion signal amino acid sequence of SEQ ID NO: 1 (husec2). In some embodiments, a ribonucleic acid sequence encoding a secretion signal included in a polyribonucleotide encoding a light chain domain of a HIV antibody agent may comprise a nucleotide sequence that encodes a human secretion signal amino acid sequence. In some embodiments, a polyribonucleotide encoding a human secretion signal comprises SEQ ID NO: 2,4, 6, or 8.

In some embodiments, an RNA sequence encodes an immunoglobulin chain of an antibody agent as described herein may comprise or otherwise be linked to a signal sequence (e.g., secretory sequence), such as those listed in Table 6, or a sequence having 1, 2, 3, 4, or 5 amino acid differences relative thereto. In some embodiments, a signal sequence such as SEQ ID NO: 1479, or a sequence having 1, 2, 3, 4, or at the most 5 amino acid differences relative thereto, is utilized. In some embodiments, a signal sequence such as SEQ ID NO: 1480, or a sequence having 1, 2, 3, 4, or at the most 5 amino acid differences relative thereto, is utilized.

In some embodiments, a signal sequence is selected from those included in the Table 7 below:

TABLE 7
Exemplary Signal sequences

| Signal | SEQ ID NO: | Sequence (Amino Acid) |
|---|---|---|
| HSV-1 gD SP | 1463 | MGGAAARLGAVILFVVIVGLHGVRSKY |
| HSV-2 gD SP | 1464 | MGRLTSGVGTAALLVVAVGLRVVCA |
| HSV-2 | 1465 | MGRLTSGVGTAALLVVAVGLRVVCAKYA |
| SARS-CoV-2-S | 1466 | MFVFLVLLPLVSSQCVNLT |
| human Ig heavy chain | 1467 | MDWIWRILFLVGAATGAHSQM |
| signal peptide | | |
| human Ig heavy chain | 1468 | MDWTWRVFCLLAVAPGAHS |
| signal peptide | | |
| HuIgGk signal peptide | 1469 | METPAQLLFLLLLWLPDTTG |
| IgE heavy chain epsilon- | 1470 | MDWTWILFLVAAATRVHS |
| 1signal peptide | | |
| Japanese encephalitis PRM | 1471 | MLGSNSGQRVVFTILLLLVAPAYS |
| signal sequence | | |
| VSVg protein signal | 1472 | MKCLLYLAFLFIGVNCA |
| sequence | | |
| Exemplary signal sequence | 1473 | MDWTWILFLVAAATRVHS |
| Exemplary signal sequence | 1474 | ETPAQLLFLLLLWLPDTTG |
| Exemplary signal sequence | 1475 | MLGSNSGQRVVFTILLLLVAPAYS |
| Exemplary signal sequence | 1476 | MKCLLYLAFLFIGVNCA |
| Exemplary signal sequence | 1477 | MWLVSLAIVTACAGA |
| Exemplary signal sequence | 1478 | MFVFLVLLPLVSSQC |
| husec Signal peptide | 1479 | MRVMAPRTLILLLSGALALTETWAGS |
| husec3 Signal peptide | 1480 | MEFGLSWLFLVAILKGVQC |

### 2. 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550 reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Zust et al. (2011) Nature Immunology, 12:137-143.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. For example, in some embodiments, a 5′-cap structure which may be suitable in the context of the present invention is a cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxoguanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

In some embodiments, a utilized 5′ caps is a cap0, a cap1, or cap2 structure. See, e.g., Fig. 1 of Ramanathan A et al., and Fig. 1 of Decroly E et al., each of which is incorporated herein by reference in its entirety. See, e.g., Fig. 1 of Ramanathan A et al., and Fig. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position pf the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, wherein each of N₁ and N₂ is as defined and described herein.

In some embodiments, a cap2 structure comprises a guanosine nucleoside methylated at the 7-position of guanine ((m⁷)G) and optionally methylated at the 2′ or 3′ position pf the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and G* comprises a structure of formula (I):

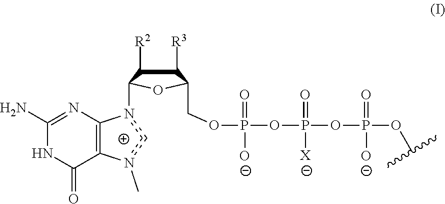

or a salt thereof,
wherein
   each R² and R³ is —OH or —OCH₃; and
   X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,3′-O})GppSpN₁, or (m₂^{7,3′}-O)GppSpN₁), wherein N₁ is as defined and described herein. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′}-O)GppSpN₁), wherein N₁ is G. In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is A, U, or C. In some embodiments, the 5′ cap is a dinucleotide cap1 structure (e.g., (m⁷)Gppp(m^{2′-O})N₁, (m₂^{7,2′-O}Gppp(m^{2′-O})N₁, (m₂^{7,3′-O})Gppp(m^{2′-O})N₁, (m⁷)GppSp(m^{2′-O})N₁, (m₂^{7,2′-O}GppSp(m^{2′-O})N₁, or (m₂^{7,3′-O})GppSp(m^{2′-O})N₁), wherein N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, the 5′ cap is (m⁷)GpppG (“Ecap0”), having a structure:

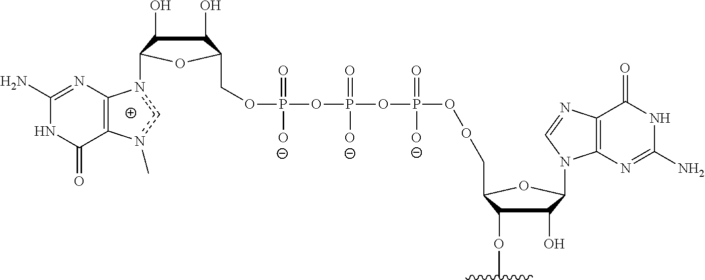

or a salt thereof.

In some embodiments, the 5′ cap is (m⁷)Gppp(m^{2′-O})G (“Ecap1”), having a structure:

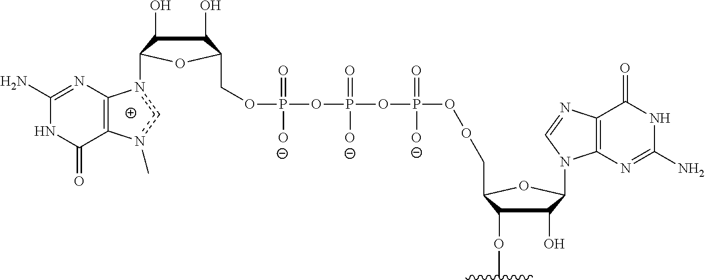

or a salt thereof.

In some embodiments, the 5′ cap is (m₂^{7,3′-O})GpppG (“ARCA” or “D1”), having a structure:

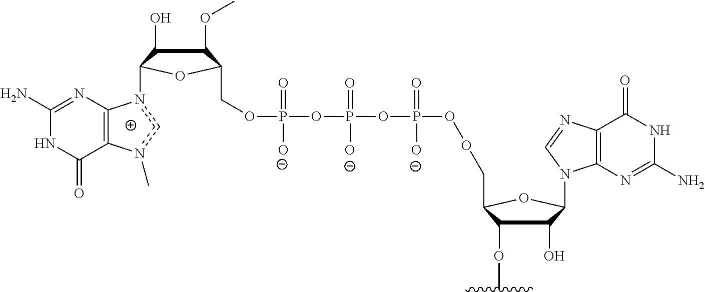

or a salt thereof.

In some embodiments, the 5′ cap is (m₂^{7,2′-O})GppSpG (“beta-S-ARCA”), having a structure:

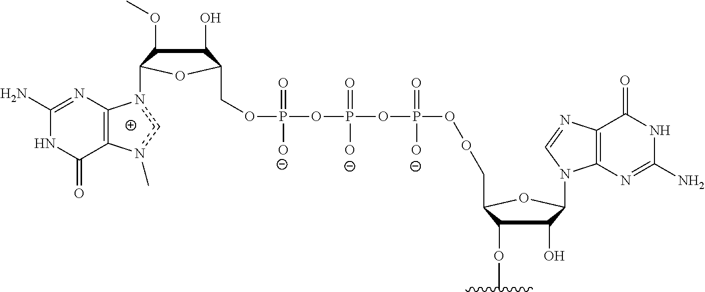

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a dinucleotide cap G*N₁pN₂, wherein N₁ and N₂ are as defined above and herein, and G* comprises a structure of formula (I):

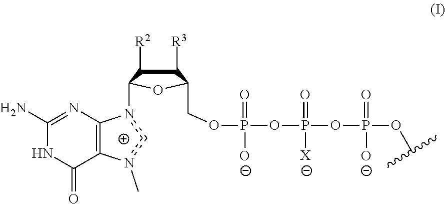

or a salt thereof, wherein R², R³, and X are as defined and described herein.

In some embodiments, the 5′ cap is a trinucleotide cap0 structure (e.g. (m⁷)GpppN₁pN₂, (m₂^{7,2′-O})GpppN₁pN₂, or (m₂^{7,3′-O})GpppN₁pN₂), wherein N₁ and N₂ are as defined and described herein). In some embodiments, the 5′ cap is a trinucleotide cap1 structure (e.g., (m⁷)Gppp(m^{2′-O})N₁pN₂, (m₂^{7,3′-O})Gppp(m^{2′-O})N₁pN₂, (m₂^{7,3′-O})Gppp(m^{2′-O})N₁pN₂), wherein N₁ and N₂ are as defined and described herein. In some embodiments, the 5′ cap is a trinucleotide cap2 structure (e.g., (m⁷)Gppp(m^{2′-O})N₁p(m^{2′-O})N₂, (m₂^{7,3′-O})Gppp(m^{2′-O})N₁p(m^{2′-O})N₂, (m₂^{7,3′-O})Gppp(m^{2′-O})N₁p(m^{2′-O})N₂), wherein N₁ and N₂ are as defined and described herein. In some embodiments, the 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, (m⁷)Gppp(m^{2′-O})GpG, (m₂^{7,3′-O})Gppp(m₂^{6,2′-O})ApG, and (m⁷)Gppp(m^{2′-O})ApU.

In some embodiments, the 5′ cap is (m₂^{7,3′-O})Gppp(m^{2′-O})ApG (“CleanCap AG”, “CC413”), having a structure:

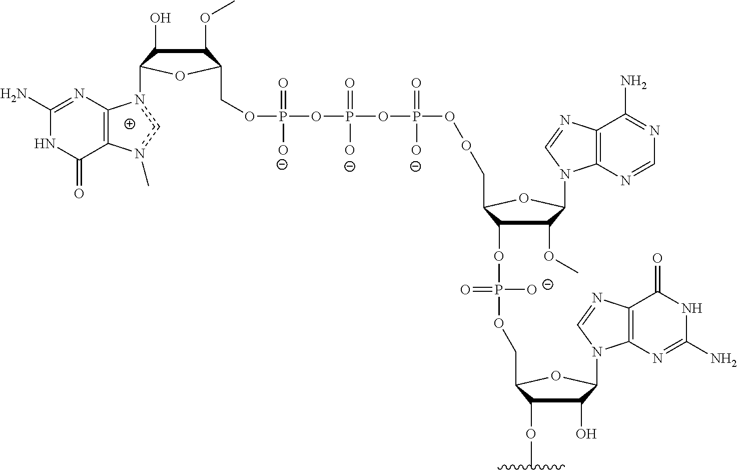

or a salt thereof.

In some embodiments, the 5′ cap is (m₂^{7,3′-O})Gppp(m^{2′-O})GpG (“CleanCap GG”), having a structure:

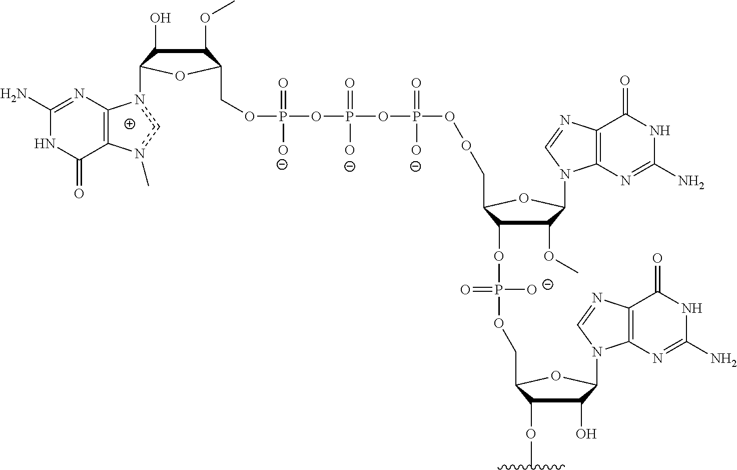

or a salt thereof.

In some embodiments, the 5′ cap is (m⁷)Gppp(m^{2′-O})ApG, having a structure:

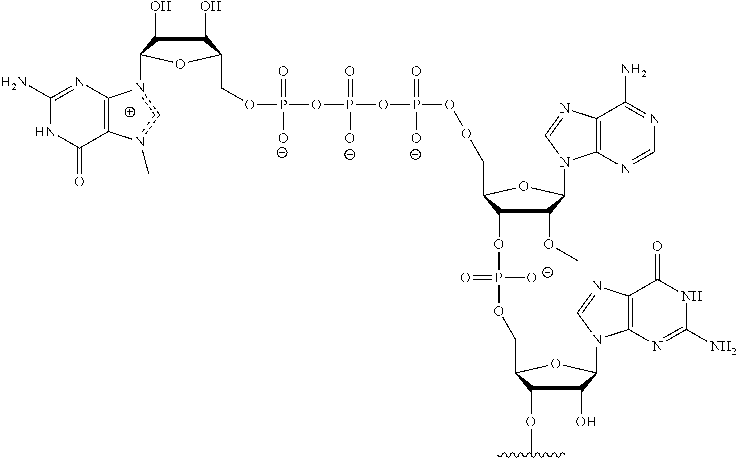

or a salt thereof.

In some embodiments, the 5′ cap is (m⁷)Gppp(m^{2′-O})GpG, having a structure:

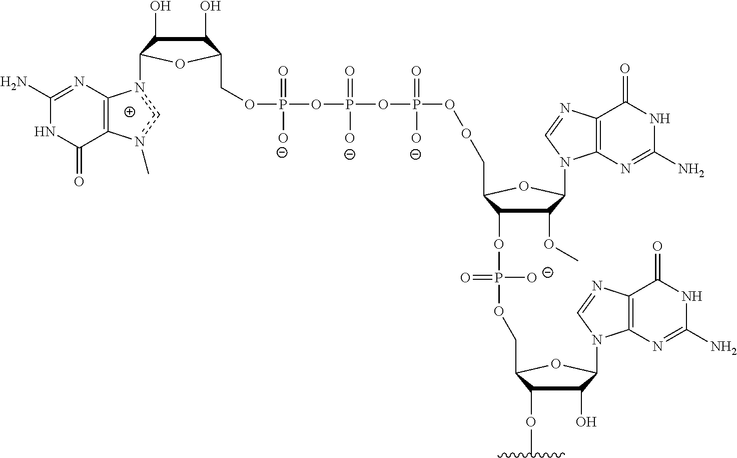

or a salt thereof.

In some embodiments, the 5′ cap is (m₂^{7,3′-O})Gppp(m₂^{6,2′-O})ApG, having a structure:

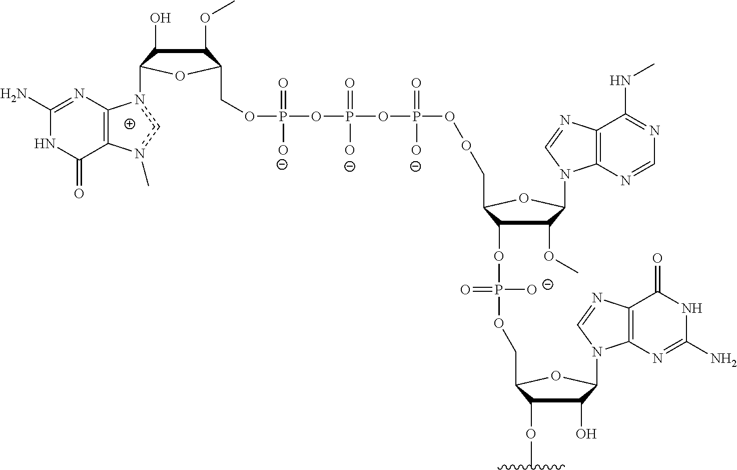

or a salt thereof.

In some embodiments, the 5′ cap is (m⁷)Gppp(m^{2′-O})ApU, having a structure:

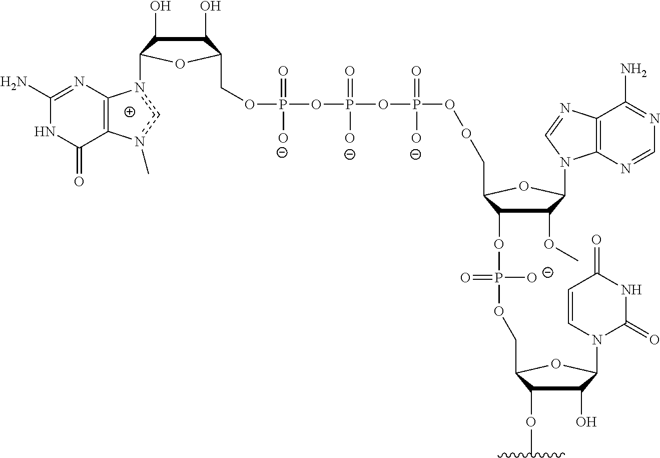

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N₁pN₂pN₃, wherein N₁, N₂, and N₃ are as defined above and herein, and G* comprises a structure of formula (I):

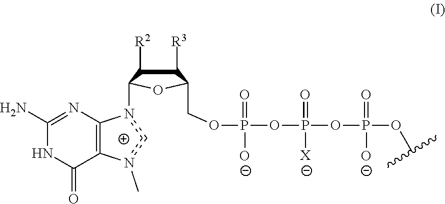

or a salt thereof, wherein R², R³, and X are as defined and described herein.

In some embodiments, the 5′ cap is a tetranucleotide cap0 structure (e.g. (m⁷)GpppN₁pN₂pN₃, (m₂^{7,3′-O})GpppN₁pN₂pN₃, or (m₂^{7,3′-O})GpppN₁N2pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,2′-O})Gppp(m^{2′-O})N₁pN₂pN₃, (m₂^{7,3′-O})Gppp(m^{2′-O})N₁pN₂N₃), wherein N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂pN₃, (m₂^{7,2′-O})Gppp(m^{2′-O})N₁p(m^{2′-O})N₂pN₃, (m₂^{7,3′-O})Gppp(m^{2′-O})N₁p(m^{2′-O})N₂pN₃), wherein N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure:

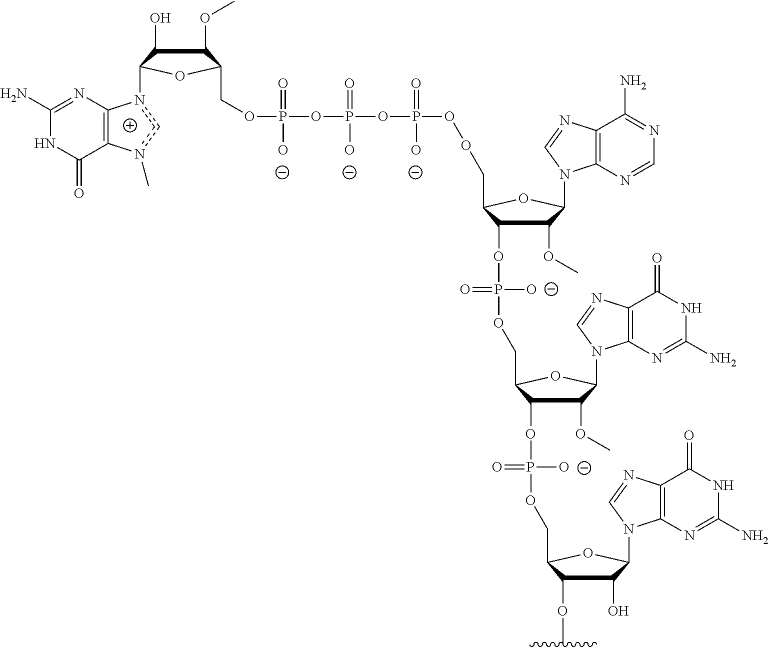

or a salt thereof.

In some embodiments, the 5′ cap is (m₂^{7,3′-O})Gppp(m^{2′-O})Gp(m^{2′-O})GpC, having a structure:

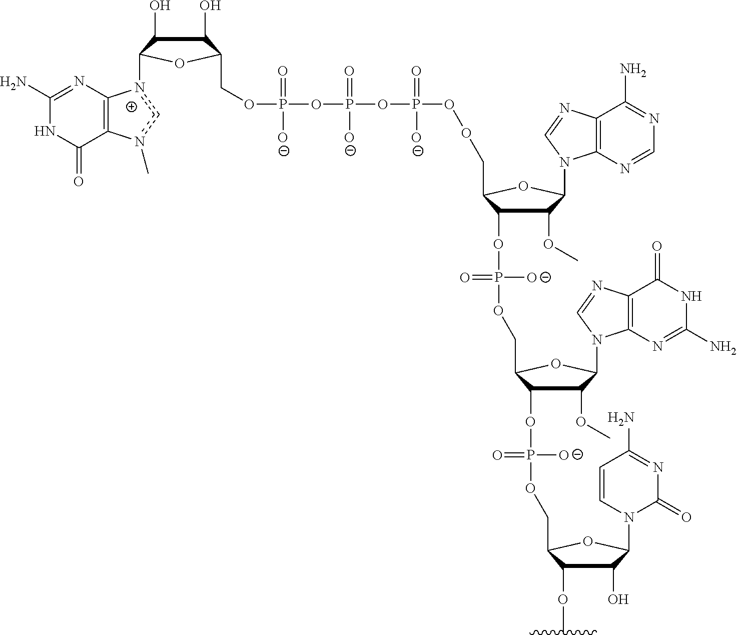

or a salt thereof.

In some embodiments, the 5′ cap is (m⁷)Gppp(m^{2′-O})Ap(m^{2′-O})UpA, having a structure:

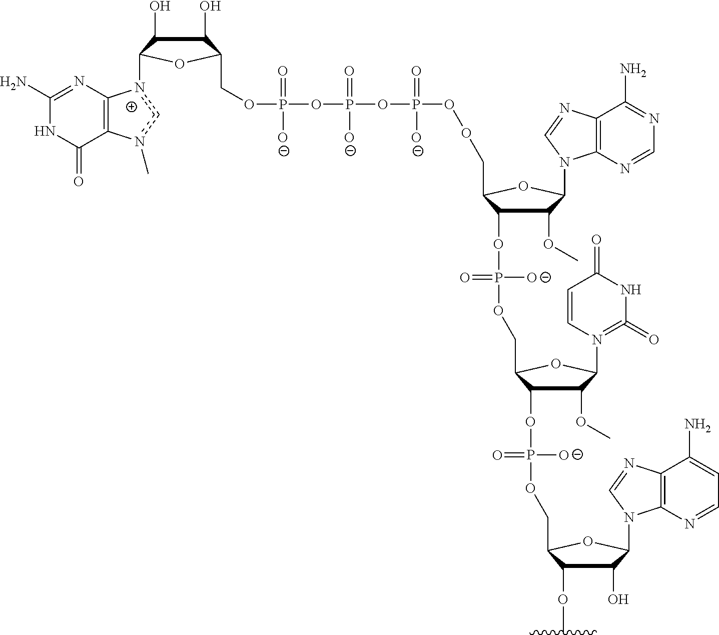

or a salt thereof.

In some embodiments, the 5′ cap is (m⁷)Gppp(m^{2′-O})Ap(m^{2′-O})GpG, having a structure:

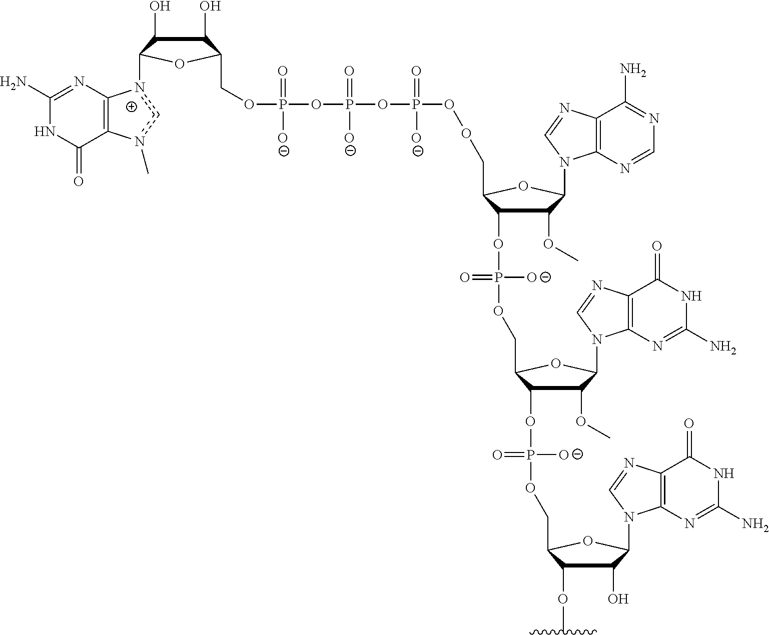

or a salt thereof.

### 3. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide+1 (N₁) of an RNA polynucleotide. In some embodiments, a cap structure comprises an m⁷ Guanosine cap and nucleotide+2 (N₂) of an RNA polynucleotide. In some embodiments, a cap structure comprises an m⁷ Guanosine cap and nucleotides+1 and +2 (N₁ and N₂) of an RNA polynucleotide. In some embodiments, a cap structure comprises an m⁷ Guanosine cap and nucleotides+1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues+1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that each of the embodiments described above and herein (e.g., for N₁ through N₅) may be taken singly or in combination and/or may be combined with other embodiments of variables described above and herein (e.g., 5′ caps).

### 4. 5′ UTR

In some embodiments, a nucleic acid (e.g., DNA, RNA) utilized in accordance with the present disclosure a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence according to SEQ ID NO: 10. In some embodiments, an RNA disclosed herein comprises a 5′ UTR provided in SEQ ID NO: 10.

### 5. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence (e.g., SEQ ID NO: 1483) in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly (A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

In some embodiments, RNA comprises a poly(A) sequence comprising the nucleotide sequence of SEQ ID NO: 16, or a nucleotide sequence having at least 99%, 98%, 97%, 96%, 95%, 90%, 85%, or 80% identity to the nucleotide sequence of SEQ ID NO: 16. In some embodiments, a poly(A) tail comprises a plurality of A residues interrupted by a linker. In some embodiments, a linker comprises the nucleotide sequence GCATATGAC (SEQ ID NO: 1461).

### 6. 3′ UTR

In some embodiments, an RNA utilized in accordance with the present disclosure comprises a 3′-UTR. As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” does preferably not include the poly(A) sequence. Thus, the 3′-UTR is upstream of the poly(A) sequence (if present), e.g., directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a XhoI site.

In some embodiments, an RNA construct comprises an F element. In some embodiments, an F element sequence is a 3′-UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence according to SEQ ID NO: 14. In some embodiments, an RNA disclosed herein comprises a 3 UTR provided in SEQ ID NO: 14.

In some embodiments, a 3′UTR is an FI element as described in WO2017/060314, which is herein incorporated by reference in its entirety.

### 7. Exemplary Polyribonucleotides

Exemplary polyribonucleotide constructs comprising coding and non-coding elements as described herein are shown in Table 8 below.

TABLE 8
Exemplary polyribonucleotide constructs (Including 5′UTR, 3′UTR and PolyA)

| SEQ ID NO | |
|---|---|
| | IgG Format - Heavy Chain |
| 850 | PGDM1400 VH G1m3 |
| 851 | PGDM1400 VH G1m3 LS |
| 852 | PGDM1400 VH G1m3 cah LS |
| 853 | PGDM1400 VH G1m3 cak LS |
| 854 | PGDM1400 VH G1m3 GAALIE LS |
| 855 | PGDM1400 VH G1m3 GAIE LS |
| 856 | PGDM1400 VH G1m3 GA LS |
| 857 | PGDM1400 VH G1m3 IE LS |
| 858 | PGDM1400 VH G1m3 GAALIE cah LS |
| 859 | PGDM1400 VH G1m3 GAIE cah LS |
| 860 | PGDM1400 VH G1m3 GA cah LS |
| 861 | PGDM1400 VH G1m3 IE cah LS |
| 862 | PGDM1400 VH G1m3 GAALIE cak LS |
| 863 | PGDM1400 VH G1m3 GAIE cak LS |
| 864 | PGDM1400 VH G1m3 GA cak LS |
| 865 | PGDM1400 VH G1m3 IE cak LS |
| 866 | PGDM1400 VH G1m17 |
| 867 | PGDM1400 VH G1m17 LS |
| 868 | PGDM1400 VH G1m17 cah LS |
| 869 | PGDM1400 VH G1m17 cak LS |
| 870 | PGDM1400 VH G1m17 GAALIE LS |
| 871 | PGDM1400 VH G1m17 GAIE LS |
| 872 | PGDM1400 VH G1m17 GA LS |
| 873 | PGDM1400 VH G1m17 IE LS |
| 874 | PGDM1400 VH G1m17 GAALIE cah LS |
| 875 | PGDM1400 VH G1m17 GAIE cah LS |
| 876 | PGDM1400 VH G1m17 GA cah LS |
| 877 | PGDM1400 VH G1m17 IE cah LS |
| 878 | PGDM1400 VH G1m17 GAALIE cak LS |
| 879 | PGDM1400 VH G1m17 GAIE cak LS |
| 880 | PGDM1400 VH G1m17 GA cak LS |
| 881 | PGDM1400 VH G1m17 IE cak LS |
| 882 | PGDM1400 VH G1m17,1 |
| 883 | PGDM1400 VH G1m17,1 LS |
| 884 | PGDM1400 VH G1m17,1 cah LS |
| 885 | PGDM1400 VH G1m17,1 cak LS |
| 886 | PGDM1400 VH G1m17,1 GAALIE LS |
| 887 | PGDM1400 VH G1m17,1 GAIE LS |
| 888 | PGDM1400 VH G1m17,1 GA LS |
| 889 | PGDM1400 VH G1m17,1 IE LS |
| 890 | PGDM1400 VH G1m17,1 GAALIE cah LS |
| 891 | PGDM1400 VH G1m17,1 GAIE cah LS |
| 892 | PGDM1400 VH G1m17,1 GA cah LS |
| 893 | PGDM1400 VH G1m17,1 IE cah LS |
| 894 | PGDM1400 VH G1m17,1 GAALIE cak LS |
| 895 | PGDM1400 VH G1m17,1 GAIE cak LS |
| 896 | PGDM1400 VH G1m17,1 GA cak LS |
| 897 | PGDM1400 VH G1m17,1 IE cak LS |
| | IgG Format - Light Chain |
| 898 | PGDM1400 VL Ck |
| | CrossMabCH1-CLx Format - Heavy Chain |
| 899 | PGDM1400 VH G1m3 Ck(AS/Q124E) del216-220 |
| 900 | PGDM1400 VH G1m3 Ck(AS/Q124E) del216-220 LS |
| 901 | PGDM1400 VH G1m3 Ck(AS/Q124E) del216-220 GAALIE LS |
| 902 | PGDM1400 VH G1m3 Ck(AS/Q124E) del216-220 GAIE LS |
| 903 | PGDM1400 VH G1m3 Ck(AS/Q124E) del216-220 GA LS |
| 904 | PGDM1400 VH G1m3 Ck(AS/Q124E) del216-220 IE LS |
| 905 | PGDM1400 VH G1m3 Ck(AS/Q124E) del216-220 cah LS |
| 906 | PGDM1400 VH G1m3 Ck(AS/Q124E) del216-220 GAALIE cah LS |
| 907 | PGDM1400 VH G1m3 Ck(AS/Q124E) del216-220 GAIE cah LS |
| 908 | PGDM1400 VH G1m3 Ck(AS/Q124E) del216-220 GA cah LS |
| 909 | PGDM1400 VH G1m3 Ck(AS/Q124E) del216-220 IE cah LS |
| 910 | PGDM1400 VH G1m3 Ck(AS/Q124E) del216-220 cak LS |
| 911 | PGDM1400 VH G1m3 Ck(AS/Q124E) del216-220 GAALIE cak LS |
| 912 | PGDM1400 VH G1m3 Ck(AS/Q124E) del216-220 GAIE cak LS |
| 913 | PGDM1400 VH G1m3 Ck(AS/Q124E) del216-220 GA cak LS |
| 914 | PGDM1400 VH G1m3 Ck(AS/Q124E) del216-220 IE cak LS |
| 915 | PGDM1400 VH G1m17 Ck(AS/Q124E) del216-220 |
| 916 | PGDM1400 VH G1m17 Ck(AS/Q124E) del216-220 LS |
| 917 | PGDM1400 VH G1m17 Ck(AS/Q124E) del216-220 GAALIE LS |
| 918 | PGDM1400 VH G1m17 Ck(AS/Q124E) del216-220 GAIE LS |
| 919 | PGDM1400 VH G1m17 Ck(AS/Q124E) del216-220 GA LS |
| 920 | PGDM1400 VH G1m17 Ck(AS/Q124E) del216-220 IE LS |
| 921 | PGDM1400 VH G1m17 Ck(AS/Q124E) del216-220 cah LS |
| 922 | PGDM1400 VH G1m17 Ck(AS/Q124E) del216-220 GAALIE cah LS |
| 923 | PGDM1400 VH G1m17 Ck(AS/Q124E) del216-220 GAIE cah LS |
| 924 | PGDM1400 VH G1m17 Ck(AS/Q124E) del216-220 GA cah LS |
| 925 | PGDM1400 VH G1m17 Ck(AS/Q124E) del216-220 IE cah LS |
| 926 | PGDM1400 VH G1m17 Ck(AS/Q124E) del216-220 cak LS |
| 927 | PGDM1400 VH G1m17 Ck(AS/Q124E) del216-220 GAALIE cak LS |
| 928 | PGDM1400 VH G1m17 Ck(AS/Q124E) del216-220 GAIE cak LS |
| 929 | PGDM1400 VH G1m17 Ck(AS/Q124E) del216-220 GA cak LS |
| 930 | PGDM1400 VH G1m17 Ck(AS/Q124E) del216-220 IE cak LS |
| 931 | PGDM1400 VH G1m17,1 Ck(AS/Q124E) del216-220 |
| 932 | PGDM1400 VH G1m17,1 Ck(AS/Q124E) del216-220 LS |
| 933 | PGDM1400 VH G1m17,1 Ck(AS/Q124E) del216-220 GAALIE LS |
| 934 | PGDM1400 VH G1m17,1 Ck(AS/Q124E) del216-220 GAIE LS |
| 935 | PGDM1400 VH G1m17,1 Ck(AS/Q124E) del216-220 GA LS |
| 936 | PGDM1400 VH G1m17,1 Ck(AS/Q124E) del216-220 IE LS |
| 937 | PGDM1400 VH G1m17,1 Ck(AS/Q124E) del216-220 cah LS |
| 938 | PGDM1400 VH G1m17,1 Ck(AS/Q124E) del216-220 GAALIE cah LS |
| 939 | PGDM1400 VH G1m17,1 Ck(AS/Q124E) del216-220 GAIE cah LS |
| 940 | PGDM1400 VH G1m17,1 Ck(AS/Q124E) del216-220 GA cah LS |
| 941 | PGDM1400 VH G1m17,1 Ck(AS/Q124E) del216-220 IE cah LS |
| 942 | PGDM1400 VH G1m17,1 Ck(AS/Q124E) del216-220 cak LS |
| 943 | PGDM1400 VH G1m17,1 Ck(AS/Q124E) del216-220 GAALIE cak LS |
| 944 | PGDM1400 VH G1m17,1 Ck(AS/Q124E) del216-220 GAIE cak LS |
| 945 | PGDM1400 VH G1m17,1 Ck(AS/Q124E) del216-220 GA cak LS |
| 946 | PGDM1400 VH G1m17,1 Ck(AS/Q124E) del216-220 IE cak LS |
| | CrossMabCH1-CLx Format - Light Chain |
| 947 | PGDM1400 VL CH1(SS) (G1m3) |
| 948 | PGDM1400 VL CH1(SS) (G1m17±,1) |
| | CrossMabCH1-CLcv Format - Heavy Chain |
| 949 | PGDM1400 VH G1m3 K147E + K213D |
| 950 | PGDM1400 VH G1m3 K147E + K213D LS |
| 951 | PGDM1400 VH G1m3 K147E + K213D GAALIE LS |
| 952 | PGDM1400 VH G1m3 K147E + K213D GAIE LS |
| 953 | PGDM1400 VH G1m3 K147E + K213D GA LS |
| 954 | PGDM1400 VH G1m3 K147E + K213D IE LS |
| 955 | PGDM1400 VH G1m3 K147E + K213D cah LS |
| 956 | PGDM1400 VH G1m3 K147E + K213D GAALIE cah LS |
| 957 | PGDM1400 VH G1m3 K147E + K213D GAIE cah LS |
| 958 | PGDM1400 VH G1m3 K147E + K213D GA cah LS |
| 959 | PGDM1400 VH G1m3 K147E + K213D IE cah LS |
| 960 | PGDM1400 VH G1m3 K147E + K213D cak LS |
| 961 | PGDM1400 VH G1m3 K147E + K213D GAALIE cak LS |
| 962 | PGDM1400 VH G1m3 K147E + K213D GAIE cak LS |
| 963 | PGDM1400 VH G1m3 K147E + K213D GA cak LS |
| 964 | PGDM1400 VH G1m3 K147E + K213D IE cak LS |
| 965 | PGDM1400 VH G1m17 K147E + K213D |
| 966 | PGDM1400 VH G1m17 K147E + K213D LS |
| 967 | PGDM1400 VH G1m17 K147E + K213D GAALIE LS |
| 968 | PGDM1400 VH G1m17 K147E + K213D GAIE LS |
| 969 | PGDM1400 VH G1m17 K147E + K213D GA LS |
| 970 | PGDM1400 VH G1m17 K147E + K213D IE LS |
| 971 | PGDM1400 VH G1m17 K147E + K213D cah LS |
| 972 | PGDM1400 VH G1m17 K147E + K213D GAALIE cah LS |
| 973 | PGDM1400 VH G1m17 K147E + K213D GAIE cah LS |
| 974 | PGDM1400 VH G1m17 K147E + K213D GA cah LS |
| 975 | PGDM1400 VH G1m17 K147E + K213D IE cah LS |
| 976 | PGDM1400 VH G1m17 K147E + K213D cak LS |
| 977 | PGDM1400 VH G1m17 K147E + K213D GAALIE cak LS |
| 978 | PGDM1400 VH G1m17 K147E + K213D GAIE cak LS |
| 979 | PGDM1400 VH G1m17 K147E + K213D GA cak LS |
| 980 | PGDM1400 VH G1m17 K147E + K213D IE cak LS |
| 981 | PGDM1400 VH G1m17,1 K147E + K213D |
| 982 | PGDM1400 VH G1m17,1 K147E + K213D LS |
| 983 | PGDM1400 VH G1m17,1 K147E + K213D GAALIE LS |
| 984 | PGDM1400 VH G1m17,1 K147E + K213D GAIE LS |
| 985 | PGDM1400 VH G1m17,1 K147E + K213D GA LS |
| 986 | PGDM1400 VH G1m17,1 K147E + K213D IE LS |
| 987 | PGDM1400 VH G1m17,1 K147E + K213D cah LS |
| 988 | PGDM1400 VH G1m17,1 K147E + K213D GAALIE cah LS |
| 989 | PGDM1400 VH G1m17,1 K147E + K213D GAIE cah LS |
| 990 | PGDM1400 VH G1m17,1 K147E + K213D GA cah LS |
| 991 | PGDM1400 VH G1m17,1 K147E + K213D IE cah LS |
| 992 | PGDM1400 VH G1m17,1 K147E + K213D cak LS |
| 993 | PGDM1400 VH G1m17,1 K147E + K213D GAALIE cak LS |
| 994 | PGDM1400 VH G1m17,1 K147E + K213D GAIE cak LS |
| 995 | PGDM1400 VH G1m17,1 K147E + K213D GA cak LS |
| 996 | PGDM1400 VH G1m17,1 K147E + K213D IE cak LS |
| | CrossMabCH1-CLv Format - Light Chain |
| 997 | PGDM1400 VL E123K + Q124R |
| 998 | PGDM1400 VL E123R + Q124K |
| | scFv-Fc Format |
| 999 | PGDM1400 VH-(G4S)4-VL G1m3 CH1del C220S |
| 1000 | PGDM1400 VH-(G4S)4-VL G1m3 CH1del C220S LS |
| 1001 | PGDM1400 VH-(G4S)4-VL G1m3 CH1del C220S GAALIE LS |
| 1002 | PGDM1400 VH-(G4S)4-VL G1m3 CH1del C220S GAIE LS |
| 1003 | PGDM1400 VH-(G4S)4-VL G1m3 CH1del C220S GA LS |
| 1004 | PGDM1400 VH-(G4S)4-VL G1m3 CH1del C220S IE LS |
| 1005 | PGDM1400 VH-(G4S)4-VL G1m3 CH1del C220S cah LS |
| 1006 | PGDM1400 VH-(G4S)4-VL G1m3 CH1del C220S GAALIE cah LS |
| 1007 | PGDM1400 VH-(G4S)4-VL G1m3 CH1del C220S GAIE cah LS |
| 1008 | PGDM1400 VH-(G4S)4-VL G1m3 CH1del C220S GA cah LS |
| 1009 | PGDM1400 VH-(G4S)4-VL G1m3 CH1del C220S IE cah LS |
| 1010 | PGDM1400 VH-(G4S)4-VL G1m3 CH1del C220S cak LS |
| 1011 | PGDM1400 VH-(G4S)4-VL G1m3 CH1del C220S GAALIE cak LS |
| 1012 | PGDM1400 VH-(G4S)4-VL G1m3 CH1del C220S GAIE cak LS |
| 1013 | PGDM1400 VH-(G4S)4-VL G1m3 CH1del C220S GA cak LS |
| 1014 | PGDM1400 VH-(G4S)4-VL G1m3 CH1del C220S IE cak LS |
| 1015 | PGDM1400 VH-(G4S)4-VL G1m17 CH1del C220S |
| 1016 | PGDM1400 VH-(G4S)4-VL G1m17 CH1del C220S LS |
| 1017 | PGDM1400 VH-(G4S)4-VL G1m17 CH1del C220S GAALIE LS |
| 1018 | PGDM1400 VH-(G4S)4-VL G1m17 CH1del C220S GAIE LS |
| 1019 | PGDM1400 VH-(G4S)4-VL G1m17 CH1del C220S GA LS |
| 1020 | PGDM1400 VH-(G4S)4-VL G1m17 CH1del C220S IE LS |
| 1021 | PGDM1400 VH-(G4S)4-VL G1m17 CH1del C220S cah LS |
| 1022 | PGDM1400 VH-(G4S)4-VL G1m17 CH1del C220S GAALIE cah LS |
| 1023 | PGDM1400 VH-(G4S)4-VL G1m17 CH1del C220S GAIE cah LS |
| 1024 | PGDM1400 VH-(G4S)4-VL G1m17 CH1del C220S GA cah LS |
| 1025 | PGDM1400 VH-(G4S)4-VL G1m17 CH1del C220S IE cah LS |
| 1026 | PGDM1400 VH-(G4S)4-VL G1m17 CH1del C220S cak LS |
| 1027 | PGDM1400 VH-(G4S)4-VL G1m17 CH1del C220S GAALIE cak LS |
| 1028 | PGDM1400 VH-(G4S)4-VL G1m17 CH1del C220S GAIE cak LS |
| 1029 | PGDM1400 VH-(G4S)4-VL G1m17 CH1del C220S GA cak LS |
| 1030 | PGDM1400 VH-(G4S)4-VL G1m17 CH1del C220S IE cak LS |
| 1031 | PGDM1400 VH-(G4S)4-VL G1m17,1 CH1del C220S |
| 1032 | PGDM1400 VH-(G4S)4-VL G1m17,1 CH1del C220S LS |
| 1033 | PGDM1400 VH-(G4S)4-VL G1m17,1 CH1del C220S GAALIE LS |
| 1034 | PGDM1400 VH-(G4S)4-VL G1m17,1 CH1del C220S GAIE LS |
| 1035 | PGDM1400 VH-(G4S)4-VL G1m17,1 CH1del C220S GA LS |
| 1036 | PGDM1400 VH-(G4S)4-VL G1m17,1 CH1del C220S IE LS |
| 1037 | PGDM1400 VH-(G4S)4-VL G1m17,1 CH1del C220S cah LS |
| 1038 | PGDM1400 VH-(G4S)4-VL G1m17,1 CH1del C220S GAALIE cah LS |
| 1039 | PGDM1400 VH-(G4S)4-VL G1m17,1 CH1del C220S GAIE cah LS |
| 1040 | PGDM1400 VH-(G4S)4-VL G1m17,1 CH1del C220S GA cah LS |
| 1041 | PGDM1400 VH-(G4S)4-VL G1m17,1 CH1del C220S IE cah LS |
| 1042 | PGDM1400 VH-(G4S)4-VL G1m17,1 CH1del C220S cak LS |
| 1043 | PGDM1400 VH-(G4S)4-VL G1m17,1 CH1del C220S GAALIE cak LS |
| 1044 | PGDM1400 VH-(G4S)4-VL G1m17,1 CH1del C220S GAIE cak LS |
| 1045 | PGDM1400 VH-(G4S)4-VL G1m17,1 CH1del C220S GA cak LS |
| 1046 | PGDM1400 VH-(G4S)4-VL G1m17,1 CH1del C220S IE cak LS |
| 1047 | PGDM1400 VL-(G4S)4-VH G1m3 CH1del C220S |
| 1048 | PGDM1400 VL-(G4S)4-VH G1m3 CH1del C220S LS |
| 1049 | PGDM1400 VL-(G4S)4-VH G1m3 CH1del C220S GAALIE LS |
| 1050 | PGDM1400 VL-(G4S)4-VH G1m3 CH1del C220S GAIE LS |
| 1051 | PGDM1400 VL-(G4S)4-VH G1m3 CH1del C220S GA LS |
| 1052 | PGDM1400 VL-(G4S)4-VH G1m3 CH1del C220S IE LS |
| 1053 | PGDM1400 VL-(G4S)4-VH G1m3 CH1del C220S cah LS |
| 1054 | PGDM1400 VL-(G4S)4-VH G1m3 CH1del C220S GAALIE cah LS |
| 1055 | PGDM1400 VL-(G4S)4-VH G1m3 CH1del C220S GAIE cah LS |
| 1056 | PGDM1400 VL-(G4S)4-VH G1m3 CH1del C220S GA cah LS |
| 1057 | PGDM1400 VL-(G4S)4-VH G1m3 CH1del C220S IE cah LS |
| 1058 | PGDM1400 VL-(G4S)4-VH G1m3 CH1del C220S cak LS |
| 1059 | PGDM1400 VL-(G4S)4-VH G1m3 CH1del C220S GAALIE cak LS |
| 1060 | PGDM1400 VL-(G4S)4-VH G1m3 CH1del C220S GAIE cak LS |
| 1061 | PGDM1400 VL-(G4S)4-VH G1m3 CH1del C220S GA cak LS |
| 1062 | PGDM1400 VL-(G4S)4-VH G1m3 CH1del C220S IE cak LS |
| 1063 | PGDM1400 VL-(G4S)4-VH G1m17 CH1del C220S |
| 1064 | PGDM1400 VL-(G4S)4-VH G1m17 CH1del C220S LS |
| 1065 | PGDM1400 VL-(G4S)4-VH G1m17 CH1del C220S GAALIE LS |
| 1066 | PGDM1400 VL-(G4S)4-VH G1m17 CH1del C220S GAIE LS |
| 1067 | PGDM1400 VL-(G4S)4-VH G1m17 CH1del C220S GA LS |
| 1068 | PGDM1400 VL-(G4S)4-VH G1m17 CH1del C220S IE LS |
| 1069 | PGDM1400 VL-(G4S)4-VH G1m17 CH1del C220S cah LS |
| 1070 | PGDM1400 VL-(G4S)4-VH G1m17 CH1del C220S GAALIE cah LS |
| 1071 | PGDM1400 VL-(G4S)4-VH G1m17 CH1del C220S GAIE cah LS |
| 1072 | PGDM1400 VL-(G4S)4-VH G1m17 CH1del C220S GA cah LS |
| 1073 | PGDM1400 VL-(G4S)4-VH G1m17 CH1del C220S IE cah LS |
| 1074 | PGDM1400 VL-(G4S)4-VH G1m17 CH1del C220S cak LS |
| 1075 | PGDM1400 VL-(G4S)4-VH G1m17 CH1del C220S GAALIE cak LS |
| 1076 | PGDM1400 VL-(G4S)4-VH G1m17 CH1del C220S GAIE cak LS |
| 1077 | PGDM1400 VL-(G4S)4-VH G1m17 CH1del C220S GA cak LS |
| 1078 | PGDM1400 VL-(G4S)4-VH G1m17 CH1del C220S IE cak LS |
| 1079 | PGDM1400 VL-(G4S)4-VH G1m17,1 CH1del C220S |
| 1080 | PGDM1400 VL-(G4S)4-VH G1m17,1 CH1del C220S LS |
| 1081 | PGDM1400 VL-(G4S)4-VH G1m17,1 CH1del C220S GAALIE LS |
| 1082 | PGDM1400 VL-(G4S)4-VH G1m17,1 CH1del C220S GAIE LS |
| 1083 | PGDM1400 VL-(G4S)4-VH G1m17,1 CH1del C220S GA LS |
| 1084 | PGDM1400 VL-(G4S)4-VH G1m17,1 CH1del C220S IE LS |
| 1085 | PGDM1400 VL-(G4S)4-VH G1m17,1 CH1del C220S cah LS |
| 1086 | PGDM1400 VL-(G4S)4-VH G1m17,1 CH1del C220S GAALIE cah LS |
| 1087 | PGDM1400 VL-(G4S)4-VH G1m17,1 CH1del C220S GAIE cah LS |
| 1088 | PGDM1400 VL-(G4S)4-VH G1m17,1 CH1del C220S GA cah LS |
| 1089 | PGDM1400 VL-(G4S)4-VH G1m17,1 CH1del C220S IE cah LS |
| 1090 | PGDM1400 VL-(G4S)4-VH G1m17,1 CH1del C220S cak LS |
| 1091 | PGDM1400 VL-(G4S)4-VH G1m17,1 CH1del C220S GAALIE cak LS |
| 1092 | PGDM1400 VL-(G4S)4-VH G1m17,1 CH1del C220S GAIE cak LS |
| 1093 | PGDM1400 VL-(G4S)4-VH G1m17,1 CH1del C220S GA cak LS |
| 1094 | PGDM1400 VL-(G4S)4-VH G1m17,1 CH1del C220S IE cak LS |
| 1095 | PGDM1400 VH-(G4S)5-VL G1m3 CH1del C220S |
| 1096 | PGDM1400 VH-(G4S)5-VL G1m3 CH1del C220S LS |
| 1097 | PGDM1400 VH-(G4S)5-VL G1m3 CH1del C220S GAALIE LS |
| 1098 | PGDM1400 VH-(G4S)5-VL G1m3 CH1del C220S GAIE LS |
| 1099 | PGDM1400 VH-(G4S)5-VL G1m3 CH1del C220S GA LS |
| 1100 | PGDM1400 VH-(G4S)5-VL G1m3 CH1del C220S IE LS |
| 1101 | PGDM1400 VH-(G4S)5-VL G1m3 CH1del C220S cah LS |
| 1102 | PGDM1400 VH-(G4S)5-VL G1m3 CH1del C220S GAALIE cah LS |
| 1103 | PGDM1400 VH-(G4S)5-VL G1m3 CH1del C220S GAIE cah LS |
| 1104 | PGDM1400 VH-(G4S)5-VL G1m3 CH1del C220S GA cah LS |
| 1105 | PGDM1400 VH-(G4S)5-VL G1m3 CH1del C220S IE cah LS |
| 1106 | PGDM1400 VH-(G4S)5-VL G1m3 CH1del C220S cak LS |
| 1107 | PGDM1400 VH-(G4S)5-VL G1m3 CH1del C220S GAALIE cak LS |
| 1108 | PGDM1400 VH-(G4S)5-VL G1m3 CH1del C220S GAIE cak LS |
| 1109 | PGDM1400 VH-(G4S)5-VL G1m3 CH1del C220S GA cak LS |
| 1110 | PGDM1400 VH-(G4S)5-VL G1m3 CH1del C220S IE cak LS |
| 1111 | PGDM1400 VH-(G4S)5-VL G1m17 CH1del C220S |
| 1112 | PGDM1400 VH-(G4S)5-VL G1m17 CH1del C220S LS |
| 1113 | PGDM1400 VH-(G4S)5-VL G1m17 CH1del C220S GAALIE LS |
| 1114 | PGDM1400 VH-(G4S)5-VL G1m17 CH1del C220S GAIE LS |
| 1115 | PGDM1400 VH-(G4S)5-VL G1m17 CH1del C220S GA LS |
| 1116 | PGDM1400 VH-(G4S)5-VL G1m17 CH1del C220S IE LS |
| 1117 | PGDM1400 VH-(G4S)5-VL G1m17 CH1del C220S cah LS |
| 1118 | PGDM1400 VH-(G4S)5-VL G1m17 CH1del C220S GAALIE cah LS |
| 1119 | PGDM1400 VH-(G4S)5-VL G1m17 CH1del C220S GAIE cah LS |
| 1120 | PGDM1400 VH-(G4S)5-VL G1m17 CH1del C220S GA cah LS |
| 1121 | PGDM1400 VH-(G4S)5-VL G1m17 CH1del C220S IE cah LS |
| 1122 | PGDM1400 VH-(G4S)5-VL G1m17 CH1del C220S cak LS |
| 1123 | PGDM1400 VH-(G4S)5-VL G1m17 CH1del C220S GAALIE cak LS |
| 1124 | PGDM1400 VH-(G4S)5-VL G1m17 CH1del C220S GAIE cak LS |
| 1125 | PGDM1400 VH-(G4S)5-VL G1m17 CH1del C220S GA cak LS |
| 1126 | PGDM1400 VH-(G4S)5-VL G1m17 CH1del C220S IE cak LS |
| 1127 | PGDM1400 VH-(G4S)5-VL G1m17,1 CH1del C220S |
| 1128 | PGDM1400 VH-(G4S)5-VL G1m17,1 CH1del C220S LS |
| 1129 | PGDM1400 VH-(G4S)5-VL G1m17,1 CH1del C220S GAALIE LS |
| 1130 | PGDM1400 VH-(G4S)5-VL G1m17,1 CH1del C220S GAIE LS |
| 1131 | PGDM1400 VH-(G4S)5-VL G1m17,1 CH1del C220S GA LS |
| 1132 | PGDM1400 VH-(G4S)5-VL G1m17,1 CH1del C220S IE LS |
| 1133 | PGDM1400 VH-(G4S)5-VL G1m17,1 CH1del C220S cah LS |
| 1134 | PGDM1400 VH-(G4S)5-VL G1m17,1 CH1del C220S GAALIE cah LS |
| 1135 | PGDM1400 VH-(G4S)5-VL G1m17,1 CH1del C220S GAIE cah LS |
| 1136 | PGDM1400 VH-(G4S)5-VL G1m17,1 CH1del C220S GA cah LS |
| 1137 | PGDM1400 VH-(G4S)5-VL G1m17,1 CH1del C220S IE cah LS |
| 1138 | PGDM1400 VH-(G4S)5-VL G1m17,1 CH1del C220S cak LS |
| 1139 | PGDM1400 VH-(G4S)5-VL G1m17,1 CH1del C220S GAALIE cak LS |
| 1140 | PGDM1400 VH-(G4S)5-VL G1m17,1 CH1del C220S GAIE cak LS |
| 1141 | PGDM1400 VH-(G4S)5-VL G1m17,1 CH1del C220S GA cak LS |
| 1142 | PGDM1400 VH-(G4S)5-VL G1m17,1 CH1del C220S IE cak LS |
| 1143 | PGDM1400 VL-(G4S)5-VH G1m3 CH1del C220S |
| 1144 | PGDM1400 VL-(G4S)5-VH G1m3 CH1del C220S LS |
| 1145 | PGDM1400 VL-(G4S)5-VH G1m3 CH1del C220S GAALIE LS |
| 1146 | PGDM1400 VL-(G4S)5-VH G1m3 CH1del C220S GAIE LS |
| 1147 | PGDM1400 VL-(G4S)5-VH G1m3 CH1del C220S GA LS |
| 1148 | PGDM1400 VL-(G4S)5-VH G1m3 CH1del C220S IE LS |
| 1149 | PGDM1400 VL-(G4S)5-VH G1m3 CH1del C220S cah LS |
| 1150 | PGDM1400 VL-(G4S)5-VH G1m3 CH1del C220S GAALIE cah LS |
| 1151 | PGDM1400 VL-(G4S)5-VH G1m3 CH1del C220S GAIE cah LS |
| 1152 | PGDM1400 VL-(G4S)5-VH G1m3 CH1del C220S GA cah LS |
| 1153 | PGDM1400 VL-(G4S)5-VH G1m3 CH1del C220S IE cah LS |
| 1154 | PGDM1400 VL-(G4S)5-VH G1m3 CH1del C220S cak LS |
| 1155 | PGDM1400 VL-(G4S)5-VH G1m3 CH1del C220S GAALIE cak LS |
| 1156 | PGDM1400 VL-(G4S)5-VH G1m3 CH1del C220S GAIE cak LS |
| 1157 | PGDM1400 VL-(G4S)5-VH G1m3 CH1del C220S GA cak LS |
| 1158 | PGDM1400 VL-(G4S)5-VH G1m3 CH1del C220S IE cak LS |
| 1159 | PGDM1400 VL-(G4S)5-VH G1m17 CH1del C220S |
| 1160 | PGDM1400 VL-(G4S)5-VH G1m17 CH1del C220S LS |
| 1161 | PGDM1400 VL-(G4S)5-VH G1m17 CH1del C220S GAALIE LS |
| 1162 | PGDM1400 VL-(G4S)5-VH G1m17 CH1del C220S GAIE LS |
| 1163 | PGDM1400 VL-(G4S)5-VH G1m17 CH1del C220S GA LS |
| 1164 | PGDM1400 VL-(G4S)5-VH G1m17 CH1del C220S IE LS |
| 1165 | PGDM1400 VL-(G4S)5-VH G1m17 CH1del C220S cah LS |
| 1166 | PGDM1400 VL-(G4S)5-VH G1m17 CH1del C220S GAALIE cah LS |
| 1167 | PGDM1400 VL-(G4S)5-VH G1m17 CH1del C220S GAIE cah LS |
| 1168 | PGDM1400 VL-(G4S)5-VH G1m17 CH1del C220S GA cah LS |
| 1169 | PGDM1400 VL-(G4S)5-VH G1m17 CH1del C220S IE cah LS |
| 1170 | PGDM1400 VL-(G4S)5-VH G1m17 CH1del C220S cak LS |
| 1171 | PGDM1400 VL-(G4S)5-VH G1m17 CH1del C220S GAALIE cak LS |
| 1172 | PGDM1400 VL-(G4S)5-VH G1m17 CH1del C220S GAIE cak LS |
| 1173 | PGDM1400 VL-(G4S)5-VH G1m17 CH1del C220S GA cak LS |
| 1174 | PGDM1400 VL-(G4S)5-VH G1m17 CH1del C220S IE cak LS |
| 1175 | PGDM1400 VL-(G4S)5-VH G1m17,1 CH1del C220S |
| 1176 | PGDM1400 VL-(G4S)5-VH G1m17,1 CH1del C220S LS |
| 1177 | PGDM1400 VL-(G4S)5-VH G1m17,1 CH1del C220S GAALIE LS |
| 1178 | PGDM1400 VL-(G4S)5-VH G1m17,1 CH1del C220S GAIE LS |
| 1179 | PGDM1400 VL-(G4S)5-VH G1m17,1 CH1del C220S GA LS |
| 1180 | PGDM1400 VL-(G4S)5-VH G1m17,1 CH1del C220S IE LS |
| 1181 | PGDM1400 VL-(G4S)5-VH G1m17,1 CH1del C220S cah LS |
| 1182 | PGDM1400 VL-(G4S)5-VH G1m17,1 CH1del C220S GAALIE cah LS |
| 1183 | PGDM1400 VL-(G4S)5-VH G1m17,1 CH1del C220S GAIE cah LS |
| 1184 | PGDM1400 VL-(G4S)5-VH G1m17,1 CH1del C220S GA cah LS |
| 1185 | PGDM1400 VL-(G4S)5-VH G1m17,1 CH1del C220S IE cah LS |
| 1186 | PGDM1400 VL-(G4S)5-VH G1m17,1 CH1del C220S cak LS |
| 1187 | PGDM1400 VL-(G4S)5-VH G1m17,1 CH1del C220S GAALIE cak LS |
| 1188 | PGDM1400 VL-(G4S)5-VH G1m17,1 CH1del C220S GAIE cak LS |
| 1189 | PGDM1400 VL-(G4S)5-VH G1m17,1 CH1del C220S GA cak LS |
| 1190 | PGDM1400 VL-(G4S)5-VH G1m17,1 CH1del C220S IE cak LS |

### B. RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or portion thereof. An saRNA has multiple ORFs.

In some embodiments, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

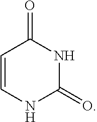

The term “uridine,” as used herein, describes one of the nucleosides that can occur in RNA. The structure of uridine is:

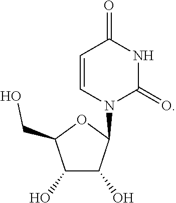 [SMILES: O=c1ccn([C@@H]2O[C@H](CO)[C@@H](O)[C@H]2O)c(=O)[nH]1]

UTP (uridine 5′-triphosphate) has the following structure:

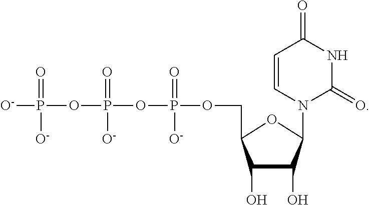

Pseudo-UTP (pseudouridine 5′-triphosphate) has the following structure:

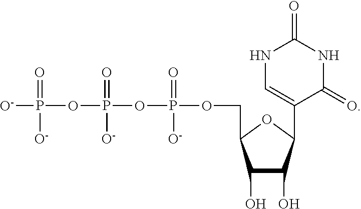

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

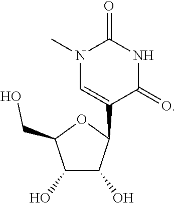 [SMILES: Cn1cc([C@@H]2O[C@H](CO)[C@@H](O)[C@H]2O)c(=O)[nH]c1=O]

N1-methyl-pseudo-UTP has the following structure:

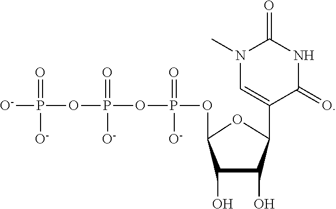

Another exemplary modified nucleoside is 5-methyl-uridine (m5U), which has the structure:

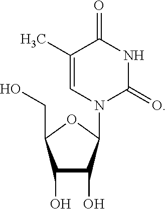

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine), 5-methyl-2-thio-uridine (m5s2U), 1-methyl-4-thio-pseudouridine (m1s4y), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 Lp), 5-(isopentenylaminomethyl)uridine (inm5U), 5-(isopentenylaminomethyl)-2-thio-uridine (inm5s2U), α-thio-uridine, 2′-O-methyl-uridine (Urn), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e., at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a protein of interest, e.g., an antibody agent. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### C. Codon Optimization and GC Enrichment

As used herein, the term “codon-optimized” refers to alteration of codons in a coding region of a nucleic acid molecule (e.g., a polyribonucleotide) to reflect the typical codon usage of a host organism (e.g., a subject receiving a nucleic acid molecule (e.g., a polyribonucleotide)) without preferably altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a polyribonucleotide) may differ from wild type sequences encoding the relevant antigen or fragment or epitope thereof, even when the amino acid sequence of the antigen or fragment or epitope thereof is wild type.

In some embodiments, strategies for codon optimization for expression in a relevant subject (e.g., a human), and even, in some cases, for expression in a particular cell or tissue.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/ and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide) of the present disclosure is codon optimized, wherein the codons in the polynucleotide (e.g., the polyribonucleotide) are adapted to human codon usage (herein referred to as “human codon optimized polynucleotide”). Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 9. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 9). Accordingly, in some embodiments, such a procedure (as exemplified for Ala) is applied for each amino acid encoded by the coding sequence of a polynucleotide to obtain sequences adapted to human codon usage.

TABLE 9
Human codon usage table with frequencies
indicated for each amino acid.

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Tor | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0 42 |
| Lou | CTC | 0.20 | Tyr | TAC* | 0.58 |
| Met | ATG* | 1 | Stop | TGA* | 0 61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an mRNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); mRNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLoS ONE 6(3): e17596; Rabb et al., “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content of which increased compared to a wild type coding sequence for a relevant antibody agent directed against an HIV (e.g., HIV-1) polypeptide, or fragment or epitope thereof (e.g., an epitope within the Env trimer apex). In some embodiments, guanosine/cytidine (G/C) content of a coding region is modified relative to a wild type coding sequence for a relevant antibody agent directed against an HIV (e.g., HIV-1) polypeptide, or fragment or epitope thereof (e.g., an epitope within the Env trimer apex), but the amino acid sequence encoded by the polyribonucleotide not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a polyribonucleotide, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a polyribonucleotide described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a polyribonucleotide described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of a polyribonucleotide may incorporate one or more elements established to contribute to stability and/or translation efficiency of the polyribonucleotide; exemplary such elements are described, for example, in PCT/EP2006/009448 incorporated herein by reference. In some embodiments, to increase expression of a polyribonucleotide used according to the present disclosure, a polyribonucleotide may be modified within the coding region, i.e., the sequence encoding the expressed peptide or protein, without altering the sequence of the expressed peptide or protein, for example so as to increase the GC-content to increase mRNA stability and/or to perform a codon optimization and, thus, enhance translation in cells.

### IV. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid-based vectors, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells (e.g., liver cells). In some embodiments, such lipid nanoparticles may be particularly useful to deliver polyribonucleotides when polyribonucleotides are intravenously or intramuscularly administered to a subject.

### A. Particles for Delivery of at Least One Polyribonucleotide

Polyribonucleotides provided herein can be delivered by particles. In the context of the present disclosure, the term “particle” relates to a structured entity formed by molecules or molecule complexes. In some embodiments, the term “particle” relates to a micro- or nano-sized structure, such as a micro- or nano-sized compact structure dispersed in a medium. In some embodiments, a particle is a nucleic acid containing particle such as a particle comprising a polyribonucleotide.

Electrostatic interactions between positively charged molecules such as polymers and lipids and negatively charged nucleic acid (e.g., a polyribonucleotide) are involved in particle formation. This results in complexation and spontaneous formation of nucleic acid particles (e.g., ribonucleic acid particles). In some embodiments, a nucleic acid particle (e.g., ribonucleic acid particle) is a nanoparticle.

A “nucleic acid particle” (e.g., a ribonucleic acid particle) are particles that encompass or contain a nucleic acid, and are used to deliver nucleic acid (e.g., a polyribonucleotide) to a target site of interest (e.g., cell, tissue, organ, and the like). A nucleic acid particle (e.g., a ribonucleic acid particle) may be formed from (i) at least one cationic or cationically ionizable lipid or lipid-like material, (ii) at least one cationic polymer such as protamine, or a mixture of (i) and (ii), and (iii) nucleic acid (e.g., a polyribonucleotide). Nucleic acid particles (e.g., a ribonucleic acid particle) include lipid nanoparticles (lipid nanoparticle) and lipoplexes (LPX).

In some embodiments, nucleic acid particles (e.g., ribonucleic acid particles) comprise more than one type of nucleic acid molecules (e.g., polyribonucleotides), where the molecular parameters of the nucleic acid molecules may be similar or different from each other, like with respect to molar mass or fundamental structural elements such as molecular architecture, capping, coding regions or other features.

In some embodiments, provided nucleic acid particles (e.g., ribonucleic acid particles) can comprise lipid nanoparticles. As used in the present disclosure, “nanoparticle” refers to a particle having an average diameter suitable for parenteral administration. In various embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

Nucleic acid particles (e.g., ribonucleic acid particles) described herein may exhibit a polydispersity index less than about 0.5, less than about 0.4, less than about 0.3, or about 0.2 or less. By way of example, the nucleic acid particles (e.g., ribonucleic acid particles) can exhibit a polydispersity index in a range of about 0.1 to about 0.3 or about 0.2 to about 0.3.

Nucleic acid particles (e.g., ribonucleic acid particles) described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a nucleic acid particle (e.g., a ribonucleic acid particle) described herein has an N/P ratio greater than or equal to 5. In some embodiments, a nucleic acid particle (e.g., a ribonucleic acid particle) described herein has an N/P ratio that is about 5, 6, 7, 8, 9, or 10. In some embodiments, an N/P ratio for a nucleic acid particle (e.g., a ribonucleic acid particle) described herein is from about 10 to about 50. In some embodiments, an N/P ratio for a nucleic acid particle (e.g., a ribonucleic acid particle) described herein is from about 10 to about 70. In some embodiments, an N/P ratio for a nucleic acid particle (e.g., a ribonucleic acid particle) described herein is from about 10 to about 120.

Nucleic acid particles (e.g., ribonucleic acid particles) described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture can be microscopic, with particle sizes between 1 and 1000 nanometers. The mixture may be termed a colloid or a colloidal suspension. Sometimes the term “colloid” only refers to the particles in the mixture and not the entire suspension.

The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Different types of nucleic acid particles have been described previously to be suitable for delivery of nucleic acid in particulate form (e.g., Kaczmarek, J. C. et al., 2017, Genome Medicine 9, 60, which is herein incorporated by reference). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid (e.g., a polyribonucleotide), at least one cationic or cationically ionizable lipid or lipid-like material, and/or at least one cationic polymer which associate with the nucleic acid (e.g., a polyribonucleotide) to form nucleic acid particles (e.g., ribonucleic acid particles, e.g., ribonucleic acid nanoparticles) and compositions comprising such particles. The nucleic acid particles (e.g., ribonucleic acid particles, e.g., ribonucleic acid nanoparticles) may comprise nucleic acid (e.g., a polyribonucleotide) which is complexed in different forms by non-covalent interactions to the particle. The particles described herein are not viral particles, in particular, they are not infectious viral particles, i.e., they are not able to virally infect cells.

Some embodiments described herein relate to compositions, methods and uses involving more than one, e.g., 2, 3, 4, 5, 6 or even more nucleic acid species (e.g., polyribonucleotide species).

In a nucleic acid particle (e.g., ribonucleic acid particle, e.g., ribonucleic acid nanoparticle) formulation, it is possible that each nucleic acid species (e.g., polyribonucleotide species) is separately formulated as an individual nucleic acid particle (e.g., ribonucleic acid particle, e.g., ribonucleic acid nanoparticle) formulation. In that case, each individual nucleic acid particle (e.g., ribonucleic acid particle, e.g., ribonucleic acid nanoparticle) formulation will comprise one nucleic acid species (e.g., polyribonucleotide species). The individual nucleic acid particle (e.g., ribonucleic acid particle, e.g., ribonucleic acid nanoparticle) formulations may be present as separate entities, e.g., in separate containers. Such formulations are obtainable by providing each nucleic acid species (e.g., polyribonucleotide species) separately (typically each in the form of a nucleic acid-containing solution) together with a particle-forming agent, thereby allowing the formation of particles. Respective particles will contain exclusively the specific nucleic acid species (e.g., polyribonucleotide species) that is being provided when the particles are formed (individual particulate formulations).

In some embodiments, a composition such as a pharmaceutical composition comprises more than one individual nucleic acid particle (e.g., ribonucleic acid particle, e.g., ribonucleic acid nanoparticle) formulation. Respective pharmaceutical compositions are referred to as “mixed particulate formulations.” Mixed particulate formulations according to the invention are obtainable by forming, separately, individual nucleic acid particle (e.g., ribonucleic acid particle, e.g., ribonucleic acid nanoparticle) formulations, as described above, followed by a step of mixing of the individual nucleic acid particle (e.g., ribonucleic acid particle, e.g., ribonucleic acid nanoparticle) formulations. By the step of mixing, a formulation comprising a mixed population of nucleic acid-containing particles is obtainable. Individual nucleic acid particle (e.g., ribonucleic acid particle, e.g., ribonucleic acid nanoparticle) populations may be together in one container, comprising a mixed population of individual nucleic acid particle (e.g., ribonucleic acid particle, e.g., ribonucleic acid nanoparticle) formulations.

Alternatively, it is possible that different nucleic acid species (e.g., polyribonucleotide species) are formulated together as a “combined particulate formulation.” Such formulations are obtainable by providing a combined formulation (typically combined solution) of different nucleic acid species (e.g., polyribonucleotide species) species together with a particle-forming agent, thereby allowing the formation of particles. As opposed to a “mixed particulate formulation,” a “combined particulate formulation” will typically comprise particles that comprise more than one nucleic acid species (e.g., polyribonucleotide species) species. In a combined particulate composition different nucleic acid species (e.g., polyribonucleotide species) are typically present together in a single particle.

In certain embodiments, nucleic acids (e.g., polyribonucleotides), when present in provided nucleic acid particles (e.g., ribonucleic acid particles, e.g., lipid nanoparticles) are resistant in aqueous solution to degradation with a nuclease.

In some embodiments, nucleic acid particles (e.g., ribonucleic acid particles) are lipid nanoparticles. In some embodiments, lipid nanoparticles are liver-targeting lipid nanoparticles. In some embodiments, lipid nanoparticles are cationic lipid nanoparticles comprising one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### 1. Cationic Polymeric Materials

Cationic polymers have been recognized as useful for developing such delivery vehicles, as reported in PCT App. Pub. No. WO 2021/001417, the entirety of which is incorporated herein by reference. As used herein, the term “polymer” refers to a composition comprising one or more molecules that comprise repeating units of one or more monomers. As used herein, “polymer,” “polymeric material,” and “polymer composition” are used interchangeably, and unless otherwise specified, refer to a composition of polymer molecules. A person of skill in the art will appreciate that a polymer composition comprises polymer molecules having molecules of different lengths (e.g., comprising varying amounts of monomers). Polymer compositions described herein are characterized by one or more of a normalized molecular weight (Mn), a weight average molecular weight (Mw), and/or a polydispersity index (PDI). In some embodiments, such repeat units can all be identical (a “homopolymer”); alternatively, in some cases, there can be more than one type of repeat unit present within the polymeric material (a “heteropolymer” or a “copolymer”). In some cases, a polymer is biologically derived, e.g., a biopolymer such as a protein. In some cases, additional moieties can also be present in the polymeric material, for example targeting moieties such as those described herein.

In some embodiments, a polymer utilized in accordance with the present disclosure may be a copolymer. Repeat units forming the copolymer can be arranged in any fashion. For example, in some embodiments, repeat units can be arranged in a random order; alternatively or additionally, in some embodiments, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, a polymeric material for use in accordance with the present disclosure is biocompatible. In certain embodiments, a biocompatible material is biodegradable, e.g., is able to degrade, chemically and/or biologically, within a physiological environment, such as within the body.

In certain embodiments, a polymeric material may be or comprise protamine or polyalkyleneimine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some embodiments, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some embodiments, a polyalkyleneimine comprises polyethylenimine (PEI) and/or polypropylenimine. In some embodiments, a preferred polyalkyleneimine is polyethyleneimine (PEI). In some embodiments, the average molecular weight of PEI is preferably 0.75.102 to 107 Da, preferably 1000 to 105 Da, more preferably 10000 to 40000 Da, more preferably 15000 to 30000 Da, even more preferably 20000 to 25000 Da.

Cationic materials (e.g., polymeric materials, including polycationic polymers) contemplated for use herein include those which are able to electrostatically bind nucleic acid. In some embodiments, cationic polymeric materials contemplated for use herein include any cationic polymeric materials with which nucleic acid can be associated, e.g., by forming complexes with the nucleic acid or forming vesicles in which the nucleic acid is enclosed or encapsulated.

In some embodiments, particles described herein may comprise polymers other than cationic polymers, e.g., non-cationic polymeric materials and/or anionic polymeric materials. Collectively, anionic and neutral polymeric materials are referred to herein as non-cationic polymeric materials.

### 2. Lipid Particles

The terms “lipid” and “lipid-like material” are used herein to refer to molecules that comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). In some embodiments, hydrophilic groups may comprise polar and/or charged groups and include carbohydrates, phosphate, carboxylic, sulfate, amino, sulfhydryl, nitro, hydroxyl, and other like groups.

Lipid nanoparticles (also referred to as “lipid nanoparticles”) of the present disclosure comprise (i) a cationic lipid; (ii) a polymer-conjugated lipid, and (iii) one or more helper lipids. Lipid nanoparticles described herein are useful for the delivery of nucleic acid cargo (e.g., a polyribonucleotide) into the cell of a subject. In some embodiments, lipid nanoparticles comprising a nucleic acid (e.g., a polyribonucleotide) described herein are useful for causing increased expression of a protein (e.g., an antibody agent) in a subject. In some embodiments, lipid nanoparticles comprising a nucleic acid (e.g., a polyribonucleotide) described herein are useful for causing a pharmacological effect induced by expression of a protein in a subject. Lipid nanoparticles described herein are characterized by molar percentage (mol %) of components in the lipid nanoparticle. A mol % used in reference to a lipid component of a lipid nanoparticle is relative to the total other lipid components in the lipid nanoparticle.

a. Cationic lipids

As described herein, lipid nanoparticles of the present disclosure comprise a cationic lipid. In some embodiments, a lipid nanoparticle for delivery of at least one polyribonucleotide described herein comprises a cationic lipid. A cationic lipid, as described herein, is a lipid that is positively charged or is ionizable, such that the cationic lipid will become positively charged when subjected to particular physiological conditions, e.g., a pH of about 7.4 or less, and can promote lipid aggregation. In some embodiments, a cationic lipid is a lipid comprising one or more amine groups which bear or are capable of bearing a positive charge.

In some embodiments, a cationic lipid may comprise a cationic, meaning positively charged, headgroup. In some embodiments, a cationic lipid may have a hydrophobic domain (e.g., one or more domains of a neutral lipid or an anionic lipid) provided that the cationic lipid has a net positive charge. In some embodiments, a cationic lipid comprises a polar headgroup, which in some embodiments may comprise one or more amine derivatives such as primary, secondary, and/or tertiary amines, quaternary ammonium, various combinations of amines, amidinium salts, or guanidine and/or imidazole groups as well as pyridinium, piperizine and amino acid headgroups such as lysine, arginine, ornithine and/or tryptophan. In some embodiments, a polar headgroup of a cationic lipid comprises one or more amine derivatives. In some embodiments, a polar headgroup of a cationic lipid comprises a quaternary ammonium. In some embodiments, a headgroup of a cationic lipid may comprise multiple cationic charges. In some embodiments, a headgroup of a cationic lipid comprises one cationic charge.

In some embodiments, a cationic lipid is selected from 1,2-dimyristoyl-sn-glycero-3-ethylphosphocholine (DMEPC); 2-dimyristoyl-3-trimethylammonium propane (DMTAP); dioleyl ether phosphatidylcholine (DOEPC); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

In some embodiments, a cationic lipid is one provided in WO2012/016184, which is incorporated herein by reference in its entirety. For example, in some embodiments, a cationic lipid is selected from 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some embodiments, a cationic lipid is one provided in WO2020/219941, WO2017/075531, WO2016/176330, WO2017/049245, or U.S. Pat. No. 9,670,152, each of which is incorporated herein by reference in its entirety.

In some embodiments, a cationic lipid is a compound of Formula I:

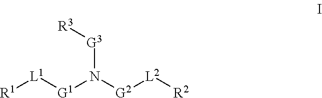

or a pharmaceutically acceptable salt thereof, wherein:
one of L¹ or L² is —OC(O)—, —C(O)O—, —C(O)—, —O—, —S(O)ₓ—, —S—S—, —C(O)S—, SC(O)—, —NR^{a}C(O)—, —C(O)NR¹—, —NR^{a}C(O)NR^{a}—, —OC(O)NR^{a}— or —NR^{a}C(O)O—, and the other of L¹ or L² is —OC(O)—, —C(O)O—, —C(O)—, —O—, —S(O)ₓ—, —S—S—, —C(O)S—, SC(O)—, —NR^{a}C(O)—, —C(O)NR^{a}—, —NR^{a}C(O)NR^{a}—, —OC(O)NR^{a}—, —NR^{a}C(O)O—, or a direct bond;
   G¹ and G² are each independently unsubstituted C₁-C₁₂ alkylene or C₁-C₁₂ alkenylene;
   G³ is C₁-C₂₄ alkylene, C₁-C₂₄ alkenylene, C₃-C₈ cycloalkylene, C₃-C₈ cycloalkenylene;
   Ra is H or C₁-C₁₂ alkyl;
   R¹ and R² are each independently C₆-C₂₄ alkyl or C₆-C₂₄ alkenyl;
   R³ is H, OR⁵, CN, —C(O)OR⁴, —OC(O)R⁴ or —R⁵C(O)R⁴;
   R⁴ is C₁-C₁₂ alkyl;
   R⁵ is H or C₁-C₆ alkyl; and
   x is 0, 1 or 2.

In some embodiments, one of L¹ or L² is —OC(O)— or —C(O)O—. In some embodiments, each of L¹ and L² is —OC(O)— or —C(O)O—.

In some embodiments, G¹ is C₁-C₁₂ alkylene. In some embodiments, G² is C₁-C₁₂ alkylene. In some embodiments G¹ and G² are each independently C₁-C₁₂ alkylene. In some embodiments G¹ and G² are each independently C₅-C₁₂ alkylene.

In some embodiments, G³ is C₁-C₂₄ alkylene. In some embodiments, G³ is C₁-C₆ alkylene.

In some embodiments, R¹ and R² are each independently selected from:

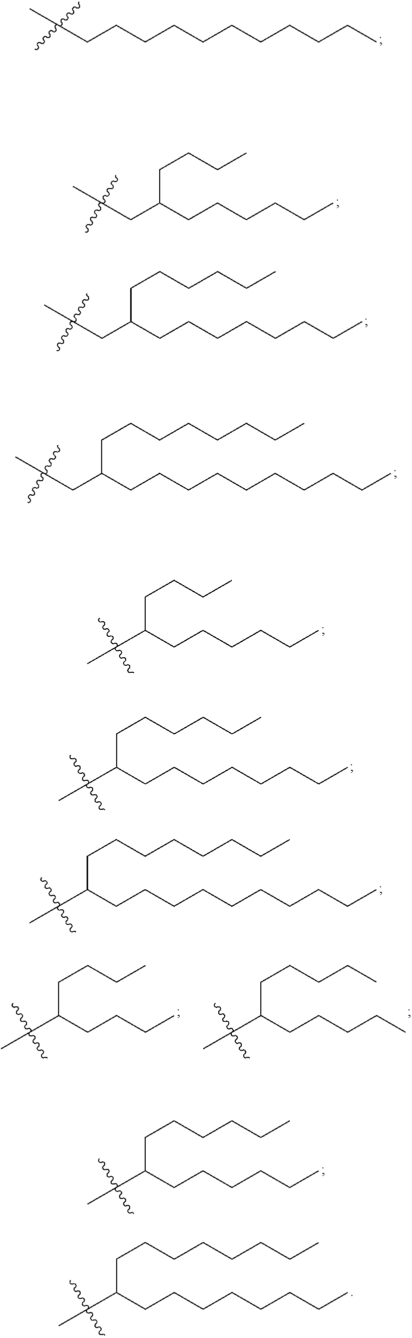

In some embodiments, R³ is OH.

In some embodiments, each of L¹ and L² is —OC(O)—, G¹ and G² are each independently C₅-C₁₂ alkylene, G³ is C₁-C₆ alkylene, R³ is OH, and R¹ and R² are each independently selected from:

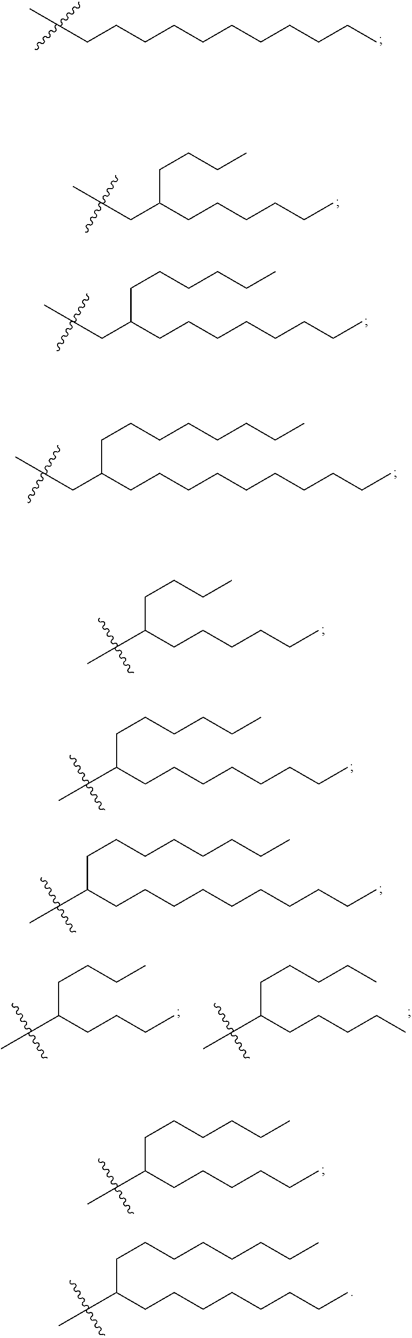

In some embodiments, a cationic lipid is a compound of Formula Ia or Ib

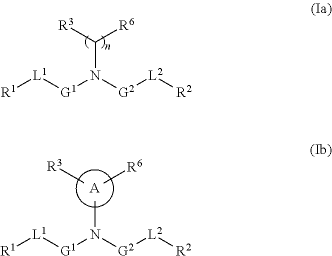

or a pharmaceutically acceptable salt thereof, where n is an integer from 1 to 15, A is C₃-C₈ cycloaliphatic, each R6 is independently selected from H, OH, and C1-C24 aliphatic, and wherein R1, R2, R3, L1, L2, G1, and G2 are as described in classes and subclasses herein, both singly and in combination.

In some embodiments, a positively charged lipid structure described herein may also include one or more other components that may be typically used in the formation of vesicles (e.g. for stabilization). Examples of such other components includes, without being limited thereto, fatty alcohols, fatty acids, and/or cholesterol esters or any other pharmaceutically acceptable excipients which may affect the surface charge, the membrane fluidity and assist in the incorporation of the lipid into the lipid assembly. Examples of sterols include cholesterol, cholesteryl hemisuccinate, cholesteryl sulfate, or any other derivatives of cholesterol. Preferably, the at least one cationic lipid comprises DMEPC and/or DOTMA.

In some embodiments, a cationic lipid is ionizable such that it can exist in a positively charged form or neutral form depending on pH. Such ionization of a cationic lipid can affect the surface charge of the lipid particle under different pH conditions, which in some embodiments may influence plasma protein absorption, blood clearance, and/or tissue distribution as well as the ability to form endosomolytic non-bilayer structures. Accordingly, in some embodiments, a cationic lipid may be or comprise a pH responsive lipid. In some embodiments a pH responsive lipid is a fatty acid derivative or other amphiphilic compound which is capable of forming a lyotropic lipid phase, and which has a pKa value between pH 5 and pH 7.5. This means that the lipid is uncharged at a pH above the pKa value and positively charged below the pKa value. In some embodiments, a pH responsive lipid may be used in addition to or instead of a cationic lipid for example by binding one or more polyribonucleotides to a lipid or lipid mixture at low pH. pH responsive lipids include, but are not limited to, 1,2-dioleyloxy-3-dimethylamino-propane (DODMA).

In some embodiments, a lipid nanoparticle may comprise one or more cationic lipids as described in WO 2017/075531 (e.g., as presented in Tables 1 and 3 therein) and WO 2018/081480 (e.g., as presented in Tables 1-4 therein), the entire contents of each of which are incorporated herein by reference for the purposes described herein.

In some embodiments, a cationic lipid that may be useful in accordance with the present disclosure is an amino lipid comprising a titratable tertiary amino head group linked via ester bonds to at least two saturated alkyl chains, which ester bonds can be hydrolyzed easily to facilitate fast degradation and/or excretion via renal pathways. In some embodiments, such an amino lipid has an apparent pKₐ of about 6.0-6.5 (e.g., in one embodiment with an apparent pKa of approximately 6.25), resulting in an essentially fully positively charged molecule at an acidic pH (e.g., pH 5). In some embodiments, such an amino lipid, when incorporated in lipid nanoparticle, can confer distinct physicochemical properties that regulate particle formation, cellular uptake, fusogenicity and/or endosomal release of polyribonucleotide(s). In some embodiments, introduction of an aqueous RNA solution to a lipid mixture comprising such an amino lipid at pH 4.0 can lead to an electrostatic interaction between the negatively charged RNA backbone and the positively charged cationic lipid. Without wishing to be bound by any particular theory, such electrostatic interaction leads to particle formation coincident with efficient encapsulation of RNA drug substance. After RNA encapsulation, adjustment of the pH of the medium surrounding the resulting lipid nanoparticle to a more neutral pH (e.g., pH 7.4) results in neutralization of the surface charge of the lipid nanoparticle. When all other variables are held constant, such charge-neutral particles display longer in vivo circulation lifetimes and better delivery to hepatocytes compared to charged particles, which are rapidly cleared by the reticuloendothelial system. Upon endosomal uptake, the low pH of the endosome renders lipid nanoparticle comprising such an amino lipid fusogenic and allows the release of the RNA into the cytosol of the target cell.

In some embodiments, a cationic lipid that may be useful in accordance with the present disclosure has one of the structures set forth in Table 10 below:

TABLE 10
Exemplary cationic lipids

| No. | Structure |
|---|---|
| I-1 | 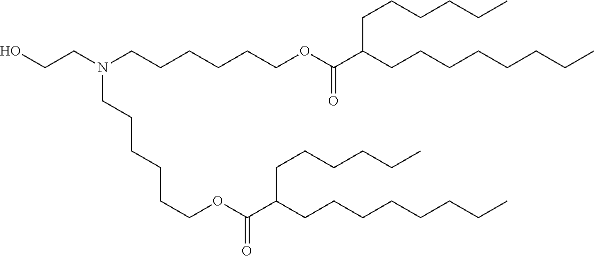 [SMILES: CCCCCCCCC(CCCCCC)C(=O)OCCCCCCN(CCO)CCCCCCOC(=O)C(CCCCCC)CCCCCCCC] |
| I-2 | 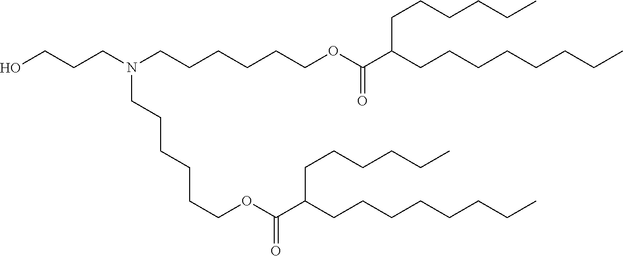 [SMILES: CCCCCCCCC(CCCCCC)C(=O)OCCCCCCN(CCCO)CCCCCCOC(=O)C(CCCCCC)CCCCCCCC] |
| I-3 | 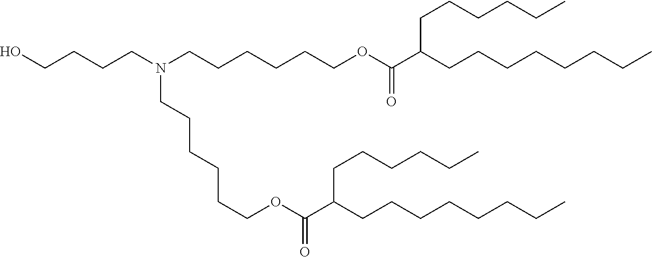 [SMILES: CCCCCCCCC(CCCCCC)C(=O)OCCCCCCN(CCCCO)CCCCCCOC(=O)C(CCCCCC)CCCCCCCC] |
| I-4 | 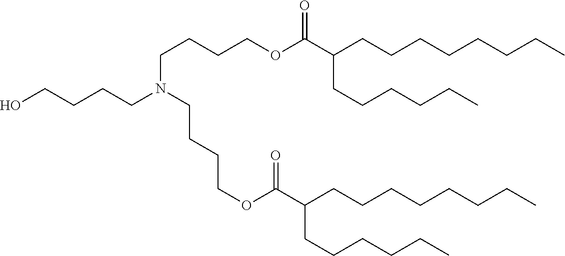 [SMILES: CCCCCCCCC(CCCCCC)C(=O)OCCCCN(CCCCO)CCCCOC(=O)C(CCCCCC)CCCCCCCC] |
| I-5 | 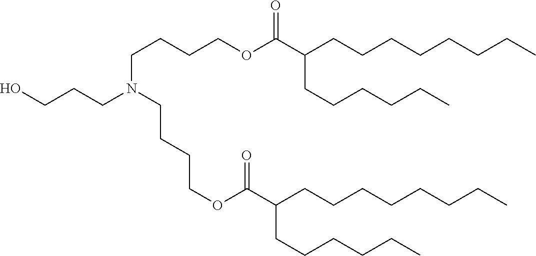 [SMILES: CCCCCCCCC(CCCCCC)C(=O)OCCCCN(CCCO)CCCCOC(=O)C(CCCCCC)CCCCCCCC] |
| I-6 | 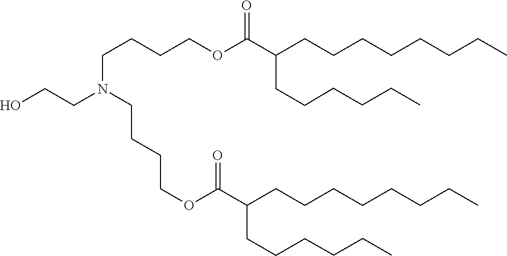 [SMILES: CCCCCCCCC(CCCCCC)C(=O)OCCCCN(CCO)CCCCOC(=O)C(CCCCCC)CCCCCCCC] |
| I-7 | 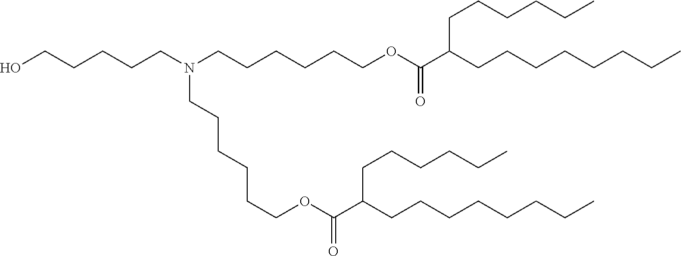 [SMILES: CCCCCCCCC(CCCCCC)C(=O)OCCCCCCN(CCCCCO)CCCCCCOC(=O)C(CCCCCC)CCCCCCCC] |
| I-8 | 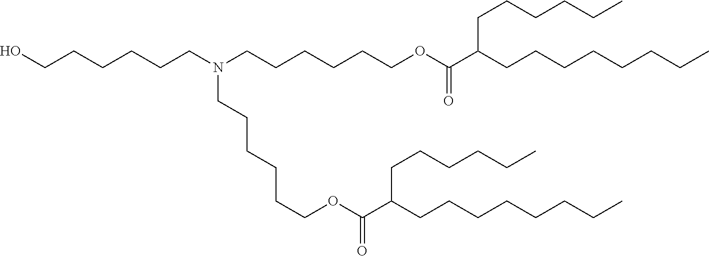 [SMILES: CCCCCCCCC(CCCCCC)C(=O)OCCCCCCN(CCCCCCO)CCCCCCOC(=O)C(CCCCCC)CCCCCCCC] |
| I-9 | 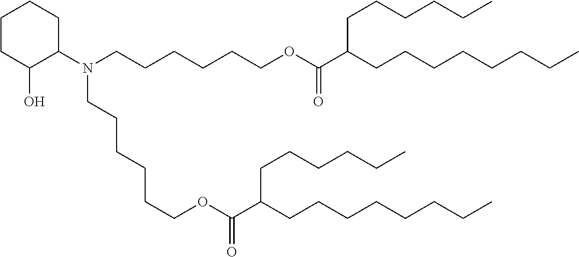 [SMILES: CCCCCCCCC(CCCCCC)C(=O)OCCCCCCN(CCCCCCOC(=O)C(CCCCCC)CCCCCCCC)C1CCCCC1O] |
| I-10 | 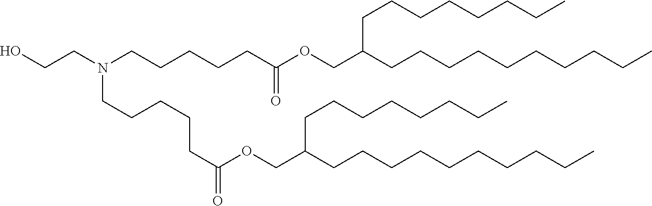 [SMILES: CCCCCCCCCCC(CCCCCCCC)COC(=O)CCCCCN(CCO)CCCCCC(=O)OCC(CCCCCCCC)CCCCCCCCCC] |
| I-11 | 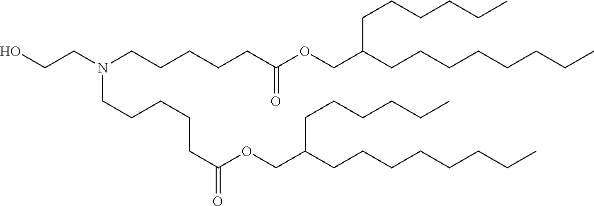 [SMILES: CCCCCCCCC(CCCCCC)COC(=O)CCCCCN(CCO)CCCCCC(=O)OCC(CCCCCC)CCCCCCCC] |
| I-12 | 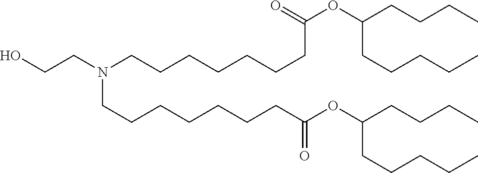 [SMILES: CCCCCC(CCCC)OC(=O)CCCCCCCN(CCO)CCCCCCCC(=O)OC(CCCC)CCCCC] |
| I-13 | 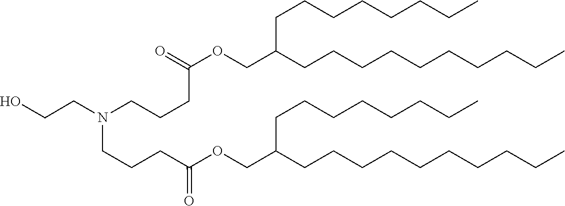 [SMILES: CCCCCCCCCCC(CCCCCCCC)COC(=O)CCCN(CCO)CCCC(=O)OCC(CCCCCCCC)CCCCCCCCCC] |
| I-14 | 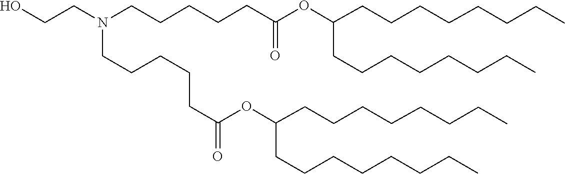 [SMILES: CCCCCCCCC(CCCCCCCC)OC(=O)CCCCCN(CCO)CCCCCC(=O)OC(CCCCCCCC)CCCCCCCC] |
| I-15 | 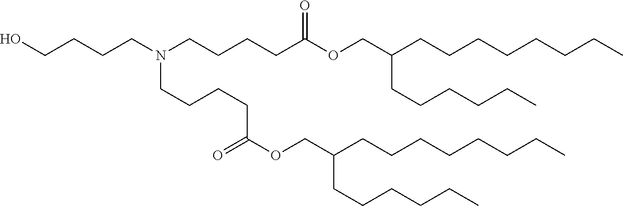 [SMILES: CCCCCCCCC(CCCCCC)COC(=O)CCCCN(CCCCO)CCCCC(=O)OCC(CCCCCC)CCCCCCCC] |
| I-16 | 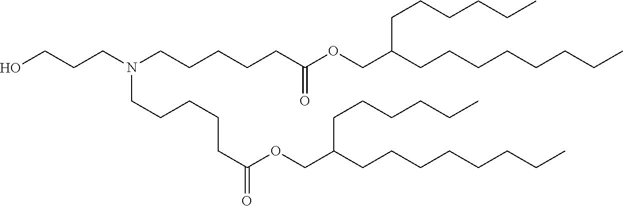 [SMILES: CCCCCCCCC(CCCCCC)COC(=O)CCCCCN(CCCO)CCCCCC(=O)OCC(CCCCCC)CCCCCCCC] |
| I-17 | 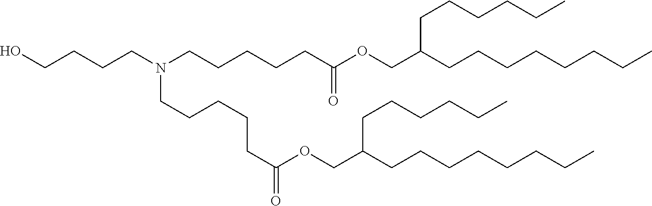 [SMILES: CCCCCCCCC(CCCCCC)COC(=O)CCCCCN(CCCCO)CCCCCC(=O)OCC(CCCCCC)CCCCCCCC] |
| I-18 | 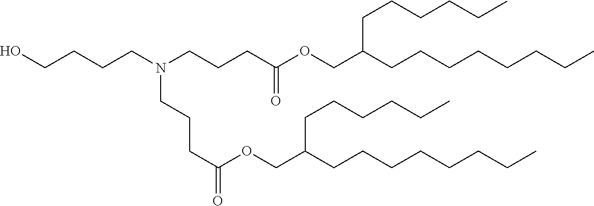 [SMILES: CCCCCCCCC(CCCCCC)COC(=O)CCCN(CCCCO)CCCC(=O)OCC(CCCCCC)CCCCCCCC] |
| I-19 | 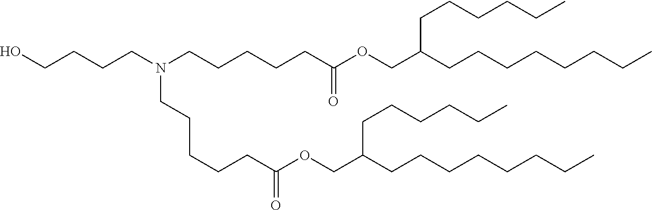 [SMILES: CCCCCCCCC(CCCCCC)COC(=O)CCCCCN(CCCCO)CCCCCC(=O)OCC(CCCCCC)CCCCCCCC] |
| I-20 | 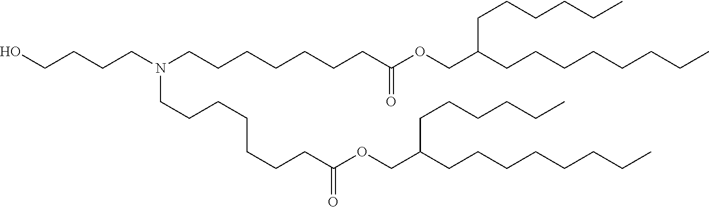 [SMILES: CCCCCCCCC(CCCCCC)COC(=O)CCCCCCCN(CCCCO)CCCCCCCC(=O)OCC(CCCCCC)CCCCCCCC] |
| I-21 | 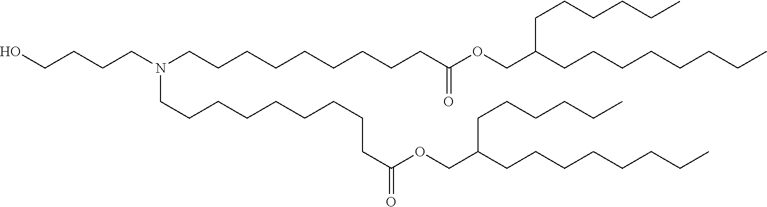 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 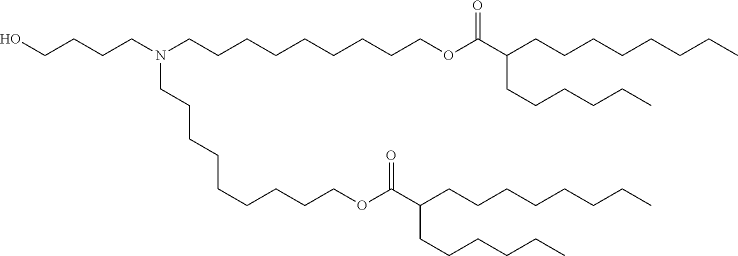 [SMILES: CCCCCCCCC(CCCCCC)C(=O)OCCCCCCCCCN(CCCCO)CCCCCCCCCOC(=O)C(CCCCCC)CCCCCCCC] |
| I-23 | 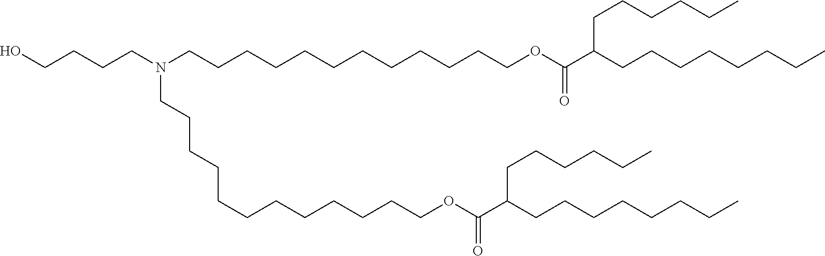 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 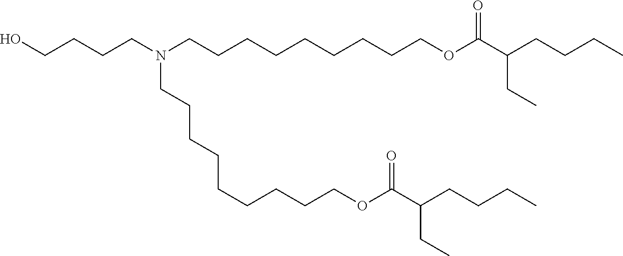 [SMILES: CCCCC(CC)C(=O)OCCCCCCCCCN(CCCCO)CCCCCCCCCOC(=O)C(CC)CCCC] |
| I-25 | 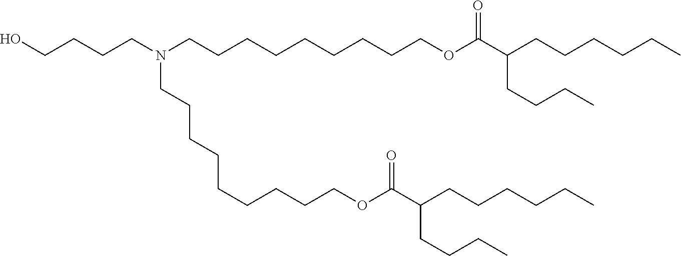 [SMILES: CCCCCCC(CCCC)C(=O)OCCCCCCCCCN(CCCCO)CCCCCCCCCOC(=O)C(CCCC)CCCCCC] |
| I-26 | 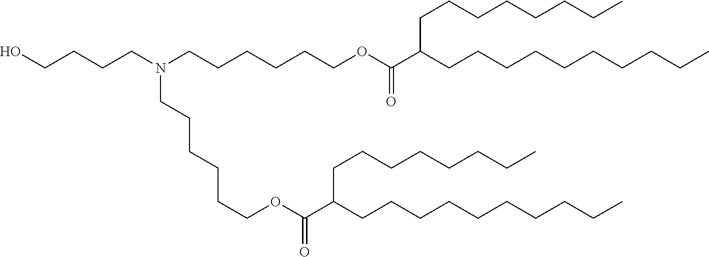 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 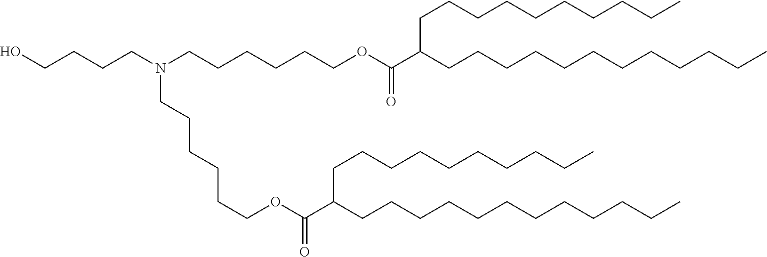 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 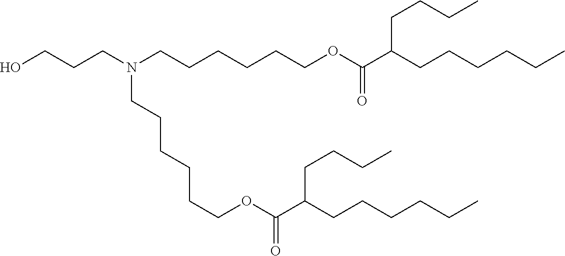 [SMILES: CCCCCCC(CCCC)C(=O)OCCCCCCN(CCCO)CCCCCCOC(=O)C(CCCC)CCCCCC] |
| I-29 | 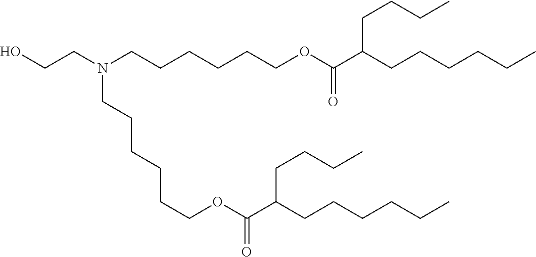 [SMILES: CCCCCCC(CCCC)C(=O)OCCCCCCN(CCO)CCCCCCOC(=O)C(CCCC)CCCCCC] |
| I-30 | 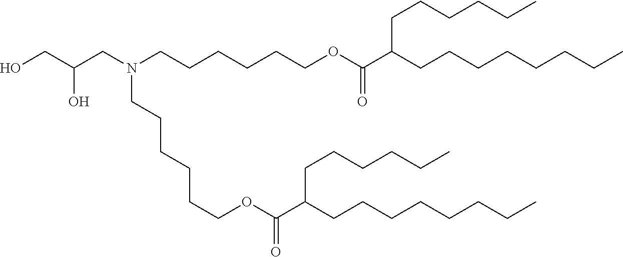 [SMILES: CCCCCCCCC(CCCCCC)C(=O)OCCCCCCN(CCCCCCOC(=O)C(CCCCCC)CCCCCCCC)CC(O)CO] |
| I-31 | 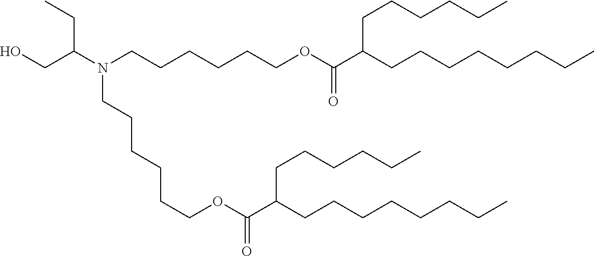 [SMILES: CCCCCCCCC(CCCCCC)C(=O)OCCCCCCN(CCCCCCOC(=O)C(CCCCCC)CCCCCCCC)C(CC)CO] |
| I-32 | 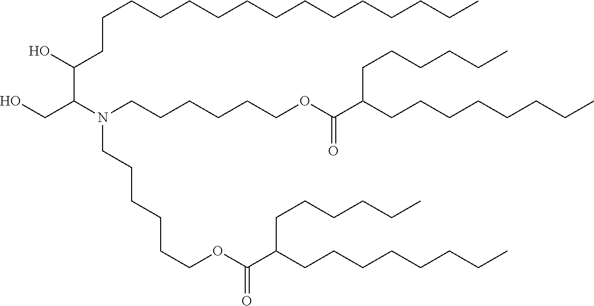 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 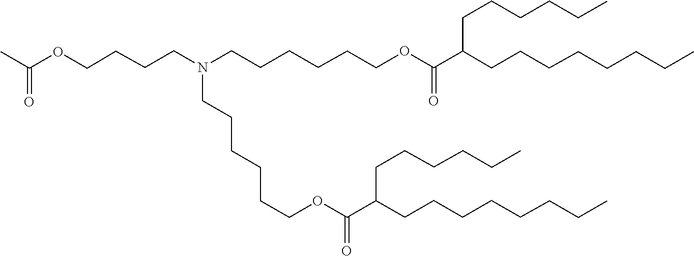 [SMILES: CCCCCCCCC(CCCCCC)C(=O)OCCCCCCN(CCCCCCOC(=O)C(CCCCCC)CCCCCCCC)CCCCOC(C)=O] |
| I-34 | 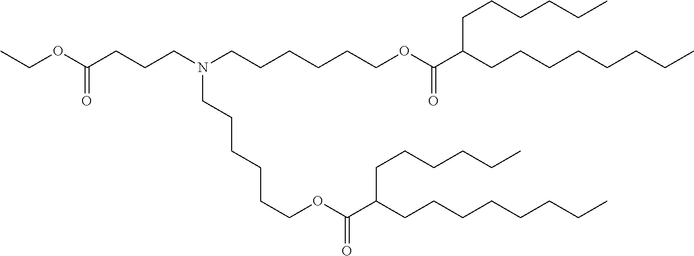 [SMILES: CCCCCCCCC(CCCCCC)C(=O)OCCCCCCN(CCCCCCOC(=O)C(CCCCCC)CCCCCCCC)CCCC(=O)OCC] |
| I-35 | 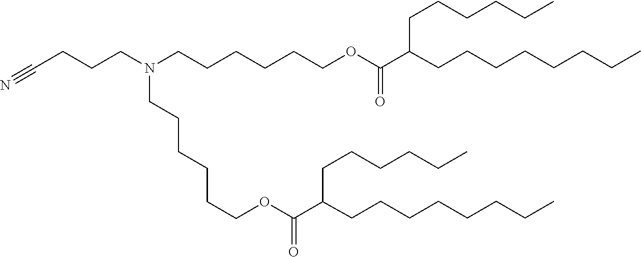 [SMILES: CCCCCCCCC(CCCCCC)C(=O)OCCCCCCN(CCCC#N)CCCCCCOC(=O)C(CCCCCC)CCCCCCCC] |
| I-36 | 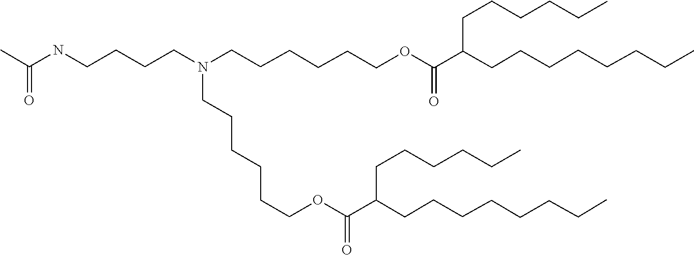 |
| I-37 | 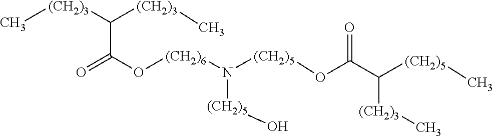 |
| I-38 | 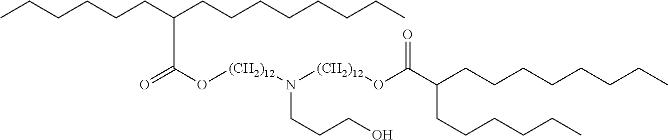 |
| I-39 | 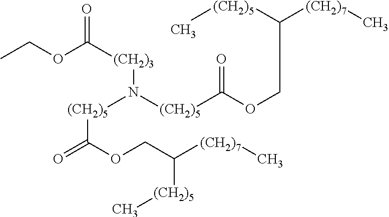 |
| I-40 | 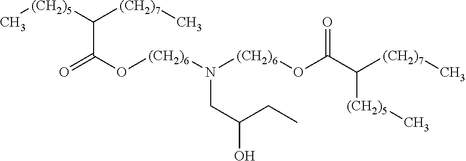 |
| I-41 | 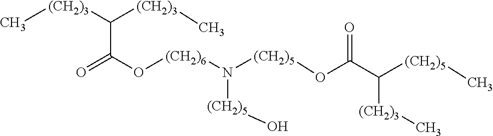 |
| I-42 | 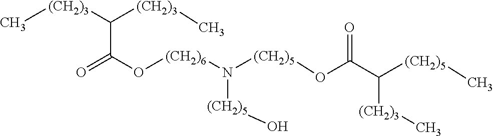 |
| I-43 | 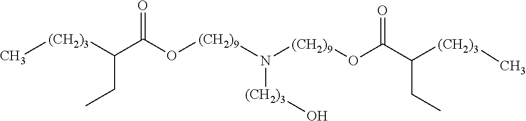 |
| I-44 | 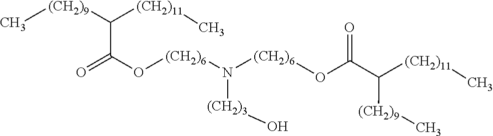 |
| I-45 | 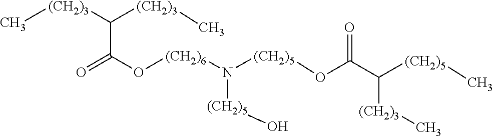 |
| I-46 | 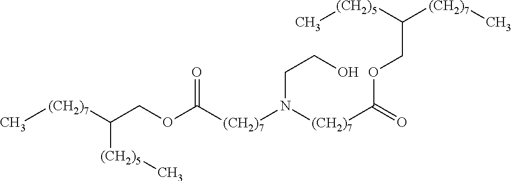 |
| I-47 | 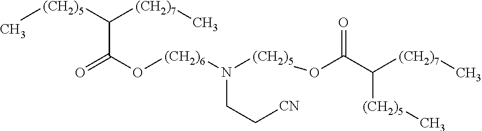 |
| I-48 | 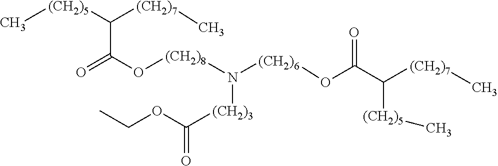 |
| I-49 | 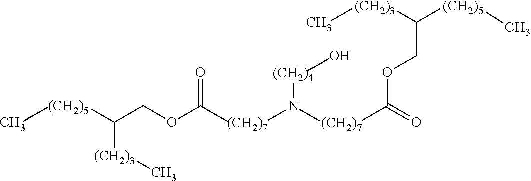 |

or a pharmaceutically acceptable salt thereof. In some embodiments, provided compounds are provided and/or utilized in a salt form (e.g., a pharmaceutically acceptable salt form). Reference to a compound provided herein is understood to include reference to salts thereof, unless otherwise indicated.

In certain embodiments, a cationic lipid that may be useful in accordance with the present disclosure is or comprises ((3-hydroxypropyl)azanediyl)bis(nonane-9,1-diyl) bis(2-butyloctanoate) with a chemical structure in Table 10 above as 1-45.

In some embodiments, a cationic lipid is selected from DODAC, DOTMA, DDAB, DOTAP, DC-Chol, DMRIE, I-3, I-45, and combinations thereof.

In some embodiments, a cationic lipid is I-3. In some embodiments, a cationic lipid is I-45. In some embodiments, a cationic lipid is SM-102. In some embodiments, a cationic lipid is DODAC. In some embodiments, a cationic lipid is DOTMA. In some embodiments, a cationic lipid is DDAB. In some embodiments, a cationic lipid is DOTAP. In some embodiments, a cationic lipid is DC-Chol.

In some embodiments, lipid nanoparticles of the present disclosure comprise about 30 to about 70 mol % of a cationic lipid. In some embodiments, an lipid nanoparticle comprises about 35 to about 65 mol % of a cationic lipid. In some embodiments, an lipid nanoparticle comprises about 40 to about 60 mol % of a cationic lipid. In some embodiments, an lipid nanoparticle comprises about 41 to about 49 mol % of a cationic lipid. In some embodiments, an lipid nanoparticle comprises about 48 mol % of a cationic lipid. In some embodiments, an lipid nanoparticle comprises about 50 mol % of a cationic lipid.

Cationic lipids may be used alone or in combination with neutral lipids, e.g., cholesterol and/or neutral phospholipids, or in combination with other known lipid assembly components.

b. Helper Lipids

As described herein, lipid nanoparticles of the present disclosure comprise one or more helper lipids. In some embodiments, a lipid nanoparticle for delivery of at least one polyribonucleotide described herein comprises one or more helper lipids. A helper lipid may be a neutral lipid, a positively charged lipid, or a negatively charged lipid. In some embodiments, a helper lipid is a lipid that are useful for increasing the effectiveness of delivery of lipid-based particles such as cationic lipid-based particles to a target cell. In some embodiments, a helper lipid may be or comprise a structural lipid with its concentration chosen to optimize lipid nanoparticle particle size, stability, and/or encapsulation.

In some embodiments, a lipid nanoparticle for delivery of polyribonucleotide(s) described herein comprises a neutral helper lipid. Examples of such neutral helper lipids include, but are not limited to phosphotidylcholines such as 1,2-distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, cholesterol, steroids such as sterols and their derivatives. In some embodiments, a steroid is a sterol. In some embodiments, a sterol is cholesterol.

Neutral lipids may be synthetic or naturally derived. Other neutral helper lipids that are known in the art, e.g., as described in WO 2017/075531 and WO 2018/081480, the entire contents of each of which are incorporated herein by reference for the purposes described herein, can also be used in lipid nanoparticles described herein. In some embodiments, a lipid nanoparticle for delivery of polyribonucleotide(s) described herein comprises DSPC and/or cholesterol.

In some embodiments, a lipid nanoparticle described herein comprises multiple neutral lipids (e.g., two neutral lipids). It is understood that reference to “a” neutral lipid is intended to refer to lipid nanoparticles that comprise one or more neutral lipids. In some embodiments, a lipid nanoparticle described herein comprises a phospholipid and/or a steroid. In some embodiments, a lipid nanoparticle described herein comprises DSPC and/or cholesterol.

In some embodiments, a lipid nanoparticle comprises about 5 to about 15 mol % of a phospholipid. In some embodiments, a lipid nanoparticle comprises about 8 to about 12 mol % of a phospholipid. In some embodiments, a lipid nanoparticle comprises about 10 mol % of a phospholipid. In some embodiments, a lipid nanoparticle comprises about 5 to about 15 mol % of DSPC. In some embodiments, a lipid nanoparticle comprises about 8 to about 12 mol % of DSPC. In some embodiments, a lipid nanoparticle comprises about 10 mol % of DSPC.

In some embodiments, a lipid nanoparticle comprises about 30 to about 50 mol % of a steroid. In some embodiments, a lipid nanoparticle comprises about 35 to about 45 mol % of a steroid. In some embodiments, a lipid nanoparticle comprises about 38 to about 40 mol % of a steroid. In some embodiments, a lipid nanoparticle comprises about 38.5 mol % of a steroid. In some embodiments, a lipid nanoparticle comprises about 40 mol % of a steroid.

In some embodiments, a lipid nanoparticle comprises about 30 to about 50 mol % of cholesterol. In some embodiments, a lipid nanoparticle comprises about 35 to about 45 mol % of cholesterol. In some embodiments, a lipid nanoparticle comprises about 38 to about 41 mol % of cholesterol. In some embodiments, a lipid nanoparticle comprises about 38.5 mol % of cholesterol. In some embodiments, a lipid nanoparticle comprises about 40.7 mol % of cholesterol.

In some embodiments, a lipid nanoparticle comprises about 5 to about 15 mol % of phospholipid and about 30 to about 50 mol % of steroid.

In some embodiments, a lipid nanoparticle for delivery of at least one polyribonucleotide described herein comprises at least two helper lipids (e.g., ones described herein). In some such embodiments, a lipid nanoparticle for delivery of at least one polyribonucleotide described herein comprises DSPC and cholesterol.

c. Polymer-Conjugated Lipids

As described herein, lipid nanoparticles of the present disclosure comprise a polymer-conjugated lipid. In some embodiments, a lipid nanoparticle for delivery of at least one polyribonucleotide described herein comprises a polymer-conjugated lipid. A polymer-conjugated lipid is typically a molecule comprising a lipid portion and a polymer portion conjugated thereto.

In some embodiments, a polymer-conjugated lipid is a PEG-conjugated lipid. In some embodiments, a PEG-conjugated lipid is designed to sterically stabilize a lipid particle by forming a protective hydrophilic layer that shields the hydrophobic lipid layer. In some embodiments, a PEG-conjugated lipid can reduce its association with serum proteins and/or the resulting uptake by the reticuloendothelial system when such lipid particles are administered in vivo.

In some embodiments, a PEG lipid is selected from pegylated diacylglycerol (PEG-DAG) such as I-(monomethoxy-polyethyleneglycol)-2,3-dimyristoylglycerol (PEG-DMG) (e.g., 1,2-dimyristoyl-rac-glycero-3-methoxypolyethylene glycol-2000 (PEG2000-DMG)), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as co-m ethoxy (polyethoxy)ethyl-N-(2,3-di(tetradecanoxy)propyl)carbamate, and 2,3-di(tetradecanoxy)propy 1-N-(co methoxy(polyethoxy)ethyl)carbamate.

Certain PEG-conjugated lipids (also known as PEGylated lipids) were clinically approved with safety demonstrated in clinical trials. PEG-conjugated lipids are known to affect cellular uptake, a prerequisite to endosomal localization and payload delivery. The present disclosure, among other things, provides an insight that the pharmacology of encapsulated nucleic acid can be controlled in a predictable manner by modulating the alkyl chain length of a PEG-lipid anchor. In some embodiments, the present disclosure, among other things, provides an insight that such PEG-conjugated lipids may be selected for an polyribonucleotide/lipid nanoparticle drug product formulation to provide optimum delivery of polyribonucleotides to the liver. In some embodiments, such PEG-conjugated lipids may be designed and/or selected based on reasonable solubility characteristics and/or its molecular weight to effectively perform the function of a steric barrier. For example, in some embodiments, such a PEGylated lipid does not show appreciable surfactant or permeability enhancing or disturbing effects on biological membranes. In some embodiments, PEG in such a PEG-conjugated lipid can be linked to diacyl lipid anchors with a biodegradable amide bond, thereby facilitating fast degradation and/or excretion. In some embodiments, a lipid nanoparticle comprising a PEG-conjugated lipid retain a full complement of a PEGylated lipid. In the blood compartment, such a PEGylated lipid dissociates from the particle over time, revealing a more fusogenic particle that is more readily taken up by cells, ultimately leading to release of the RNA payload.

In some embodiments, a PEG-lipid is PEG2000-DMG:

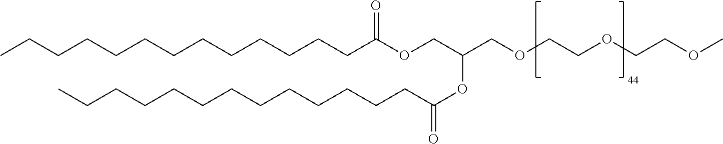

In some embodiments, a lipid nanoparticle may comprise one or more PEG-conjugated lipids or pegylated lipids as described in WO 2017/075531 and WO 2018/081480, the entire contents of each of which are incorporated herein by reference for the purposes described herein. For example, in some embodiments, a PEG-conjugated lipid that may be useful in accordance with the present disclosure can have a structure

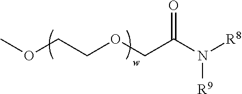

as described in WO 2017/075531, or a pharmaceutically acceptable salt, tautomer or stereoisomer thereof, wherein: R₈ and R₉ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R₈ and R₉ are each independently straight, saturated alkyl chains containing from 12 to 16 carbon atoms. In some embodiments, w has a mean value ranging from 43 to 53. In some embodiments, w is an integer from 40 to 50. In some embodiments, w is 45 to 47. In other embodiments, the average w is about 45. In some embodiments, a PEG-conjugated lipid is or comprises 2-[(Polyethylene glycol)-2000]-N,N-ditetradecylacetamide with a chemical structure as shown as I-3 in Table 9 above and below:

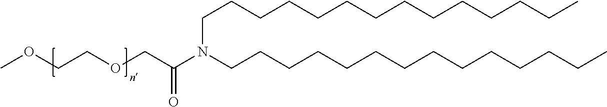

or a pharmaceutically acceptable salt thereof, where n′ is an integer from 45 to 50.

In some embodiments, a PEG-lipid is selected from PEG-DAG, PEG-PE, PEG-S-DAG, PEG2000-DMG, PEG-cer, a PEG dialkyoxypropylcarbamate, ALC-0159, and combinations thereof. In some embodiments, a PEG-lipid is ALC-0159 or PEG2000-DMG. In some embodiments, a PEG-lipid is ALC-0159. In some embodiments, a PEG-lipid is PEG2000-DMG. In some embodiments, a PEG-lipid is PEG-DAG. In some embodiments, a PEG-lipid is PEG-PE. In some embodiments, a PEG-lipid is PEG-S-DAG. In some embodiments, a PEG-lipid is PEG-cer. In some embodiments, a PEG-lipid is a PEG dialkyoxypropylcarbamate.

In some embodiments, a PEG group that is part of a PEG-lipid has, on average in a composition comprising one or more PEG-lipid molecules, a number average molecular weight (Mn) of about 2000 g/mol.

In some embodiments, a PEG-lipid is about 0.5 to about 5 mol % relative to total lipids in the lipid nanoparticle. In some embodiments, a lipid nanoparticle comprises about 1.0 to about 2.5 mol % of a PEG-lipid. In some embodiments, a lipid nanoparticle comprises about 1.5 to about 2.0 mol % of a PEG-lipid. In some embodiments, a lipid nanoparticle comprises about 1.5 to about 1.8 mol % of a PEG-lipid.

In some embodiments, a molar ratio of total cationic lipid to total polymer-conjugated lipid (e.g., PEG-conjugated lipid) is from about 100:1 to about 20:1. In some embodiments, a molar ratio of total cationic lipid to total polymer-conjugated lipid (e.g., PEG-conjugated lipid) is from about 50:1 to about 20:1. In some embodiments, a molar ratio of total cationic lipid to total polymer-conjugated lipid (e.g., PEG-conjugated lipid) is from about 40:1 to about 20:1. In some embodiments, a molar ratio of total cationic lipid to total polymer-conjugated lipid (e.g., PEG-conjugated lipid) is from about 35:1 to about 25:1.

In some embodiments, a lipid nanoparticle comprises i) about 30 to about 50 mol % of the cationic lipid; ii) about 1 to about 5 mol % of a PEG-lipid; iii) about 5 to about 15 mol % of a neutral lipid; and iv) about 30 to about 50 mol % of a steroid. In some embodiments, a lipid nanoparticle comprises: i) about 30% to about 50% by weight of ALC-0315; ii) about 1% to about 5% by weight of a ALC-0159; iii) about 5% to about 15% by weight of DSPC; and iv) about 30 to about 50 mol % of cholesterol.

In some embodiments, an lipid nanoparticle comprises: i) about 47.5 mol % of ALC-0315; ii) about 1.8 mol % of a ALC-0159; iii) about 10 mol % of DSPC; and iv) about 40.7 mol % of cholesterol.

In some embodiments, a lipid nanoparticle comprises: i) about 30 to about 50 mol % of SM-102; ii) about 1 to about 5 mol % of a PEG2000-DMG; iii) about 5 to about 15 mol % of DSPC; and iv) about 30 to about 50 mol % of a steroid. In some embodiments, a lipid nanoparticle comprises i) about 50 mol % of SM-102; ii) about 1.5 mol % of PEG2000-DMG; iii) about 10 mol % of DSPC; and iv) about 38.5 mol % of cholesterol.

### 3. Exemplary Lipid Nanoparticle Compositions

In some embodiments, lipids that form lipid nanoparticles described herein comprise: a polymer-conjugated lipid; a cationic lipid; and at least one helper lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, about 46-48 mol %, or about 47.2-47.8 mol % of the total lipids. In certain embodiments, total cationic lipid is present in about 47.0, 47.1, 47.2, 47.3, 47.4, 47.5, 47.6, 47.7, 47.8, 47.9 or 48.0 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid is present in about 9.5, 10 or 10.5 mol % of the total lipids. In some embodiments, the molar ratio of the total cationic lipid to the non-steroid neutral lipid ranges from about 4.1: 1.0 to about 4.9: 1.0, from about 4.5: 1.0 to about 4.8: 1.0, or from about 4.7: 1.0 to 4.8: 1.0. In some embodiments, total steroid neutral lipid (e.g., cholesterol) is present in about 35-50 mol %, about 39-49 mol %, about 40-46 mol %, about 40-44 mol %, or about 40-42 mol % of the total lipids. In certain embodiments, total steroid neutral lipid (e.g., cholesterol) is present in about 39, 40, 41, 42, 43, 44, 45, or 46 mol % of the total lipids. In certain embodiments, the molar ratio of total cationic lipid to total steroid neutral lipid is about 1.5:1 to 1: 1.2, or about 1.2: 1 to 1: 1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol % of the total lipids; the cationic lipid is present in 35-65 mol % of the total lipids; and the neutral lipid is present in 35-65 mol % of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol % of the total lipids; the cationic lipid is present in 45-48.5 mol % of the total lipids; and the neutral lipid is present in 45-55 mol % of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol % of the total lipids; the cationic lipid is present in 45-48.5 mol % of the total lipids; the non-steroid neutral lipid is present in 9-11 mol % of the total lipids; and the steroid neutral lipid is present in about 36-44 mol % of the total lipids. In many of such embodiments, a PEG-conjugated lipid is or comprises 2-[(polyethylene glycol)-2000]-N,N-ditetradecylacetamide or a derivative thereof. In many of such embodiments, a cationic lid is or comprises ((3-hydroxypropyl)azanediyl)bis(nonane-9,1-diyl) bis(2-butyloctanoate) or a derivative thereof. In many of such embodiments, a neutral lipid comprises DSPC and cholesterol, wherein DSPC is a non-steroid neutral lipid and cholesterol is a steroid neutral lipid.

In some embodiments, lipids that form the lipid nanoparticles comprise: (a) 2-[(polyethylene glycol)-2000]-N,N-ditetradecylacetamide at about 1-2.5 mol % of the total lipids; (b) DPSC and cholesterol, wherein together DPSC and cholesterol are about 35-65 mol % of the total lipids; and (c) ((3-hydroxypropyl)azanediyl)bis(nonane-9,1-diyl) bis(2-butyloctanoate) at about 35-65 mol % of the total lipids.

### B. Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583,2011/0311582, 2011/0262527,2011/0216622,2011/0117125, 2011/0091525,2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or with ssRNs) may be monitored by appropriate techniques such as, e.g., small-angle X-ray scattering (SAXS) and/or transmission electron cryomicroscopy (CryoTEM).

### V. Pharmaceutical Compositions

The present disclosure provides compositions, e.g., pharmaceutical compositions comprising one or more polyribonucleotides described herein. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition described herein is formulated (e.g., but not limited to, for intravenous, intramuscular, or subcutaneous administration) to deliver an active dose that confers a plasma concentration of an antibody agent encoded by at least one polyribonucleotide (e.g., ones described herein) that mediates pharmacological activity via its dominant mode of action, viral neutralization. In some embodiments, the antibody agent is directed against a CD4 binding site of HIV. In some embodiments, a pharmaceutical composition described herein is formulated (e.g., but not limited to, for intravenous, intramuscular, or subcutaneous administration) to deliver an active dose that confers a plasma concentration of at least about 500 ng/mL of an anti-HIV antibody agent (e.g., an anti-HIV antibody agent targeting a CD4 binding site) encoded by at least one polyribonucleotide (e.g., ones described herein) that mediates pharmacological activity via its dominant mode of action, viral neutralization.

In some embodiments, a pharmaceutical composition is formulated (e.g., but not limited to, for intravenous, intramuscular, or subcutaneous administration) to deliver a dose of 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition described herein may further comprises one or more active agents other than at least one polyribonucleotide encoding an antibody agent directed against an HIV CD4 binding site or an HIV Env trimer apex site. For example, in some embodiments, such an other active agent may be or comprise an antiviral agent. In some embodiments, an exemplary antiviral agent may be one included in Table 1 herein.

The present disclosure provides the recognition that anti-HIV antibodies may be useful in combination with polyribonucleotides and/or compositions provided herein, for example, for treating or preventing HIV. Exemplary anti-HIV antibodies that can be used with compositions described herein include, but are not limited to, 1-18, PGT121, 3BNC117, b12, 10-1074, 10E8, 10E8v4, VRC01, VRC07-523-L/S, or PGDM1400, fragments thereof, or combinations thereof.

The present disclosure further provides the insight that combinations of anti-HIV antibody agents can be encoded in polyribonucleotides. Delivery of a combination of anti-HIV antibody agents via delivery of polyribonucleotides can achieve similar titers of antibody agent in the serum or plasma of a subject. Moreover, a combination of anti-HIV antibody agents via delivery of polyribonucleotides can also broadly neutralize various HIV strains.

In view of these insights, the present disclosure provides compositions comprising one or more polyribonucleotides encoding the heavy chain complementarity determining regions (CDRs) and/or light chain CDRs of PGT121, 3BNC117, b12, 10-1074, 10E8, 10E8v4, VRC01, VRC07-523-L/S, or 1-18 can be used in combination with a polyribonucleotide and/or composition provided herein described. For instance, in some embodiments, one or more polyribonucleotides encoding the heavy chain complementarity determining regions (HCDRs) and/or light chain CDRs (LCDRs) of PGT121, 3BNC117, b12, 10-1074, 10E8, 10E8v4, VRC01, VRC07-523-L/S, or 1-18 can be used in combination with a polyribonucleotide encoding an immunoglobulin chain of an antibody agent, wherein the immunoglobulin chain comprises a heavy chain variable (VH) domain, and the VH domain comprises (a) an HCDR1 comprising an amino acid sequence according to SEQ ID NO: 18; (b) an HCDR2 comprising an amino acid sequence according to SEQ ID NO: 21; and (c) an HCDR3 comprising an amino acid sequence according to SEQ ID NO: 24. In some embodiments, one or more polyribonucleotides encoding the HCDRs and/or LCDRs of PGT121, 3BNC117, b12, 10-1074, 10E8, 10E8v4, VRC01, VRC07-523-L/S, or 1-18 can be used in combination with a polyribonucleotide encoding an immunoglobulin chain of an antibody agent, wherein the immunoglobulin chain comprises a light chain variable (VL) domain, and the VL domain comprises (a) an LCDR1 comprising an amino acid sequence according to SEQ ID NO: 27; (b) an LCDR2 comprising an amino acid sequence according to SEQ ID NO: 30; and (c) an LCDR3 comprising an amino acid sequence according to SEQ ID NO: 33. In some embodiments, one or more polyribonucleotides encoding the HCDRs and/or LCDRs of PGT121, 3BNC117, b12, 10-1074, 10E8, 10E8v4, VRC01, VRC07-523-L/S, or 1-18 can be used in combination with a polyribonucleotide encoding an immunoglobulin chain of an antibody agent, wherein the immunoglobulin chain comprises a heavy chain variable (VH) domain and a light chain variable (VL) domain, wherein the VH domain comprises (a) an HCDR1 comprising an amino acid sequence according to SEQ ID NO: 18; (b) an HCDR2 comprising an amino acid sequence according to SEQ ID NO: 21; and (c) an HCDR3 comprising an amino acid sequence according to SEQ ID NO: 24; and the VL domain comprises (a) an LCDR1 comprising an amino acid sequence according to SEQ ID NO: 27; (b) an LCDR2 comprising an amino acid sequence according to SEQ ID NO: 30; and (c) an LCDR3 comprising an amino acid sequence according to SEQ ID NO: 33. The present disclosure further provides the insight that compositions comprising a combination of such polyribonucleotides can be useful for treating or preventing HIV.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### VI. Patient Populations

Technologies provided herein can be useful for treatment and/or prevention of HIV infection. As described herein, technologies include polyribonucleotides encoding anti-HIV antibody agents, immunoglobulin chains thereof, or fragments thereof. Accordingly, the present disclosure provides pharmaceutical compositions for treatment and/or prevention of HIV. In some embodiments, a pharmaceutical composition comprises a polyribonucleotides as described herein.

In some embodiments, a subject is one suffering from and/or is susceptible to HIV infection. In some embodiments, a subject may be defined by one or more criterion such as age group, gender, genetic background, preexisting clinical conditions, and/or prior exposure to therapy.

In some embodiments, a subject may be determined to be classified as needing a pharmaceutical composition described herein in accordance with the screening tools for HIV. For example, in some embodiments, a subject may be determined to be classified as needing a pharmaceutical composition described herein according to the results obtained in HIV-1 enzyme immunoassays (EIA), HIV-1 western blot, an HIV viral neutralization assay (VNT), and/or PCR test.

In some embodiments, a subject is a model organism. In preferred embodiments, a subject is a human. In some embodiments, a subject is between 18-65 years of age. In some embodiments, a subject is an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old, or from about 95 to about 100 years old.

In some embodiments, a subject is a human infant. In some embodiments, a subject is a human toddler. In some embodiments, a subject is a human child. In some embodiments, a subject is a human adult. In some embodiments, a subject is an elderly human.

In some embodiments, a subject comprises a specific HIV viral load. In some embodiments, a subject comprises a viral load that is greater than 400 copies/mL (e.g., greater than 400, greater than 500, greater than 1,000, greater than 2,000, greater than 4,000, greater than 8,000, greater than 10,000, greater than 20,000, greater than 40,000, greater than 100,000, or greater than 400,000 copies/mL) in plasma or serum. In some embodiments, a subject comprises an undetectable viral load. In some embodiments, a subject comprises a viral load of <400 copies/mL (e.g., less than 400, less than 300, less than 200, less than 100, less than 75, or less than 50 copies/mL) in plasma or serum.

In some embodiments, a subject comprises a normal CD4+ T-cell count (i.e., greater than 350 cells/mm³). In some embodiments, a subject comprises an abnormal CD4+ T-cell count. In some embodiments, a subject comprises a CD4+ T-cell count of at least 450 cells/mm³.

In some embodiments, a subject has not been exposed to HIV. In some embodiments, a subject has or it as risk of developing an HIV infection. In some embodiments, a subject may have had an exposure to HIV. In some embodiments, a subject may have a latent HIV infection.

In some embodiments, a subject is in the primary HIV infection or acute phase. In some embodiments, a subject is suffering from fever, lymph node enlargement, fatigue, rash, gastrointestinal symptoms, acute neuropathy, myalgias and/or malaise, or any other symptom of acute HIV infection. In some embodiments, a subject is in the primary stage of HIV infection and is asymptomatic.

In some embodiments, a subject is suffering from additional co-morbidities related or unrelated to HIV infection, including any of the following: cardiovascular disease, bone disease, renal and hepatic dysfunction. With the emergence of antiretroviral therapies and long term treatment of HIV, cancer has become an increasingly larger cause of mortality in HIV subjects (see Uldrick, Thomas S., et al. J. of Clinical Oncology, 35.33: 3774, 2017, which is herein incorporated by reference). In some embodiments, a subject is additionally suffering from and/or is susceptible to cancer. Exemplary cancers include AIDS-associated cancers such as aggressive B-cell lymphomas (i.e., diffuse large B-cell lymphoma, Burkitt's lymphoma, plasmablastic lymphoma, primary effusion lymphoma, and primary CNS lymphoma), Kaposi sarcoma, and cervical cancer, and non-AIDS-associated cancers, such as classic Hodgkin lymphoma, lung cancer, anal cancer, liver cancer, and head and neck cancers.

In some embodiments, a subject has not previously received an HIV therapy.

In some embodiments, a subject is under analytical treatment interruption (ATI).

In some embodiments, a subject suffering from an HIV infection may have received or is currently receiving other HIV therapy. In some embodiments, a subject is or has received antiretroviral therapy (ART). In some embodiments, a subject is currently receiving or has received one or more ART treatments listed in Table 1.

In some embodiments, a subject has been receiving ART for greater than 1 week, greater than 2 weeks, greater than 3 weeks, greater than 4 weeks, greater than 5 weeks, greater than 6 weeks, greater than 7 weeks, greater than 8 weeks, greater than 12 weeks, greater than 4 months, greater than 5 months, greater than 6 months, greater than 7 months, greater than 8 months, greater than 9 months, greater than 10 months, or greater than 1 year. In some embodiments, a subject is responsive to ART at the time a polyribonucleotide, composition or pharmaceutical composition described herein is administered. In some embodiments, a subject is not responsive to one or more ART agents at the time a polyribonucleotide, composition or pharmaceutical composition described herein is administered.

In some embodiments a subject received ART within less than 6 months of receiving an HIV diagnosis (e.g., less than 12 weeks of receiving an HIV diagnosis).

In some embodiments, a subject has previously received ART. In some embodiments, a subject received prior ART treatment for greater than 1 week, greater than 2 weeks, greater than 3 weeks, greater than 4 weeks, greater than 5 weeks, greater than 6 weeks, greater than 7 weeks, greater than 8 weeks, greater than 12 weeks, greater than 4 months, greater than 5 months, greater than 6 months, greater than 7 months, greater than 8 months, greater than 9 months, greater than 10 months, or greater than 1 year. In some embodiments, a subject was responsive to the prior ART treatment. In some embodiments, a subject was not responsive to the prior ART treatment.

In some embodiments, a subject with an HIV infection is clinically stable. In some embodiments, a subject is characterized by any one of the following characteristics: an absolute neutrophil count>1,000/mm³; hemoglobin levels>10.0 g/dL for men and >9.0 g/dL for women; platelet count>100,000/mm³; an estimated or a measured glomerular filtration rate>60 mL/min/1.73 m² as determined by the National Institutes of Health (NIH) Clinical Center laboratory; aspartate aminotransferase (AST) and alanine aminotransferase (ALT) levels of <2.5 times upper limit of normal (ULN); or direct bilirubin within the normal range for the NIH Clinical Center laboratory.

In some embodiments, a subject has a primary HIV infection. A primary HIV infection may be defined by one or more of the following: a detectable plasma HIV-1 RNA levels of >2000 copies/mL with a negative result from an HIV-1 enzyme immunoassays (EIA); a positive result from an HIV-1 EIA with a negative or indeterminate result from an HIV-1 western blot or another confirmatory antibody test that subsequently evolves to a confirmed positive result; a negative result from an HIV-1 EIA within the past 4 months and HIV-1 RNA levels of >400,000 copies/mL, in the setting of a potential exposure to HIV-1, or a negative result from an HIV-1 EIA within 6 months prior to a positive result from an HIV-1 EIA and an HIV-1 western blot or another confirmatory antibody test; presence of low level of HIV antibodies as determined by having a positive EIA or a positive Western blot with a non-reactive detuned EIA according to a serologic testing algorithm for recent infection.

In some embodiments, a subject is receiving continuous ART treatment and maintains a plasma HIV level that is undetectable or less than 400 copies/mL for greater than 1 month (e.g., greater than 1 month, greater than 2 months, greater than 3 months, greater than 4 months, greater than 6 months, greater than 1 year). In some embodiments, a subject has an HIV viral load (i.e., plasma viremia) of between about 200 and 5,000 copies/mL. In some embodiments, a subject has at least two documented viral levels in plasma of at least 200 copies/mL in the 12 months before treatment.

In some embodiments, a subject has received no ART in the last month. In some embodiments, a subject has received no ART in the last year. In some embodiments, a subject has received no ART in the last 2 years.

In some embodiments, a subject does not meet any one of the following criteria: chronic hepatitis B infection, as evidenced by a positive test for hepatitis B surface antigen (HBsAg), or chronic hepatitis C virus (HCV) infection, as evidenced by a positive test for HCV RNA; have had an HIV immunotherapy or vaccine(s) received within 1 year prior to treatment have received any licensed or experimental non-HIV vaccination (e.g., hepatitis B, influenza, pneumococcal polysaccharide) received within 2 weeks prior to treatment; have received other investigational study agent within 28 days of treatment; have an any active malignancy that may require systemic chemotherapy or radiation therapy; have received immunosuppressive medications within 3 months prior to enrollment (not including corticosteroid nasal spray or inhaler, topical corticosteroids for mild, uncomplicated dermatitis; or oral/parenteral corticosteroids administered for non-chronic conditions not expected to recur); have a history or other clinical evidence of: significant or unstable cardiac or cerebrovascular disease (e.g., angina, congestive heart failure, recent stroke or myocardial infarction); severe illness, malignancy, immunodeficiency other than HIV; or documented multiclass ART drug resistance.

### VII. Treatment Methods

In some embodiments, a pharmaceutical composition described herein can be taken up by cells for production of an encoded antibody agent at therapeutically relevant serum concentrations. Accordingly, the present disclosure provides methods of using pharmaceutical compositions described herein. For example, in some embodiments, a method provided herein comprises administering a pharmaceutical composition described herein to a subject.

As used herein, the term “administering” or “administration” typically refers to the administration of a composition to a subject to achieve delivery of an agent (e.g., at least one polyribonucleotide encoding an antibody agent described herein) that is, or is included in, a composition to a target site or a site to be treated. Those of ordinary skill in the art will be aware of a variety of routes that may, in appropriate circumstances, be utilized for administration to a subject, for example a human. Administration may be, for example, bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In preferred embodiments, administration may be intramuscular, intravenous, or subcutaneous.

In some embodiments, administration of a pharmaceutical composition results in delivery of one or more polyribonucleotides as described herein (e.g., encoding an immunoglobulin chain of an antibody agent) to a subject. In some embodiments, administering a pharmaceutical composition to a subject results in expression in the subject of an immunoglobulin chain of an antibody agent encoded by an administered polyribonucleotide. In some embodiments, administering a pharmaceutical composition to a subject results in expression in the subject of an antibody agent encoded by an administered polyribonucleotide.

In some embodiments, a therapeutically relevant concentration of an antibody agent or immunoglobulin chain thereof can be at least 1 μg/ml (e.g., at least 10 μg/ml) in the subject's serum or plasma.

In some embodiments, an antibody agent expressed following administration of a pharmaceutical composition described herein is characterized in that it exhibits a geometric mean IC50 of less than 1 μg/ml against at least five sensitive HIV-1 clones when tested in the TZM-bl cell pseudovirus neutralization assay. In some embodiments, an antibody agent expressed following administration of a pharmaceutical composition described herein is characterized in that it exhibits a geomean IC50 of less than 0.15 μg/mL against a global reference panel when tested in the TZM-bl cell pseudovirus neutralization assay. In some embodiments, an antibody agent expressed following administration of a pharmaceutical composition described herein is characterized in that it is capable of neutralizing one or more HIV strains when tested in the TZM-bl cell pseudovirus neutralization assay at antibody agent concentrations up to 25 μg/ml. In some embodiments, an antibody agent expressed following administration of a pharmaceutical composition described herein is characterized in that it is capable of neutralizing one or more HIV strains at a level that is within 3-fold of a level of an equivalent amount of recombinant benchmark antibody. In some embodiments, a recombinant benchmark antibody is an unmodified wild-type IgG antibody comprising the same HCDR1, HCDR2, HCDR2, LCDR1, LCDR2, and LCDR3 as the antibody agent.

In some embodiments, administration may involve only a single dose. In some embodiments, administration may involve application of a fixed number of doses.

In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Those skilled in the art are aware that immunotherapies (e.g., polyribonucleotides encoding antibody agents) can be administered in dosing cycles. In some embodiments, pharmaceutical compositions described herein are administered in one or more dosing cycles.

In some embodiments, one dosing cycle is at least 3 or more days (including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 days. In some embodiments, one dosing cycle is at least 21 days.

In some embodiments, one dosing cycle may involve multiple doses, e.g., according to a pattern such as, for example, a dose may be administered daily within a dosing cycle, or a dose may be administered every 2 days, every 3 days, every 4 days, every 5 days, every 6 days, every 7 days, every 2 weeks, monthly, every 2 months within a cycle.

In some embodiments, multiple dosing cycles may be administered. For example, in some embodiments, at least 2 dosing cycles (including, e.g., at least 3 dosing cycles, at least 4 dosing cycles, at least 5 dosing cycles, at least 6 dosing cycles, at least 7 dosing cycles, at least 8 dosing cycles, at least 9 dosing cycles, at least 10 dosing cycles, or more) can be administered. In some embodiments, the number of dosing cycles to be administered may vary with types of treatment (e.g., monotherapy vs. combination therapy). In some embodiments, at least 3-8 dosing cycles may be administered.

In some embodiments, there may be a “rest period” between dosing cycles; in some embodiments, there may be no rest period between dosing cycles. In some embodiments, there may be sometimes a rest period and sometimes no rest period between dosing cycles.

In some embodiments, a rest period may have a length within a range of several days to several months. For example, in some embodiments, a rest period may have a length of at least 3 days or more, including, e.g., at least 4 days, at least 5 days, at least 6 days, at least 7 days, at least 8 days, at least 9 days, at least 10 days, at least 11 days, at least 12 days, at least 13 days, at least 14 days or more. In some embodiments, a rest period may have a length of at least 1 week or more, including, e.g., at least 2 weeks, at least 3 weeks, at least 4 weeks, or more.

Dosage of pharmaceutical compositions described herein may vary with a number of factors including, e.g., but not limited to body weight of a subject to be treated, cancer types and/or cancer stages, and/or monotherapy or combination therapy. In some embodiments, a dosing cycle involves administration of a set number and/or pattern of doses. For example, in some embodiments, a pharmaceutical composition described herein is administered at least one dose per dosing cycle, including, e.g., at least two doses per dosing cycle, at least three doses per dosing cycle, at least four doses per dosing cycle, or more.

In some embodiments, a dosing cycle involves administration of a set cumulative dose, e.g., over a particular period of time, and optionally via multiple doses, which may be administered, for example, at set interval(s) and/or according to a set pattern. In some embodiments, a set cumulative dose may be administered via multiple doses at set intervals such that there is at least some temporal overlap in biological and/or pharmacokinetics effects generated by such multiple doses on a target cell or on a subject being treated. In some embodiments, a set cumulative dose may be administered via multiple doses at set intervals such that biological and/or pharmacokinetics effects generated by such multiple doses on a target cell or on a subject being treated may be additive. By way of example only, in some embodiments, a set cumulative dose of X mg may be administered via two doses with each dose of X/2 mg, wherein such two doses are administered sufficiently close in time such that biological and/or pharmacokinetics effects generated by each X/2-mg dose on a target cell or on a subject being treated may be additive.

The present disclosure provides the recognition that the timing of administration of multiple doses of an antibody agent described herein, alone or in combination with another anti-HIV antibody can impact therapeutic efficacy. For example, without wishing to be bound to any particular theory, in the event a dose of a second RibobNAb is administered too long after a dose of a first RibobNAb is administered, a period of time exists when the treatment essentially involves a monotherapy, which could put the first RibobNAb in jeopardy of viral escape.

In some embodiments, dosing may be adjusted based on response of a subject receiving the therapy. For example, in some embodiments, dosing may involve administration of a higher dose followed later by administration of a lower dose if one or more parameters for safety pharmacology assessment indicates that the prior dose may not satisfy the medical safety requirement according to a physician. In some embodiments, dose escalation may be performed at one or more of the levels. Without wishing to be bound by any particular theory, the present disclosure, among other things, provides an insight that a pharmaceutically guided dose escalation (PGDE) method may be applied to determine an appropriate dose of pharmaceutical compositions described herein.

In some embodiments, pharmaceutical compositions described herein can be administered to subjects as monotherapy.

In some embodiments, a pharmaceutical composition provided herein may be administered as part of combination therapy. In some embodiments, a pharmaceutical composition provided herein may be administered as part of a combination therapy comprising the pharmaceutical composition and one or more anti-HIV antibody agents or one or more anti-HIV RibobNAbs. In some embodiments, one or more HIV antibody agents can each comprise CDRs from 1-18, PGT121, 3BNC117, b12, 10-1074, 10E8, 10E8v4, VRC01, VRC07-523-L/S, or PGDM1400. In some embodiments, one or more HIV RibobNAbs can each comprise CDRs from 1-18, PGT121, 3BNC117, b12, 10-1074, 10E8, 10E8v4, VRC01, VRC07-523-L/S, PGDM1400, or a combination thereof.

In some embodiments, a pharmaceutical composition provided herein may be administered as part of a combination therapy comprising the pharmaceutical composition and ART. In some embodiments, the ART may comprise one or more agents provided in Table 1.

In some embodiments, subjects receiving a composition provided herein (e.g., a pharmaceutical composition) may be monitored periodically over a dosing regimen to assess efficacy of the administered treatment. For example, in some embodiments, efficacy of an administered treatment may be assessed periodically, e.g., weekly, biweekly, every 4 weeks, every 5 weeks, every 6 weeks, every 7 weeks, every 8 weeks, or longer.

### VIII. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (ψ), N1-methyl-pseudouridine (m1ψ), or 5-methyl-uridine (m5U) can be used to replace uridine triphosphate (UTP). In some embodiments, pseudouridine (ψ) can be used to replace uridine triphosphate (UTP). In some embodiments, N1-methyl-pseudouridine (m1ψ) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. For example, in some embodiments, a poly(A) tail comprises a nucleotide sequence according to SEQ ID NO: 1461. In some embodiments, a poly(A) tail comprises a plurality of A residues interrupted by a linker. In some embodiments, a linker comprises the nucleotide sequence GCATATGAC (SEQ ID NO: 1461).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7(3′OMeG)(5′)ppp(5′)(2′OMeA)pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments where an antibody agent is encoded by a first polyribonucleotide encoding a first immunoglobulin chain and a second polyribonucleotide encoding a second immunoglobulin chain such that both, when both translated and expressed, form a full antibody agent, a batch of a first polyribonucleotides and a batch of a second polyribonucleotides, each after purification (e.g., as described herein) can be mixed in an appropriate ratio. For example, in some embodiments, such a first polyribonucleotide batch and a second polyribonucleotide batch may be mixed in a molar ratio of about 1:1.5 to about 1.5:1, e.g., in some embodiments in molar ratio of about 1:1.

In some embodiments, polyribonucleotides may be processed through 0.2 μm filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising two or more RNAs (e.g., one encoding a HC of an antibody and another encoding a LC of the antibody) can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

### IX. DNA Constructs

Among other things, the present disclosure provides DNA constructs, for example that may encode one or more antibody agents as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.).

Expression vectors, which may be plasmid or viral or other vectors, typically include an expressible sequence of interest (e.g., a coding sequence) that is functionally linked with one or more control elements (e.g., promoters, enhancers, transcription terminators, etc.). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc.).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce an RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### EXEMPLARY NUMBERED EMBODIMENTS

Embodiment 1. A polyribonucleotide encoding an immunoglobulin chain of an antibody agent, wherein the immunoglobulin chain comprises a heavy chain variable (VH) domain, and the VH domain comprises: (a) a heavy chain complementarity determining region (HCDR)1 comprising an amino acid sequence according to SEQ ID NO: 18; (b) an HCDR2 comprising an amino acid sequence according to SEQ ID NO: 21; and (c) an HCDR3 comprising an amino acid sequence according to SEQ ID NO: 24.
Embodiment 2. The polyribonucleotide of embodiment 1, wherein the polyribonucleotide comprises a VH domain-encoding sequence, and wherein the VH domain-encoding sequence comprises: (a) the HCDR1-encoding sequence that comprises or consists of the ribonucleic acid sequence according to SEQ ID NO: 19; (b) the HCDR2-encoding sequence that comprises or consists of the ribonucleic acid sequence according to SEQ ID NO: 22; and (c) the HCDR3-encoding sequence that comprises or consists of the ribonucleic acid sequence according to SEQ ID NO: 25.
Embodiment 3. The polyribonucleotide of embodiment 1 or 2, wherein the VH domain comprises or consists of an amino acid sequence according to SEQ ID NO: 36.
Embodiment 4. The polyribonucleotide of any one of embodiments 1-3, wherein the VH domain-encoding sequence comprises or consists of a ribonucleic acid sequence according to SEQ ID NO: 37.
Embodiment 5. The polyribonucleotide of any one of embodiments 1-3, wherein the VH domain-encoding sequence comprises or consists of a ribonucleic acid sequence according to SEQ ID NO: 39 or SEQ ID NO: 41.
Embodiment 6. The polyribonucleotide of any one of embodiments 1-5, wherein the immunoglobulin chain further comprises one or more constant domains, and wherein the VH domain is operably linked to the one or more constant domains.
Embodiment 7. The polyribonucleotide of embodiment 6, wherein the one or more constant domains comprise a CH₂ domain.
Embodiment 8. The polyribonucleotide of embodiment 7, wherein the CH₂ domain comprises or consists of an amino acid sequence according to SEQ ID NO: 96.
Embodiment 9. The polyribonucleotide of embodiment 7 or 8, wherein the polyribonucleotide comprises a ribonucleic acid sequence that encodes the CH₂ domain and comprises or consists of a sequence according to SEQ ID NO: 97.
Embodiment 10. The polyribonucleotide of embodiment 7, wherein the CH₂ domain comprises one or more substitution mutations, wherein the one or more substitution mutations comprise or consist of G236A, A330L, I332E, or a combination thereof, and wherein the substitution mutation positions are according to EU numbering.
Embodiment 11. The polyribonucleotide of embodiment 7 or 10, wherein the CH₂ domain comprises one or more substitution mutations, wherein the one or more substitution mutations comprise or consist of G236A, and wherein the substitution mutation positions are according to EU numbering.
Embodiment 12. The polyribonucleotide of embodiment 7 or 10, wherein the CH₂ domain comprises one or more substitution mutations, wherein the one or more substitution mutations comprise or consist of I332E, and wherein the substitution mutation positions are according to EU numbering.
Embodiment 13. The polyribonucleotide of embodiment 7 or 10, wherein the CH₂ domain comprises one or more substitution mutations, wherein the one or more substitution mutations comprise or consist of G236A and I332E, and wherein the substitution mutation positions are according to EU numbering.
Embodiment 14. The polyribonucleotide of embodiment 7 or 10, wherein the CH₂ domain comprises one or more substitution mutations, wherein the one or more substitution mutations comprise or consist of G236A, A330L, and I332E, and wherein the substitution mutation positions are according to EU numbering.
Embodiment 15. The polyribonucleotide of any one of embodiments 7, 10, and 14, wherein the CH₂ domain comprises or consists of an amino acid sequence according to SEQ ID NO: 99.
Embodiment 16. The polyribonucleotide of any one of embodiments 7, 10, 14, and 15, wherein the polyribonucleotide comprises a ribonucleic acid sequence that encodes the CH₂ domain and comprises or consists of a sequence according to SEQ ID NO: 100.
Embodiment 17. The polyribonucleotide of any one of embodiments 7, 10, and 13, wherein the CH₂ domain comprises or consists of an amino acid sequence according to SEQ ID NO: 104.
Embodiment 18. The polyribonucleotide of any one of embodiments 7, 10, 13, and 17, wherein the polyribonucleotide comprises a ribonucleic acid sequence that encodes the CH₂ domain and comprises or consists of a sequence according to SEQ ID NO: 105.
Embodiment 19. The polyribonucleotide of any one of embodiments 7, 10, and 11, wherein the CH₂ domain comprises or consists of an amino acid sequence according to SEQ ID NO: 107.
Embodiment 20. The polyribonucleotide of any one of embodiments 7, 10, 11, and 19, wherein the polyribonucleotide comprises a ribonucleic acid sequence that encodes the CH₂ domain and comprises or consists of a sequence according to SEQ ID NO: 108.
Embodiment 21. The polyribonucleotide of any one of embodiments 7, 10, and 12, wherein the CH₂ domain comprises or consists of an amino acid sequence according to SEQ ID NO: 110.
Embodiment 22. The polyribonucleotide of any one of embodiments 7, 10, 12, and 21, wherein the polyribonucleotide comprises a ribonucleic acid sequence that encodes the CH₂ domain and comprises or consists of a sequence according to SEQ ID NO: 111.
Embodiment 23. The polyribonucleotide of any one of embodiments 6-22, wherein the one or more constant domains comprise a CH₃ domain.
Embodiment 24. The polyribonucleotide of embodiment 23, wherein the CH₃ domain comprises or consists of an amino acid sequence according to SEQ ID NO: 113.
Embodiment 25. The polyribonucleotide of embodiment 23 or 24, wherein the polyribonucleotide comprises a ribonucleic acid sequence that encodes the CH₃ domain and comprises or consists of a sequence according to SEQ ID NO: 114.
Embodiment 26. The polyribonucleotide of embodiment 23, wherein the CH₃ domain comprises or consists of an amino acid sequence according to SEQ ID NO: 116.
Embodiment 27. The polyribonucleotide of embodiment 23 or 26, wherein the polyribonucleotide comprises a ribonucleic acid sequence that encodes the CH₃ domain and comprises or consists of a sequence according to SEQ ID NO: 117.
Embodiment 28. The polyribonucleotide of embodiment 23, wherein the CH₃ domain comprises one or more substitution mutations, wherein the one or more substitution mutations comprise or consist of M428L, N434S, or a combination thereof, and wherein the substitution mutation positions are according to EU numbering.
Embodiment 29. The polyribonucleotide of embodiment 23 or 28, wherein the CH₃ domain comprises or consists of an amino acid sequence according to SEQ ID NO: 116.
Embodiment 30. The polyribonucleotide of any one of embodiments 23, 28, and 29, wherein the polyribonucleotide comprises a ribonucleic acid sequence that encodes the CH₃ domain and comprises or consists of a sequence according to SEQ ID NO: 117.
Embodiment 31. The polyribonucleotide of embodiment 23 or 28, wherein the CH₃ domain comprises or consists of an amino acid sequence according to SEQ ID NO: 134.
Embodiment 32. The polyribonucleotide of any one of embodiments 23, 28, and 31, wherein the polyribonucleotide comprises a ribonucleic acid sequence that encodes the CH₃ domain and comprises or consists of a sequence according to SEQ ID NO: 135.
Embodiment 33. The polyribonucleotide of embodiment 23, wherein the CH₃ domain comprises one or more substitution mutations, wherein the one or more substitution mutations comprise or consist of Y349C, T366S, L368A, Y407V, or a combination thereof, and wherein the substitution mutation positions are according to EU numbering.
Embodiment 34. The polyribonucleotide of embodiment 23, wherein the CH₃ domain comprises one or more substitution mutations, wherein the one or more substitution mutations comprise or consist of Y349C, T366S, L368A, Y407V, M428L, N434S, or a combination thereof, and wherein the substitution mutation positions are according to EU numbering.
Embodiment 35. The polyribonucleotide of embodiment 23, wherein the CH₃ domain comprises one or more substitution mutations, wherein the one or more substitution mutations further comprise or consist of Y349C, T366S, L368A, Y407V, M428L, and N434S, and wherein the substitution mutation positions are according to EU numbering.
Embodiment 36. The polyribonucleotide of any one of embodiments 28 and 33-35, wherein the CH₃ domain comprises or consists of an amino acid sequence according to SEQ ID NO: 122.
Embodiment 37. The polyribonucleotide of any one of embodiments 28 and 33-36, wherein the polyribonucleotide comprises a ribonucleic acid sequence that encodes the CH₃ domain and comprises or consists of a sequence according to SEQ ID NO: 123.
Embodiment 38. The polyribonucleotide of any one of embodiments 28 and 33-35, wherein the CH₃ domain comprises or consists of an amino acid sequence according to SEQ ID NO: 140.
Embodiment 39. The polyribonucleotide of any one of embodiments 28, 33-35, and 38, wherein the polyribonucleotide comprises a ribonucleic acid sequence that encodes the CH₃ domain and comprises or consists of a sequence according to SEQ ID NO: 141.
Embodiment 40. The polyribonucleotide of embodiment 23, wherein the CH₃ domain comprises one or more substitution mutations, wherein the one or more substitution mutations comprise or consist of S354C, T366W, or a combination thereof, and wherein the substitution mutation positions are according to EU numbering.
Embodiment 41. The polyribonucleotide of embodiment 23, wherein the CH₃ domain comprises one or more substitution mutations, wherein the one or more substitution mutations comprise S354C, T366W, M428L, N434S, or a combination thereof, and wherein the substitution mutation positions are according to EU numbering.
Embodiment 42. The polyribonucleotide of embodiment 23, wherein the CH₃ domain comprises one or more substitution mutations, wherein the one or more substitution mutations further comprise S354C, T366W, M428L, and N434S, and wherein the substitution mutation positions are according to EU numbering.
Embodiment 43. The polyribonucleotide of any one of embodiments 28 and 40-42, wherein the CH₃ domain comprises or consists of an amino acid sequence according to SEQ ID NO: 128.
Embodiment 44. The polyribonucleotide of any one of embodiments 28 and 40-43, wherein the polyribonucleotide comprises a ribonucleic acid sequence that encodes the CH₃ domain and comprises or consists of a sequence according to SEQ ID NO: 129.
Embodiment 45. The polyribonucleotide of any one of embodiments 28 and 40-42, wherein the CH₃ domain comprises or consists of an amino acid sequence according to SEQ ID NO: 146.
Embodiment 46. The polyribonucleotide of any one of embodiments 28, 40-42, and 45, wherein the polyribonucleotide comprises a ribonucleic acid sequence that encodes the CH₃ domain and comprises or consists of a sequence according to SEQ ID NO: 147.
Embodiment 47. The polyribonucleotide of any one of embodiments 6-46, wherein the one or more constant domains comprise a hinge domain.
Embodiment 48. The polyribonucleotide of embodiment 47, wherein the hinge domain comprises or consists of an amino acid sequence according to SEQ ID NO: 161.
Embodiment 49. The polyribonucleotide of embodiment 47 or 48, wherein the polyribonucleotide comprises a ribonucleic acid sequence that encodes the hinge domain and comprises or consists of a sequence according to SEQ ID NO: 162.
Embodiment 50. The polyribonucleotide of embodiment 47, wherein the hinge domain comprises or consists of an amino acid sequence according to SEQ ID NO: 164.
Embodiment 51. The polyribonucleotide of embodiment 47 or 50, wherein the polyribonucleotide comprises a ribonucleic acid sequence that encodes the hinge domain and comprises or consists of a sequence according to SEQ ID NO: 165.
Embodiment 52. The polyribonucleotide of embodiment 47, wherein the hinge domain comprises or consists of an amino acid sequence according to SEQ ID NO: 167.
Embodiment 53. The polyribonucleotide of embodiment 47 or 52, wherein the polyribonucleotide comprises a ribonucleic acid sequence that encodes the hinge domain and comprises or consists of a sequence according to SEQ ID NO: 168.
Embodiment 54. The polyribonucleotide of any one of embodiments 6-53, wherein the one or more constant domains comprise a CH₁ domain.
Embodiment 55. The polyribonucleotide of embodiment 54, wherein one or more constant domains comprise, in order, the CH₁ domain, the hinge domain, the CH₂ domain, and the CH₃ domain.
Embodiment 56. The polyribonucleotide of embodiment 54 or 55, wherein the CH₁ domain comprises or consists of an amino acid sequence according to SEQ ID NO: 76.
Embodiment 57. The polyribonucleotide of any one of embodiments 54-56, wherein the polyribonucleotide comprises a ribonucleic acid sequence that encodes the CH₁ domain and comprises or consists of a sequence according to SEQ ID NO: 77.
Embodiment 58. The polyribonucleotide of embodiment 54 or 55, wherein the CH₁ domain comprises or consists of an amino acid sequence according to SEQ ID NO: 81.
Embodiment 59. The polyribonucleotide of any one of embodiments 54-56, wherein the polyribonucleotide comprises a ribonucleic acid sequence that encodes the CH₁ domain and comprises or consists of a sequence according to SEQ ID NO: 82.
Embodiment 60. The polyribonucleotide of embodiment 54 or 55, wherein the CH₁ domain comprises one or more substitution mutations, wherein the one or more substitution mutations comprise or consist of K147E, K213D, or a combination thereof, and wherein the substitution mutation positions are according to EU numbering.
Embodiment 61. The polyribonucleotide of any one of embodiments 54, 55, and 60, wherein the CH₁ domain comprises or consists of an amino acid sequence according to SEQ ID NO: 84.
Embodiment 62. The polyribonucleotide of any one of embodiments 54, 55, 60, and 61, wherein the polyribonucleotide comprises a ribonucleic acid sequence that encodes the CH₁ domain and comprises or consists of a sequence according to SEQ ID NO: 85.
Embodiment 63. The polyribonucleotide of embodiment 54 or 55, wherein the immunoglobulin chain comprises or consists of an amino acid sequence according to SEQ ID NO: 1307.
Embodiment 64. The polyribonucleotide of any one of embodiments 54, 55, and 63, wherein the polyribonucleotide comprises or consists of a ribonucleic acid sequence according to SEQ ID NO: 1306.
Embodiment 65. The polyribonucleotide of embodiment 54 or 55, wherein the immunoglobulin chain comprises or consists of an amino acid sequence according to SEQ ID NO: 1310.
Embodiment 66. The polyribonucleotide of any one of embodiments 54, 55, and 65, wherein the polyribonucleotide comprises or consists of a ribonucleic acid sequence according to SEQ ID NO: 1309.
Embodiment 67. The polyribonucleotide of embodiment 54 or 55, wherein the immunoglobulin chain comprises or consists of an amino acid sequence according to SEQ ID NO: 1316.
Embodiment 68. The polyribonucleotide of any one of embodiments 54, 55, and 67, wherein the polyribonucleotide comprises or consists of a ribonucleic acid sequence according to SEQ ID NO: 1315.
Embodiment 69. The polyribonucleotide of embodiment 54 or 55, wherein the immunoglobulin chain comprises or consists of an amino acid sequence according to SEQ ID NO: 1325.
Embodiment 70. The polyribonucleotide of any one of embodiments 54, 55, and 69, wherein the polyribonucleotide comprises or consists of a ribonucleic acid sequence according to SEQ ID NO: 1324.
Embodiment 71. The polyribonucleotide of any one of embodiments 6-53, wherein the one or more constant domains comprise a light chain constant (CL) domain.
Embodiment 72. The polyribonucleotide of embodiment 71, wherein one or more constant domains comprise, in order, the CL domain, the hinge domain, the CH₂ domain, and the CH₃ domain.
Embodiment 73. The polyribonucleotide of embodiment 71 or 72, wherein the CL domain is a kappa constant domain.
Embodiment 74. The polyribonucleotide of any one of embodiments 71-73, wherein the CL domain comprises or consists of an amino acid sequence according to SEQ ID NO: 149.
Embodiment 75. The polyribonucleotide of any one of embodiments 71-74, wherein the polyribonucleotide comprises a ribonucleic acid sequence that encodes the light chain constant domain and comprises or consists of a sequence according to SEQ ID NO: 150.
Embodiment 76. The polyribonucleotide of any one of embodiments 71-73, wherein the CL domain comprises one or more substitution mutations, wherein the one or more substitution mutations comprise or consist of Q124E, and wherein the substitution mutation positions are according to EU numbering.
Embodiment 77. The polyribonucleotide of any one of embodiments 71-73, wherein the CL domain comprises one or more substitution mutations, wherein the one or more substitution mutations comprise or consist of R108A, T109S, or a combination thereof, and wherein the substitution mutation positions are according to EU numbering.
Embodiment 78. The polyribonucleotide of any one of embodiments 71-73, wherein the CL domain comprises one or more substitution mutations, wherein the one or more substitution mutations comprise or consist of R108A, T109S, Q124E or a combination thereof, and wherein the substitution mutation positions are according to EU numbering.
Embodiment 79. The polyribonucleotide of any one of embodiments 71-73 and 76, wherein the CL domain comprises or consists of an amino acid sequence according to SEQ ID NO: 152.
Embodiment 80. The polyribonucleotide of any one of embodiments 71-73, 76 and 79, wherein the polyribonucleotide comprises a ribonucleic acid sequence that encodes the light chain constant domain and comprises or consists of a sequence according to SEQ ID NO: 153.
Embodiment 81. The polyribonucleotide of embodiment 71 or 72, wherein the immunoglobulin chain comprises or consists of an amino acid sequence according to SEQ ID NO: 1319.
Embodiment 82. The polyribonucleotide of any one of embodiments 71, 72, and 81, wherein the polyribonucleotide comprises or consists of a ribonucleic acid sequence according to SEQ ID NO: 1318.
Embodiment 83. The polyribonucleotide of embodiment 71 or 72, wherein the immunoglobulin chain comprises or consists of an amino acid sequence according to SEQ ID NO: 1331.
Embodiment 84. The polyribonucleotide of any one of embodiments 71, 72, and 83, wherein the polyribonucleotide comprises or consists of a ribonucleic acid sequence according to SEQ ID NO: 1330.
Embodiment 85. The polyribonucleotide of embodiment 71 or 72, wherein the immunoglobulin chain comprises or consists of an amino acid sequence according to SEQ ID NO: 1334.
Embodiment 86. The polyribonucleotide of any one of embodiments 71, 72, and 85, wherein the polyribonucleotide comprises or consists of a ribonucleic acid sequence according to SEQ ID NO: 1333.
Embodiment 87. The polyribonucleotide of embodiment 71 or 72, wherein the immunoglobulin chain comprises or consists of an amino acid sequence according to SEQ ID NO: 1337.
Embodiment 88. The polyribonucleotide of any one of embodiments 71, 72, and 87, wherein the polyribonucleotide comprises or consists of a ribonucleic acid sequence according to SEQ ID NO: 1336.
Embodiment 89. The polyribonucleotide of embodiment 71 or 72, wherein the immunoglobulin chain comprises or consists of an amino acid sequence according to SEQ ID NO: 1340.
Embodiment 90. The polyribonucleotide of any one of embodiments 71, 72, and 89, wherein the polyribonucleotide comprises or consists of a ribonucleic acid sequence according to SEQ ID NO: 1339.
Embodiment 91. A polyribonucleotide encoding an immunoglobulin chain of an antibody agent, wherein the immunoglobulin chain comprises a light chain variable (VL) domain, and the VL domain comprises:
(a) a light chain complementarity determining region (LCDR)1 comprising an amino acid sequence according to SEQ ID NO: 27;
(b) an LCDR2 comprising an amino acid sequence according to SEQ ID NO: 30; and
(c) an LCDR3 comprising an amino acid sequence according to SEQ ID NO: 33.
Embodiment 92. The polyribonucleotide of embodiment 91, wherein the polyribonucleotide comprises a VL domain-encoding sequence, and wherein the VL domain-encoding sequence comprises:
(a) an LCDR1-encoding sequence that comprises or consists of a ribonucleic acid sequence according to SEQ ID NO: 28;
(b) an LCDR2-encoding sequence that comprises or consists of a ribonucleic acid sequence according to SEQ ID NO: 31; and
(c) an LCDR3-encoding sequence that comprises or consists of a ribonucleic acid sequence according to SEQ ID NO: 34.
Embodiment 93. The polyribonucleotide of embodiment 91 or 92, wherein the VL domain comprises or consists of an amino acid sequence according to SEQ ID NO: 43.
Embodiment 94. The polyribonucleotide of any one of embodiments 91-93, wherein the VL domain-encoding sequence comprises or consists of a ribonucleic acid sequence according to SEQ ID NO: 44 or 46.
Embodiment 95. The polyribonucleotide of any one of embodiments 91-94, wherein the immunoglobulin chain further comprises a CL domain, and wherein the VL domain is operably linked to the CL domain.
Embodiment 96. The polyribonucleotide of embodiment 95, wherein the CL domain is a kappa light chain constant domain.
Embodiment 97. The polyribonucleotide of embodiment 95 or 96, wherein the CL domain comprises or consists of an amino acid sequence according to SEQ ID NO: 149.
Embodiment 98. The polyribonucleotide of any one of embodiments 95-97, wherein the polyribonucleotide comprises a ribonucleic acid sequence that encodes the CL domain and comprises or consists of a sequence according to SEQ ID NO: 150.
Embodiment 99. The polyribonucleotide of embodiment 95 or 96, wherein the CL domain comprises one or more substitution mutations, wherein the one or more substitution mutations comprise or consist of E123K, Q124R, or a combination thereof, and wherein the substitution mutation positions are according to EU numbering.
Embodiment 100. The polyribonucleotide of any one of embodiments 95, 96, and 99, wherein the CL domain comprises or consists of an amino acid sequence according to SEQ ID NO: 155.
Embodiment 101. The polyribonucleotide of any one of embodiments 95, 96, 91, and 100, wherein the polyribonucleotide comprises a ribonucleic acid sequence that encodes the light chain constant domain and comprises or consists of a sequence according to SEQ ID NO: 156.
Embodiment 102. The polyribonucleotide of embodiment 95 or 96, wherein the CL domain comprises one or more substitution mutations, wherein the one or more substitution mutations comprise or consist of E123R, Q124K, or a combination thereof, and wherein the substitution mutation positions are according to EU numbering.
Embodiment 103. The polyribonucleotide of any one of embodiments 95, 96, and 102, wherein the CL domain comprises or consists of an amino acid sequence according to SEQ ID NO: 158.
Embodiment 104. The polyribonucleotide of any one of embodiments 95, 96, 102, and 103, wherein the polyribonucleotide comprises a ribonucleic acid sequence that encodes the light chain constant domain and comprises or consists of a sequence according to SEQ ID NO: 159.
Embodiment 105. The polyribonucleotide of embodiment 95 or 96, wherein the immunoglobulin chain comprises or consists of an amino acid sequence according to SEQ ID NO: 1313.
Embodiment 106. The polyribonucleotide of any one of embodiments 87, 88, and 97, wherein the polyribonucleotide comprises or consists of a ribonucleic acid sequence according to SEQ ID NO: 1312.
Embodiment 107. The polyribonucleotide of embodiment 95 or 96, wherein the immunoglobulin chain comprises or consists of an amino acid sequence according to SEQ ID NO: 1328.
Embodiment 108. The polyribonucleotide of any one of embodiments 95, 96, and 107, wherein the polyribonucleotide comprises or consists of a ribonucleic acid sequence according to SEQ ID NO: 1327.
Embodiment 109. The polyribonucleotide of embodiment 95 or 96, wherein the immunoglobulin chain comprises or consists of an amino acid sequence according to SEQ ID NO: 1355.
Embodiment 110. The polyribonucleotide of any one of embodiments 95, 96, and 109, wherein the polyribonucleotide comprises or consists of a ribonucleic acid sequence according to SEQ ID NO: 1354.
Embodiment 111. The polyribonucleotide of any one of embodiments 91-94, wherein the immunoglobulin chain further comprises a CH₁ domain, and wherein the VL domain is operably linked to the CH₁ domain.
Embodiment 112. The polyribonucleotide of embodiment 111, wherein the CH₁ domain comprises or consists of an amino acid sequence according to SEQ ID NO: 76.
Embodiment 113. The polyribonucleotide of embodiment 111 or 112, wherein the polyribonucleotide comprises a ribonucleic acid sequence that encodes the CH₁ domain and comprises or consists of a sequence according to SEQ ID NO: 77.
Embodiment 114. The polyribonucleotide of embodiment 111, wherein the CH₁ domain comprises or consists of an amino acid sequence according to SEQ ID NO: 81.
Embodiment 115. The polyribonucleotide of embodiment 111 or 112, wherein the polyribonucleotide comprises a ribonucleic acid sequence that encodes the CH₁ domain and comprises or consists of a sequence according to SEQ ID NO: 82.
Embodiment 116. The polyribonucleotide of embodiment 111, wherein the immunoglobulin chain comprises or consists of an amino acid sequence according to SEQ ID NO: 1322.
Embodiment 117. The polyribonucleotide of embodiment 111 or 112, wherein the polyribonucleotide comprises or consists of a ribonucleic acid sequence according to SEQ ID NO: 1321.
Embodiment 118. A polyribonucleotide encoding an immunoglobulin chain of an antibody agent, wherein the immunoglobulin chain comprises a heavy chain variable (VH) domain and a light chain variable (VL) domain,
wherein the VH domain comprises:
(a) an HCDR1 comprising an amino acid sequence according to SEQ ID NO: 18;
(b) an HCDR2 comprising an amino acid sequence according to SEQ ID NO: 21; and
(c) an HCDR3 comprising an amino acid sequence according to SEQ ID NO: 24; and
wherein the VL domain comprises:
(d) an LCDR1 comprising an amino acid sequence according to SEQ ID NO: 27;
(e) an LCDR2 comprising an amino acid sequence according to SEQ ID NO: 30; and
(f) an LCDR3 comprising an amino acid sequence according to SEQ ID NO: 33.
Embodiment 119. The polyribonucleotide of embodiment 118, wherein the polyribonucleotide comprises a VH domain-encoding sequence and a VL domain-encoding sequence,
wherein the VH domain-encoding sequence comprises:
(a) an HCDR1-encoding sequence that comprises or consists of the ribonucleic acid sequence according to SEQ ID NO: 19;
(b) the HCDR2-encoding sequence that comprises or consists of the ribonucleic acid sequence according to SEQ ID NO: 22; and
(c) the HCDR3-encoding sequence that comprises or consists of the ribonucleic acid sequence according to SEQ ID NO: 25; and
wherein the VL domain-encoding sequence comprises:
(d) an LCDR1-encoding sequence that comprises or consists of a ribonucleic acid sequence according to SEQ ID NO: 28;
(e) an LCDR2-encoding sequence that comprises or consists of a ribonucleic acid sequence according to SEQ ID NO: 31; and
(f) an LCDR3-encoding sequence that comprises or consists of a ribonucleic acid sequence according to SEQ ID NO: 34.
Embodiment 120. The polyribonucleotide of embodiment 118 or 119, wherein the immunoglobulin chain comprises a single chain fragment variable (scFv), and the scFv comprises the VH domain, a linker, and the VL domain.
Embodiment 121. The polyribonucleotide of embodiment 120, wherein the scFv comprises, in order:
(i) the VH domain comprising or consisting of an amino acid sequence according to SEQ ID NO: 36,
(ii) the linker, and
(iii) the VL domain comprising or consisting of an amino acid sequence according to SEQ ID NO: 43.
Embodiment 122. The polyribonucleotide of embodiment 120, wherein the scFv comprises, in order:
(i) the VL domain comprising or consisting of an amino acid sequence according to SEQ ID NO: 43,
(ii) the linker, and
(iii) the VH domain comprising or consisting of an amino acid sequence according to SEQ ID NO: 36.
Embodiment 123. The polyribonucleotide of any one of embodiments 120-122, wherein the immunoglobulin chain comprises a hinge domain following the scFv.
Embodiment 124. The polyribonucleotide of embodiment 123, wherein the hinge domain comprises or consists of an amino acid sequence according to SEQ ID NO: 161.
Embodiment 125. The polyribonucleotide of embodiment 123 or 124, wherein the polyribonucleotide comprises a ribonucleic acid sequence that encodes the hinge domain and comprises or consists of a sequence according to SEQ ID NO: 162.
Embodiment 126. The polyribonucleotide of embodiment 123, wherein the hinge domain comprises or consists of an amino acid sequence according to SEQ ID NO: 164.
Embodiment 127. The polyribonucleotide of embodiment 123 or 124, wherein the polyribonucleotide comprises a ribonucleic acid sequence that encodes the hinge domain and comprises or consists of a sequence according to SEQ ID NO: 165.
Embodiment 128. The polyribonucleotide of embodiment 123, wherein the hinge domain comprises or consists of an amino acid sequence according to SEQ ID NO: 167.
Embodiment 129. The polyribonucleotide of embodiment 123 or 124, wherein the polyribonucleotide comprises a ribonucleic acid sequence that encodes the hinge domain and comprises or consists of a sequence according to SEQ ID NO: 168.
Embodiment 130. The polyribonucleotide of any one of embodiments 118-129, wherein the immunoglobulin chain comprises one or more constant domains, and wherein the scFv is operably linked to the one or more constant domains.
Embodiment 131. The polyribonucleotide of any one of embodiments 123-130, wherein the immunoglobulin chain comprises one or more constant domain, and the hinge domain is between the scFv and the one or more constant domains.
Embodiment 132. The polyribonucleotide of embodiment 122 or 123, wherein the one or more constant domains comprise a CH₂ domain.
Embodiment 133. The polyribonucleotide of embodiment 132, wherein the CH₂ domain comprises or consists of a sequence according to SEQ ID NO: 96.
Embodiment 134. The polyribonucleotide of embodiment 132 or 133, wherein the polyribonucleotide comprises a ribonucleic acid sequence that encodes the CH₂ domain and comprises or consists of a sequence according to SEQ ID NO: 97.
Embodiment 135. The polyribonucleotide of embodiment 132, wherein the CH₂ domain comprises one or more substitution mutations, wherein the one or more substitution mutations comprise or consist of G236A, A330L, I332E, or a combination thereof, and wherein the substitution mutation positions are according to EU numbering.
Embodiment 136. The polyribonucleotide of embodiment 132 or 135, wherein the CH₂ domain comprises one or more substitution mutations, wherein the one or more substitution mutations comprise or consist of G236A, and wherein the substitution mutation positions are according to EU numbering.
Embodiment 137. The polyribonucleotide of embodiment 132 or 135, wherein the CH₂ domain comprises one or more substitution mutations, wherein the one or more substitution mutations comprise or consist of I332E, and wherein the substitution mutation positions are according to EU numbering.
Embodiment 138. The polyribonucleotide of embodiment 132 or 135, wherein the CH₂ domain comprises one or more substitution mutations, wherein the one or more substitution mutations comprise or consist of G236A and I332E, and wherein the substitution mutation positions are according to EU numbering.
Embodiment 139. The polyribonucleotide of embodiment 132 or 135, wherein the CH₂ domain comprises one or more substitution mutations, wherein the one or more substitution mutations comprise or consist of G236A, A330L, and I332E, and wherein the substitution mutation positions are according to EU numbering.
Embodiment 140. The polyribonucleotide of any one of embodiments 132, 135, and 139, wherein the CH₂ domain comprises or consists of an amino acid sequence according to SEQ ID NO: 99 or 102.
Embodiment 141. The polyribonucleotide of any one of embodiments 132, 135, 139, and 140, wherein the polyribonucleotide comprises a ribonucleic acid sequence that encodes the CH₂ domain and comprises or consists of a sequence according to SEQ ID NO: 100 or 103.
Embodiment 142. The polyribonucleotide of any one of embodiments 132, 135, and 138, wherein the CH₂ domain comprises or consists of an amino acid sequence according to SEQ ID NO: 104.
Embodiment 143. The polyribonucleotide of any one of embodiments 132, 135, 130 and 134, wherein the polyribonucleotide comprises a ribonucleic acid sequence that encodes the CH₂ domain and comprises or consists of a sequence according to SEQ ID NO: 105.
Embodiment 144. The polyribonucleotide of any one of embodiments 132, 135, and 136, wherein the CH₂ domain comprises or consists of an amino acid sequence according to SEQ ID NO: 107.
Embodiment 145. The polyribonucleotide of any one of embodiments 132, 135, 136, and 144, wherein the polyribonucleotide comprises a ribonucleic acid sequence that encodes the CH₂ domain and comprises or consists of a sequence according to SEQ ID NO: 108.
Embodiment 146. The polyribonucleotide of any one of embodiments 132, 135, and 137, wherein the CH₂ domain comprises or consists of an amino acid sequence according to SEQ ID NO: 110.
Embodiment 147. The polyribonucleotide of any one of embodiments 132, 135, 137, and 146, wherein the polyribonucleotide comprises a ribonucleic acid sequence that encodes the CH₂ domain and comprises or consists of a sequence according to SEQ ID NO: 111.
Embodiment 148. The polyribonucleotide of any one of embodiments 130-147, wherein the one or more constant domains comprise a CH₃ domain.
Embodiment 149. The polyribonucleotide of embodiment 148, wherein the CH₃ domain comprises or consists of a sequence according to SEQ ID NO: 113.
Embodiment 150. The polyribonucleotide of embodiment 148 or 149, wherein the polyribonucleotide comprises a ribonucleic acid sequence that encodes the CH₃ domain and comprises or consists of a sequence according to SEQ ID NO: 114.
Embodiment 151. The polyribonucleotide of embodiment 148, wherein the CH₃ domain comprises or consists of an amino acid sequence according to SEQ ID NO: 131.
Embodiment 152. The polyribonucleotide of embodiment 148 or 151, wherein the polyribonucleotide comprises a ribonucleic acid sequence that encodes the CH₃ domain and comprises or consists of a sequence according to SEQ ID NO: 132.
Embodiment 153. The polyribonucleotide of embodiment 148, wherein the CH₃ domain comprises one or more substitution mutations, wherein the one or more substitution mutations comprise or consist of M428L, N434S, or a combination thereof, and wherein the substitution mutation positions are according to EU numbering.
Embodiment 154. The polyribonucleotide of embodiment 148 or 153, wherein the CH₃ domain comprises or consists of an amino acid sequence according to SEQ ID NO: 116.
Embodiment 155. The polyribonucleotide of any one of embodiments 148, 153 and 154, wherein the polyribonucleotide comprises a ribonucleic acid sequence that encodes the CH₃ domain and comprises or consists of a sequence according to SEQ ID NO: 117.
Embodiment 156. The polyribonucleotide of embodiment 148 or 153, wherein the CH₃ domain comprises or consists of an amino acid sequence according to SEQ ID NO: 134.
Embodiment 157. The polyribonucleotide of any one of embodiments 148, 153 and 156, wherein the polyribonucleotide comprises a ribonucleic acid sequence that encodes the CH₃ domain and comprises or consists of a sequence according to SEQ ID NO: 135.
Embodiment 158. The polyribonucleotide of embodiment 148, wherein the CH₃ domain comprises one or more substitution mutations, wherein the one or more substitution mutations comprise or consist of Y349C, T366S, L368A, Y407V, or a combination thereof, and wherein the substitution mutation positions are according to EU numbering.
Embodiment 159. The polyribonucleotide of any one of embodiments 148, 153, and 148, wherein the CH₃ domain comprises one or more substitution mutations, wherein the one or more substitution mutations comprise or consist of Y349C, T366S, L368A, Y407V, M428L, N434S, or a combination thereof, and wherein the substitution mutation positions are according to EU numbering.
Embodiment 160. The polyribonucleotide of any one of embodiments 148, 153, 158, and 159, wherein the CH₃ domain comprises one or more substitution mutations, wherein the one or more substitution mutations comprise or consist of Y349C, T366S, L368A, Y407V, M428L, and N434S, and wherein the substitution mutation positions are according to EU numbering.
Embodiment 161. The polyribonucleotide of any one of embodiments 148, 153, and 158-160, wherein the CH₃ domain comprises or consists of an amino acid sequence according to SEQ ID NO: 122.
Embodiment 162. The polyribonucleotide of any one of embodiments 148, 153, and 158-161, wherein the polyribonucleotide comprises a ribonucleic acid sequence that encodes the CH₃ domain and comprises or consists of a sequence according to SEQ ID NO: 123.
Embodiment 163. The polyribonucleotide of any one of embodiments 148, 153, and 158-160, wherein the CH₃ domain comprises or consists of an amino acid sequence according to SEQ ID NO: 140.
Embodiment 164. The polyribonucleotide of any one of embodiments 148, 153, and 155, 158-160 wherein the polyribonucleotide comprises a ribonucleic acid sequence that encodes the CH₃ domain and comprises or consists of a sequence according to SEQ ID NO: 141.
Embodiment 165. The polyribonucleotide of embodiment 148, wherein the CH₃ domain comprises one or more substitution mutations, wherein the one or more substitution mutations comprise or consist of S354C, T366W, or a combination thereof, and wherein the substitution mutation positions are according to EU numbering.
Embodiment 166. The polyribonucleotide of any one of embodiments 148, 153, and 165, wherein the CH₃ domain comprises one or more substitution mutations, wherein the one or more substitution mutations comprise S354C, T366W, M428L, N434S, or a combination thereof, and wherein the substitution mutation positions are according to EU numbering.
Embodiment 167. The polyribonucleotide of any one of embodiments 148, 153, 165, and 166, wherein the CH₃ domain comprises one or more substitution mutations, wherein the one or more substitution mutations further comprise S354C, T366W, M428L, and N434S, and wherein the substitution mutation positions are according to EU numbering.
Embodiment 168. The polyribonucleotide of any one of embodiments 148, 153, and 165-167, wherein the CH₃ domain comprises or consists of an amino acid sequence according to SEQ ID NO: 128.
Embodiment 169. The polyribonucleotide of any one of embodiments 148, 153, and 165-168, wherein the polyribonucleotide comprises a ribonucleic acid sequence that encodes the CH₃ domain and comprises or consists of a sequence according to SEQ ID NO: 129.
Embodiment 170. The polyribonucleotide of any one of embodiments 148, 153, and 165-167, wherein the CH₃ domain comprises or consists of an amino acid sequence according to SEQ ID NO: 146.
Embodiment 171. The polyribonucleotide of embodiment any one of embodiments 148, 153, 165-167, and 170, wherein the polyribonucleotide comprises a ribonucleic acid sequence that encodes the CH₃ domain and comprises or consists of a sequence according to SEQ ID NO: 147.
Embodiment 172. The polyribonucleotide of any one of embodiments 121-171, wherein the linker comprises an amino acid sequence according to SEQ ID NO: 48.
Embodiment 173. The polyribonucleotide of any one of embodiments 121-172, wherein the polyribonucleotide comprises a ribonucleic acid sequence that encodes the linker and comprises or consists of a sequence according to SEQ ID NO: 49, 51, 53, 55, or 57.
Embodiment 174. The polyribonucleotide of any one of embodiments 121-171, wherein the linker comprises an amino acid sequence according to SEQ ID NO: 59.
Embodiment 175. The polyribonucleotide of any one of embodiments 121-171 and 174, wherein the polyribonucleotide comprises a ribonucleic acid sequence that encodes the linker and comprises or consists of a sequence according to SEQ ID NO: 60 or 62.
Embodiment 176. The polyribonucleotide of embodiment 120, wherein the immunoglobulin chain comprises or consists of a sequence according to SEQ ID NO: 1343.
Embodiment 177. The polyribonucleotide of embodiment 120 or 176, wherein the polyribonucleotide comprises or consists of a ribonucleic acid sequence according to SEQ ID NO: 1342.
Embodiment 178. The polyribonucleotide of embodiment 120, wherein the immunoglobulin chain comprises or consists of a sequence according to SEQ ID NO: 1346.
Embodiment 179. The polyribonucleotide of embodiment 120 or 178, wherein the polyribonucleotide comprises or consists of a ribonucleic acid sequence according to SEQ ID NO: 1345.
Embodiment 180. The polyribonucleotide of embodiment 120, wherein the immunoglobulin chain comprises or consists of a sequence according to SEQ ID NO: 1349.
Embodiment 181. The polyribonucleotide of embodiment 120 or 180, wherein the polyribonucleotide comprises or consists of a ribonucleic acid sequence according to SEQ ID NO: 1348.
Embodiment 182. The polyribonucleotide of embodiment 120, wherein the immunoglobulin chain comprises or consists of a sequence according to SEQ ID NO: 1352.
Embodiment 183. The polyribonucleotide of embodiment 120 or 182, wherein the polyribonucleotide comprises or consists of a ribonucleic acid sequence according to SEQ ID NO: 1351.
Embodiment 184. The polyribonucleotide of embodiment 120, wherein the immunoglobulin chain comprises or consists of a sequence according to SEQ ID NO: 1358, 1361, 1364, 1367, 1370, 1373, 1376, 1379, 1382, 1385, 1388, 1391, 1394, 1397, 1400, 1403, 1406, 1409, 1412, 1415, 1418, 1421, 1424, 1427, 1430, 1433, 1436, 1439, 1442, 1445, 1448, 1451, 1454, 1457, or 1460.
Embodiment 185. The polyribonucleotide of embodiment 120 or 184, wherein the polyribonucleotide comprises or consists of a ribonucleic acid sequence according to SEQ ID NO: 1357, 1360, 1363, 1366, 1369, 1372, 1375, 1378, 1381, 1384, 1387, 1390, 1393, 1396, 1399, 1402, 1405, 1408, 1411, 1414, 1417, 1420, 1423, 1426, 1429, 1432, 1435, 1438, 1441, 1444, 1447, 1450, 1453, 1456, or 1459.
Embodiment 186. The polyribonucleotide of any one of embodiments 1-185, wherein the polyribonucleotide comprises a ribonucleic acid sequence encoding a secretion signal.
Embodiment 187. The polyribonucleotide of embodiment 186, wherein the secretion signal comprises a ribonucleic acid sequence according to SEQ ID NO: 2, 4, 6, or 8.
Embodiment 188. The polyribonucleotide of any one of embodiments 1-187, wherein the polyribonucleotide comprises one or more non-coding sequence elements.
Embodiment 189. The polyribonucleotide of embodiment 188, wherein the one or more non-coding sequence elements enhances RNA stability and/or translation efficiency.
Embodiment 190. The polyribonucleotide of embodiment 188 or 189, wherein the one or more non-coding sequence elements comprise a 3′ untranslated region (UTR), a 5′ UTR, a 5′-cap, a polyadenine (polyA) tail, or combination thereof.
Embodiment 191. The polyribonucleotide of embodiment 190, wherein the polyA tail is or comprises a modified polyA sequence, preferably an interrupted polyA tail.
Embodiment 192. The polyribonucleotide of embodiment 191, wherein the polyA tail comprises or consists of a sequence that is at least 90% identical to SEQ ID NO: 16.
Embodiment 193. The polyribonucleotide of any one of embodiments 190-192, wherein the 3′ UTR comprises or consists of a nucleic acid sequence that is at least 90% identical to SEQ ID NO: 14.
Embodiment 194. The polyribonucleotide of any one of embodiments 190-193, wherein the 5′ UTR comprises or consists of a nucleic acid sequence that is at least 90% identical to SEQ ID NO: 10.
Embodiment 195. The polyribonucleotide of any one of embodiments 190-192, wherein the 5′-cap is (m₂^{7,3′}-O)Gppp(m^{2′-O})ApG.
Embodiment 196. The polyribonucleotide of any one of embodiments 1-195, wherein the polyribonucleotide comprises one or more modified ribonucleotides.
Embodiment 197. The polyribonucleotide of embodiment 196, wherein the one or more modified ribonucleotides comprise pseudouridine.
Embodiment 198. The polyribonucleotide of any one of embodiments 1-5, 91-94 and 118-122,
wherein the immunoglobulin chain further comprises:
(a) a CH₁ domain, a CH₂ domain, and a CH₃ domain,
(b) a CL domain, a CH₂ domain, and a CH₃ domain,
(c) a CL domain, or
(d) a CH₁ domain,
and wherein:
(i) the polyribonucleotide comprises a ribonucleic acid sequence that encodes a CH₂ domain, and the CH₂ domain comprises one or more substitution mutations, wherein the one or more substitution mutations comprise or consist of G236A, A330L, I332E, or a combination thereof,
(ii) the polyribonucleotide comprises a ribonucleic acid sequence that encodes a CH₃ domain, and the CH₃ domain comprises one or more substitution mutations, wherein the one or more substitution mutations comprise or consist of Y349C, S354C, T366S, T366W, L368A, Y407V, M428L, N434S, or a combination thereof,
(iii) the polyribonucleotide comprises a ribonucleic acid sequence that encodes a CH₁ domain, and the CH₁ domain comprises one or more substitution mutations, wherein the one or more substitution mutations comprise or consist of K147E, K213D, or a combination thereof,
(iv) the polyribonucleotide comprises a ribonucleic acid sequence that encodes a CL domain, and the CL domain comprises one or more substitution mutations, wherein the one or more substitution mutations comprise or consist of R108A, T109S, E123K, E123R, Q124E, Q124K, Q124R, or a combination thereof, or
(v) a combination thereof; wherein the substitution mutation positions are according to EU numbering.
Embodiment 199. The polyribonucleotide of any one of embodiments 1-4, wherein the polyribonucleotide comprises or consists of a ribonucleic acid sequence according to SEQ ID NO: 850.
Embodiment 200. The polyribonucleotide of any one of embodiments 1-4, wherein the polyribonucleotide comprises or consists of a ribonucleic acid sequence according to SEQ ID NO: 851.
Embodiment 201. The polyribonucleotide of any one of embodiments 1-4, wherein the polyribonucleotide comprises or consists of a ribonucleic acid sequence according to SEQ ID NO: 853.
Embodiment 202. The polyribonucleotide of any one of embodiments 1-4, wherein the polyribonucleotide comprises or consists of a ribonucleic acid sequence according to SEQ ID NO: 900.
Embodiment 203. The polyribonucleotide of any one of embodiments 1-4, wherein the polyribonucleotide comprises or consists of a ribonucleic acid sequence according to SEQ ID NO: 901.
Embodiment 204. The polyribonucleotide of any one of embodiments 1-4, wherein the polyribonucleotide comprises or consists of a ribonucleic acid sequence according to SEQ ID NO: 905.
Embodiment 205. The polyribonucleotide of any one of embodiments 1-4, wherein the polyribonucleotide comprises or consists of a ribonucleic acid sequence according to SEQ ID NO: 906.
Embodiment 206. The polyribonucleotide of any one of embodiments 1-4, wherein the polyribonucleotide comprises or consists of a ribonucleic acid sequence according to SEQ ID NO: 910.
Embodiment 207. The polyribonucleotide of any one of embodiments 1-4, wherein the polyribonucleotide comprises or consists of a ribonucleic acid sequence according to SEQ ID NO: 950.
Embodiment 208. The polyribonucleotide of any one of embodiments 91-94, wherein the polyribonucleotide comprises or consists of a ribonucleic acid sequence according to SEQ ID NO: 898.
Embodiment 209. The polyribonucleotide of any one of embodiments 91-94, wherein the polyribonucleotide comprises or consists of a ribonucleic acid sequence according to SEQ ID NO: 947.
Embodiment 210. The polyribonucleotide of any one of embodiments 91-94, wherein the polyribonucleotide comprises or consists of a ribonucleic acid sequence according to SEQ ID NO: 997.
Embodiment 211. The polyribonucleotide of any one of embodiments 118-122, wherein the polyribonucleotide comprises or consists of a ribonucleic acid sequence according to SEQ ID NO: 1000.
Embodiment 212. The polyribonucleotide of any one of embodiments 118-122, wherein the polyribonucleotide comprises or consists of a ribonucleic acid sequence according to SEQ ID NO: 1048.
Embodiment 213. The polyribonucleotide of any one of embodiments 118-122, wherein the polyribonucleotide comprises or consists of a ribonucleic acid sequence according to SEQ ID NO: 1096.
Embodiment 214. The polyribonucleotide of any one of embodiments 118-112, wherein the polyribonucleotide comprises or consists of a ribonucleic acid sequence according to SEQ ID NO: 1144.
Embodiment 215. A polyribonucleotide comprising or consisting of a ribonucleic acid sequence according to any one of SEQ ID NO: 850-1190.
Embodiment 216. The polyribonucleotide of any one of embodiments 1-215, wherein the polyribonucleotide is a non-natural polyribonucleotide.
Embodiment 217. The polyribonucleotide of any one of embodiments 1-216, wherein the polyribonucleotide is an engineered polyribonucleotide.
Embodiment 218. The polyribonucleotide of any one of embodiments 1-217, wherein the polyribonucleotide is an isolated polyribonucleotide.
Embodiment 219. A plurality of polyribonucleotides comprising three or more different polyribonucleotides,
wherein each polyribonucleotide of the three or more different polyribonucleotides encodes an immunoglobulin chain,
wherein each of the three or more different polyribonucleotides encode a different immunoglobulin chain,
wherein at least one of the polyribonucleotides of the plurality is a polyribonucleotide of any one of embodiments 1-218, and
wherein, when immunoglobulin chains are expressed from the three or more different polyribonucleotides in a cell, the immunoglobulin chains form:
(a) at least two different antibody agents; and
(b) at most (2x+n+1)/2 different antibody agents, wherein x is equal to the number of polyribonucleotides in the three or more polyribonucleotides that encode an immunoglobulin chain comprising an scFV, and n is equal to the number of polyribonucleotides in the three or more polyribonucleotides minus x.
Embodiment 220. A composition comprising one or more polyribonucleotides of any one of embodiments 1-218.
Embodiment 221. The composition of embodiment 220, wherein the one or more polyribonucleotides comprise or consist of:
(a) the polyribonucleotide of embodiment 63 or 64; and
(b) the polyribonucleotide of embodiment 105 or 106.
Embodiment 222. The composition of embodiment 220, wherein the one or more polyribonucleotides comprise or consist of:
(a) the polyribonucleotide of embodiment 65 or 66; and
(b) the polyribonucleotide of embodiment 105 or 106.
Embodiment 223. The composition of embodiment 220, wherein the one or more polyribonucleotides comprise or consist of:
(a) the polyribonucleotide of embodiment 67 or 68; and
(b) the polyribonucleotide of embodiment 105 or 106.
Embodiment 224. The composition of embodiment 220, wherein the one or more polyribonucleotides comprise or consist of:
(a) the polyribonucleotide of embodiment 81 or 82; and
(b) the polyribonucleotide of embodiment 116 or 117.
Embodiment 225. The composition of embodiment 220, wherein the one or more polyribonucleotides comprise or consist of:
(a) the polyribonucleotide of embodiment 83 or 84; and
(b) the polyribonucleotide of embodiment 116 or 117.
Embodiment 226. The composition of embodiment 220, wherein the one or more polyribonucleotides comprise or consist of:
(a) the polyribonucleotide of embodiment 85 or 86; and
(b) the polyribonucleotide of embodiment 116 or 117.
Embodiment 227. The composition of embodiment 220, wherein the one or more polyribonucleotides comprise or consist of:
(a) the polyribonucleotide of embodiment 87 or 88; and
(b) the polyribonucleotide of embodiment 116 or 117.
Embodiment 228. The composition of embodiment 220, wherein the one or more polyribonucleotides comprise or consist of:
(a) the polyribonucleotide of embodiment 89 or 90; and
(b) the polyribonucleotide of embodiment 116 or 117.
Embodiment 229. The composition of embodiment 220, wherein the one or more polyribonucleotides comprise or consist of:
(a) the polyribonucleotide of embodiment 69 or 70; and
(b) the polyribonucleotide of embodiment 107 or 108.
Embodiment 230. The composition of embodiment 220, wherein the one or more polyribonucleotides comprise or consist of:
(a) the polyribonucleotide of embodiment 69 or 70; and
(b) the polyribonucleotide of embodiment 109 or 110.
Embodiment 231. The composition of any one of embodiments 220-230, wherein the composition further comprises lipid nanoparticles, polyplexes (PLX), lipidated polyplexes (LPLX), or liposomes, wherein the one or more polyribonucleotides are fully or partially encapsulated within the lipid nanoparticles, polyplexes (PLX), lipidated polyplexes (LPLX), or liposomes.
Embodiment 232. The composition of any one of embodiments 220-231, wherein the composition further comprises lipid nanoparticles, wherein the one or more polyribonucleotides are encapsulated within the lipid nanoparticles.
Embodiment 233. The composition of embodiment 232, wherein the lipid nanoparticles target liver cells.
Embodiment 234. The composition of embodiment 232, wherein the lipid nanoparticles target secondary lymphoid organ cells.
Embodiment 235. The composition of embodiment 232, wherein the lipid nanoparticles target lung cells.
Embodiment 236. The composition of embodiment any one of embodiments 231-235, wherein the lipid nanoparticles are cationic lipid nanoparticles.
Embodiment 237. The composition of any one of embodiments 231-236, wherein the lipid nanoparticles each comprise:
(a) a polymer-conjugated lipid;
(b) a cationic lipid; and
(c) one or more neutral lipids.
Embodiment 238. The composition of embodiment 237, wherein the polymer-conjugated lipid comprises a PEG-conjugated lipid.
Embodiment 239. The composition of embodiment 237 or 238, wherein the polymer-conjugated lipid comprises 2-[(polyethylene glycol)-2000]-N,N-ditetradecylacetamide.
Embodiment 240. The composition of any one of embodiments 237-239, wherein the one or more neutral lipids comprise 1,2-Distearoyl-sn-glycero-3-phosphocholine (DPSC).
Embodiment 241. The composition of any one of embodiments 237-240, wherein the one or more neutral lipids comprise cholesterol.
Embodiment 242. The composition of any one of embodiments 237-241, wherein the cationic lipid comprises ((3-hydroxypropyl)azanediyl)bis(nonane-9,1-diyl) bis(2-butyloctanoate).
Embodiment 243. The composition of any one of embodiments 237-242, wherein the lipid nanoparticles each comprise:
(a) 2-[(polyethylene glycol)-2000]-N,N-ditetradecylacetamide;
(b) DPSC;
(c) cholesterol; and
(d) ((3-hydroxypropyl)azanediyl)bis(nonane-9,1-diyl) bis(2-butyloctanoate).
Embodiment 244. The composition of any one of embodiments 237-243, wherein the lipid nanoparticles comprise:
(a) the polymer-conjugated lipid at about 1-2.5 mol % of the total lipids;
(b) the cationic lipid at 35-65 mol % of the total lipids; and
(c) the one or more neutral lipids are present in 35-65 mol % of the total lipids.
Embodiment 245. The composition of any one of embodiments 220-244, wherein the lipid nanoparticles have an average diameter of about 50-150 nm.
Embodiment 246. A pharmaceutical composition comprising the composition of any one of embodiments 220-245 and at least one pharmaceutically acceptable excipient.
Embodiment 247. The pharmaceutical composition of embodiment 246, wherein the pharmaceutical comprises a cryoprotectant.
Embodiment 248. The pharmaceutical composition of embodiment 246 or 247, wherein the pharmaceutical comprises an aqueous buffered solution.
Embodiment 249. A method comprising administering the pharmaceutical composition of any one of embodiments 246-248 to a subject.
Embodiment 250. The pharmaceutical composition of any one of embodiments 246-248 for use in the treatment of HIV comprising administering the pharmaceutical composition to a subject.
Embodiment 251. The pharmaceutical composition of any one of embodiments 246-248 for use in the prevention of HIV comprising administering the pharmaceutical composition to a subject.
Embodiment 252. The method of embodiment 249 or the pharmaceutical composition for use according to embodiment 250 or 251, wherein administering the pharmaceutical composition to the subject results in expression in the subject of:
(a) the immunoglobulin chain of antibody agent;
(b) the antibody agent; or
(c) both.
Embodiment 253. The method of embodiment 249 or 252 or the pharmaceutical composition for use according to any one of embodiments 250-252, wherein the immunoglobulin chain of antibody agent, the antibody agent, or both is expressed in the subject at a titer of:
(a) at least 1 μg/ml in plasma; or
(b) at least 1 μg/ml in serum.
Embodiment 254. The method of any one of embodiments 249, 252, and 253 or the pharmaceutical composition for use according to any one of embodiments 261-264, wherein the antibody agent exhibits a geometric mean IC50 of five neutralized strains of less than 0.3 μg/ml against the neutralized strains of a global reference panel when tested in the TZM-bl cell pseudovirus neutralization assay at antibody agent concentrations up to 25 μg/ml.
Embodiment 255. The method of any one of embodiments 249 and 252-254 or the pharmaceutical composition for use according to any one of embodiments 250-254, wherein the antibody agent is capable of neutralizing one or more HIV strains when tested in the TZM-bl cell pseudovirus neutralization assay at antibody agent concentrations up to 25 μg/ml.
Embodiment 256. The method of any one of embodiments 249 and 252-255 or the pharmaceutical composition for use according to any one of embodiments 250-255, wherein the antibody agent is capable of neutralizing one or more HIV strains at a level that is within 3-fold of a level of an equivalent amount of recombinant benchmark antibody.
Embodiment 257. The method or the pharmaceutical composition for use of embodiment 256, wherein the recombinant benchmark antibody is an unmodified wild-type IgG antibody comprising the same HCDR1, HCDR2, HCDR2, LCDR1, LCDR2, and LCDR3 as the antibody agent.
Embodiment 258. The method of any one of embodiments 249 and 252-257 or the pharmaceutical for use according to any one of embodiments 250-257, wherein administering the pharmaceutical composition to the subject comprises administering one or more doses of the pharmaceutical composition to the subject.
Embodiment 259. The method or the pharmaceutical composition for use of embodiment 258, wherein the one or more doses of the pharmaceutical composition are administered to the subject weekly.
Embodiment 260. The method or the pharmaceutical composition for use of embodiment 258, wherein the one or more doses of the pharmaceutical composition are administered to the subject bi-weekly.
Embodiment 261. The method of any one of embodiments 249 and 252-260 or the pharmaceutical composition for use according to any one of embodiments 250-260, wherein the pharmaceutical composition is administered intravenously.
Embodiment 262. The method of any one of embodiments 249 and 252-260 or the pharmaceutical composition for use according to any one of embodiments 250-260, wherein the pharmaceutical composition is administered intramuscularly.
Embodiment 263. The method of any one of embodiments 249 and 252-260 or the pharmaceutical composition for use according to any one of embodiments 250-260, wherein the pharmaceutical composition is administered subcutaneously.
Embodiment 264. The method of any one of embodiments 249 and 252-263 or the pharmaceutical composition for use according to any one of embodiments 250-263, wherein the subject has or is at risk of developing an HIV infection.
Embodiment 265. The method of any one of embodiments 249 and 252-264, wherein the method is a method of treating an HIV infection.
Embodiment 266. The method of any one of embodiments 249 and 252-264, wherein the method is a method of preventing an HIV infection.
Embodiment 267. Use of the composition of any one of embodiments 220-245 or the pharmaceutical composition of any one of embodiments 246-248 for the treatment of HIV in a subject.
Embodiment 268. Use of the composition of any one of embodiments 220-245 or the pharmaceutical composition of any one of embodiments 246-248 for the prevention of HIV in a subject.
Embodiment 269. The use of embodiment 267 or 268, wherein the subject has or is at risk of developing an HIV infection.
Embodiment 270. A method of producing an antibody agent comprising administering to cells the composition of any one of embodiments 220-245 or the pharmaceutical composition of any one of embodiments 246-248 so that the cells express and secrete the antibody agent.
Embodiment 271. The method of embodiment 270, wherein the cells are liver cells.
Embodiment 272. The method of embodiment 270 or 271, wherein the cells are in a subject.
Embodiment 273. The method of embodiment 270 or 271, wherein the cells are ex vivo cells.
Embodiment 274. The method of embodiment 272, wherein the antibody agent is produced at a therapeutically relevant plasma concentration or a therapeutically relevant serum concentration.
Embodiment 275. The method of embodiment 274, wherein the therapeutically relevant plasma concentration or the therapeutically relevant serum concentration is at least 1 μg/ml.
Embodiment 276. A method comprising a step of:
determining one or more features of an antibody agent expressed from the polyribonucleotide of any one of embodiments 1-218, the composition of any one of embodiments 220-245, or the pharmaceutical composition of any one of embodiments 246-248 introduced into cells, wherein the one or more features comprises: (i) protein expression level of the antibody agent; (ii) binding specificity of the antibody agent to the Apex binding site of HIV; (iii) efficacy of the antibody agent to mediate target cell death through antibody-dependent cellular cytotoxicity (ADCC); and (iv) efficacy of the antibody agent to mediate target cell death through complement dependent cytotoxicity (CDC).
Embodiment 277. A method comprising the steps of:
contacting cells with the polyribonucleotide of any one of embodiments 1-218, the composition of any one of embodiments 220-245, or the pharmaceutical composition of any one of embodiments 246-245 introduced into cells; and
detecting the antibody agent produced by the cells.
Embodiment 278. The method of embodiment 276, the method further comprising steps of:
contacting cells with the polyribonucleotide of any one of embodiments 1-218, the composition of any one of embodiments 220-245, or the pharmaceutical composition of any one of embodiments 246-248 introduced into cells; and
detecting the antibody agent produced by the cells.
Embodiment 279. The method of embodiment 277 or 278, wherein the cells are liver cells.
Embodiment 280. The method of any one of embodiments 277-279, wherein the step of determining comprises comparing the one or more features of the antibody agent with that of a reference antibody that specifically binds to a Apex binding site of HIV.
Embodiment 281. The method of any one of embodiments 277-280, wherein the step of determining comprises assessing the protein expression level of the antibody agent above a threshold level.
Embodiment 282. The method of embodiment 281, wherein the threshold level is a level that is sufficient to induce ADCC.
Embodiment 283. The method of any one of embodiments 276-282, wherein the step of determining comprises assessing binding of the antibody agent to a Apex binding site of HIV.
Embodiment 284. The method of any one of embodiments 276-283, wherein the step of determining comprises assessing the antibody agent in the TZM-bl cell pseudovirus neutralization assay at antibody agent concentrations up to 25 μg/ml.
Embodiment 285. The method of any one of embodiments 276-284, wherein the cells are present in a subject.
Embodiment 286. The method of any one of embodiments 276-284, wherein the cells are ex vivo cells.
Embodiment 287. The method of embodiment 285, wherein the one or more features include antibody level in one or more tissues in the subject.
Embodiment 288. A method of manufacture, the method comprising steps of:
(A) determining one or more features of a polyribonucleotide of any one of embodiments 1-218, the composition of any one of embodiments 220-245, or the pharmaceutical composition of any one of embodiments 246-248, which one or more features comprise or consist of:
   (i) length and/or sequence of the polyribonucleotide;
   (ii) integrity of the polyribonucleotide;
   (iii) presence and/or location of one or more chemical moieties of the polyribonucleotide;
   (iv) extent of expression of the antibody agent when the polyribonucleotide is introduced into a cell;
   (v) stability of the polyribonucleotide or composition thereof;
   (vi) level of antibody agent in a biological sample from an organism into which the polyribonucleotide has been introduced;
   (vii) binding specificity of the antibody agent expressed from the polyribonucleotide, optionally to a Apex binding site of HIV;
   (viii) efficacy of the antibody agent to mediate target cell death through ADCC;
   (ix) efficacy of the antibody agent to mediate target cell death through complement dependent cytotoxicity (CDC);
   (x) lipid identity and amount/concentration within the composition;
   (xi) size of lipid nanoparticles within the composition;
   (xii) polydispersity of lipid nanoparticles within the composition;
   (xiii) amount/concentration of the polyribonucleotide within the composition;
   (xiv) extent of encapsulation of the polyribonucleotide within lipid nanoparticles;
   (xv) a level of double stranded RNA; and
   (xvi) combinations thereof;
(B) comparing the one or more features of the polyribonucleotide with that of an appropriate reference standard; and
(C) (i) designating the polyribonucleotide or composition thereof for one or more further steps of manufacturing and/or distribution if the comparison demonstrates that the polyribonucleotide or composition thereof meets or exceeds the reference standard; or
   (ii) taking an alternative action if the comparison demonstrates that the polyribonucleotide or composition thereof does not meet or exceed the reference standard.
Embodiment 289. The method of embodiment 288, wherein the polyribonucleotide is assessed and the one or more further steps of step (C)(i) are or comprise at least formulation of the polyribonucleotide.
Embodiment 290. The method of embodiment 288 or 289, wherein the composition or the pharmaceutical composition is assessed, and the one or more further steps of step (C)(i) are or comprise release and distribution of the composition or the pharmaceutical composition.

### EXEMPLIFICATION

### Example 1: Selection of Exemplary Antibody Agents

The present Example demonstrates exemplary antibody agents specific for HIV that can be selected to be expressed (alone or in combination) from one or more polyribonucleotides, and formulations comprising said polyribonucleotides encoding antibody agent(s). This Example also provides methods by which specific antibody agents can be selected to be encoded by one or more polyribonucleotides, wherein the one or more polyribonucleotides can be used in a composition, e.g., for administration as a therapeutic for treatment or prevention of HIV.

As discussed herein, HIV has an extremely high mutational rate, which enables HIV to evade a subject's immune response, as well as treatments targeting certain epitopes. Thus, an antibody agent must be able to target a breadth of HIV polypeptides and/or polypeptide variants. Antibody agents selected herein are chosen for their ability to target a sufficient number of HIV polypeptides and/or polypeptide variants at a relatively low concentration to control viremia and clear infected cells. Antibody agents selected herein can be used alone, but also can be used in combination to increase the breadth of HIV polypeptides and/or polypeptide variants it is able to target.

The broadly neutralizing antibody (bNAb) “PGDM1400” was identified as a candidate for further experiments alone or in combination with other bNAbs. As discussed herein, PGDM1400 is a natural antibody isolated from an HIV subject. It has been reported that, as compared with other bNAbs (e.g., PGT121, PGT128, or PGT151, which are among the most potent bNAbs to date), PGDM1400 exhibits high neutralization breadth and potency measured on a 106-viral panel (Sok, Devin, et al., “Recombinant HIV envelope trimer selects for quaternary-dependent antibodies targeting the trimer apex.” Proceedings of the National Academy of Sciences 111.49 (2014): 17624-17629; van der Velden, Yme U., et al. “Diverse HIV-1 escape pathways from broadly neutralizing antibody PGDM1400 in humanized mice.” Mabs. 12.1 (2020) e1845908, which are incorporated herein by reference in their entirety).

Because PGDM1400 has been able to outperform other anti-HIV antibodies, PGDM1400 was selected for further assessment, including modification of its antibody format to permit administration of polyribonucleotides encoding an antibody agent having at least the PGDM1400 variable domains, particularly when administered with polyribonucleotides encoding other anti-HIV antibody agents. Development of polyribonucleotides encoding PGDM1400 antibody agents was also performed, as described herein.

Combinations of antibody agents comprising PGDM1400 were also assessed. As discussed herein, the present disclosure provides the insight that administering polyribonucleotides encoding multiple anti-HIV antibody agents can be a particularly powerful approach for broadly neutralizing a number of HIV strains and for minimizing evasion of the antibody agents by HIV variants. Accordingly, combinations including PGDM1400 with other antibody agents were tested for potency. Selection of additional antibody agents for use in combination with PGDM1400 was assessed using Combinaber, an online tool based on Bliss-Hill combination prediction model on an HIV global panel (Wagh et al., Plos Path 2016). (https://www.hiv.lanl.gov/content/sequence/COMBINABER/combinaber.html). In order apply the Bliss-Hill model, the IC80 values at <0.1 μg/ml, <1 μg/ml and <10 μg/ml of the antibody agents (here, bNAbs) were determined. Data from the exemplary combinations of bNAbs with PGDM1400 are shown in the Table 11 below.

TABLE 11
IC80 data obtained with various combinations including PGDM1400 and other antibody agents.

| | | % Coverage at (overall Rank*) | | |
|---|---|---|---|---|
| | | IC80 < 0.1 μg/ml | IC80 < 1 μg/ml | IC80 < 10 μ′g/ml |
| 2 bNAb | PGDM1400 + bNAb002 | 61.24% (2) | 91.01% (2) | 97.19% (2) |
| 3 bNAb | PGDM1400 + bNAb002 + bNAb003 | 69.66% (5) | 98.31% (1) | 100.00% (6) |
| | PGDM1400 + bNAb002 + bNAb004 | 70.22% (6) | 96.63% (12) | 99.44% (11) |
| | PGDM1400 + bNAb002 + bNAb005 | 68.54% (11) | 96.07% (9) | 99.44% (4) |
| 4 bNAb | PGDM1400 + bNAb002 + bNAb005 + | 79.78% (10) | 100.00% (5) | 100.00% (10) |
| | bNAb003 | | | |
| | PGDM1400 + bNAb002 + bNAb005 + | 78.65% (13) | 99.43% (9) | 100.00% (14) |
| | bNAb004 | | | |

This data confirms that PGDM1400, while having excellent breadth and potency, can be used with other anti-HIV antibody agents to further increase breadth and potency that can be achieved by a pharmaceutical composition.

### Example 2: Generation of Polyribonucleotides Encoding PGDM1400 IgG Antibody Agents

The present Example demonstrates the generation of polyribonucleotide sequences encoding an anti-HIV antibody agent. The present Example further demonstrates that such polyribonucleotides can be designed so that transient in vivo antibody agent production following i.v./i.m./s.c. delivery of the one or more polyribonucleotides can be achieved.

Specifically, ribonucleic acid sequences encoding the heavy and light chain variable domains of PGDM1400 can be recombinantly combined with other immunoglobulin domains to form polyribonucleotides that encoded PGDM1400 antibody agent immunoglobulin chains. The resulting polyribonucleotides were cloned into the JR81 DNA plasmid. The polyribonucleotides that encoded PGDM1400 antibody agents having different formats, including a conventional IgG1, an scFv-Fc, a CrossMab^{CH1-CLx}, a CrossMab^{CH1-CLcv}, and knob-in-hole formats (see e.g., FIGS. 4-5). Exemplary PGDM1400 antibody agent configurations and component immunoglobulin chains are shown in Table 2.

Methods of the present example include:
(1) Cloning of DNA fragments into a DNA plasmid appropriate to use for RNA expression (e.g., JR81). An appropriate DNA plasmid may encode RNA features including, for example, a 5′ untranslated region (e.g., one that is derived from human α-globin mRNA (hAg)), a Kozak sequence, a signal peptide sequence (e.g., husec2)), and/or a 3′ untranslated region (e.g., one that is a combination of two sequence elements (FI element) and/or a polyA tail 30Linker70 (A30L70)). Examples of appropriate DNA plasmids can be found in WO2021214204A1, which is hereby incorporated by reference in its entirety; and
(2) Verification of selected clones by control digestion and sequencing.

### Codon Optimization

For optimal expression of the antibody in human cells, the genes for the IgG light chain and heavy chain were generated based on the amino acid sequence of PGDM1400. Sok, Devin, et al., “Recombinant HIV envelope trimer selects for quaternary-dependent antibodies targeting the trimer apex.” Proceedings of the National Academy of Sciences 111.49 (2014): 17624-17629, which is incorporated herein by reference in its entirety. The amino acid sequence was translated to DNA nucleotide sequence. Restriction sites for Eam1104I (GAAGAG), BamHI (GGATCC), PstI (CTGCAG), SbfI (CCTGCAGG), XhoI (CTCGAG), SpeI (ACTAGT), BspEI (TCCGGA), SacI (GAGCTC), Earl (CTCTTCN\hat{ }NNN) and NheI (GCTAGC) were eliminated after optimization, because these enzymes were intended to be used either for linearization (Eam1104I) or for cloning (other listed enzymes) of the plasmid. Sequences were also examined for the presence of regions showing high homology to the T7 RNA polymerase termination signal sequence “ATCTGTT” followed by multiple “T” residues.

Optimization was carried out with the GeneOptimizer® software provided by Life Technologies GmbH GeneArt®. This software adjusts the codon usage by using the most frequent codons and adapts the GC-content of an uploaded sequence for a chosen expression system, in this case Homo sapiens. At the same time, GeneOptimizer® removes sequence repeats, introns, cryptic splice sites, internal ribosome entry sites and RNA destabilizing sequence elements (e.g., UpA-dinucleotides), adds RNA stabilizing sequence elements (e.g., CpG-dinucleotides) and avoids stable RNA secondary structures as well as unwanted sequences such as restriction sites. The output sequence was not further manipulated and used as it was for analysis and ordering of DNA fragment strings. Those of skill in the art would be aware that alternative methods for codon optimization are available. Moreover, additional information on codon optimization approaches is provided herein.

### Cloning Strategy

Each light chain and heavy chain sequence was cloned into JR81 (pST1-4-AGA-ATAA-hAg-Kozak-GOI-Bam-F-I-No_Lig3-A30LA70) for putative clinical application via SpeI and BamHI restriction sites. For this purpose, the parental JR81 plasmid was digested with the restriction enzymes SpeI and BamHI. The resulting product was analyzed via agarose gel, purified and its concentration measured by UV spectroscopy. The antibody encoding light chain and heavy chain sequences containing complimentary 5′ and 3′ overhangs matching the SpeI and BamHI adjacent plasmid regions, were cloned into the JR81 plasmid by in vivo assembly. Garcia-Nafria, “IVA cloning: A single-tube universal cloning system exploiting bacterial In Vivo Assembly,” Scientific Reports volume 6, Article number: 27459 (2016), which is incorporated herein by reference in its entirety.

In short, digested JR81 plasmid and antibody encoding sequences were mixed and competent Escherichia coli cells were transformed via heat shock. Clones were sequence verified by Sanger sequencing. Sequencing results were aligned to the reference sequence. The nucleotide sequence of the resulting heavy chain- and light chain-encoding plasmid constructs as well as the sequence of the backbone can be found in SEQ ID NOs: 1305, 1308, and 1314 (heavy chain) and SEQ ID NO: 1311 (light chain). Such sequences encode heavy chain amino acid sequences shown in SEQ ID NOs: 1307, 1310, and 1316 and encode light chain amino acid sequence shown in SEQ ID NO: 1313.

Exemplary sequences used in the cloning methods above are shown in Table 12 below.

TABLE 12
Exemplary sequences used in PGDM1400 antibody agent encoding_polyribonucleotides.

| Feature | Fragment | Nucleotide Sequence |
|---|---|---|
| General | CH1-Hinge-CH2-CH3 | GCCTCTACAAAGGGCCCTAGCGTGTTCCCTCTGGCTCCTAGCAGCAAGT |
| | (G1m3) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGCGCGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCCGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACACTGCCTCCAAGCCGCGAGGAAATGACCAAGAACCAGGTGTCCC |
| | | TGACCTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACTGTGGATA |
| | | AGTCCCGGTGGCAGCAGGGCAACGTGTTCAGCTGTTCTGTGATGCACG |
| | | AAGCCCTGCACAACCACTACACCCAGAAAAGCCTGTCTCTGAGCCCCGG |
| | | CAAGTGATAA (SEQ ID NO: 1191) |
| | CH1-Hinge-CH2-CH3 | GCCTCCACAAAGGGCCCTAGCGTGTTCCCTCTGGCTCCTAGCAGCAAGT |
| | (LS) (G1m3) | CTACAAGCGGAGGAACAGCTGCCCTGGGCTGCCTGGTCAAGGATTACT |
| | | TTCCTGAGCCTGTGACCGTGTCTTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACACTGCCTCCAAGCCGCGAGGAAATGACCAAGAACCAGGTGTCCC |
| | | TGACCTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACCGTGGACA |
| | | AGTCCAGATGGCAGCAGGGCAACGTGTTCAGCTGTAGCGTGCTGCACG |
| | | AAGCCCTGCACAGCCACTACACCCAGAAAAGCCTGTCTCTGAGCCCCGG |
| | | CAAATGATAA (SEQ ID NO: 1192) |
| | CH1-Hinge-CH2-CH3 | GCCTCTACAAAGGGCCCCAGCGTGTTCCCACTGGCTCCTAGCAGCAAGT |
| | (cak-LS) (G1m3) | CTACAAGCGGAGGAACAGCTGCCCTGGGCTGCCTGGTCAAGGATTACT |
| | | TTCCTGAGCCTGTGACCGTGTCTTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACCCTGCCTCCATGCCGCGAGGAAATGACCAAGAACCAGGTGTCCC |
| | | TGTGGTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACCGTGGACA |
| | | AGTCCAGATGGCAGCAGGGCAACGTGTTCAGCTGTAGCGTGCTGCACG |
| | | AAGCCCTGCACAGCCACTACACCCAGAAAAGCCTGTCTCTGAGCCCCGG |
| | | CAAGTGATAA (SEQ ID NO: 1193) |
| | CH1-Hinge-CH2 | GCCTCCACAAAGGGCCCTAGCGTGTTCCCACTGGCTCCTAGCAGCAAGT |
| | (GAALIE)-CH3 (LS) | CTACAAGCGGAGGAACAGCCGCTCTGGGCTGCCTGGTCAAGGATTACT |
| | (G1m3) | TTCCCGAGCCTGTGACCGTGTCCTGGAATTCTGGCGCTCTGACAAGCGG |
| | | CGTGCACACCTTTCCAGCTGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1194) |
| | κ-CL | CGTACGGTGGCCGCTCCTAGCGTGTTCATCTTTCCACCTAGCGACGAGC |
| | | AGCTGAAGTCTGGCACAGCCTCTGTCGTGTGCCTGCTCAACAACTTCTA |
| | | CCCCAGAGAAGCCAAGGTGCAGTGGAAGGTGGACAACGCCCTGCAGAG |
| | | CGGCAATAGCCAAGAGAGCGTGACCGAGCAGGACAGCAAGGACTCTAC |
| | | CTACAGCCTGAGCAGCACACTGACCCTGAGCAAGGCCGACTACGAGAAG |
| | | CACAAAGTGTACGCCTGCGAAGTGACCCACCAGGGCCTTTCTAGCCCTG |
| | | TGACCAAGAGCTTCAACCGGGGCGAGTGCTGATAA (SEQ ID NO: 151) |
| | husec2 | ATGGATTGGATTTGGAGAATCCTGTTCCTCGTGGGCGCCGCTACCGGA |
| | | GCCCACTCC (SEQ ID NO: 3) |
| | husec2 v3 | ATGGACTGGATCTGGCGCATCCTGTTTCTTGTGGGAGCCGCCACAGGC |
| | | GCCCATTCT (SEQ ID NO: 7) |
| | 5′UTR (cap proximal | AGAATAAACTAGTATTCTTCTGGTCCCCACAGACTCAGAGAGAACCCGC |
| | sequence + human | CACC (SEQ ID NO: 11) |
| | alpha-Globin + | |
| | Kozak) | |
| | 3′UTR (FI-Element | CTGGTACTGCATGCACGCAATGCTAGCTGCCCCTTTCCCGTCCT |
| | + polyA30LA70) | GGGTACCCCGAGTCTCCCCCGACCTCGGGTCCCAGGTATGCTCC |
| | | CACCTCCACCTGCCCCACTCACCACCTCTGCTAGTTCCAGACACC |
| | | TCCCAAGCACGCAGCAATGCAGCTCAAAACGCTTAGCCTAGCCA |
| | | CACCCCCACGGGAAACAGCAGTGATTAACCTTTAGCAATAAACG |
| | | AAAGTTTAACTAAGCTATACTAACCCCAGGGTTGGTCAATTTCGT |
| | | GCCAGCCACACCCTCGAGCTAGCAAAAAAAAAAAAAAAAAAAAAAAAA |
| | | AAAAAGCATATGACTAAAAAAAAAAAAAAAAAAAAAAAAAAAAAAAAAAA |
| | | AAAAAAAAAAAAAAAAAAAAAAAAAAAAAAAAAAA (SEQ ID NO: 1195) |
| | 3′UTR | CTGGTACTGCATGCACGCAATGCTAGCTGCCCCTTTCCCGTCCTGGGTA |
| | | CCCCGAGTCTCCCCCGACCTCGGGTCCCAGGTATGCTCCCACCTCCACC |
| | | TGCCCCACTCACCACCTCTGCTAGTTCCAGACACCTCCCAAGCACGCAG |
| | | CAATGCAGCTCAAAACGCTTAGCCTAGCCACACCCCCACGGGAAACAGC |
| | | AGTGATTAACCTTTAGCAATAAACGAAAGTTTAACTAAGCTATACTAACC |
| | | CCAGGGTTGGTCAATTTCGTGCCAGCCACACC (SEQ ID NO: 15) |
| | polyA30LA70 | AAAAAAAAAAAAAAAAAAAAAAAAAAAAAAGCATATGACTAAAAAAAAAA |
| | | AAAAAAAAAAAAAAAAAAAAAAAAAAAAAAAAAAAAAAAAAAAAAAAAAA |
| | | AAAAAAAAAA (SEQ ID NO: 17) |
| PGDM1400 | VH | CAGGTGCATCTGACGCAGTCTGGGCCTGAGGTGAGGAAGCCTGGGACC |
| | | TCCGTAAAGGTCTCCTGCAAGGCCCCTGGAAACACATTGAAGACTTATG |
| | | ATCTACACTGGGTGCGCAGCGTCCCTGGACAAGGCCTTCAGTGGATGG |
| | | GATGGATAAGCCATGAGGGCGACAAGAAGGTCATTGTGGAAAGATTCA |
| | | AGGCCAAAGTCACCATTGATTGGGACAGGTCCACCAATACGGCCTATCT |
| | | CCAACTGAGCGGCCTCACATCTGGCGACACGGCCGTCTATTATTGTGCG |
| | | AAAGGCTCAAAACACAGGCTGCGAGATTACGCTCTCTACGACGACGACG |
| | | GCGCATTGAATTGGGCTGTCGATGTTGACTACCTTTCGAACTTGGAATT |
| | | CTGGGGCCAAGGGACCGCCGTCACCGTCTCTTCA |
| | | (SEQ ID NO: 38) |
| | VH v3 | CAAGTTCATCTGACACAGAGCGGCCCTGAAGTGCGGAAGCCTGGCACAA |
| | | GCGTGAAGGTGTCCTGTAAAGCCCCTGGCAACACCCTGAAAACCTACGA |
| | | CCTGCACTGGGTCCGATCTGTGCCTGGACAAGGACTGCAGTGGATGGG |
| | | CTGGATTAGCCACGAGGGCGACAAGAAAGTGATCGTGGAACGGTTCAA |
| | | GGCCAAAGTGACCATCGACTGGGACAGAAGCACCAACACCGCCTACCTG |
| | | CAGCTGAGCGGACTGACATCTGGCGATACCGCCGTGTACTACTGTGCCA |
| | | AGGGCAGCAAGCACCGGCTGAGAGATTACGCCCTGTACGACGATGACG |
| | | GCGCCCTGAATTGGGCCGTCGATGTGGACTACCTGAGCAACCTGGAATT |
| | | CTGGGGCCAGGGAACAGCCGTGACAGTGTCTAGC (SEQ ID NO: 42) |
| | VL | GATTTTGTCCTGACTCAGTCTCCACACTCTCTGTCCGTCACCCCTGGAG |
| | | AGTCGGCCTCCATCTCCTGCAAGTCTAGTCACAGCCTCATTCATGGTGA |
| | | TAGGAACAATTATTTGGCTTGGTACGTACAGAAGCCAGGGCGGTCTCCA |
| | | CAACTCCTGATCTATTTGGCTTCCAGTCGGGCCTCCGGGGTCCCTGACA |
| | | GGTTCAGTGGCAGTGGATCGGACAAAGATTTTACACTGAAGATCAGCAG |
| | | AGTGGAGACTGAGGATGTTGGGACGTATTACTGCATGCAAGGTCGAGA |
| | | AAGTCCCTGGACGTTCGGCCAAGGGACCAAGGTGGACATCAAA (SEQ |
| | | ID NO: 45) |

### Plasmid DNA Preparation

Plasmid DNA was prepared by selecting E. coli clones for inoculation in Luria-Bertani (LB) medium containing kanamycin. The cultures were grown overnight at 37° C. and ~150 to 200 rpm. Following cell harvest, purification was done using the QIAGEN Plasmid Plus Maxi Kit according to the manufacturer's instructions. Finally, the concentration was determined by UV spectroscopy. DNA was stored in certified RNase- and DNase-free reaction tubes.

An overview of the individual plasmids are shown in Table 13 below.

TABLE 13
Exemplary plasmids encoding PGDM1400 Immunoglobulin Chains

| | Target | | |
|---|---|---|---|
| Molecule | Site | Format | Plasmid Insert |
| PGDM1400 | V1V2Apex | IgG1 | PGDM1400-HC |
| | | | (SEQ ID NO: 1305) |
| | | | PGDM1400-LC |
| | | | (SEQ ID NO: 1311) |
| | | | PGDM1400-HC(LS) |
| | | | (SEQ ID NO: 1308) |
| | | | PGDM1400 HC(cak-LS) |
| | | | (SEQ ID NO: 1314) |

### Linearization and DNA Purification

Linearization of plasmid DNA was performed using appropriate restriction enzymes, followed by purification of the linearized DNA template using magnetic beads (DynabeadS™ MyOne™ Carboxylic Acid) according to the manufacturer's protocol. The DNA concentration was measured by UV spectroscopy.

### In Vitro Transcription

CleanCap 413 ((m7(3′OMeG)(5′)ppp(5′)(2′OMeA)pG) capped RNA was produced following the process as disclosed, e.g., in Kreiter, S. et al. Cancer Immunol. Immunother. 56, 1577-87 (2007) and WO 2021214204A1, each of which is incorporated herein by reference in its entirety. Methyl pseudo-uridine was used in the in vitro transcription reaction and incorporated into the produced RNA. Cellulose purification of the resulting RNA was performed to isolate single-stranded RNA, followed by concentration measurement by UV spectroscopy. The RNA integrity was determined by microfluidic-based electrophoresis. The resulting RNA constructs are shown in SEQ ID NOs: 850, 851, and 853 (heavy chain) and 898 (light chain).

### Example 3: Generation of Polyribonucleotides Encoding scFv-Fc PGDM1400

In this Example, polyribonucleotides encoding an PGDM1400 antibody agent was designed in silico as an scFv with different VH-to-VL orientations as well as interconnecting linkers having two different lengths (e.g., (G4S)₄ and (G4S)₅). The nucleotide sequences encoding the scFvs were cloned with a nucleotide sequence encoding an Fc domain to produce Fc-fusion constructs, which encode an antibody agent referred to as an “scFv-Fc” (illustrated in, e.g., FIG. 4, Panel C). The Fc-fusion constructs were cloned into the JR81 vector. Cloning of additional scFv-Fc variants with and without an “LS” mutation was performed. Lead candidates were further optimized by introducing one of two different sequences encoding linkers (e.g., a (G4S)4 or (G4S)5 linker) between the scFv and Fc domain.

Objectives of the present Example include:
(1) Cloning scFv-Fc PGDM1400 L/S-encoding DNA into JR81 plasmid; and
(2) Verification of selected clones by control digestion and sequencing.

### Codon Optimization and Cloning Strategy

Codon optimization and cloning were performed as described above in Example 2. The nucleotide sequence of the resulting scFv-Fc chain-encoding plasmid constructs as well as the sequence of the backbone can be found in SEQ ID NOs: 1341, 1344, 1347 and 1350. Such sequences encode scFv-Fc chain amino acid sequences shown in SEQ ID NOs: 1343, 1346, 1349, and 1352.

Exemplary sequences used in the cloning methods above are shown in Table 14 below.

TABLE 14
Exemplary sequences used in scFv-Fc PGDM1400 encoding polyribonucleotides.

| Feature | Fragment | Nucleotide Sequence |
|---|---|---|
| General | Hinge (C/S)-CH2- | GAACCTAAGAGCAGCGACAAGACCCACACCTGTCCTCCATGTCCTGCTC |
| | CH3 (LS) (G1m3) | CAGAACTGCTCGGCGGACCTTCCGTGTTCCTGTTTCCTCCAAAGCCTAA |
| | | GGACACCCTGATGATCAGCAGAACCCCTGAAGTGACCTGCGTGGTGGTG |
| | | GATGTGTCTCACGAGGACCCCGAAGTGAAGTTCAATTGGTACGTGGACG |
| | | GCGTGGAAGTGCACAACGCCAAGACCAAGCCTAGAGAGGAACAGTACAA |
| | | CAGCACCTACAGAGTGGTGTCCGTGCTGACAGTGCTGCACCAGGATTGG |
| | | CTGAACGGCAAAGAGTACAAGTGCAAGGTGTCCAACAAGGCCCTGCCTG |
| | | CTCCTATCGAGAAAACCATCAGCAAGGCCAAGGGCCAGCCTAGGGAACC |
| | | CCAGGTTTACACACTGCCTCCAAGCCGCGAGGAAATGACCAAGAACCAG |
| | | GTGTCCCTGACCTGCCTCGTGAAGGGCTTCTACCCTTCCGATATCGCCG |
| | | TGGAATGGGAGAGCAATGGCCAGCCTGAGAACAACTACAAGACAACCCC |
| | | TCCTGTGCTGGACAGCGACGGCTCATTCTTCCTGTACAGCAAGCTGACC |
| | | GTGGACAAGTCCAGATGGCAGCAGGGCAACGTGTTCAGCTGTAGCGTG |
| | | CTGCACGAAGCCCTGCACAGCCACTACACCCAGAAAAGCCTGTCTCTGA |
| | | GCCCCGGCAAATGATAA (SEQ ID NO: 1196) |
| | husec2 v2 | ATGGATTGGATCTGGCGCATCCTGTTTCTCGTGGGAGCTGCCACAGGCG |
| | | CCCACTCT (SEQ ID NO: 5) |
| | husec2 v4 | ATGGATTGGATCTGGCGCATCCTGTTTCTCGTGGGAGCTGCCACAGGCG |
| | | CCCACAGC (SEQ ID NO: 9) |
| | 5′UTR (cap proximal | AGAATAAACTAGTATTCTTCTGGTCCCCACAGACTCAGAGAGAACCCGC |
| | sequence + human | CACC (SEQ ID NO: 11) |
| | alpha-Globin + | |
| | Kozak) | |
| | 3′UTR (FI-Element | CTGGTACTGCATGCACGCAATGCTAGCTGCCCCTTTCCCGTCCTG |
| | + polyA30LA70) | GGTACCCCGAGTCTCCCCCGACCTCGGGTCCCAGGTATGCTCCC |
| | | ACCTCCACCTGCCCCACTCACCACCTCTGCTAGTTCCAGACACCT |
| | | CCCAAGCACGCAGCAATGCAGCTCAAAACGCTTAGCCTAGCCAC |
| | | ACCCCCACGGGAAACAGCAGTGATTAACCTTTAGCAATAAACGA |
| | | AAGTTTAACTAAGCTATACTAACCCCAGGGTTGGTCAATTTCGTG |
| | | CCAGCCACACCCTCGAGCTAGCAAAAAAAAAAAAAAAAAAAAAAAAAAA |
| | | AAAGCATATGACTAAAAAAAAAAAAAAAAAAAAAAAAAAAAAAAAAAAAA |
| | | AAAAAAAAAAAAAAAAAAAAAAAAAAAAAAAAA (SEQ ID NO: 1195) |
| | Linker LL4 v1 | GGTGGCGGAGGAAGCGGAGGCGGAGGTTCAGGCGGCGGAGGCTCTGG |
| | | CGGTGGTGGATCT (SEQ ID NO: 50) |
| | Linker LL4 v2 | GGTGGCGGTGGTTCTGGCGGAGGCGGAAGTGGCGGCGGTGGGTCCGG |
| | | AGGTGGTGGATCT(SEQ ID NO: 52) |
| | Linker LL4 v3 | GGCGGAGGTGGAAGTGGTGGCGGCGGATCTGGCGGCGGTGGGTCCGG |
| | | AGGTGGTGGATCT(SEQ ID NO: 54) |
| | Linker LL4 v4 | GGCGGAGGTGGAAGTGGTGGTGGCGGTAGCGGAGGCGGTGGGTCCGG |
| | | AGGTGGTGGATCT(SEQ ID NO:56) |
| | Linker LL4 v5 | GGCGGCGGAGGAAGCGGAGGCGGAGGTTCTGGTGGCGGAGGCTCAGG |
| | | CGGTGGCGGATCT(SEQ ID NO: 58) |
| | Linker LL5 v1 | GGCGGCGGAGGAAGCGGAGGCGGAGGTTCTGGTGGTGGTGGCTCTGG |
| | | TGGCGGAGGCTCAGGCGGTGGCGGATCT (SEQ ID NO: 61) |
| | Linker LL5 v2 | GGCGGCGGAGGAAGCGGAGGGGGAGGCTCTGGAGGTGGTGGCTCTGG |
| | | TGGCGGAGGCTCAGGCGGTGGCGGATCT(SEQ ID NO: 63) |
| PGDM1400 | VH_LL4_VL_LL4 | CAAGTTCACCTGACACAGTCTGGCCCCGAAGTGCGGAAGCCTGGCACAA |
| | | GCGTGAAGGTGTCCTGTAAAGCCCCTGGCAACACCCTGAAAACCTACGA |
| | | CCTGCACTGGGTCCGATCTGTGCCTGGACAAGGACTGCAGTGGATGGG |
| | | CTGGATTAGCCACGAGGGCGACAAGAAAGTGATCGTGGAACGGTTCAA |
| | | GGCCAAAGTGACCATCGACTGGGACAGAAGCACCAACACCGCCTACCTG |
| | | CAGCTGAGCGGACTGACATCTGGCGATACCGCCGTGTACTACTGTGCCA |
| | | AGGGCAGCAAGCACCGGCTGAGAGATTACGCCCTGTACGACGATGACG |
| | | GCGCCCTGAATTGGGCCGTCGATGTGGACTACCTGAGCAACCTGGAATT |
| | | CTGGGGCCAGGGAACAGCCGTGACAGTGTCTTCCGGTGGCGGAGGAAG |
| | | CGGAGGCGGAGGTTCAGGCGGCGGAGGCTCTGGCGGTGGTGGATCTGA |
| | | TTTTGTGCTGACACAGAGCCCTCACAGCCTGAGCGTTACACCTGGCGAG |
| | | AGCGCCAGCATCAGCTGCAAGTCTAGCCACTCTCTGATCCACGGCGACC |
| | | GGAACAACTACCTGGCTTGGTACGTGCAGAAGCCCGGCAGATCTCCTCA |
| | | GCTGCTGATCTACCTGGCCAGCAGCAGAGCTAGCGGCGTGCCAGATAGA |
| | | TTTTCTGGCAGCGGCAGCGACAAGGATTTCACCCTGAAGATCTCCAGAG |
| | | TGGAAACCGAGGACGTGGGCACCTACTACTGTATGCAGGGCAGAGAGA |
| | | GCCCCTGGACATTTGGCCAGGGCACCAAGGTGGACATCAAAGGTGGCG |
| | | GTGGTTCTGGCGGAGGCGGAAGTGGCGGCGGTGGGTCCGGAGGTGGT |
| | | GGATCT (SEQ ID NO: 66) |
| | VL_LL4_VH_LL4 | GATTTTGTGCTGACACAGTCTCCCCACAGCCTGAGCGTTACACCTGGCG |
| | | AGTCTGCCAGCATCAGCTGCAAGTCTAGCCACTCTCTGATCCACGGCGA |
| | | CCGGAACAACTACCTGGCTTGGTACGTGCAGAAGCCCGGCAGATCTCCT |
| | | CAGCTGCTGATCTACCTGGCCAGCAGCAGAGCTTCTGGCGTGCCCGATA |
| | | GATTTTCTGGCAGCGGCAGCGACAAGGACTTCACCCTGAAGATCTCCAG |
| | | AGTGGAAACCGAGGACGTGGGCACCTACTACTGTATGCAGGGCAGAGA |
| | | GAGCCCCTGGACATTTGGCCAGGGCACCAAGGTGGACATCAAAGGCGG |
| | | CGGAGGAAGCGGAGGCGGAGGTTCTGGTGGCGGAGGCTCAGGCGGTG |
| | | GCGGATCTCAAGTTCATCTGACACAGAGCGGCCCTGAAGTGCGGAAGCC |
| | | TGGCACAAGCGTGAAGGTGTCCTGTAAAGCCCCTGGCAACACCCTGAAA |
| | | ACCTACGACCTGCACTGGGTCCGATCTGTGCCTGGACAGGGACTGCAAT |
| | | GGATGGGCTGGATTAGCCACGAGGGCGACAAGAAAGTGATCGTGGAAC |
| | | GGTTCAAGGCCAAAGTGACCATCGACTGGGACAGAAGCACCAACACCGC |
| | | CTACCTGCAGCTGAGCGGACTGACATCTGGCGATACCGCCGTGTACTAC |
| | | TGTGCCAAGGGCAGCAAGCACCGGCTGAGAGATTACGCCCTGTACGACG |
| | | ATGACGGCGCCCTGAATTGGGCCGTCGATGTGGACTACCTGAGCAACCT |
| | | GGAATTCTGGGGCCAGGGAACAGCCGTGACAGTGTCTTCCGGCGGAGG |
| | | TGGAAGTGGTGGTGGCGGTAGCGGAGGCGGTGGGTCCGGAGGTGGTG |
| | | GATCT (SEQ ID NO: 69) |
| | VH_LL5_VL_LL4 | CAAGTTCACCTGACACAGTCTGGCCCCGAAGTGCGGAAGCCTGGCACAA |
| | | GCGTGAAGGTGTCCTGTAAAGCCCCTGGCAACACCCTGAAAACCTACGA |
| | | CCTGCACTGGGTCCGATCTGTGCCTGGACAAGGACTGCAGTGGATGGG |
| | | CTGGATTAGCCACGAGGGCGACAAGAAAGTGATCGTGGAACGGTTCAA |
| | | GGCCAAAGTGACCATCGACTGGGACAGAAGCACCAACACCGCCTACCTG |
| | | CAGCTGAGCGGACTGACATCTGGCGATACCGCCGTGTACTACTGTGCCA |
| | | AGGGCAGCAAGCACCGGCTGAGAGATTACGCCCTGTACGACGATGACG |
| | | GCGCCCTGAATTGGGCCGTCGATGTGGACTACCTGAGCAACCTGGAATT |
| | | CTGGGGCCAGGGAACAGCCGTGACAGTGTCTTCCGGGGGGGGAGGAAG |
| | | CGGAGGGGGAGGCTCTGGAGGTGGTGGCTCTGGTGGCGGAGGCTCAG |
| | | GCGGTGGCGGATCTGATTTTGTGCTGACACAGAGCCCTCACAGCCTGAG |
| | | CGTTACACCTGGCGAGAGCGCCAGCATCAGCTGCAAGTCTAGCCACTCT |
| | | CTGATCCACGGCGACCGGAACAACTACCTGGCTTGGTACGTGCAGAAGC |
| | | CCGGCAGATCTCCTCAGCTGCTGATCTACCTGGCCAGCAGCAGAGCTAG |
| | | CGGCGTGCCAGATAGATTTTCTGGCAGCGGCAGCGACAAGGATTTCACC |
| | | CTGAAGATCTCCAGAGTGGAAACCGAGGACGTGGGCACCTACTACTGTA |
| | | TGCAGGGCAGAGAGAGCCCCTGGACATTTGGCCAGGGCACCAAGGTGG |
| | | ACATCAAAGGCGGAGGTGGAAGTGGTGGTGGCGGTAGCGGAGGCGGTG |
| | | GGTCCGGAGGTGGTGGATCT (SEQ ID NO: 72) |
| | VL_LL5_VH_LL4 | GATTTTGTGCTGACACAGTCTCCCCACAGCCTGAGCGTTACACCTGGCG |
| | | AGTCTGCCAGCATCAGCTGCAAGTCTAGCCACTCTCTGATCCACGGCGA |
| | | CCGGAACAACTACCTGGCTTGGTACGTGCAGAAGCCCGGCAGATCTCCT |
| | | CAGCTGCTGATCTACCTGGCCAGCAGCAGAGCTTCTGGCGTGCCCGATA |
| | | GATTTTCTGGCAGCGGCAGCGACAAGGACTTCACCCTGAAGATCTCCAG |
| | | AGTGGAAACCGAGGACGTGGGCACCTACTACTGTATGCAGGGCAGAGA |
| | | GAGCCCCTGGACATTTGGCCAGGGCACCAAGGTGGACATCAAAGGCGG |
| | | CGGAGGAAGCGGAGGCGGAGGTTCTGGTGGTGGTGGCTCTGGTGGCG |
| | | GAGGCTCAGGCGGTGGCGGATCTCAAGTTCATCTGACACAGAGCGGCC |
| | | CTGAAGTGCGGAAGCCTGGCACAAGCGTGAAGGTGTCCTGTAAAGCCCC |
| | | TGGCAACACCCTGAAAACCTACGACCTGCACTGGGTCCGATCTGTGCCT |
| | | GGACAGGGACTGCAATGGATGGGCTGGATTAGCCACGAGGGCGACAAG |
| | | AAAGTGATCGTGGAACGGTTCAAGGCCAAAGTGACCATCGACTGGGACA |
| | | GAAGCACCAACACCGCCTACCTGCAGCTGAGCGGACTGACATCTGGCGA |
| | | TACCGCCGTGTACTACTGTGCCAAGGGCAGCAAGCACCGGCTGAGAGAT |
| | | TACGCCCTGTACGACGATGACGGCGCCCTGAATTGGGCCGTCGATGTGG |
| | | ACTACCTGAGCAACCTGGAATTCTGGGGCCAGGGAACAGCCGTGACAGT |
| | | GTCTTCCGGCGGAGGTGGAAGTGGTGGCGGCGGATCTGGCGGCGGTG |
| | | GGTCCGGAGGTGGTGGATCT (SEQ ID NO: 75) |

### Plasmid DNA Preparation

Plasmid DNA was prepared by selecting 1. c40clones for inoculation in Luria-Bertani (LB) medium containing kanamycin. The cultures were grown overnight at 37° C. and −150 to 200 rpm. Following cell harvest, purification was done using the QIAGEN Plasmid Plus Maxi Kit according to the manufacturer's instructions. Finally, the concentration was determined by UV spectroscopy. DNA was stored in certified RNase- and DNase-free reaction tubes.

An overview of the individual plasmids are shown in Table 15 below.

TABLE 15
Exemplary plasmids encoding scFv-Fc PGDM1400 chains

| | Target | | |
|---|---|---|---|
| Molecule | site | Format | Plasmid Insert |
| PGDM1400 | V1V2Apex | scFv-Fc | scFv-PGDM1400 (VH-LL4-VL)- |
| | | | Fc(LS) (SEQ ID NO: 1341) |
| | | | scFv- PGDM1400 (VH-LL5-VL)- |
| | | | Fc(LS) (SEQ ID NO: 1344) |
| | | | scFv- PGDM1400 (VL-LL4-VH)- |
| | | | Fc(LS) (SEQ ID NO: 1347) |
| | | | scFv- PGDM1400 (VL-LL5-VH)- |
| | | | Fc(LS) (SEQ ID NO: 1350) |

### Linearization and DNA Purification

Linearization of plasmid DNA was performed using appropriate restriction enzymes, followed by purification of the linearized DNA template using magnetic beads (Dynabeads™ MyOne™ Carboxylic Acid) according to the manufacturer's protocol. The DNA concentration was measured by UV spectroscopy.

### In Vitro Transcription

CleanCap 413 (m7(3′OMeG)(5′)ppp(5′)(2′OMeA)pG) capped RNA was produced following the process as disclosed, e.g., in WO 2021214204A1, which is incorporated herein by reference in its entirety. Methyl pseudo-uridine was used in the in vitro transcription reaction and incorporated into the produced RNA. Cellulose purification of the resulting RNA was performed to isolate single-stranded RNA, followed by concentration measurement by UV spectroscopy. The RNA integrity was determined by microfluidic-based electrophoresis. The resulting RNA constructs are shown in SEQ ID NOs: 1000, 1048, 1096, and 929.

### Example 4: Generation of Polyribonucleotides Encoding PGDM1400 Monospecific CrossMab^{CH1-CLx} and PGDM1400 Monospecific CrossMab^{CH1-CLcv}

In this Example, polyribonucleotides encoding PGDM1400 in CrossMab^{CH1-CLx} format (as illustrated e.g., in FIG. 4, Panel B) were generated. In CrossMab^{CH1-CLx} format, a CH₁ domain is fused to a VL and a CL domain is fused in between a VH and a CH₂-CH₃ set of domains. By introducing these domain swaps, the resulting CrossMab^{CH1-CLx} chains will specifically pair, while strongly inhibiting binding to wild-type or unmodified immunoglobulin chains. Thus, with this format, non-functional mispairings are strongly reduced.

Polyribonucleotides encoding PGDM1400 in CrossMab^{CH1-CLcv} format (as illustrated e.g., in FIG. 4, Panel D) were generated. In CrossMab^{CH1-CLcv} format, a VL is operably linked to a CL domain and a VH is operably linked to a CH₁-CH₂-CH₃ set of domains. To minimize incorrect chain pairing, the CL domain of the immunoglobulin light chain and the CH₁ domain of the immunoglobulin heavy chain are modified to include charge variants, which promote pairing of a constant domain with a positive charge to a constant domain with a negative charge.

Polyribonucleotides encoding the CrossMab^{CH1-CLx} and CrossMab^{CH1-CLcv} formats were cloned into the JR81 vector.

Objectives of the present Example include:
(1) Clone PGDM1400 CrossMab^{CH1-CLx}-encoding DNA Fragment Strings into JR81 backbone; and
(2) Clone PGDM1400 CrossMab^{CH1/CLcv}-encoding DNA Fragment Strings into JR81 backbone.

The methods of the present example include: (1) cloning of DNA fragments into appropriate RNA-expression vector; and (2) verification of selected clones by control digestion and sequencing.

### Codon Optimization and Cloning Strategy

Codon optimization and cloning were performed as described above in Example 2. The nucleotide sequence of the resulting heavy chain- and light chain-encoding plasmid constructs as well as the sequence of the backbone can be found in SEQ ID NOs: 1317, 1323, 1329, 1332, 1335, and 1338 (heavy chain) and SEQ ID NO: 1320 and 1326 (light chain). Such sequences encode heavy chain amino acid sequences shown in SEQ ID NOs: 1319, 1325, 1331, 1334, 1337, and 1340 and encode light chain amino acid sequence shown in SEQ ID NO: 1322 and 1328.

Exemplary sequences used in the cloning methods for PGDM1400 CrossMab^{CH1-CLx}-encoding DNA Fragment Strings are shown in Table 16 below.

TABLE 16
Exemplary sequences used in PGDM1400 CrossMab^{CH1-CLx} encoding polyribonucleotides.

| Feature | Fragment | Nucleotide Sequence |
|---|---|---|
| General | Hinge (ΔEPKSC)-CH2-CH3 | GATAAGACCCACACCTGTCCTCCATGTCCAGCTCCAGAACTGCTC |
| | (LS) (G1m3) | GGCGGACCTTCCGTGTTCCTGTTTCCTCCAAAGCCTAAGGACACC |
| | | CTGATGATCAGCAGAACCCCTGAAGTGACCTGCGTGGTGGTGGAT |
| | | GTGTCTCACGAGGACCCCGAAGTGAAGTTCAATTGGTACGTGGAC |
| | | GGCGTGGAAGTGCACAACGCCAAGACCAAGCCTAGAGAGGAACA |
| | | GTACAACAGCACCTACAGAGTGGTGTCCGTGCTGACAGTGCTGCA |
| | | CCAGGATTGGCTGAACGGCAAAGAGTACAAGTGCAAGGTGTCCAA |
| | | CAAGGCCCTGCCTGCTCCTATCGAGAAAACCATCAGCAAGGCCAA |
| | | GGGCCAGCCTAGGGAACCCCAGGTTTACACACTGCCTCCAAGCCG |
| | | CGAGGAAATGACCAAGAACCAGGTGTCCCTGACCTGCCTCGTGAA |
| | | GGGCTTCTACCCTTCCGATATCGCCGTGGAATGGGAGAGCAATGG |
| | | CCAGCCTGAGAACAACTACAAGACAACCCCTCCTGTGCTGGACAG |
| | | CGACGGCTCATTCTTCCTGTACAGCAAGCTGACCGTGGACAAGTC |
| | | CAGATGGCAGCAGGGCAACGTGTTCAGCTGTAGCGTGCTGCACG |
| | | AAGCCCTGCACAGCCACTACACCCAGAAAAGCCTGTCTCTGAGCC |
| | | CCGGCAAATGATAA |
| | | (SEQ ID NO: 1197) |
| | Hinge (ΔEPKSC)-CH2-CH3 | GATAAGACCCACACCTGTCCTCCATGTCCAGCTCCAGAACTGCTC |
| | (cah-LS) (G1m3) | GGCGGACCTTCCGTGTTCCTGTTTCCTCCAAAGCCTAAGGACACC |
| | | CTGATGATCAGCAGAACCCCTGAAGTGACCTGCGTGGTGGTGGAT |
| | | GTGTCTCACGAGGACCCCGAAGTGAAGTTCAATTGGTACGTGGAC |
| | | GGCGTGGAAGTGCACAACGCCAAGACCAAGCCTAGAGAGGAACA |
| | | GTACAACAGCACCTACAGAGTGGTGTCCGTGCTGACAGTGCTGCA |
| | | CCAGGATTGGCTGAACGGCAAAGAGTACAAGTGCAAGGTGTCCAA |
| | | CAAGGCCCTGCCTGCTCCTATCGAGAAAACCATCAGCAAGGCCAA |
| | | GGGCCAGCCTAGGGAACCCCAAGTCTGTACACTGCCTCCTAGCCG |
| | | CGAGGAAATGACCAAGAACCAGGTGTCCCTGAGCTGCGCCGTGAA |
| | | GGGCTTTTACCCTTCCGATATCGCCGTGGAATGGGAGAGCAATGG |
| | | CCAGCCTGAGAACAACTACAAGACAACCCCTCCTGTGCTGGACAG |
| | | CGACGGCTCATTCTTCCTGGTGTCCAAGCTGACAGTGGACAAGAG |
| | | CAGATGGCAGCAGGGCAACGTGTTCAGCTGCTCTGTGCTGCACGA |
| | | AGCCCTGCACAGCCACTACACCCAGAAGTCCCTGTCTCTGAGCCC |
| | | CGGCAAGTGATAA (SEQ ID NO: 1198) |
| | Hinge (ΔEPKSC)-CH2-CH3 | GATAAGACCCACACCTGTCCTCCATGTCCAGCTCCAGAACTGCTC |
| | (cak-LS) (G1m3) | GGCGGACCTTCCGTGTTCCTGTTTCCTCCAAAGCCTAAGGACACC |
| | | CTGATGATCAGCAGAACCCCTGAAGTGACCTGCGTGGTGGTGGAT |
| | | GTGTCTCACGAGGACCCCGAAGTGAAGTTCAATTGGTACGTGGAC |
| | | GGCGTGGAAGTGCACAACGCCAAGACCAAGCCTAGAGAGGAACA |
| | | GTACAACAGCACCTACAGAGTGGTGTCCGTGCTGACAGTGCTGCA |
| | | CCAGGATTGGCTGAACGGCAAAGAGTACAAGTGCAAGGTGTCCAA |
| | | CAAGGCCCTGCCTGCTCCTATCGAGAAAACCATCAGCAAGGCCAA |
| | | GGGCCAGCCTAGGGAACCCCAGGTTTACACCCTGCCTCCATGCCG |
| | | CGAGGAAATGACCAAGAACCAGGTGTCCCTGTGGTGCCTCGTGAA |
| | | GGGCTTCTACCCTTCCGATATCGCCGTGGAATGGGAGAGCAATGG |
| | | CCAGCCTGAGAACAACTACAAGACAACCCCTCCTGTGCTGGACAG |
| | | CGACGGCTCATTCTTCCTGTACAGCAAGCTGACCGTGGACAAGTC |
| | | CAGATGGCAGCAGGGCAACGTGTTCAGCTGTAGCGTGCTGCACG |
| | | AAGCCCTGCACAGCCACTACACCCAGAAGTCCCTGTCTCTGAGCC |
| | | CCGGCAAGTGATAA(SEQ ID NO: 1199) |
| | Hinge (ΔEPKSC)-CH2 | GATAAGACCCACACCTGTCCTCCATGTCCAGCTCCAGAACTGCTG |
| | (GAALIE)-CH3 (LS) | GCCGGACCTTCCGTGTTCCTGTTTCCTCCAAAGCCTAAGGACACC |
| | (G1m3) | CTGATGATCAGCAGAACCCCTGAAGTGACCTGCGTGGTGGTGGAT |
| | | GTGTCTCACGAGGACCCCGAAGTGAAGTTCAATTGGTACGTGGAC |
| | | GGCGTGGAAGTGCACAATGCCAAGACCAAGCCTAGAGAGGAACA |
| | | GTACAACAGCACCTACAGAGTGGTGTCCGTGCTGACCGTGCTGCA |
| | | CCAGGATTGGCTGAACGGCAAAGAGTACAAGTGCAAGGTGTCCAA |
| | | CAAGGCCCTGCCTCTGCCTGAGGAAAAGACCATCAGCAAGGCCAA |
| | | GGGCCAGCCTAGGGAACCCCAGGTTTACACACTGCCTCCAAGCCG |
| | | CGAGGAAATGACCAAGAACCAGGTGTCCCTGACCTGCCTCGTGAA |
| | | GGGCTTCTACCCTTCCGATATCGCCGTGGAATGGGAGAGCAATGG |
| | | CCAGCCTGAGAACAACTACAAGACAACCCCTCCTGTGCTGGACAG |
| | | CGACGGCTCATTCTTCCTGTACAGCAAGCTGACCGTGGACAAGTC |
| | | CAGATGGCAGCAGGGCAACGTGTTCAGCTGTAGCGTGCTGCACG |
| | | AAGCCCTGCACAGCCACTACACCCAGAAAAGCCTGTCTCTGAGCC |
| | | CCGGCAAATGATAA(SEQ ID NO: 1200) |
| | Hinge (ΔEPKSC)-CH2 | GATAAGACCCACACCTGTCCTCCATGTCCAGCTCCAGAACTGCTC |
| | (GAALIE)-CH3 (cah-LS) | GGCGGACCTTCCGTGTTCCTGTTTCCTCCAAAGCCTAAGGACACC |
| | (G1m3) | CTGATGATCAGCAGAACCCCTGAAGTGACCTGCGTGGTGGTGGAT |
| | | GTGTCTCACGAGGACCCCGAAGTGAAGTTCAATTGGTACGTGGAC |
| | | GGCGTGGAAGTGCACAACGCCAAGACCAAGCCTAGAGAGGAACA |
| | | GTACAACAGCACCTACAGAGTGGTGTCCGTGCTGACAGTGCTGCA |
| | | CCAGGATTGGCTGAACGGCAAAGAGTACAAGTGCAAGGTGTCCAA |
| | | CAAGGCCCTGCCTGCTCCTATCGAGAAAACCATCAGCAAGGCCAA |
| | | GGGCCAGCCTAGGGAACCCCAAGTCTGTACACTGCCTCCTAGCCG |
| | | CGAGGAAATGACCAAGAACCAGGTGTCCCTGAGCTGCGCCGTGAA |
| | | GGGCTTTTACCCTTCCGATATCGCCGTGGAATGGGAGAGCAATGG |
| | | CCAGCCTGAGAACAACTACAAGACAACCCCTCCTGTGCTGGACAG |
| | | CGACGGCTCATTCTTCCTGGTGTCCAAGCTGACAGTGGACAAGAG |
| | | CAGATGGCAGCAGGGCAACGTGTTCAGCTGCTCTGTGCTGCACGA |
| | | AGCCCTGCACAGCCACTACACCCAGAAGTCCCTGTCTCTGAGCCC |
| | | CGGCAAGTGATAA(SEQ ID NO: 1201) |
| | κ-CL (AS-QE) | GCATCTGTGGCCGCTCCTAGCGTGTTCATCTTCCCACCTAGCGAC |
| | | GAGGAACTGAAGTCTGGCACAGCCAGCGTCGTGTGCCTGCTGAAC |
| | | AACTTCTACCCCAGAGAAGCCAAGGTGCAGTGGAAGGTGGACAAC |
| | | GCCCTGCAGAGCGGCAATAGCCAAGAGAGCGTGACCGAGCAGGA |
| | | CAGCAAGGACTCTACCTACAGCCTGAGCAGCACCCTGACACTGAG |
| | | CAAGGCCGACTACGAGAAGCACAAAGTGTACGCCTGCGAAGTGAC |
| | | CCACCAGGGCCTTTCTAGCCCTGTGACCAAGAGCTTCAACCGGGG |
| | | CGAGTGC (SEQ ID NO: 154) |
| | CH1(SS) (G1m3) | AGCAGCGCCTCTACAAAGGGCCCCAGCGTTTTCCCACTGGCTCCT |
| | | AGCAGCAAGAGCACATCTGGCGGAACAGCCGCTCTGGGCTGTCT |
| | | GGTCAAGGACTACTTTCCCGAGCCTGTGACCGTGTCCTGGAATTC |
| | | TGGCGCTCTGACATCCGGCGTGCACACCTTTCCAGCTGTGCTGCA |
| | | AAGCAGCGGCCTGTACTCTCTGAGCAGCGTGGTCACAGTGCCAAG |
| | | CTCTAGCCTGGGCACCCAGACCTACATCTGCAATGTGAACCACAA |
| | | GCCTAGCAACACGAAGGTCGACAAGAGAGTGGAACCCAAGTCCTG |
| | | CTGATAA (SEQ ID NO: 1202) |
| | husec2 v2 | ATGGATTGGATCTGGCGCATCCTGTTTCTCGTGGGAGCTGCCACA |
| | | GGCGCCCACTCT (SEQ ID NO: 5) |
| | husec2 v4 | ATGGATTGGATCTGGCGCATCCTGTTTCTCGTGGGAGCTGCCACA |
| | | GGCGCCCACAGC (SEQ ID NO: 9) |
| | 5′UTR (cap proximal | AGAATAAACTAGTATTCTTCTGGTCCCCACAGACTCAGAGAGAAC |
| | sequence + human alpha- | CCGCCACC (SEQ ID NO: 11) |
| | Globin + Kozak) | |
| | 3′UTR (FI-Element + | CTGGTACTGCATGCACGCAATGCTAGCTGCCCCTTTCCCGT |
| | polyA30LA70) | CCTGGGTACCCCGAGTCTCCCCCGACCTCGGGTCCCAGGT |
| | | ATGCTCCCACCTCCACCTGCCCCACTCACCACCTCTGCTAG |
| | | TTCCAGACACCTCCCAAGCACGCAGCAATGCAGCTCAAAAC |
| | | GCTTAGCCTAGCCACACCCCCACGGGAAACAGCAGTGATT |
| | | AACCTTTAGCAATAAACGAAAGTTTAACTAAGCTATACTAA |
| | | CCCCAGGGTTGGTCAATTTCGTGCCAGCCACACCCTCGAGCT |
| | | AGCAAAAAAAAAAAAAAAAAAAAAAAAAAAAAAGCATATGACTAAA |
| | | AAAAAAAAAAAAAAAAAAAAAAAAAAAAAAAAAAAAAAAAAAAAAA |
| | | AAAAAAAAAAAAAAAAAAAAA (SEQ ID NO: 1195) |
| PGDM1400 | VH v2 | CAAGTTCACCTGACACAGTCTGGCCCCGAAGTGCGGAAGCCTGGC |
| | | ACAAGCGTGAAGGTGTCCTGTAAAGCCCCTGGCAACACCCTGAAA |
| | | ACCTACGACCTGCACTGGGTCCGATCTGTGCCTGGACAAGGACTG |
| | | CAGTGGATGGGCTGGATTAGCCACGAGGGCGACAAGAAAGTGAT |
| | | CGTGGAACGGTTCAAGGCCAAAGTGACCATCGACTGGGACAGAA |
| | | GCACCAACACCGCCTACCTGCAGCTGAGCGGACTGACATCTGGCG |
| | | ATACCGCCGTGTACTACTGTGCCAAGGGCAGCAAGCACCGGCTGA |
| | | GAGATTACGCCCTGTACGACGATGACGGCGCCCTGAATTGGGCCG |
| | | TCGATGTGGACTACCTGAGCAACCTGGAATTCTGGGGCCAGGGAA |
| | | CAGCCGTGACAGTGTCTAGT(SEQ ID NO: 40) |
| | VL v2 | GATTTTGTGCTGACACAGTCTCCCCACAGCCTGAGCGTTACACCT |
| | | GGCGAGTCTGCCAGCATCAGCTGCAAGTCTAGCCACTCTCTGATC |
| | | CACGGCGACCGGAACAACTACCTGGCTTGGTACGTGCAGAAGCCC |
| | | GGCAGATCTCCTCAGCTGCTGATCTACCTGGCCAGCAGCAGAGCT |
| | | AGCGGCGTGCCAGATAGATTTTCTGGCAGCGGCAGCGACAAGGA |
| | | CTTCACCCTGAAGATCTCCAGAGTGGAAACCGAGGACGTGGGCAC |
| | | CTACTACTGTATGCAGGGCAGAGAGAGCCCCTGGACATTTGGCCA |
| | | GGGCACCAAGGTGGACATCAAG(SEQ ID NO: 47) |

Exemplary sequences used in the cloning methods for PGDM1400 CrossMab^{CH1-CLcv}-encoding DNA Fragment Strings are shown in Table 17 below.

TABLE 17
Exemplary sequences used in PGDM1400 CrossMab^{CH1-CLcv} encoding polyribonucleotides.

| Feature | Fragment | Nucleotide Sequence |
|---|---|---|
| General | CH1 | GCTTCCACAAAGGGCCCTAGCGTGTTCCCTCTGGCTCCTAGCAGC |
| | (K147E + K213D)(G1m3) | AAGTCTACAAGCGGAGGAACAGCTGCCCTGGGCTGCCTGGTGGA |
| | | AGATTACTTTCCTGAGCCTGTGACCGTGTCCTGGAACAGCGGTGC |
| | | TCTGACTTCTGGCGTGCACACCTTTCCAGCCGTGCTGCAAAGCAG |
| | | CGGCCTGTACTCTCTGAGCAGCGTGGTCACAGTGCCAAGCTCTAG |
| | | CCTGGGCACCCAGACCTACATCTGCAATGTGAACCACAAGCCTAG |
| | | CAACACCAAGGTGGACGACAGAGTG(SEQ ID NO: 86) |
| | Ck (E123K + Q124R) | CGTACGGTGGCCGCTCCTAGCGTGTTCATCTTTCCACCTAGCGAC |
| | | AAGAGACTGAAGTCTGGCACAGCCTCTGTCGTGTGCCTGCTCAAC |
| | | AACTTCTACCCCAGAGAAGCCAAGGTGCAGTGGAAGGTGGACAAC |
| | | GCCCTGCAGAGCGGCAATAGCCAAGAGAGCGTGACCGAGCAGGA |
| | | CAGCAAGGACTCTACCTACAGCCTGAGCAGCACACTGACCCTGAG |
| | | CAAGGCCGACTACGAGAAGCACAAAGTGTACGCCTGCGAAGTGAC |
| | | CCACCAGGGCCTTTCTAGCCCTGTGACCAAGAGCTTCAACCGGGG |
| | | CGAGTGCTGATAA (SEQ ID NO: 157) |
| | Hinge-CH2-CH3 (LS) | GAACCCAAGAGCTGCGACAAGACCCACACCTGTCCTCCATGTCCT |
| | (G1m3) | GCTCCAGAACTGCTCGGCGGACCTTCCGTGTTCCTGTTTCCTCCA |
| | | AAGCCTAAGGACACCCTGATGATCAGCAGAACCCCTGAAGTGACC |
| | | TGCGTGGTGGTGGATGTGTCTCACGAGGACCCCGAAGTGAAGTT |
| | | CAATTGGTACGTGGACGGCGTGGAAGTGCACAACGCCAAGACCAA |
| | | GCCTAGAGAGGAACAGTACAACAGCACCTACAGAGTGGTGTCCGT |
| | | GCTGACAGTGCTGCACCAGGATTGGCTGAACGGCAAAGAGTACAA |
| | | GTGCAAGGTGTCCAACAAGGCCCTGCCTGCTCCTATCGAGAAAAC |
| | | CATCAGCAAGGCCAAGGGCCAGCCTAGGGAACCCCAGGTTTACAC |
| | | ACTGCCTCCAAGCCGCGAGGAAATGACCAAGAACCAGGTGTCCCT |
| | | GACCTGCCTCGTGAAGGGCTTCTACCCTTCCGATATCGCCGTGGA |
| | | ATGGGAGAGCAATGGCCAGCCTGAGAACAACTACAAGACAACCCC |
| | | TCCTGTGCTGGACAGCGACGGCTCATTCTTCCTGTACAGCAAGCT |
| | | GACCGTGGACAAGTCCAGATGGCAGCAGGGCAACGTGTTCAGCT |
| | | GTAGCGTGCTGCACGAAGCCCTGCACAGCCACTACACCCAGAAAA |
| | | GCCTGTCTCTGAGCCCCGGCAAATGATAA (SEQ ID NO: 1204) |
| | husec2 v1 | ATGGATTGGATTTGGAGAATCCTGTTCCTCGTGGGCGCCGCTACC |
| | | GGAGCCCACTCC (SEQ ID NO: 3) |
| | husec2 v2 | ATGGATTGGATCTGGCGCATCCTGTTTCTCGTGGGAGCTGCCACA |
| | | GGCGCCCACTCT (SEQ ID NO: 5) |
| | 5′UTR (cap proximal | AGAATAAACTAGTATTCTTCTGGTCCCCACAGACTCAGAGAGAAC |
| | sequence + human alpha- | CCGCCACC (SEQ ID NO: 11) |
| | Globin + Kozak) | |
| | 3′UTR (FI-Element + | CTGGTACTGCATGCACGCAATGCTAGCTGCCCCTTTCCCGT |
| | polyA30LA70) | CCTGGGTACCCCGAGTCTCCCCCGACCTCGGGTCCCAGGT |
| | | ATGCTCCCACCTCCACCTGCCCCACTCACCACCTCTGCTAG |
| | | TTCCAGACACCTCCCAAGCACGCAGCAATGCAGCTCAAAAC |
| | | GCTTAGCCTAGCCACACCCCCACGGGAAACAGCAGTGATT |
| | | AACCTTTAGCAATAAACGAAAGTTTAACTAAGCTATACTAA |
| | | CCCCAGGGTTGGTCAATTTCGTGCCAGCCACACCCTCGAGCT |
| | | AGCAAAAAAAAAAAAAAAAAAAAAAAAAAAAAAGCATATGACTAAA |
| | | AAAAAAAAAAAAAAAAAAAAAAAAAAAAAAAAAAAAAAAAAAAAAA |
| | | AAAAAAAAAAAAAAAAAAAAA (SEQ ID NO: 1195) |
| PGDM1400 | VH v2 | CAAGTTCACCTGACACAGTCTGGCCCCGAAGTGCGGAAGCCTGGC |
| | | ACAAGCGTGAAGGTGTCCTGTAAAGCCCCTGGCAACACCCTGAAA |
| | | ACCTACGACCTGCACTGGGTCCGATCTGTGCCTGGACAAGGACTG |
| | | CAGTGGATGGGCTGGATTAGCCACGAGGGCGACAAGAAAGTGAT |
| | | CGTGGAACGGTTCAAGGCCAAAGTGACCATCGACTGGGACAGAA |
| | | GCACCAACACCGCCTACCTGCAGCTGAGCGGACTGACATCTGGCG |
| | | ATACCGCCGTGTACTACTGTGCCAAGGGCAGCAAGCACCGGCTGA |
| | | GAGATTACGCCCTGTACGACGATGACGGCGCCCTGAATTGGGCCG |
| | | TCGATGTGGACTACCTGAGCAACCTGGAATTCTGGGGCCAGGGAA |
| | | CAGCCGTGACAGTGTCTAGT (SEQ ID NO: 40) |
| | VL | GATTTTGTCCTGACTCAGTCTCCACACTCTCTGTCCGTCACCCCTG |
| | | GAGAGTCGGCCTCCATCTCCTGCAAGTCTAGTCACAGCCTCATTC |
| | | ATGGTGATAGGAACAATTATTTGGCTTGGTACGTACAGAAGCCAG |
| | | GGCGGTCTCCACAACTCCTGATCTATTTGGCTTCCAGTCGGGCCT |
| | | CCGGGGTCCCTGACAGGTTCAGTGGCAGTGGATCGGACAAAGAT |
| | | TTTACACTGAAGATCAGCAGAGTGGAGACTGAGGATGTTGGGAC |
| | | GTATTACTGCATGCAAGGTCGAGAAAGTCCCTGGACGTTCGGCCA |
| | | AGGGACCAAGGTGGACATCAAA |
| | | (SEQ ID NO: 45) |

An overview of the individual plasmids are shown in Table 18 below.

TABLE 18
Exemplary plasmids encoding CrossMab chains

| Molecule | Target Site | Format | Plasmid Insert |
|---|---|---|---|
| PGDM1400 | V1V2Apex | IgG1 | VH(PGDM1400)-CK-Fc(LS) (SEQ ID NO: 1317) |
| | | CrossMab^{CH1-CLx} | VH(PGDM1400)-CK-Fc(GAALIE LS) (SEQ ID NO: 1329) |
| | | | VH(PGDM1400)-CK-Fc(cah LS) (SEQ ID NO: 1332) |
| | | | VH(PGDM1400)-CK-Fc(GAALIE cah LS) (SEQ ID NO: |
| | | | 1335) |
| | | | VH(PGDM1400)-CK-Fc(cak LS) (SEQ ID NO: 1338) |
| | | | VL(PGDM1400)-CH1 (SEQ ID NO: 1320) |
| | | IgG1 | VH(PGDM1400)-CH1cv-Fc (LS) (SEQ ID NO: 1323) |
| | | CrossMab^{CH1-CLcv} | VL(PGDM1400)-CKcv (SEQ ID NO: 1326) |

### Linearization and DNA Purification

Linearization of plasmid DNA was performed using appropriate restriction enzymes, followed by purification of the linearized DNA template using magnetic beads (Dynabeads™ MyOne™ Carboxylic Acid) according to the manufacturer's protocol. The DNA concentration was measured by UV spectroscopy.

### In Vitro Transcription

CleanCap 413 ((m7(3′OMeG)(5′)ppp(5′)(2′OMeA)pG) capped RNA was produced following the process as disclosed, e.g., in Kreiter, S. et al. Cancer Immunol. Immunother. 56, 1577-87 (2007) and WO 2021214204A1, each of which is incorporated herein by reference in its entirety. Methyl pseudo-uridine was used in the in vitro transcription reaction and incorporated into the produced RNA. Cellulose purification of the resulting RNA was performed to isolate single-stranded RNA, followed by concentration measurement by UV spectroscopy. The RNA integrity was determined by microfluidic-based electrophoresis. The resulting RNA constructs are shown in SEQ ID NOs: 900, 901, 905, 906, 910, 950 (heavy chain) and 947 and 997 (light chain).

### Example 5: In Vitro Validation of PGDM1400 IgG1 Antibody Agents Delivered to Cells Using RNA Constructs

### Expression and Neutralization Analysis of PGDM1400 and PGDM1400 L/S Antibody Agents Delivered to Cells Using RNA Constructs

In order to determine whether polyribonucleotides encoding PGDM1400 IgG antibody agents, as described herein, can induce the production of the PGDM1400 IgG antibody agents, the expression and neutralization capabilities of the PGDM1400 IgG antibody agents were tested.

Additionally, it is advantageous that antibody agents delivered by polyribonucleotides may be able to reach a high antibody titer in the serum of subjects, e.g., over a period of time. Thus, a mutation was introduced (“L/S”) in the CH₃ domain of certain antibody agents to improve binding to the FcRn receptor and thereby improve the terminal kinetic and increase the half-life of the antibody agent (see, for example, Zalevsky et al., 2010, Nature biotechnology, which is incorporated herein by reference in its entirety). In this Example, PGDM1400 antibody agents designed with this mutation were evaluated for whether the mutation impacts certain parameters like expression or neutralization abilities. Antibody agents may also be modified with GAALIE mutations (G236A/A330L/I332E) in the Fc region so as to enhance dendritic cell maturation and induce protective CD8+ T cell responses (see, for example, Corti et al., 2021, Cell, which is incorporated herein by reference).

In this experiment, the expression of PGDM1400 and PGDM1400 L/S IgG antibody agents from polyribonucleotides described herein was tested.

Objectives of this Example include: Evaluating the expression and purity of PGDM1400 and PGDM1400 L/S IgG antibody agents by Gyros and Western blot.

In this Example, HEK293T/17 cells were electroporated with polyribonucleotides encoding an PGDM1400 IgG antibody agent (polyribonucleotides shown in SEQ ID NOs: 1306 (heavy chain) and 1312 (light chain)), an PGDM1400 L/S IgG antibody agent (polyribonucleotides shown in SEQ ID NOs: 1309 (heavy chain) and 1312 (light chain)), and a control antibody agent (referred to as “RiboMab01”) (1.5:1 HC/LC). The respective antibody agent concentrations in the supernatant were determined by GYROS ELISA. Reducing and non-reducing denaturing Western blot analyses were also performed to check the ratio of antibody agents produced versus high molecular weight (HMW) aggregates and free chains.

### Electroporation

25 μg RNA (1.5/1 HC/LC, 15 μg HC+10 μg LC) of polyribonucleotides encoding PGDM1400 HC, PGDM1400 L/S HC and PGDM1400 LC in IgG1 format were delivered to HEK293T/17 cells electroporation in triplicate. RiboMab01 was used as a control. First, HEK293T/17 cells were washed 2×with 20 ml cooled medium. Electroporation was performed in triplicate in pre-cooled 0.4 cm cuvettes. Cells in each sample were in a concentration of 2*10⁶ cells/250 μl or 8*10⁶ cells/ml. Electroporation conditions were 250V, 2 pulses for 5 ms. Cells were incubated on ice for 10 minutes after electroporation. 0.75 ml of Expi293 Expression Medium was prepared in a well of a 12-well plate for resuspension of each sample.

Other cells were then transferred into Expi293 Medium and counted. Cells were seeded at 2*10⁶ cells/ml or 1 ml/well and incubated for 48 hours at 37° C. Supernatants were harvested by centrifugation of the cells at 300g for 10 minutes, followed by careful aspiration so as to not disturb the cell pellet, and then stored at 4° C.

### Gyros ELISA for RibobNAb Quantitation

PGDM1400 antibody agents expressed from polyribonucleotides delivered to a cell (referred to herein as “RibobNAbs”), as described in Examples 2-4, were quantified in serum using Gyros xPand™ XPA1025 ELISA device (Gyros Protein Technologies AB, Uppsala, Sweden). A sandwich immunoassay format was performed using a biotinylated anti-human IgG Fc antibody (Reagent A, ready-to-use solution) for capture and Alexa® Fluor® 647 labeled anti-human IgG (Reagent B, ready-to use solution, both from Generic PK Kit-Low Titer or Generic TK Kit-High Titer, Gyros Protein Technologies AB) for the detection of RibobNab IgG, IgG L/S formats.

A sandwich immunoassay format was also performed to determine intact (fully assembled) RibobNAbs IgG, IgG L/S, IgG CrossMab or scFv-Fc content in cell culture supernatant samples from HEK293T17 cells transfected with mRNA. The RibobNAbs were captured by a biotinylated derivative of protein A from Staphylococcus aureus (Reagent A, ready-to-use solution from Gyrolab huIgG Kit, Gyros Protein Technologies AB). An Alexa Fluor 647 labeled F(ab′)2 fragment of anti-human IgG (Reagent B, ready-to-use solution from Gyrolab huIgG Kit, Gyros Protein Technologies AB) was used for detection of RibobNAb IgG, IgG L/S or IgG CrossMab formats and 25 nM of Goat anti-Human IgG (H+L) Cross-Adsorbed Secondary Antibody, Alexa Fluor 647 (Thermo Fisher Scientific, Darmstadt) in Rexxip F (Gyros Protein Technologies AB) for scFv-Fc format, respectively.

The assay was processed for low titer in a Gyrolab Bioaffy 1000 HC CD (Gyros Protein Technologies AB) with a dynamic range of 1.4 to 333 ng/mL (IgG, IgG L/S formats in serum) and 12.3 to 9,000 ng/mL (IgG, IgG L/S, IgG CrossMab formats in cell culture supernatant), or for high titer in a Gyrolab Bioaffy 20 HC CD (Gyros Protein Technologies AB) with a dynamic range of 111 to 234,000 ng/mL (IgG, IgG L/S in serum) and 111 to 9,000 ng/mL (scFv-Fc Format in cell culture supernatant), respectively.

All samples, reference proteins and reagents were centrifuged for 4 minutes at 12,000×g to sediment any aggregates. Serum samples containing the respective RibobNAb molecules were diluted 10-fold in Reagent F buffer (Gyrolab PK Low Titer/TK Kit High Titer, Gyros Protein Technologies AB) and RibobNAbs molecules in cell culture supernatant diluted 5 to 10-fold in Reagent E buffer (Gyrolab huIgG Kit, Gyros Protein Technologies AB), respectively. The CD columns were washed with Reagent C and D (Gyrolab PK/TK Kit, Gyrolab huIgG Kit, Gyros Protein Technologies AB).

The Gyrolab huIgG Standard (Gyros Protein Technologies AB), recombinant PGT121 L/S antibody (Lake Pharma Inc., San Carlos, CA) and the recombinant Anti-APRIL, human IgG1 lambda scFv-Fc (Biomol GmbH, Hamburg) served as reference proteins to calculate IgG, IgG L/S, IgG CrossMab or scFv-Fc RibobNAbs concentration in the assays, respectively.

For RibobNAb quantitation, all materials prepared for the Gyros ELISA assay were loaded onto a 96-well plate (Gyros Protein Technologies AB) according to the Gyrolab loading list. Data were generated for low RibobNAb titers with the Gyrolab Generic PK kit method v1 (serum) and huIgG Low Titer method (cell culture supernatant) and for high RibobNab titers with Gyrolab Generic TK kit method v1 (serum) and huIgG High Titer v2 method (cell culture supernatant), respectively. Results were evaluated using the Gyrolab Evaluator software.

All materials prepared for the Gyros ELISA assay were loaded onto a 96-well plate (Gyros Protein Technologies AB) according to the Gyrolab Loading list.

Data was generated for low RibobNAb titers with the Gyrolab Generic PK kit method v1 and the results evaluated using Gyrolab Evaluator software.

### Western Blot Analysis of RibobNAbs in Cell Culture Supernatants

Cell culture supernatant (SN) samples were first mixed with varying volumes of SN from mock-transfected HEK293T/17 cells, a fixed volume of water as well as 4× Laemmli buffer and heated to 95° C. for 5 minutes under non-reducing conditions. Two different samples of the reference protein (5726/5725p) were used for gauging RibobNAb quality in the SN, containing (i) 0.23% or (ii) 96.5% HMW intermediates. As negative control, SN from Mock-transfected HEK-293T-17 cells was used. The samples were separated by SDS-PAGE for 40 minutes followed by transfer of the separated bands onto a blotting membrane (Bio-Rad). For western blotting, the membrane was incubated with two HRP-conjugated detection antibodies: goat anti-human kappa LC at a 1:500 dilution (ID176, Thermo Fisher Scientific) and goat anti-human IgG Fd region pAb (Cell Sciences, Newburyport, MA, USA) at a 1:2,000 dilution in 3% BSA Fraction V (Eurobio Scientific, Les Ulis, France). The membrane was visualized with Clarity Western ECL Reagent (Bio-Rad) on a Vilber Fusion FX imaging device (Vilber, Collégien, France) for 4 seconds and the data analyzed with Image Lab Software (Bio-Rad).

### Results

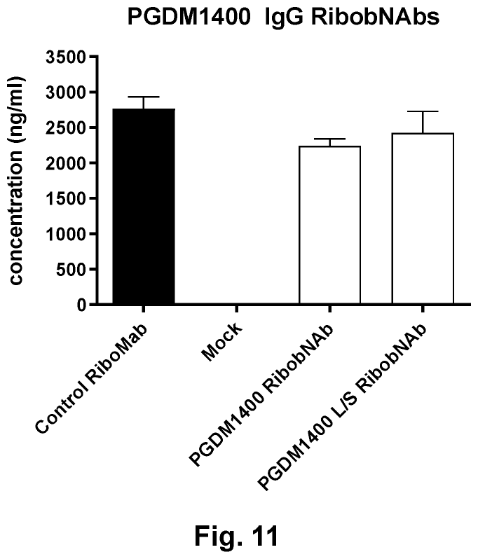

The results from the Gyros ELISA in this Example show that PGDM1400 and PGDM1400 L/S IgG antibody agents were produced at a similar level to the control RiboMab “RiboMab01” (see FIG. 11).

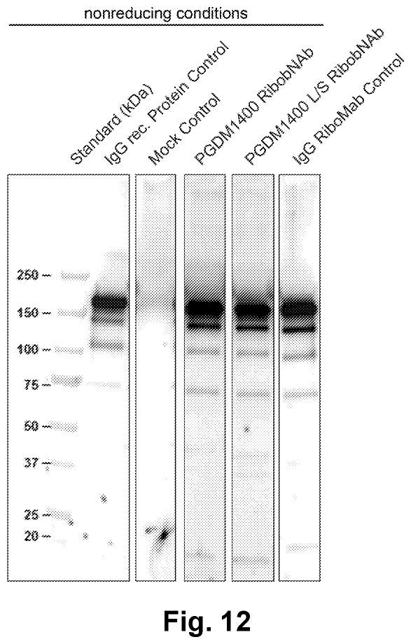

The results from the Western blot analysis are shown in FIG. 12. The non-reducing denaturing Western blot of PGDM1400 and PGDM1400 L/S IgG antibody agents did not show aggregates, HMW aggregates, or free (unbound) light chains. The Western blot results of PGDM1400 IgG antibody agent, PGDM1400 L/S IgG antibody agent, and the RiboMab01 control displayed a similar expression pattern with a main fraction of the antibodies as fully assembled monomeric (>80%) at the correct height. Reducing conditions revealed HC and LC bands were found at the correct height. Non-reducing conditions revealed PGDM1400 IgG antibody agent and PGDM1400 L/S IgG antibody agent monomer bands. These results suggest that PGDM1400 IgG antibody agent and PGDM1400 L/S IgG antibody agent were correctly assembled.

### Neutralization Studies

The TZM-bl assay was used to determine the neutralization capacity of PGDM1400 and PGDM1400 L/S IgG antibody agents (e.g., as described in Sarzotti-Kelsoe et al., 2014. Journal of immunological methods, which is incorporated herein by reference). TZM-bl cells are engineered to express CD4, CCR5, and CXCR4 and to contain integrated reporter genes for firefly Luc and E. coli β-galactosidase under the control of an HIV-1 long terminal repeat.

First, HEK293T/17 cells were transfected with RNA which encoded for IgG RibobNAb antibody agent. HEK293T/17 cells were then cultured under suitable conditions and for a suitable length of time so as to allow for RibobNAb production and secretion into the cell culture supernatant. HEK293T/17 cell culture supernatant was then harvested and added to TZM-bl cells at the same time that samples containing virus were added to TZM-bl cells. The TZM-bl cells were then incubated for about 48 hours, followed by addition of a Luc reporter gene assay system reagent. Virus infectivity was measured in relative luminescence units (RLU).

Initial HIV neutralization studies showed that PGDM1400 IgG antibody agent and PGDM1400 L/S IgG antibody agents had good breadth and potency (data not shown). Additional HIV neutralization studies assessing capabilities of the PGDM1400 and PGDM1400 L/S IgG antibody agents using a Viral Neutralization Test (VNT) will be performed. The supernatant from this Experiment will also be used to test the HIV neutralization capabilities using a VNT.

### Example 6: In Vitro Validation of scFv-Fc PGDM1400 L/S Antibody Agents Delivered to Cells Using RNA Constructs

Expression and Neutralization Analysis of scFv-Fc PGDM1400 L/S Antibody Agents Delivered to Cells Using RNA Constructs

Generation of polyribonucleotides encoding for PGDM1400 antibody agents as described herein allows the PGDM1400 antibody agents to be delivered in combination with other polyribonucleotides that encode additional anti-HIV antibody agents. Use of the technologies and approaches described herein also allows for simultaneous production of different antibody agents from polyribonucleotides. The formats of antibody agents have been designed to minimize or eliminate the risk of immunoglobulin chain mispairing. Being able to combine multiple antibody agent formulations as described herein (e.g., including an PGDM1400 antibody agent) allows for the development of a composition (e.g., a pharmaceutical composition) that delivers multiple antibody agents together so that they can bind different epitopes of the HIV virus, thereby minimizing viral escape through mutations.

In order to determine whether polyribonucleotides encoding scFv-Fc PGDM1400 L/S antibody agents, as described herein, can induce the production of the scFv-Fc PGDM1400 L/S antibody agents, the expression and neutralization capabilities of the scFv-Fc PGDM1400 L/S antibody agents were tested. Different domain orientations of VH:VL and linkers lengths were utilized in the scFv-Fc PGDM1400 L/S antibody agents tested.

Objectives of this Example include:
(1) Evaluate the expression and aggregation of scFv-Fc PGDM1400 L/S antibody agents by Gyros and Western Blot; and
(2) Test the influence of different domain orientations and linker length on the production and functionality of scFv-Fc PGDM1400 L/S antibody agents.

In this Example, HEK293T/17 cells were electroporated with polyribonucleotides encoding various scFv-Fc PGDM1400 L/S antibody agents (polyribonucleotides as shown in SEQ ID NO: 1342 (“VH-LL4-VL”), SEQ ID NO: 1345 (“VH-LL5-VL”), SEQ ID NO: 1348 (“VL-LL4-VH”), and SEQ ID NO: 1351 (“VL-LL5-VH”)) and a control RiboMab01 (1.5:1 HC/LC). The scFv-Fc PGDM1400 L/S antibody agent concentration in the supernatant was determined by GYROS ELISA. A non-reducing denaturing Western blot analysis was also performed to check the ratio of antibody agents produced as compared to HMW aggregates and free chains.

### Methods

Electroporation, Gyros ELISA for RibobNAb Quantification, and Western Blot Analysis of RibobNAbs in Cell Culture Supernatants were performed as described above in Example 5.

### Results

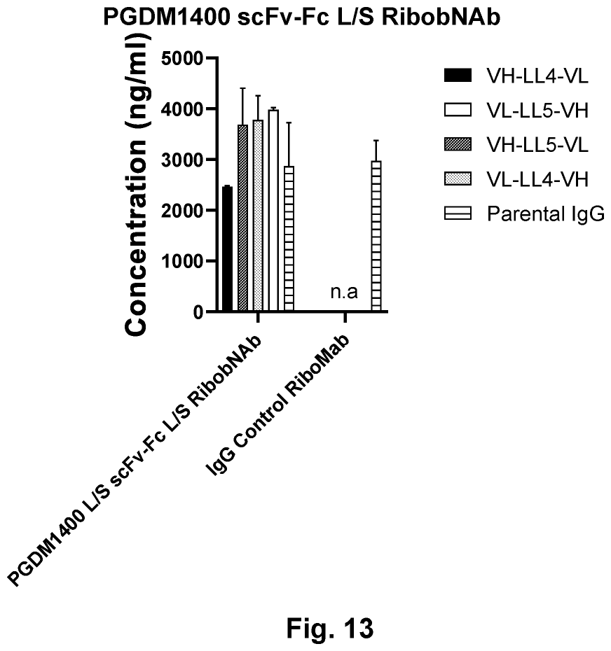

The results from the Gyros ELISA in this Example show that all scFv-Fc PGDM1400 L/S antibody agents were successfully expressed. VL-LL5-VH orientation was optimal for expression of a scFv-Fc PGDM1400L/S antibody agent. FIG. 13 shows the results relative to parental IgG.

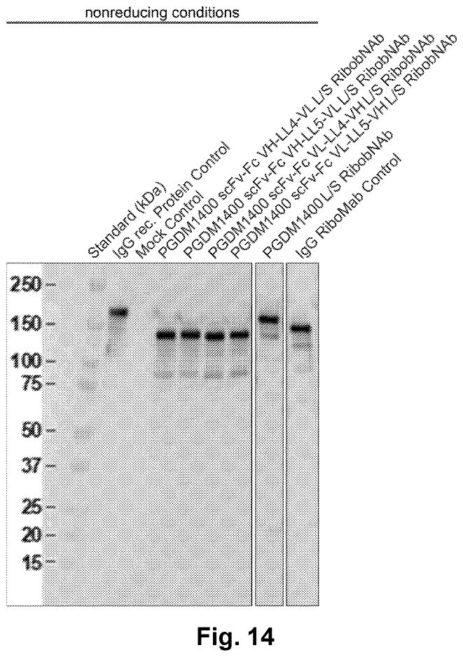

The results from the Western Blot Analysis are shown in FIG. 14. The results from the Western Blot analysis show that all scFv-Fc PGDM1400L/S antibody agents are correctly assembled and show no Aggregates and LMWs.

### Neutralization Studies

The TZM-bl assay was used to determine the neutralization capacity of scFv-Fc PGDM1400 antibody agents (e.g., as described in Sarzotti-Kelsoe et al., 2014. Journal of immunological methods). TZM-bl cells are engineered to express CD4, CCR5, and CXCR4 and to contain integrated reporter genes for firefly Luc and E. coli β-galactosidase under the control of an HIV-1 long terminal repeat.

Briefly, HEK293T/17 cells were transfected with RNA that encoded for IgG RibobNAb antibody agent (PGDM1400 scFv-Fc and PGDM1400 parental IgG control). HEK293T/17 cells were then cultured under suitable conditions and for a suitable length of time so as to allow for RibobNAb production and secretion into the cell culture supernatant. HEK293T/17 cell culture supernatant was then harvested and added to TZM-bl cells at the same time that samples containing virus were added to TZM-bl cells. The TZM-bl cells were then incubated for about 48 hours, followed by addition of a Luc reporter gene assay system reagent. Virus infectivity was measured in relative luminescence units (RLU).

In detail: Pseudovirus generation: HIV-1 Env pseudotyped viruses were prepared by co-transfecting exponentially-dividing HEK293T/17 cells (5×10⁶ cells in 15 ml growth medium in a T-75 culture flask) with 4 μg of rev/env expression plasmid and 8 μg of an env-deficient HIV-1 backbone vector (pSG3Δenv) using FuGENE 6 reagent (Promega, USA) in growth medium, as described by the manufacturer. Virus-containing culture supernatants were recovered from the flasks and filtered through a 0.45 μm filter. Env-pseudotyped virus stocks were titrated by performing serial 5-fold dilutions in growth medium in 96-well culture plates (11 dilution steps total). Freshly trypsinized TZM-bl cells (10,000 cells in 100 μl volume) were added to each well in growth medium containing an optimized concentration of DEAE-dextran and incubated for 48 hrs. 100 μl of culture supernatant was removed from each well and replaced with a Luc reporter gene assay system reagent (Brite-Glo, Promega, used as per manufacturer's recommendation). After a 2-min incubation at room temperature to allow cell lysis, 150 μl of cell lysate was transferred to 96-well black solid plates (Corning-Costar) for measurements of luminescence. For each Env pseudotyped virus an optimal dilution to use in the TZM-bl assay (expressed as RLU equivalent) was calculated to ensure a standardized virus dose.

pVNT assay: HEK293T/17 cells were transfected with RNA that encoded for PGDM1400 RibobNAb L/S antibody agents (VH-LL4-VL, VL-LL5-VH, VH-LL5-VL, and VL-LL4-VH configurations). HEK293T/17 cells were then cultured under suitable conditions and for a suitable length of time so as to allow for RibobNAb production and secretion into the cell culture supernatant. Supernatants from cells transfected with polyribonucleotides or purified IgG samples were tested at indicated primary dilutions/concentrations and serially diluted 3-fold 7-times in duplicate wells. Diluted samples were mixed with an optimal titer of Env-pseudotyped virus and co-incubated at 37° C. for 1 h. TZM-bl cells were then added at a final concentration of 10⁴ cells per well in a 96-well plate in medium supplemented with DEAE-dextran for 48 hrs at 37° C. and 5% CO2. 100 μl of culture supernatant was removed from each well and replaced with a Luc reporter gene assay system reagent (Brite-Glo, Promega, used as per manufacturer's recommendation). After a 2-min incubation at room temperature to allow cell lysis, 150 μl of cell lysate was transferred to 96-well black solid plates (Corning-Costar) for measurements of luminescence. After subtracting background relative luminescence units (RLUs) of non-infected TZM-bl cells, 50% and 80% inhibitory concentrations (IC50s and IC80s) were determined as the antibody/IgG concentrations resulting in a 50/o/80% RLU reduction compared to untreated virus control wells. The following HIV-1 Env pseudovirus isolates were selected for testing against cell supernatants or purified IgG: ZM53M.PB12, Du156.12, Q769.d22, 0330.v4.c3, R2184.c04, 89-F1_2_25, CAP304_2_00_F6_6, Ce1176_A3, 6980.v0.c31, 1012_11_TC21_3257, and PVO.4. A Murine Leukemia Virus (MuLV) pseudovirus was used as a negative control.

Initial HIV neutralization studies showed that scFv-Fc PGDM1400 L/S antibody agents had good breadth and potency (data not shown). Additional HIV neutralization studies assessing capabilities of the scFv-Fc PGDM1400 L/S antibody agents using a Viral Neutralization Test (VNT) will be performed. The supernatant from this Experiment will also be used to test the HIV neutralization capabilities using a VNT.

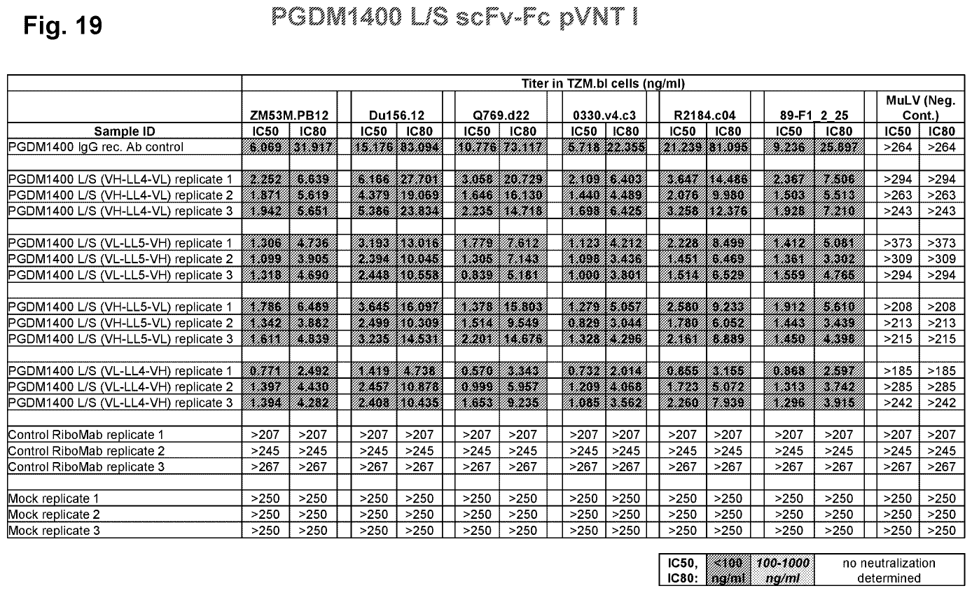

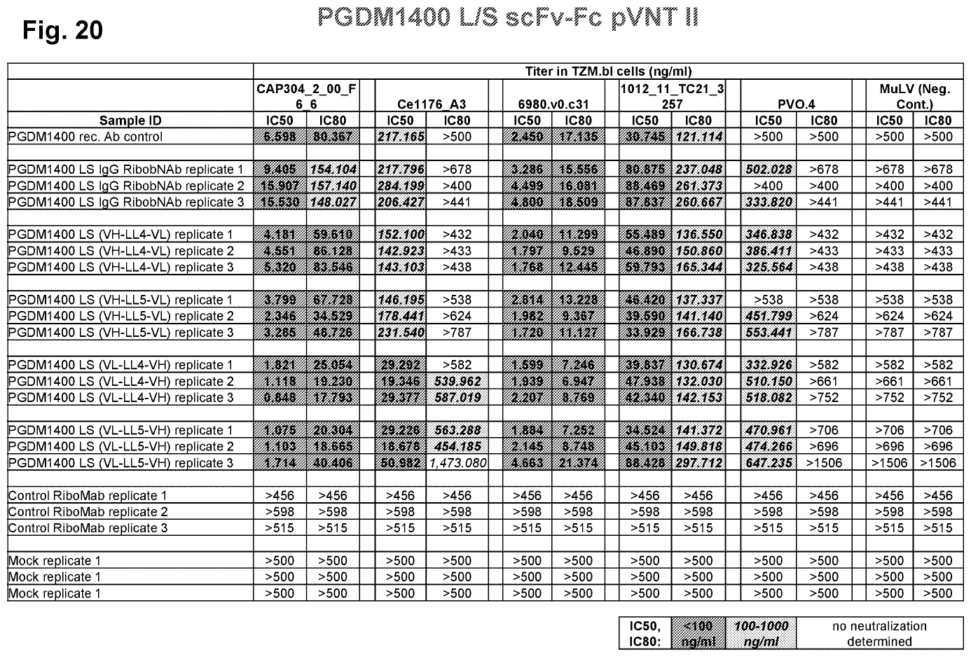

FIG. 19 and FIG. 20 show results from pVNT assays and confirm that scFv-Fc PGDM1400 L/S antibody agents had good breadth and potency. Specifically, PGDM1400 L/S scFv-Fc RibobNAb variants are functional and neutralize HIV strains more efficiently than the parental PGDM1400 IgG control. Additionally, PGDM1400 L/S scFv-Fc VL-VH RibobNAb variants (VL-LL4-VH and VL-LL5-VH) have increased potency against a PGDM1400 insensitive strain Ce1176_A3 compared to the parental PGDM1400 IgG control and the PGDM1400 L/S scFv-Fc VH-VL RibobNAb variants (VH-LL4-VL and VH-LL5-VL) (FIG. 20).

Additionally, pVNT assays were performed according to the same methods described above, testing further neutralization capacity of scFv-Fc VL-VH RibobNAb variants VL-LL5-VH (the full RNA construct sequence as shown in SEQ ID NO: 603; the polyribonucleotide encoding the scFv-Fc chain as shown in SEQ ID NO: 1351, which encodes the amino acid sequence as shown in SEQ ID NO: 1352) against the following HIV-1 Env pseudovirus isolates: CAP45, X2088_c9, CNE8, Du172.17, T250-4, QH0692.42, 3103.v3.c10, C1080_c3, T278-50, ZM109F.PB4, and Du156.12. It had previously been observed that PGDM1400 does not neutralize strains X2088_C9 and QH0692.42. Accordingly, these strains served as negative controls.

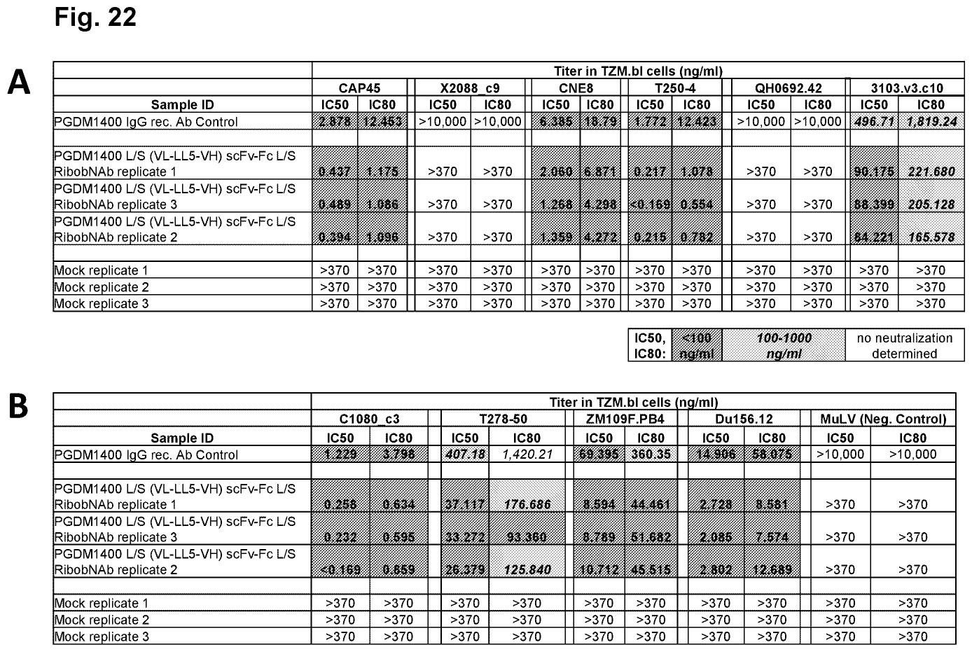

FIG. 22 shows results from the pVNT assay. scFv-Fc LS RibobNAbs (VL-LL5-VH) showed good breadth and potency against the additional strains tested. The scFv-Fc LS RibobNAb construct (VL-LL5-VH) did not neutralize strains X2088_C9 and QH0692.42 as expected. Additionally, scFv-Fc LS RibobNAbs (VL-LL5-VH) showed improved neutralization with respect to the parental IgG control.

### Example 7: In Vitro Validation of CrossMab PGDM1400 L/S Antibody Agents Delivered to Cells Using RNA Constructs

Expression and Neutralization Analysis of PGDM1400 CrossMab^{CH1-CLx} and PGDM1400 CrossMab^{CH1-CLcv} Antibody Agents Delivered to Cells Using RNA Constructs

Generation of polyribonucleotides encoding for PGDM1400 antibody agents as described herein allows the PGDM1400 antibody agents to be delivered in combination with other polyribonucleotides that encode additional anti-HIV antibody agents. Use of the technologies and approaches described herein also allows for simultaneous production of different antibody agents from polyribonucleotides. The formats of antibody agents have been designed to minimize or eliminate the risk of immunoglobulin chain mispairing. Being able to combine multiple antibody agent formulations as described herein (e.g., including a PGDM1400antibody agent) allows for the development of a composition (e.g., a pharmaceutical composition) that delivers multiple antibody agents together so that they can bind different epitopes of the HIV virus, thereby minimizing viral escape through mutations.

In order to determine whether polyribonucleotides encoding CrossMab PGDM1400 L/S antibody agents, as described herein, can induce the production of the CrossMab PGDM1400 L/S antibody agents, the expression and neutralization capabilities of the CrossMab PGDM1400 L/S antibody agents were tested.

Objectives of this Example include:
(1) Evaluate the expression and aggregation of CrossMab PGDM1400 L/S antibody agents by Gyros and Western Blot; and
(2) Test the influence of different domain swaps and charge variations on the production and functionality of scFv-Fc PGDM1400 L/S antibody agents.

In this Example, HEK293T/17 cells were electroporated with polyribonucleotides encoding various CrossMab^{CH1-CLx} PGDM1400 L/S antibody agents (polyribonucleotides as shown in SEQ ID NO: 1318 (VH(PGDM1400)-CK-Fc(LS)) and 1321 (VL(PGDM1400)-CH₁); SEQ ID NO: 1330 VH(PGDM1400)-CK-Fc(GAALIE LS) and SEQ ID NO: 1321; SEQ ID NO: 1333 (VH(PGDM1400)-CK-Fc(cah LS)) and SEQ ID NO: 1321; and SEQ ID NO: 1339 (VH(PGDM1400)-CK-Fc(cak LS)) and SEQ ID NO: 1321) and CrossMab^{CH1-CLcv} (SEQ ID NO: 1324 (VH(PGDM1400)-CH₁cv-Fc (LS))) and SEQ ID NO: 1327) and a control RiboMab01 (1.5:1 HC/LC). The CrossMab PGDM1400 L/S antibody agent concentration in the supernatant was determined by GYROS ELISA. A non-reducing denaturing Western blot analysis was also performed to check the ratio of antibody agents produced as compared to HMW aggregates and free chains.

### Methods

Electroporation, Gyros ELISA for RibobNAb Quantification, and Western Blot Analysis of RibobNAbs in Cell Culture Supernatants were performed as described above in Example 5.

### Results

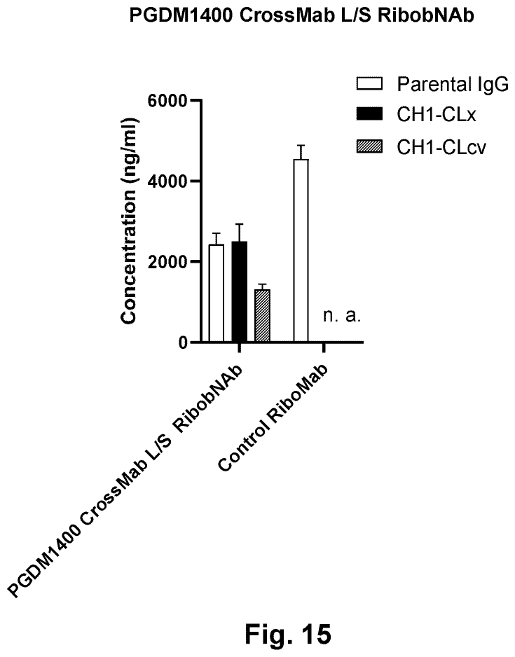

The results from the Gyros ELISA are shown in FIG. 15. The results from the Gyros ELISA showed that all PGDM1400 CrossMab^{CH1-CLx} and PGDM1400 CrossMab^{CH1-CLcv} antibody agents were successfully expressed and produced in HEK-293T/17 cells. Additionally, results showed that PGDM1400 CrossMab^{CH1-CLcv} L/S was expressed at a level about 3 times lower compared to the control RiboMab01 and at about half the level of expression of the parental control (PGDM1400 L/S IgG antibody agent), and that the PGDM1400CrossMab^{CH1-CLcx} antibody agent was expressed at about half the level of the control RiboMab01 and at a similar level as the parental control (PGDM1400 LS IgG antibody agent).

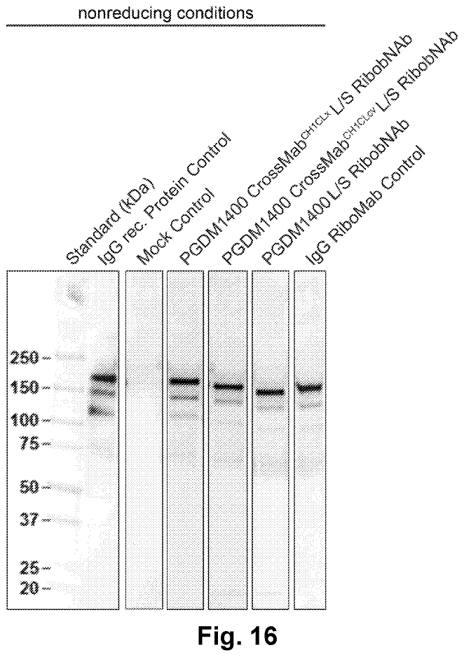

The results from the Western Blot analysis are shown in FIG. 16. The Western Blot Analysis showed that all PGDM1400 CrossMab^{CH1-CLx} and PGDM1400 CrossMab^{CH1-CLcv} antibody agents were correctly assembled and show no aggregates or HMW aggregates.

### Neutralization Studies

A TZM-bl assay was used to determine the neutralization capacity of PGDM1400 CrossMab antibody agents (e.g., as described in Sarzotti-Kelsoe et al., 2014. Journal of immunological methods). TZM-bl cells are engineered to express CD4, CCR5, and CXCR4 and to contain integrated reporter genes for firefly Luc and E. coli β-galactosidase under the control of an HIV-1 long terminal repeat.

Briefly, HEK293T/17 cells were transfected with RNA that encoded for IgG RibobNAb antibody agent (PGDM1400 CH₁-CHx L/S, PGDM1400 CH₁-CLcv, and PGDM1400 parental IgG control). HEK293T/17 cells were then cultured under suitable conditions and for a suitable length of time so as to allow for RibobNAb production and secretion into the cell culture supernatant. HEK293T/17 cell culture supernatant was then harvested and added to TZM-bl cells at the same time that samples containing virus were added to TZM-bl cells. The TZM-bl cells were then incubated for about 48 hours, followed by addition of a Luc reporter gene assay system reagent. Virus infectivity was measured in relative luminescence units (RLU).

In detail: Pseudovirus generation: HIV-1 Env pseudotyped viruses were prepared by co-transfecting exponentially-dividing HEK293T/17 cells (5×10⁶ cells in 15 ml growth medium in a T-75 culture flask) with 4 μg of rev/env expression plasmid and 8 μg of an env-deficient HIV-1 backbone vector (pSG3Δenv) using FuGENE 6 reagent (Promega, USA) in growth medium, as described by the manufacturer. Virus-containing culture supernatants were recovered from the flasks and filtered through a 0.45 μm filter. Env-pseudotyped virus stocks were titrated by performing serial 5-fold dilutions in growth medium in 96-well culture plates (11 dilution steps total). Freshly trypsinized TZM-bl cells (10,000 cells in 100 μl volume) were added to each well in growth medium containing an optimized concentration of DEAE-dextran and incubated for 48 hrs. 100 μl of culture supernatant was removed from each well and replaced with a Luc reporter gene assay system reagent (Brite-Glo, Promega, used as per manufacturer's recommendation). After a 2-min incubation at room temperature to allow cell lysis, 150 μl of cell lysate was transferred to 96-well black solid plates (Corning-Costar) for measurements of luminescence. For each Env pseudotyped virus an optimal dilution to use in the TZM-bl assay (expressed as RLU equivalent) was calculated to ensure a standardized virus dose.

pVNT assay: HEK293T/17 cells were transfected with RNA which encoded for IgG RibobNAb antibody agent (PGDM1400 CH₁-CHx L/S, PGDM1400 CH₁-CLcv, and parental IgG control). HEK293T/17 cells were then cultured under suitable conditions and for a suitable length of time so as to allow for RibobNAb production and secretion into the cell culture supernatant.

Supernatants from cells electroporated with polyribonucleotides or purified IgG samples were tested at indicated primary dilutions/concentrations and serially diluted 3-fold 7-times in duplicate wells. Diluted samples were mixed with an optimal titer of Env-pseudotyped virus and co-incubated at 37° C. for 1 h. TZM-bl cells were then added at a final concentration of 10⁴ cells per well in a 96-well plate in medium supplemented with DEAE-dextran for 48 hrs at 37° C. and 5% C02. 100 μl of culture supernatant was removed from each well and replaced with a Luc reporter gene assay system reagent (Brite-Glo, Promega, used as per manufacturer's recommendation). After a 2-min incubation at room temperature to allow cell lysis, 150 μl of cell lysate was transferred to 96-well black solid plates (Corning-Costar) for measurements of luminescence. After subtracting background relative luminescence units (RLUs) of non-infected TZM-bl cells, 50% and 80% inhibitory concentrations (IC50s and IC80s) were determined as the antibody/IgG concentrations resulting in a 50%/80% RLU reduction compared to untreated virus control wells. The following HIV-1 Env pseudovirus isolates were selected for testing against cell supernatants or purified IgG: ZM53M.PB12, Du156.12, Q769.d22, 0330.v4.c3, R2184.c04, 89-F1_2_25. A Murine Leukemia Virus (MuLV) pseudovirus was used as a negative control.

Initial HIV neutralization studies showed that PGDM1400 CrossMab^{CH1-CLx} antibody agent and PGDM1400 CrossMab^{CH1-CLcv} antibody agents had good breadth and potency (data not shown). Additional HIV neutralization studies assessing capabilities of the PGDM1400 CrossMab antibody agents using a Viral Neutralization Test (VNT) will be performed. The supernatant from this Experiment will also be used to test the HIV neutralization capabilities using a VNT.

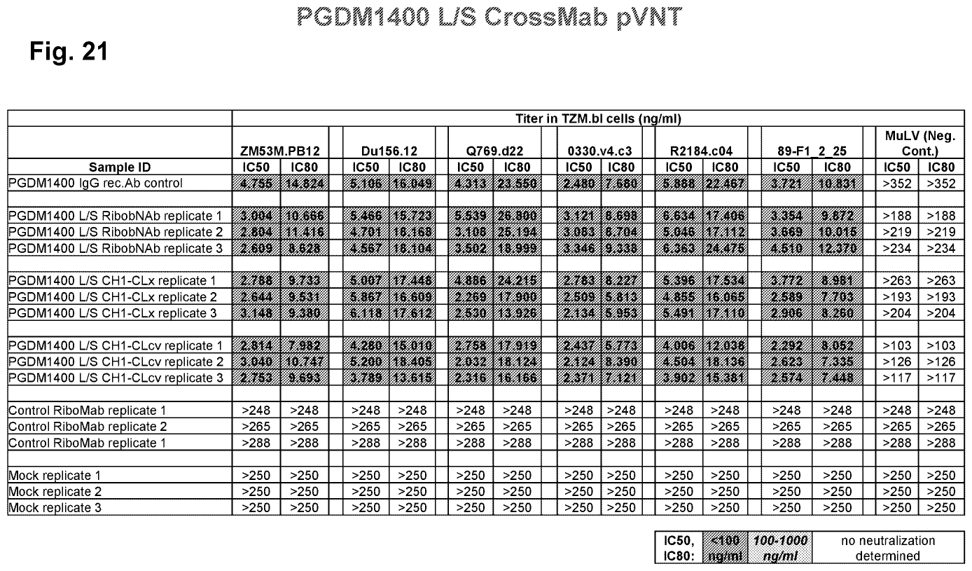

FIG. 21 shows results from pVNT assays and confirm that PGDM1400 CrossMab^{CH1-CLx} antibody agents and PGDM1400 CrossMab^{CH1-CLcv} antibody agents had good breadth and retain potency.

### Example 8: In Vivo Validation of Parental and Mutated PGDM1400 RibobNAbs

Generation of polyribonucleotides encoding for PGDM1400 antibody agents as described herein allows the PGDM1400 antibody agents to be delivered in combination with other polyribonucleotides that encode additional anti-HIV antibody agents. Use of the technologies and approaches described herein also allows for simultaneous production of different antibody agents from polyribonucleotides. The formats of antibody agents have been designed to minimize or eliminate the risk of immunoglobulin chain mispairing. Being able to combine multiple antibody agent formulations as described herein (e.g., including an PGDM1400 antibody agent) allows for the development of a composition (e.g., a pharmaceutical composition) that delivers multiple antibody agents together so that they can bind different epitopes of the HIV virus, thereby minimizing viral escape through mutations.

The present Example shows that polyribonucleotides encoding PGDM1400 antibody agents can induce production of PGDM1400 antibody agents in vivo. Furthermore, the present Example shows that polyribonucleotides encoding PGDM1400 antibody agents can drive a high antibody titer in the serum of test subjects over a period of time. A mutation (“L/S”) was introduced in the CH₃ domain of certain antibody agents to improve binding to the FcRn receptor and thereby improve the terminal kinetic and increase the half-life of the antibody agent (see, for example, Zalevsky et al., 2010, Nature biotechnology). In this Example, the pharmacokinetic properties of PGDM1400 and PGDM1400 L/S IgG antibody agents were evaluated to assess the mutation's effect on such antibody qualities. NSG and NSG hFcRn TG32 mice were used. NSG hFcRn TG32 mice include a genetic modification where the murine FcRn receptor has been exchanged to the human version.

The concentration of the PGDM1400 Antibody and L/S mutated PGDM1400 Antibody in serum of NSG and NSG hFcRn Tg32 mice was analyzed.

NSG and NSG hFcRn TG32 mice were injected intravenously with LNP-encapsulated polyribonucleotides encoding PGDM1400 and PGDM1400 L/S IgG antibody agents (1.5:1 HC/LC) (polyribonucleotides as shown in SEQ ID NO: 1306 and 1312 (PGDM1400 IgG HC and LC, respectively) and SEQ ID NO: 1309 and 1312 (PGDM1400 L/S IgG HC and LC, respectively)) in 150 μL of PBS. Blood was sampled from the both mice strains at various time points, and serum was analyzed for the abundance of the RibobNAbs over a time period of either 25 (NSG mice) or 33 days (NSG hFcRn TG32 mice) with Gyros ELISA, as described herein.

### Results

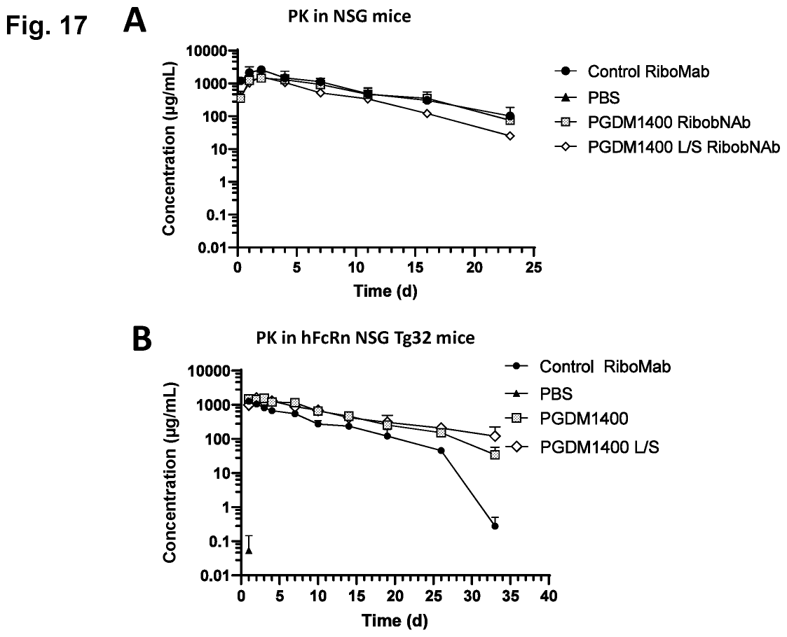

The results in NSG and NSG hFcRn TG32 mice are presented in FIG. 17 (panel A and panel B, respectively), respectively, in log scale. In NSG mice, the PGDM1400 IgG antibody agent had a similar pharmacokinetic profile to the control, RiboMab01. Furthermore, the PGDM1400 L/S IgG antibody agent displayed similar pharmacokinetics as compared to the PGDM1400 IgG antibody. In the NSG hFcRn T32 mice, PGDM1400 IgG antibody agent had improved pharmacokinetic profile compared to the control, RiboMab01. Additionally, the PGDM1400 L/S IgG antibody agent displayed improved pharmacokinetics as compared to the PGDM1400 IgG antigen agent, confirming that the L/S mutation can indeed extend antibody half-life in NSG hFcRn T32 mice. In particular, the PGDM1400 L/S IgG antibody agent had, on average, a concentration of 121 μg/ml in vivo on day 33 post-injection and the PGDM1400 IgG antibody agent had, on average, a concentration of 34 μg/ml in vivo on day 33 post-injection.

### Example 9: Generation of Polyribonucleotides Encoding PGDM1400 IgG Antibody Agents

The present Example demonstrates the generation of polyribonucleotide sequences encoding an anti-HIV antibody agent. The present Example further demonstrates that such polyribonucleotides are designed so that transient in vivo antibody agent production following i.v./i.m./s.c. delivery of the one or more polyribonucleotides is achieved.

Specifically, ribonucleic acid sequences encoding the heavy and light chain variable domains of PGDM1400 recombinantly combine with other immunoglobulin domains to form polyribonucleotides that encode PGDM1400 antibody agent immunoglobulin chains. The resulting polyribonucleotides are cloned into the JR81 DNA plasmid. The polyribonucleotides encode PGDM1400 antibody agents having different formats, including a conventional IgG1, an scFv-Fc, a CrossMab^{CH1-CLx}, a CrossMab^{CH1-CLcv}, and knob-in-hole formats (see e.g., FIGS. 4-5). Exemplary PGDM1400 antibody agent configurations and component immunoglobulin chains are shown in Table 2.

Methods of the present example include:
(1) Cloning of DNA fragments into a DNA plasmid appropriate to use for RNA expression (e.g., JR81); and
(2) Verification of selected clones by control digestion and sequencing.

An appropriate DNA plasmid may encode RNA features including, for example, a 5′ untranslated region (e.g., one that is derived from human α-globin mRNA (hAg)), a Kozak sequence, a signal peptide sequence (e.g., husec2)), and/or a 3′ untranslated region (e.g., one that is a combination of two sequence elements (FI element) and/or a polyA tail 30Linker7O (A30L70)). Examples of appropriate DNA plasmids can be found in WO2021214204A1, which is hereby incorporated by reference in its entirety.

### Codon Optimization and Cloning Strategy

Codon optimization and cloning are performed as described above in Example 2.

Exemplary sequences used in the cloning methods above are shown in Table 19 below.

TABLE 19
Exemplary sequences in PGDM1400 antibody agent encoding polyribonucleotides.

| Feature | Fragment | Nucleotide Sequence |
|---|---|---|
| General | CH1-Hinge-CH2 | GCCTCTACAAAGGGCCCTAGCGTGTTCCCTCTGGCTCCTAGCAGCAAGT |
| | (GAIE)-CH3 (LS) | CGACATCTGGCGGAACAGCCGCTCTGGGCTGTCTGGTCAAGGACTACTT |
| | (G1m3) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1205) |
| | CH1-Hinge-CH2-CH3 | GCCTCTACAAAGGGCCCTAGCGTGTTCCCTCTGGCTCCTAGCAGCAAGT |
| | (G1m17) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACACTGCCTCCAAGCCGCGAGGAAATGACCAAGAACCAGGTGTCCC |
| | | TGACCTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACTGTGGATA |
| | | AGTCCCGGTGGCAGCAGGGCAACGTGTTCAGCTGTTCTGTGATGCACG |
| | | AAGCCCTGCACAACCACTACACCCAGAAAAGCCTGTCTCTGAGCCCCGG |
| | | CAAGTGATAA (SEQ ID NO: 1206) |
| | CH1-Hinge-CH2-CH3 | GCCTCTACAAAGGGCCCTAGCGTGTTCCCTCTGGCTCCTAGCAGCAAGT |
| | (LS) (G1m17) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACACTGCCTCCAAGCCGCGAGGAAATGACCAAGAACCAGGTGTCCC |
| | | TGACCTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACCGTGGACA |
| | | AGTCCAGATGGCAGCAGGGCAACGTGTTCAGCTGTAGCGTGCTGCACG |
| | | AAGCCCTGCACAGCCACTACACCCAGAAAAGCCTGTCTCTGAGCCCCGG |
| | | CAAATGATAA (SEQ ID NO: 1207) |
| | CH1-Hinge-CH2-CH3 | GCCTCTACAAAGGGCCCTAGCGTGTTCCCTCTGGCTCCTAGCAGCAAGT |
| | (cak-LS) (G1m17) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACCCTGCCTCCATGCCGCGAGGAAATGACCAAGAACCAGGTGTCCC |
| | | TGTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1208) |
| | CH1-Hinge-CH2 | GCCTCTACAAAGGGCCCTAGCGTGTTCCCTCTGGCTCCTAGCAGCAAGT |
| | (GAALIE)-CH3 (LS) | CGACATCTGGCGGAACAGCCGCTCTGGGCTGTCTGGTCAAGGACTACTT |
| | (G1m17) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1209) |
| | CH1-Hinge-CH2 | GCCTCTACAAAGGGCCCTAGCGTGTTCCCTCTGGCTCCTAGCAGCAAGT |
| | (GAIE)-CH3 (LS) | CGACATCTGGCGGAACAGCCGCTCTGGGCTGTCTGGTCAAGGACTACTT |
| | (G1m17) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1210) |
| | CH1-Hinge-CH2-CH3 | GCCTCTACAAAGGGCCCTAGCGTGTTCCCTCTGGCTCCTAGCAGCAAGT |
| | (G1m17,1) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACACTGCCTCCAAGCCGCGACGAACTGACCAAGAACCAGGTGTCCC |
| | | TGACCTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACTGTGGATA |
| | | AGTCCCGGTGGCAGCAGGGCAACGTGTTCAGCTGTTCTGTGATGCACG |
| | | AAGCCCTGCACAACCACTACACCCAGAAAAGCCTGTCTCTGAGCCCCGG |
| | | CAAGTGATAA (SEQ ID NO: 1211) |
| | CH1-Hinge-CH2-CH3 | GCCTCTACAAAGGGCCCTAGCGTGTTCCCTCTGGCTCCTAGCAGCAAGT |
| | (LS) (G1m17,1) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACACTGCCTCCAAGCCGCGACGAACTGACCAAGAACCAGGTGTCCC |
| | | TGACCTGCCTCGTGAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACCGTGGACA |
| | | AGTCCAGATGGCAGCAGGGCAACGTGTTCAGCTGTAGCGTGCTGCACG |
| | | AAGCCCTGCACAGCCACTACACCCAGAAAAGCCTGTCTCTGAGCCCCGG |
| | | CAAATGATAA (SEQ ID NO: 1212) |
| | CH1-Hinge-CH2-CH3 | GCCTCTACAAAGGGCCCTAGCGTGTTCCCTCTGGCTCCTAGCAGCAAGT |
| | (cak-LS) (G1m17,1) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTAGGGAACCCCAGGTT |
| | | TACACCCTGCCTCCATGCCGCGACGAACTGACCAAGAACCAGGTGTCCC |
| | | TGTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1213) |
| | CH1-Hinge-CH2 | GCCTCTACAAAGGGCCCTAGCGTGTTCCCTCTGGCTCCTAGCAGCAAGT |
| | (GAALIE)-CH3 (LS) | CGACATCTGGCGGAACAGCCGCTCTGGGCTGTCTGGTCAAGGACTACTT |
| | (G1m17,1) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTAGGGAACCCCAGGTTT |
| | | ACACACTGCCTCCAAGCCGCGACGAACTGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1214) |
| | CH1-Hinge-CH2 | GCCTCTACAAAGGGCCCTAGCGTGTTCCCTCTGGCTCCTAGCAGCAAGT |
| | (GAIE)-CH3 (LS) | CGACATCTGGCGGAACAGCCGCTCTGGGCTGTCTGGTCAAGGACTACTT |
| | (G1m17,1) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACACTGCCTCCAAGCCGCGACGAACTGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1215) |
| | CH1-Hinge-CH2(GA)- | GCCTCTACAAAGGGCCCTAGCGTGTTCCCTCTGGCTCCTAGCAGCAAGT |
| | CH3 (LS) (G1m3) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTATCGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1216) |
| | CH1-Hinge-CH2(IE)- | GCCTCTACAAAGGGCCCTAGCGTGTTCCCTCTGGCTCCTAGCAGCAAGT |
| | CH3 (LS) (G1m3) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1217) |
| | CH1-Hinge-CH2-CH3 | GCCTCTACAAAGGGCCCTAGCGTGTTCCCTCTGGCTCCTAGCAGCAAGT |
| | (cah-LS) (G1m3) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTCGCGAACCTCAAGTC |
| | | TGTACACTGCCTCCTAGCCGCGAGGAAATGACCAAGAACCAGGTGTCCC |
| | | TGAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1218) |
| | CH1-Hinge- | GCCTCTACAAAGGGCCCTAGCGTGTTCCCTCTGGCTCCTAGCAGCAAGT |
| | CH2(GAALIE)-CH3 | CGACATCTGGCGGAACAGCCGCTCTGGGCTGTCTGGTCAAGGACTACTT |
| | (cah-LS) (G1m3) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTCGCGAACCTCAAGTCT |
| | | GTACACTGCCTCCTAGCCGCGAGGAAATGACCAAGAACCAGGTGTCCCT |
| | | GAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1219) |
| | CH1-Hinge- | GCCTCTACAAAGGGCCCTAGCGTGTTCCCTCTGGCTCCTAGCAGCAAGT |
| | CH2(GAIE)-CH3 (cah- | CGACATCTGGCGGAACAGCCGCTCTGGGCTGTCTGGTCAAGGACTACTT |
| | LS) (G1m3) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTCGCGAACCTCAAGTCT |
| | | GTACACTGCCTCCTAGCCGCGAGGAAATGACCAAGAACCAGGTGTCCCT |
| | | GAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1220) |
| | CH1-Hinge- | GCCTCTACAAAGGGCCCTAGCGTGTTCCCTCTGGCTCCTAGCAGCAAGT |
| | CH2(GAALIE)-CH3 | CGACATCTGGCGGAACAGCCGCTCTGGGCTGTCTGGTCAAGGACTACTT |
| | (cak-LS) (G1m3) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTAGGGAACCCCAGGTTT |
| | | ACACCCTGCCTCCATGCCGCGAGGAAATGACCAAGAACCAGGTGTCCCT |
| | | GTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1221) |
| | CH1-Hinge- | GCCTCTACAAAGGGCCCTAGCGTGTTCCCTCTGGCTCCTAGCAGCAAGT |
| | CH2(GAIE)-CH3 (cak- | CGACATCTGGCGGAACAGCCGCTCTGGGCTGTCTGGTCAAGGACTACTT |
| | LS) (G1m3) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAGAGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACCCTGCCTCCATGCCGCGAGGAAATGACCAAGAACCAGGTGTCCCT |
| | | GTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1222) |
| | CH1-Hinge-CH2(GA)- | GCCTCTACAAAGGGCCCTAGCGTGTTCCCTCTGGCTCCTAGCAGCAAGT |
| | CH3 (LS) (G1m17) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTATCGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1223) |
| | CH1-Hinge-CH2(IE)- | GCCTCTACAAAGGGCCCTAGCGTGTTCCCTCTGGCTCCTAGCAGCAAGT |
| | CH3 (LS) (G1m17) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACACTGCCTCCAAGCCGCGAGGAAATGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1224) |
| | CH1-Hinge-CH2-CH3 | GCCTCTACAAAGGGCCCTAGCGTGTTCCCTCTGGCTCCTAGCAGCAAGT |
| | (cah-LS) (G1m17) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTCGCGAACCTCAAGTC |
| | | TGTACACTGCCTCCTAGCCGCGAGGAAATGACCAAGAACCAGGTGTCCC |
| | | TGAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1225) |
| | CH1-Hinge- | GCCTCTACAAAGGGCCCTAGCGTGTTCCCTCTGGCTCCTAGCAGCAAGT |
| | CH2(GAALIE)-CH3 | CGACATCTGGCGGAACAGCCGCTCTGGGCTGTCTGGTCAAGGACTACTT |
| | (cah-LS) (G1m17) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTCGCGAACCTCAAGTCT |
| | | GTACACTGCCTCCTAGCCGCGAGGAAATGACCAAGAACCAGGTGTCCCT |
| | | GAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1226) |
| | CH1-Hinge- | GCCTCTACAAAGGGCCCTAGCGTGTTCCCTCTGGCTCCTAGCAGCAAGT |
| | CH2(GAIE)-CH3 (cah- | CGACATCTGGCGGAACAGCCGCTCTGGGCTGTCTGGTCAAGGACTACTT |
| | LS) (G1m17) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTCGCGAACCTCAAGTCT |
| | | GTACACTGCCTCCTAGCCGCGAGGAAATGACCAAGAACCAGGTGTCCCT |
| | | GAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1227) |
| | CH1-Hinge- | GCCTCTACAAAGGGCCCTAGCGTGTTCCCTCTGGCTCCTAGCAGCAAGT |
| | CH2(GAALIE)-CH3 | CGACATCTGGCGGAACAGCCGCTCTGGGCTGTCTGGTCAAGGACTACTT |
| | (cak-LS) (G1m17) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTAGGGAACCCCAGGTTT |
| | | ACACCCTGCCTCCATGCCGCGAGGAAATGACCAAGAACCAGGTGTCCCT |
| | | GTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1228) |
| | CH1-Hinge- | GCCTCTACAAAGGGCCCTAGCGTGTTCCCTCTGGCTCCTAGCAGCAAGT |
| | CH2(GAIE)-CH3 (cak- | CGACATCTGGCGGAACAGCCGCTCTGGGCTGTCTGGTCAAGGACTACTT |
| | LS) (G1m17) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACCCTGCCTCCATGCCGCGAGGAAATGACCAAGAACCAGGTGTCCCT |
| | | GTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1229) |
| | CH1-Hinge-CH2(GA)- | GCCTCTACAAAGGGCCCTAGCGTGTTCCCTCTGGCTCCTAGCAGCAAGT |
| | CH3 (LS) (G1m17,1) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTATCGAA |
| | | AAGACCATCAGCAAGGCCAAGGGCCAGCCTAGGGAACCCCAGGTTT |
| | | ACACACTGCCTCCAAGCCGCGACGAACTGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1230) |
| | CH1-Hinge-CH2(IE)- | GCCTCTACAAAGGGCCCTAGCGTGTTCCCTCTGGCTCCTAGCAGCAAGT |
| | CH3 (LS) (G1m17,1) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACACTGCCTCCAAGCCGCGACGAACTGACCAAGAACCAGGTGTCCCT |
| | | GACCTGCCTCGTGAAGGGCTTCTACCCTTCCGATATCGCCGTGGAATGG |
| | | GAGAGCAATGGCCAGCCTGAGAACAACTACAAGACAACCCCTCCTGTGC |
| | | TGGACAGCGACGGCTCATTCTTCCTGTACAGCAAGCTGACCGTGGACAA |
| | | GTCCAGATGGCAGCAGGGCAACGTGTTCAGCTGTAGCGTGCTGCACGA |
| | | AGCCCTGCACAGCCACTACACCCAGAAAAGCCTGTCTCTGAGCCCCGGC |
| | | AAATGATAA (SEQ ID NO: 1231) |
| | CH1-Hinge-CH2-CH3 | GCCTCTACAAAGGGCCCTAGCGTGTTCCCTCTGGCTCCTAGCAGCAAGT |
| | (cah-LS) (G1m17,1) | CGACATCTGGCGGAACAGCCGCTCTGGGCTGTCTGGTCAAGGACTACTT |
| | | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTCGGCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCAGAAGTGAAGTT |
| | | CAATTGGTACGTGGACGGCGTGGAAGTGCACAACGCCAAGACC |
| | | AAGCCTAGAGAGGAACAGTACAACAGCACCTACAGAGTGGTGTC |
| | | CGTGCTGACAGTGCTGCACCAGGATTGGCTGAACGGCAAAGAG |
| | | TACAAGTGCAAGGTGTCCAACAAGGCCCTGCCTGCTCCTATCGA |
| | | GAAAACCATCAGCAAGGCCAAGGGCCAGCCTCGCGAACCTCAAGTC |
| | | TGTACACTGCCTCCTAGCCGCGACGAACTGACCAAGAACCAGGTGTCCC |
| | | TGAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1232) |
| | CH1-Hinge- | GCCTCTACAAAGGGCCCTAGCGTGTTCCCTCTGGCTCCTAGCAGCAAGT |
| | CH2(GAALIE)-CH3 | CGACATCTGGCGGAACAGCCGCTCTGGGCTGTCTGGTCAAGGACTACTT |
| | (cah-LS) (G1m17,1) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTCGCGAACCTCAAGTCT |
| | | GTACACTGCCTCCTAGCCGCGACGAACTGACCAAGAACCAGGTGTCCCT |
| | | GAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1233) |
| | CH1-Hinge- | GCCTCTACAAAGGGCCCTAGCGTGTTCCCTCTGGCTCCTAGCAGCAAGT |
| | CH2(GAIE)-CH3 (cah- | CGACATCTGGCGGAACAGCCGCTCTGGGCTGTCTGGTCAAGGACTACTT |
| | LS) (G1m17,1) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTCGCGAACCTCAAGTCT |
| | | GTACACTGCCTCCTAGCCGCGACGAACTGACCAAGAACCAGGTGTCCCT |
| | | GAGCTGCGCCGTGAAGGGCTTTTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGGTGTCCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1234) |
| | CH1-Hinge- | GCCTCTACAAAGGGCCCTAGCGTGTTCCCTCTGGCTCCTAGCAGCAAGT |
| | CH2(GAALIE)-CH3 | CGACATCTGGCGGAACAGCCGCTCTGGGCTGTCTGGTCAAGGACTACTT |
| | (cak-LS) (G1m17,1) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTCTGCCTGAGGAA |
| | | AAGACCATCAGCAAGGCCAAGGGCCAGCCTAGGGAACCCCAGGTTT |
| | | ACACCCTGCCTCCATGCCGCGACGAACTGACCAAGAACCAGGTGTCCCT |
| | | GTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1235) |
| | CH1-Hinge- | GCCTCTACAAAGGGCCCTAGCGTGTTCCCTCTGGCTCCTAGCAGCAAGT |
| | CH2(GAIE)-CH3 (cak- | CGACATCTGGCGGAACAGCCGCTCTGGGCTGTCTGGTCAAGGACTACTT |
| | LS) (G1m17,1) | TCCCGAGCCTGTGACAGTGTCCTGGAACTCTGGCGCTCTGACAAGCGG |
| | | CGTGCACACATTTCCAGCCGTGCTGCAAAGCAGCGGCCTGTACTCTCTG |
| | | AGCAGCGTGGTCACAGTGCCAAGCTCTAGCCTGGGCACCCAGACCTACA |
| | | TCTGCAATGTGAACCACAAGCCTAGCAACACCAAGGTGGACAAGAAGGT |
| | | GGAACCCAAGAGCTGCGACAAGACCCACACCTGTCCTCCATGTCCTGCT |
| | | CCAGAACTGCTGGCCGGACCTTCCGTGTTCCTGTTTCCTCCAAA |
| | | GCCTAAGGACACCCTGATGATCAGCAGAACCCCTGAAGTGACCT |
| | | GCGTGGTGGTGGATGTGTCTCACGAGGACCCTGAAGTGAAGTTC |
| | | AATTGGTACGTGGACGGCGTGGAAGTGCACAACGCCAAGACCA |
| | | AGCCTAGAGAGGAACAGTACAACAGCACCTACAGAGTGGTGTCC |
| | | GTGCTGACCGTGCTGCACCAGGATTGGCTGAACGGCAAAGAGT |
| | | ACAAGTGCAAGGTGTCCAACAAGGCCCTGCCTGCCCCTGAGGAA |
| | | AAGACCATCAGCAAGGCCAAGGGCCAGCCTAGGGAACCCCAGGTTT |
| | | ACACCCTGCCTCCATGCCGCGACGAACTGACCAAGAACCAGGTGTCCCT |
| | | GTGGTGCCTGGTCAAGGGCTTCTACCCTTCCGATATCGCCGTGGAATG |
| | | GGAGAGCAATGGCCAGCCTGAGAACAACTACAAGACAACCCCTCCTGTG |
| | | CTGGACAGCGACGGCTCATTCTTCCTGTACAGCAAGCTGACAGTGGACA |
| | | AGAGCAGATGGCAGCAGGGCAACGTGTTCAGCTGCAGCGTGCTGCATG |
| | | AAGCCCTGCACAGCCACTACACCCAGAAGTCCCTGTCTCTGAGCCCCGG |
| | | CAAGTGATAA (SEQ ID NO: 1236) |

### Plasmid DNA Preparation

Plasmid DNA is prepared by selecting E. coli clones for inoculation in Luria-Bertani (LB) medium containing kanamycin. The cultures are grown overnight at 37° C. and ~150 to 200 rpm. Following cell harvest, purification is done using the QIAGEN Plasmid Plus Maxi Kit according to the manufacturer's instructions. Finally, the concentration is determined by UV spectroscopy. DNA was stored in certified RNase- and DNase-free reaction tubes.

### Linearization and DNA Purification

Linearization of plasmid DNA is performed using appropriate restriction enzymes, followed by purification of the linearized DNA template using magnetic beads (Dynabeads™ MyOne™ Carboxylic Acid) according to the manufacturer's protocol. The DNA concentration is measured by UV spectroscopy.

### In Vitro Transcription

CleanCap 413 (m7(3′OMeG)(5′)ppp(5′)(2′OMeA)pG) capped RNA is produced following the process as disclosed, e.g., in WO 2021214204A1, which is incorporated herein by reference in its entirety. Methyl pseudo-uridine is used in the in vitro transcription reaction and incorporated into the produced RNA. Cellulose purification of the resulting RNA is performed to isolate single-stranded RNA, followed by concentration measurement by UV spectroscopy. The RNA integrity is determined by microfluidic-based electrophoresis.

### Example 10: Generation of Polyribonucleotides Encoding scFv-Fc PGDM1400

In this Example, polyribonucleotides encoding an PGDM1400 antibody agent are designed in silico as an scFv with different VH-to-VL orientations as well as interconnecting linkers having two different lengths (e.g., (G4S)₄ and (G4S)₅). The nucleotide sequences encoding the scFvs are cloned with a nucleotide sequence encoding an Fc domain to produce Fc-fusion constructs, which encode an antibody agent referred to as an “scFv-Fc” (illustrated in, e.g., FIG. 4, Panel C). The Fc-fusion constructs are cloned into the JR81 vector. scFv-Fc variants are cloned with and without an “LS” mutation. Lead candidates are further optimized by (i) introducing one of two different sequences encoding linkers (e.g., a (G4S)4 or (G4S)5 linker) between the scFv and Fc domain. Exemplary PGDM1400 antibody agent configurations and component immunoglobulin chains are shown in Table 5.

Objectives of the present Example include:
(1) Cloning scFv-Fc PGDM1400 L/S-encoding DNA into JR81 plasmid; and
(2) Verification of selected clones by control digestion and sequencing.

### Codon Optimization and Cloning Strategy

Codon optimization and cloning are performed as described above in Example 2. The nucleotide sequence of the resulting scFv-Fc chain-encoding plasmid constructs as well as the sequence of the backbone can be found in SEQ ID NOs: 1356, 1359, 1362, 1365, 1368, 1371, 1374, 1377, 1380, 1383, 1386, 1389, 1392, 1395, 1398, 1401, 1404, 1407, 1410, 1413, 1416, 1419, 1422, 1425, 1428, 1431, 1434, 1437, 1440, 1443, 1446, 1449, 1452, 1455, or 1458. Such sequences encode scFv-Fc chain amino acid sequences shown in SEQ ID NO: 1358, 1361, 1364, 1367, 1370, 1373, 1376, 1379, 1382, 1385, 1388, 1391, 1394, 1397, 1400, 1403, 1406, 1409, 1412, 1415, 1418, 1421, 1424, 1427, 1430, 1433, 1436, 1439, 1442, 1445, 1448, 1451, 1454, 1457, and 1460.

Exemplary sequences used in the cloning methods above are shown in Table 20 below.

TABLE 20
Exemplary sequences in scFv-Fc PGDM1400 encoding_polyribonucleotides.

| Feature | Fragment | Nucleotide Sequence |
|---|---|---|
| General | Hinge (C/S)-CH2- | GAACCTAAGAGCAGCGACAAGACCCACACCTGTCCTCCATGTCCTGCTCCAG |
| | CH3 (cah-LS) | AACTGCTCGGCGGACCTTCCGTGTTCCTGTTTCCTCCAAAGCCTAAG |
| | (G1m3) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTCGCGAACCTCAAGTCTGTACACTGCCTCCTAGCCGCGAGGAAATGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1237) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (cah-LS) (G1m3) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTCGCGAACCTCAAGTCTGTACACTGCCTCCTAGCCGCGAGGAAATGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1238) |
| | Hinge (C/S)-CH2 | GAACCTAAGAGCAGCGACAAGACCCACACCTGTCCTCCATGTCCTGCTCCAG |
| | (GAIE)-CH3 (cah- | AACTGCTGGCCGGACCTTCCGTGTTCCTGTTTCCTCCAAAGCCTAAG |
| | LS) (G1m3) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTCGCGAACCTCAAGTCTGTACACTGCCTCCTAGCCGCGAGGAAATGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1239) |
| | Hinge (C/S)-CH2- | GAACCTAAGAGCAGCGACAAGACCCACACCTGTCCTCCATGTCCTGCTCCAG |
| | CH3 (cak-LS) | AACTGCTCGGCGGACCTTCCGTGTTCCTGTTTCCTCCAAAGCCTAAG |
| | (G1m3) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACCCTGCCTCCATGCCGCGAGGAAATGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1240) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (cak-LS) (G1m3) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTAGGGAACCCCAGGTTTACACCCTGCCTCCATGCCGCGAGGAAATGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1241) |
| | Hinge (C/S)-CH2 | GAACCTAAGAGCAGCGACAAGACCCACACCTGTCCTCCATGTCCTGCTCCAG |
| | (GAIE)-CH3 (cak- | AACTGCTGGCCGGACCTTCCGTGTTCCTGTTTCCTCCAAAGCCTAAG |
| | LS) (G1m3) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTAGGGAACCCCAGGTTTACACCCTGCCTCCATGCCGCGAGGAAATGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1242) |
| | Hinge (C/S)-CH2- | GAACCTAAGAGCAGCGACAAGACCCACACCTGTCCTCCATGTCCTGCTCCAG |
| | CH3 (cah-LS) | AACTGCTCGGCGGACCTTCCGTGTTCCTGTTTCCTCCAAAGCCTAAG |
| | (G1m17) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTCGCGAACCTCAAGTCTGTACACTGCCTCCTAGCCGCGAGGAAATGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1243) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (cah-LS) (G1m17) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTCGCGAACCTCAAGTCTGTACACTGCCTCCTAGCCGCGAGGAAATGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1244) |
| | Hinge (C/S)-CH2 | GAACCTAAGAGCAGCGACAAGACCCACACCTGTCCTCCATGTCCTGCTCCAG |
| | (GAIE)-CH3 (cah- | AACTGCTGGCCGGACCTTCCGTGTTCCTGTTTCCTCCAAAGCCTAAG |
| | LS) (G1m17) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTCGCGAACCTCAAGTCTGTACACTGCCTCCTAGCCGCGAGGAAATGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1245) |
| | Hinge (C/S)-CH2- | GAACCTAAGAGCAGCGACAAGACCCACACCTGTCCTCCATGTCCTGCTCCAG |
| | CH3 (cak-LS) | AACTGCTCGGCGGACCTTCCGTGTTCCTGTTTCCTCCAAAGCCTAAG |
| | (G1m17) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACCCTGCCTCCATGCCGCGAGGAAATGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1246) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (cak-LS) (G1m17) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTAGGGAACCCCAGGTTTACACCCTGCCTCCATGCCGCGAGGAAATGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1247) |
| | Hinge (C/S)-CH2 | GAACCTAAGAGCAGCGACAAGACCCACACCTGTCCTCCATGTCCTGCTCCAG |
| | (GAIE)-CH3 (cak- | AACTGCTGGCCGGACCTTCCGTGTTCCTGTTTCCTCCAAAGCCTAAG |
| | LS) (G1m17) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTAGGGAACCCCAGGTTTACACCCTGCCTCCATGCCGCGAGGAAATGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1248) |
| | Hinge (C/S)-CH2- | GAACCTAAGAGCAGCGACAAGACCCACACCTGTCCTCCATGTCCTGCTCCAG |
| | CH3 (cah-LS) | AACTGCTCGGCGGACCTTCCGTGTTCCTGTTTCCTCCAAAGCCTAAG |
| | (G1m17,1) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTCGCGAACCTCAAGTCTGTACACTGCCTCCTAGCCGCGACGAACTGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1249) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (cah-LS) | GACACCCTGATGATCAGCAGAACCCCTGAAGTGACCTGCGTGGTGGT |
| | (G1m17,1) | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTCGCGAACCTCAAGTCTGTACACTGCCTCCTAGCCGCGACGAACTGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1250) |
| | Hinge (C/S)-CH2 | GAACCTAAGAGCAGCGACAAGACCCACACCTGTCCTCCATGTCCTGCTCCAG |
| | (GAIE)-CH3 (cah- | AACTGCTGGCCGGACCTTCCGTGTTCCTGTTTCCTCCAAAGCCTAAG |
| | LS) (G1m17,1) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTCGCGAACCTCAAGTCTGTACACTGCCTCCTAGCCGCGACGAACTGAC |
| | | CAAGAACCAGGTGTCCCTGAGCTGCGCCGTGAAGGGCTTTTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGGTGTCCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1251) |
| | Hinge (C/S)-CH2- | GAACCTAAGAGCAGCGACAAGACCCACACCTGTCCTCCATGTCCTGCTCCAG |
| | CH3 (cak-LS) | AACTGCTCGGCGGACCTTCCGTGTTCCTGTTTCCTCCAAAGCCTAAG |
| | (G1m17,1) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACCCTGCCTCCATGCCGCGACGAACTGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1252) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (cak-LS) | GACACCCTGATGATCAGCAGAACCCCTGAAGTGACCTGCGTGGTGGT |
| | (G1m17,1) | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTAGGGAACCCCAGGTTTACACCCTGCCTCCATGCCGCGACGAACTGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1253) |
| | Hinge (C/S)-CH2 | GAACCTAAGAGCAGCGACAAGACCCACACCTGTCCTCCATGTCCTGCTCCAG |
| | (GAIE)-CH3 (cak- | AACTGCTGGCCGGACCTTCCGTGTTCCTGTTTCCTCCAAAGCCTAAG |
| | LS) (G1m17,1) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTAGGGAACCCCAGGTTTACACCCTGCCTCCATGCCGCGACGAACTGAC |
| | | CAAGAACCAGGTGTCCCTGTGGTGCCTGGTCAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | AGTGGACAAGAGCAGATGGCAGCAGGGCAACGTGTTCAGCTGCAGCGTGCT |
| | | GCATGAAGCCCTGCACAGCCACTACACCCAGAAGTCCCTGTCTCTGAGCCCC |
| | | GGCAAGTGATAA (SEQ ID NO: 1254) |
| General | Hinge (C/S)-CH2- | GAACCTAAGAGCAGCGACAAGACCCACACCTGTCCTCCATGTCCTGCTCCAG |
| | CH3 (G1m3) | AACTGCTCGGCGGACCTTCCGTGTTCCTGTTTCCTCCAAAGCCTAAG |
| | | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | TGTGGATAAGTCCCGGTGGCAGCAGGGCAACGTGTTCAGCTGTTCTGTGATG |
| | | CACGAAGCCCTGCACAACCACTACACCCAGAAAAGCCTGTCTCTGAGCCCCG |
| | | GCAAGTGATAA (SEQ ID NO: 1255) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (LS) (G1m3) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1256) |
| | Hinge (C/S)-CH2 | GAACCTAAGAGCAGCGACAAGACCCACACCTGTCCTCCATGTCCTGCTCCAG |
| | (GAIE)-CH3 (LS) | AACTGCTGGCCGGACCTTCCGTGTTCCTGTTTCCTCCAAAGCCTAAG |
| | (G1m3) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1257) |
| | Hinge (C/S)-CH2- | GAACCTAAGAGCAGCGACAAGACCCACACCTGTCCTCCATGTCCTGCTCCAG |
| | CH3 (G1m17) | AACTGCTCGGCGGACCTTCCGTGTTCCTGTTTCCTCCAAAGCCTAAG |
| | | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | TGTGGATAAGTCCCGGTGGCAGCAGGGCAACGTGTTCAGCTGTTCTGTGATG |
| | | CACGAAGCCCTGCACAACCACTACACCCAGAAAAGCCTGTCTCTGAGCCCCG |
| | | GCAAGTGATAA (SEQ ID NO: 1258) |
| | Hinge (C/S)-CH2- | GAACCTAAGAGCAGCGACAAGACCCACACCTGTCCTCCATGTCCTGCTCCAG |
| | CH3 (LS) (G1m17) | AACTGCTCGGCGGACCTTCCGTGTTCCTGTTTCCTCCAAAGCCTAAG |
| | | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1259) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (LS) (G1m17) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1260) |
| | Hinge (C/S)-CH2 | GAACCTAAGAGCAGCGACAAGACCCACACCTGTCCTCCATGTCCTGCTCCAG |
| | (GAIE)-CH3 (LS) | AACTGCTGGCCGGACCTTCCGTGTTCCTGTTTCCTCCAAAGCCTAAG |
| | (G1m17) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTAGGGAACCCCAGGTTTACACACTGCCTCCAAGCCGCGAGGAAATGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1261) |
| | Hinge (C/S)-CH2- | GAACCTAAGAGCAGCGACAAGACCCACACCTGTCCTCCATGTCCTGCTCCAG |
| | CH3 (G1m17,1) | AACTGCTCGGCGGACCTTCCGTGTTCCTGTTTCCTCCAAAGCCTAAG |
| | | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACACTGCCTCCAAGCCGCGACGAACTGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | TGTGGATAAGTCCCGGTGGCAGCAGGGCAACGTGTTCAGCTGTTCTGTGATG |
| | | CACGAAGCCCTGCACAACCACTACACCCAGAAAAGCCTGTCTCTGAGCCCCG |
| | | GCAAGTGATAA (SEQ ID NO: 1262) |
| | Hinge (C/S)-CH2- | GAACCTAAGAGCAGCGACAAGACCCACACCTGTCCTCCATGTCCTGCTCCAG |
| | CH3 (LS) | AACTGCTCGGCGGACCTTCCGTGTTCCTGTTTCCTCCAAAGCCTAAG |
| | (G1m17,1) | GACACCCTGATGATCAGCAGAACCCCTGAAGTGACCTGCGTGGTGGT |
| | | GGATGTGTCTCACGAGGACCCAGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACAGTGCTGCACC |
| | | AGGATTGGCTGAACGGCAAAGAGTACAAGTGCAAGGTGTCCAACAA |
| | | GGCCCTGCCTGCTCCTATCGAGAAAACCATCAGCAAGGCCAAGGGCC |
| | | AGCCTAGGGAACCCCAGGTTTACACACTGCCTCCAAGCCGCGACGAACTGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1263) |
| | Hinge (C/S)-CH2 | GAACCTAAGAGCAGCGACAAGACCCACACCTGTCCTCCATGTCCTGCTCCAG |
| | (GAALIE)-CH3 | AACTGCTGGCCGGACCTTCCGTGTTCCTGTTTCCTCCAAAGCCTAAG |
| | (LS) (G1m17,1) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTCTGCCTGAGGAAAAGACCATCAGCAAGGCCAAGGGCC |
| | | AGCCTAGGGAACCCCAGGTTTACACACTGCCTCCAAGCCGCGACGAACTGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1264) |
| | Hinge (C/S)-CH2 | GAACCTAAGAGCAGCGACAAGACCCACACCTGTCCTCCATGTCCTGCTCCAG |
| | (GAIE)-CH3 (LS) | AACTGCTGGCCGGACCTTCCGTGTTCCTGTTTCCTCCAAAGCCTAAG |
| | (G1m17,1) | GACACCCTGATGATCAGCAGAACCCCTGAAGTGACCTGCGTGGTGGT |
| | | GGATGTGTCTCACGAGGACCCTGAAGTGAAGTTCAATTGGTACGTGG |
| | | ACGGCGTGGAAGTGCACAACGCCAAGACCAAGCCTAGAGAGGAACA |
| | | GTACAACAGCACCTACAGAGTGGTGTCCGTGCTGACCGTGCTGCACC |
| | | AGGATTGGCTGAACGGCAAAGAGTACAAGTGCAAGGTGTCCAACAA |
| | | GGCCCTGCCTGCCCCTGAGGAAAAGACCATCAGCAAGGCCAAGGGCC |
| | | AGCCTAGGGAACCCCAGGTTTACACACTGCCTCCAAGCCGCGACGAACTGAC |
| | | CAAGAACCAGGTGTCCCTGACCTGCCTCGTGAAGGGCTTCTACCCTTCCGAT |
| | | ATCGCCGTGGAATGGGAGAGCAATGGCCAGCCTGAGAACAACTACAAGACAA |
| | | CCCCTCCTGTGCTGGACAGCGACGGCTCATTCTTCCTGTACAGCAAGCTGAC |
| | | CGTGGACAAGTCCAGATGGCAGCAGGGCAACGTGTTCAGCTGTAGCGTGCT |
| | | GCACGAAGCCCTGCACAGCCACTACACCCAGAAAAGCCTGTCTCTGAGCCCC |
| | | GGCAAATGATAA (SEQ ID NO: 1265) |

### Plasmid DNA Preparation

Plasmid DNA is prepared by selecting E. coli clones for inoculation in Luria-Bertani (LB) medium containing kanamycin. The cultures are grown overnight at 37° C. and ~150 to 200 rpm. Following cell harvest, purification is done using the QIAGEN Plasmid Plus Maxi Kit according to the manufacturer's instructions. Finally, the concentration is determined by UV spectroscopy. DNA is stored in certified RNase- and DNase-free reaction tubes.

### Linearization and DNA Purification

Linearization of plasmid DNA is performed using appropriate restriction enzymes, followed by purification of the linearized DNA template using magnetic beads (Dynabeads™ MyOne™ Carboxylic Acid) according to the manufacturer's protocol. The DNA concentration is measured by UV spectroscopy.

### In Vitro Transcription

CleanCap 413 (m7(3′OMeG)(5′)ppp(5′)(2′OMeA)pG) capped RNA is produced following the process as disclosed, e.g., in WO 2021214204A1, which is incorporated herein by reference in its entirety. Methyl pseudo-uridine is used in the in vitro transcription reaction and incorporated into the produced RNA. Cellulose purification of the resulting RNA is performed to isolate single-stranded RNA, followed by concentration measurement by UV spectroscopy. The RNA integrity is determined by microfluidic-based electrophoresis.

### Example 11: Generation of Polyribonucleotides Encoding PGDM1400 Monospecific CrossMab^{CH1-CLx} and PGDM1400 Monospecific CrossMab^{CH1-CLcv}

In this Example, polyribonucleotides encoding PGDM1400 in CrossMab^{CH1-CLx} format (as illustrated e.g., in FIG. 4, Panel B) are generated. In CrossMab^{CH1-CLx} format, a CH₁ domain is fused to a VL and a CL domain is fused in between a VH and a CH₂-CH₃ set of domains. By introducing these domain swaps, the resulting CrossMab^{CH1-CLx} Chains will specifically pair, while strongly inhibiting binding to wild-type or unmodified immunoglobulin chains. Thus, with this format, non-functional mispairings are strongly reduced.

Polyribonucleotides encoding PGDM1400 in CrossMab^{CH1-CLcv} format (as illustrated e.g., in FIG. 4, Panel D) are also generated. In CrossMab^{CH1-CLcv} format, a VL is operably linked to a CL domain and a VH is operably linked to a CH₁-CH₂-CH₃ set of domains. To minimize incorrect chain pairing, the CL domain of the immunoglobulin light chain and the CH₁ domain of the immunoglobulin heavy chain are modified to include charge variants, which promote pairing of a constant domain with a positive charge to a constant domain with a negative charge. Exemplary PGDM1400 antibody agent configurations and component immunoglobulin chains are shown in Tables 3 and 4.

Polyribonucleotides encoding the CrossMab^{CH1-CLx} and CrossMab^{CH1-CLcv} formats are cloned into the JR81 vector.

Objectives of the present Example include:
(1) Clone PGDM1400 CrossMab^{CH1-CLx}-encoding DNA Fragment Strings into JR81 backbone; and
(2) Clone PGDM1400 CrossMab^{CH1/CLcv}-encoding DNA Fragment Strings into JR81 backbone.

The methods of the present example include: (1) cloning of DNA fragments into appropriate RNA-expression vector; and (2) verification of selected clones by control digestion and sequencing.

### Codon Optimization and Cloning Strategy

Codon optimization and cloning are performed as described above in Example 9.

Exemplary sequences used in the cloning methods for PGDM1400 CrossMab^{CH1-CLx}-encoding DNA Fragment Strings are shown in Table 21 below.

TABLE 21
Exemplary sequences in PGDM1400 CrossMabCH1-CLx encoding polyribonucleotides.

| Feature | Fragment | Nucleotide Sequence |
|---|---|---|
| General | CH1(SS) (G1m17) | AGCAGCGCCTCTACAAAGGGCCCCAGCGTTTTCCCACTGGCTCCT |
| | | AGCAGCAAGAGCACATCTGGCGGAACAGCCGCTCTGGGCTGTCT |
| | | GGTCAAGGACTACTTTCCCGAGCCTGTGACCGTGTCCTGGAATTC |
| | | TGGCGCTCTGACATCCGGCGTGCACACCTTTCCAGCTGTGCTGCA |
| | | AAGCAGCGGCCTGTACTCTCTGAGCAGCGTGGTCACAGTGCCAAG |
| | | CTCTAGCCTGGGCACCCAGACCTACATCTGCAATGTGAACCACAA |
| | | GCCTAGCAACACGAAGGTCGACAAGAAGGTGGAACCCAAGTCCTG |
| | | CTGATAA (SEQ ID NO: 1266) |
| | Hinge (ΔEPKSC)-CH2-CH3 | GATAAGACCCACACCTGTCCTCCATGTCCAGCTCCAGAACTGCT |
| | (G1m3) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGAGGAA |
| | | ATGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACTGTGGATAAGTCCCGGTGG |
| | | CAGCAGGGCAACGTGTTCAGCTGTTCTGTGATGCACGAAGCCCTG |
| | | CACAACCACTACACCCAGAAAAGCCTGTCTCTGAGCCCCGGCAAG |
| | | TGATAA (SEQ ID NO: 1267) |
| | Hinge (ΔEPKSC)-CH2-CH3 | GATAAGACCCACACCTGTCCTCCATGTCCAGCTCCAGAACTGCT |
| | (G1m17) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGAGGAA |
| | | ATGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACTGTGGATAAGTCCCGGTGG |
| | | CAGCAGGGCAACGTGTTCAGCTGTTCTGTGATGCACGAAGCCCTG |
| | | CACAACCACTACACCCAGAAAAGCCTGTCTCTGAGCCCCGGCAAG |
| | | TGATAA (SEQ ID NO: 1268) |
| | Hinge (ΔEPKSC)-CH2-CH3 | GATAAGACCCACACCTGTCCTCCATGTCCAGCTCCAGAACTGCT |
| | (LS) (G1m17) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGAGGAA |
| | | ATGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACCGTGGACAAGTCCAGATGG |
| | | CAGCAGGGCAACGTGTTCAGCTGTAGCGTGCTGCACGAAGCCCTG |
| | | CACAGCCACTACACCCAGAAAAGCCTGTCTCTGAGCCCCGGCAAA |
| | | TGATAA (SEQ ID NO: 1269) |
| | Hinge (ΔEPKSC)-CH2-CH3 | GATAAGACCCACACCTGTCCTCCATGTCCAGCTCCAGAACTGCT |
| | (G1m17,1) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGACGAA |
| | | CTGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACTGTGGATAAGTCCCGGTGG |
| | | CAGCAGGGCAACGTGTTCAGCTGTTCTGTGATGCACGAAGCCCTG |
| | | CACAACCACTACACCCAGAAAAGCCTGTCTCTGAGCCCCGGCAAG |
| | | TGATAA (SEQ ID NO: 1270) |
| | Hinge (ΔEPKSC)-CH2-CH3 | GATAAGACCCACACCTGTCCTCCATGTCCAGCTCCAGAACTGCT |
| | (LS) (G1m17,1) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGACGAA |
| | | CTGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACCGTGGACAAGTCCAGATGG |
| | | CAGCAGGGCAACGTGTTCAGCTGTAGCGTGCTGCACGAAGCCCTG |
| | | CACAGCCACTACACCCAGAAAAGCCTGTCTCTGAGCCCCGGCAAA |
| | | TGATAA (SEQ ID NO: 1271) |
| General | CH1(SS) (G1m17) | AGCAGCGCCTCTACAAAGGGCCCCAGCGTTTTCCCACTGGCTCCT |
| | | AGCAGCAAGAGCACATCTGGCGGAACAGCCGCTCTGGGCTGTCT |
| | | GGTCAAGGACTACTTTCCCGAGCCTGTGACCGTGTCCTGGAATTC |
| | | TGGCGCTCTGACATCCGGCGTGCACACCTTTCCAGCTGTGCTGCA |
| | | AAGCAGCGGCCTGTACTCTCTGAGCAGCGTGGTCACAGTGCCAAG |
| | | CTCTAGCCTGGGCACCCAGACCTACATCTGCAATGTGAACCACAA |
| | | GCCTAGCAACACGAAGGTCGACAAGAAGGTGGAACCCAAGTCCTG |
| | | CTGATAA (SEQ ID NO: 1272) |
| | Hinge (ΔEPKSC)-CH2-CH3 | GATAAGACCCACACCTGTCCTCCATGTCCAGCTCCAGAACTGCT |
| | (G1m3) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGAGGAA |
| | | ATGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACTGTGGATAAGTCCCGGTGG |
| | | CAGCAGGGCAACGTGTTCAGCTGTTCTGTGATGCACGAAGCCCTG |
| | | CACAACCACTACACCCAGAAAAGCCTGTCTCTGAGCCCCGGCAAG |
| | | TGATAA (SEQ ID NO: 1273) |
| | Hinge (ΔEPKSC)-CH2-CH3 | GATAAGACCCACACCTGTCCTCCATGTCCAGCTCCAGAACTGCT |
| | (G1m17) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGAGGAA |
| | | ATGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACTGTGGATAAGTCCCGGTGG |
| | | CAGCAGGGCAACGTGTTCAGCTGTTCTGTGATGCACGAAGCCCTG |
| | | CACAACCACTACACCCAGAAAAGCCTGTCTCTGAGCCCCGGCAAG |
| | | TGATAA (SEQ ID NO: 1274) |
| | Hinge (ΔEPKSC)-CH2-CH3 | GATAAGACCCACACCTGTCCTCCATGTCCAGCTCCAGAACTGCT |
| | (LS) (G1m17) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGAGGAA |
| | | ATGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACCGTGGACAAGTCCAGATGG |
| | | CAGCAGGGCAACGTGTTCAGCTGTAGCGTGCTGCACGAAGCCCTG |
| | | CACAGCCACTACACCCAGAAAAGCCTGTCTCTGAGCCCCGGCAAA |
| | | TGATAA (SEQ ID NO: 1275) |
| | Hinge (ΔEPKSC)-CH2-CH3 | GATAAGACCCACACCTGTCCTCCATGTCCAGCTCCAGAACTGCT |
| | (G1m17,1) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGACGAA |
| | | CTGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACTGTGGATAAGTCCCGGTGG |
| | | CAGCAGGGCAACGTGTTCAGCTGTTCTGTGATGCACGAAGCCCTG |
| | | CACAACCACTACACCCAGAAAAGCCTGTCTCTGAGCCCCGGCAAG |
| | | TGATAA (SEQ ID NO: 1276) |
| | Hinge (ΔEPKSC)-CH2-CH3 | GATAAGACCCACACCTGTCCTCCATGTCCAGCTCCAGAACTGCT |
| | (LS) (G1m17,1) | CGGCGGACCTTCCGTGTTCCTGTTTCCTCCAAAGCCTAAGG |
| | | ACACCCTGATGATCAGCAGAACCCCTGAAGTGACCTGCGT |
| | | GGTGGTGGATGTGTCTCACGAGGACCCAGAAGTGAAGTTC |
| | | AATTGGTACGTGGACGGCGTGGAAGTGCACAACGCCAAGA |
| | | CCAAGCCTAGAGAGGAACAGTACAACAGCACCTACAGAGT |
| | | GGTGTCCGTGCTGACAGTGCTGCACCAGGATTGGCTGAAC |
| | | GGCAAAGAGTACAAGTGCAAGGTGTCCAACAAGGCCCTGC |
| | | CTGCTCCTATCGAGAAAACCATCAGCAAGGCCAAGGGCCAG |
| | | CCTAGGGAACCCCAGGTTTACACACTGCCTCCAAGCCGCGACGAA |
| | | CTGACCAAGAACCAGGTGTCCCTGACCTGCCTCGTGAAGGGCTTC |
| | | TACCCTTCCGATATCGCCGTGGAATGGGAGAGCAATGGCCAGCCT |
| | | GAGAACAACTACAAGACAACCCCTCCTGTGCTGGACAGCGACGGC |
| | | TCATTCTTCCTGTACAGCAAGCTGACCGTGGACAAGTCCAGATGG |
| | | CAGCAGGGCAACGTGTTCAGCTGTAGCGTGCTGCACGAAGCCCTG |
| | | CACAGCCACTACACCCAGAAAAGCCTGTCTCTGAGCCCCGGCAAA |
| | | TGATAA (SEQ ID NO: 1277) |

Exemplary sequences used in the cloning methods for PGDM1400 CrossMabCH1-CLx-encoding DNA Fragment Strings are shown in Table 22 below.

TABLE 22
Exemplary sequences in PGDM1400 CrossMab^{CH1-CLcv} encoding_polyribonucleotides.

| Feature | Fragment | Nucleotide Sequence |
|---|---|---|
| General | CH1cv (G1m17) | GCTTCCACAAAGGGCCCTAGCGTGTTCCCTCTGGCTCCTAGCAGC |
| | | AAGTCTACAAGCGGAGGAACAGCTGCCCTGGGCTGCCTGGTGGA |
| | | AGATTACTTTCCTGAGCCTGTGACCGTGTCCTGGAACAGCGGTGC |
| | | TCTGACTTCTGGCGTGCACACCTTTCCAGCCGTGCTGCAAAGCAG |
| | | CGGCCTGTACTCTCTGAGCAGCGTGGTCACAGTGCCAAGCTCTAG |
| | | CCTGGGCACCCAGACCTACATCTGCAATGTGAACCACAAGCCTAG |
| | | CAACACCAAGGTGGACGACAAGGTG (SEQ ID NO: 1278) |
| | CH1cv (G1m17,1) | GCTTCCACAAAGGGCCCTAGCGTGTTCCCTCTGGCTCCTAGCAGC |
| | | AAGTCTACAAGCGGAGGAACAGCTGCCCTGGGCTGCCTGGTGGA |
| | | AGATTACTTTCCTGAGCCTGTGACCGTGTCCTGGAACAGCGGTGC |
| | | TCTGACTTCTGGCGTGCACACCTTTCCAGCCGTGCTGCAAAGCAG |
| | | CGGCCTGTACTCTCTGAGCAGCGTGGTCACAGTGCCAAGCTCTAG |
| | | CCTGGGCACCCAGACCTACATCTGCAATGTGAACCACAAGCCTAG |
| | | CAACACCAAGGTGGACGACAAGGTG (SEQ ID NO: 1279) |
| | Hinge-CH2-CH3 (G1m3) | GAACCCAAGAGCTGCGACAAGACCCACACCTGTCCTCCATGTCCT |
| | | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACACTGCCTC |
| | | CAAGCCGCGAGGAAATGACCAAGAACCAGGTGTCCCTGACCTGCC |
| | | TCGTGAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACTGTGG |
| | | ATAAGTCCCGGTGGCAGCAGGGCAACGTGTTCAGCTGTTCTGTGA |
| | | TGCACGAAGCCCTGCACAACCACTACACCCAGAAAAGCCTGTCTC |
| | | TGAGCCCCGGCAAGTGATAA (SEQ ID NO: 1280) |
| | Hinge-CH2 (GAIE)-CH3 | GAACCCAAGAGCTGCGACAAGACCCACACCTGTCCTCCATGTCCT |
| | (LS) (G1m3) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTAGGGAACCCCAGGTTTACACACTGCCT |
| | | CCAAGCCGCGAGGAAATGACCAAGAACCAGGTGTCCCTGACCTGC |
| | | CTCGTGAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACCGTG |
| | | GACAAGTCCAGATGGCAGCAGGGCAACGTGTTCAGCTGTAGCGT |
| | | GCTGCACGAAGCCCTGCACAGCCACTACACCCAGAAAAGCCTGTC |
| | | TCTGAGCCCCGGCAAATGATAA (SEQ ID NO: 1281) |
| | Hinge-CH2-CH3 (G1m17) | GAACCCAAGAGCTGCGACAAGACCCACACCTGTCCTCCATGTCCT |
| | | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACACTGCCTC |
| | | CAAGCCGCGAGGAAATGACCAAGAACCAGGTGTCCCTGACCTGCC |
| | | TCGTGAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACTGTGG |
| | | ATAAGTCCCGGTGGCAGCAGGGCAACGTGTTCAGCTGTTCTGTGA |
| | | TGCACGAAGCCCTGCACAACCACTACACCCAGAAAAGCCTGTCTC |
| | | TGAGCCCCGGCAAGTGATAA (SEQ ID NO: 1282) |
| | Hinge-CH2-CH3 (LS) | GAACCCAAGAGCTGCGACAAGACCCACACCTGTCCTCCATGTCCT |
| | (G1m17) | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACACTGCCTC |
| | | CAAGCCGCGAGGAAATGACCAAGAACCAGGTGTCCCTGACCTGCC |
| | | TCGTGAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACCGTGG |
| | | ACAAGTCCAGATGGCAGCAGGGCAACGTGTTCAGCTGTAGCGTGC |
| | | TGCACGAAGCCCTGCACAGCCACTACACCCAGAAAAGCCTGTCTC |
| | | TGAGCCCCGGCAAATGATAA (SEQ ID NO: 1283) |
| | Hinge-CH2 (GAALIE)-CH3 | GAACCCAAGAGCTGCGACAAGACCCACACCTGTCCTCCATGTCCT |
| | (LS) (G1m17) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTCTGCCTGAGGAAAAGACCATCAGCA |
| | | AGGCCAAGGGCCAGCCTAGGGAACCCCAGGTTTACACACTGCCT |
| | | CCAAGCCGCGAGGAAATGACCAAGAACCAGGTGTCCCTGACCTGC |
| | | CTCGTGAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACCGTG |
| | | GACAAGTCCAGATGGCAGCAGGGCAACGTGTTCAGCTGTAGCGT |
| | | GCTGCACGAAGCCCTGCACAGCCACTACACCCAGAAAAGCCTGTC |
| | | TCTGAGCCCCGGCAAATGATAA (SEQ ID NO: 1284) |
| | Hinge-CH2 (GAIE)-CH3 | GAACCCAAGAGCTGCGACAAGACCCACACCTGTCCTCCATGTCCT |
| | (LS) (G1m17) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTAGGGAACCCCAGGTTTACACACTGCCT |
| | | CCAAGCCGCGAGGAAATGACCAAGAACCAGGTGTCCCTGACCTGC |
| | | CTCGTGAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACCGTG |
| | | GACAAGTCCAGATGGCAGCAGGGCAACGTGTTCAGCTGTAGCGT |
| | | GCTGCACGAAGCCCTGCACAGCCACTACACCCAGAAAAGCCTGTC |
| | | TCTGAGCCCCGGCAAATGATAA (SEQ ID NO: 1285) |
| | Hinge-CH2-CH3 (G1m17,1) | GAACCCAAGAGCTGCGACAAGACCCACACCTGTCCTCCATGTCCT |
| | | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACACTGCCTC |
| | | CAAGCCGCGACGAACTGACCAAGAACCAGGTGTCCCTGACCTGCC |
| | | TCGTGAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACTGTGG |
| | | ATAAGTCCCGGTGGCAGCAGGGCAACGTGTTCAGCTGTTCTGTGA |
| | | TGCACGAAGCCCTGCACAACCACTACACCCAGAAAAGCCTGTCTC |
| | | TGAGCCCCGGCAAGTGATAA (SEQ ID NO: 1286) |
| | Hinge-CH2-CH3 (LS) | GAACCCAAGAGCTGCGACAAGACCCACACCTGTCCTCCATGTCCT |
| | (G1m17,1) | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACACTGCCTC |
| | | CAAGCCGCGACGAACTGACCAAGAACCAGGTGTCCCTGACCTGCC |
| | | TCGTGAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACCGTGG |
| | | ACAAGTCCAGATGGCAGCAGGGCAACGTGTTCAGCTGTAGCGTGC |
| | | TGCACGAAGCCCTGCACAGCCACTACACCCAGAAAAGCCTGTCTC |
| | | TGAGCCCCGGCAAATGATAA (SEQ ID NO: 1287) |
| | Hinge-CH2 (GAALIE)-CH3 | GAACCCAAGAGCTGCGACAAGACCCACACCTGTCCTCCATGTCCT |
| | (LS) (G1m17,1) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTCTGCCTGAGGAAAAGACCATCAGCA |
| | | AGGCCAAGGGCCAGCCTAGGGAACCCCAGGTTTACACACTGCCT |
| | | CCAAGCCGCGACGAACTGACCAAGAACCAGGTGTCCCTGACCTGC |
| | | CTCGTGAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACCGTG |
| | | GACAAGTCCAGATGGCAGCAGGGCAACGTGTTCAGCTGTAGCGT |
| | | GCTGCACGAAGCCCTGCACAGCCACTACACCCAGAAAAGCCTGTC |
| | | TCTGAGCCCCGGCAAATGATAA (SEQ ID NO: 1288) |
| | Hinge-CH2 (GAIE)-CH3 | GAACCCAAGAGCTGCGACAAGACCCACACCTGTCCTCCATGTCCT |
| | (LS) (G1m17,1) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTAGGGAACCCCAGGTTTACACACTGCCT |
| | | CCAAGCCGCGACGAACTGACCAAGAACCAGGTGTCCCTGACCTGC |
| | | CTCGTGAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACCGTG |
| | | GACAAGTCCAGATGGCAGCAGGGCAACGTGTTCAGCTGTAGCGT |
| | | GCTGCACGAAGCCCTGCACAGCCACTACACCCAGAAAAGCCTGTC |
| | | TCTGAGCCCCGGCAAATGATAA (SEQ ID NO: 1289) |
| | κ-CLcv variant 2 | ATGGATTGGATTTGGAGAATCCTGTTCCTCGTGGGCGCCGCTACC |
| | | GGAGCCCACTCCGAAGTTGTGCTGACACAGAGCCCCGCCATCCTG |
| | | AGTGTTAGCCCTGGCGATAGAGTGATCCTGAGCTGCAGAGCCTCT |
| | | CAGGGCCTCGATTCTTCTCACCTGGCCTGGTACAGATTCAAGCGG |
| | | GGACAGATCCCCACACTGGTCATCTTCGGCACCAGCAACAGAGCC |
| | | AGAGGCACCCCTGATAGATTTTCCGGCTCTGGCAGCGGAGCCGAC |
| | | TTCACCCTGACAATCAGCAGAGTGGAACCCGAGGACTTCGCCACC |
| | | TACTACTGCCAGAGATACGGCGGCACCCCTATCACATTTGGCGGC |
| | | GGAACCACACTGGACAAGAAACGTACGGTGGCCGCTCCTAGCGTG |
| | | TTCATCTTTCCACCTAGCGACAGAAAGCTGAAGTCTGGCACAGCC |
| | | TCTGTCGTGTGCCTGCTCAACAACTTCTACCCCAGAGAAGCCAAG |
| | | GTGCAGTGGAAGGTGGACAACGCCCTGCAGAGCGGCAATAGCCA |
| | | AGAGAGCGTGACCGAGCAGGACAGCAAGGACTCTACCTACAGCCT |
| | | GAGCAGCACACTGACCCTGAGCAAGGCCGACTACGAGAAGCACAA |
| | | AGTGTACGCCTGCGAAGTGACCCACCAGGGCCTTTCTAGCCCTGT |
| | | GACCAAGAGCTTCAACCGGGGCGAGTGCTGATAA (SEQ ID NO: |
| | | 1290) |
| General | Hinge-CH2 (GAIE)-CH3 | GAACCCAAGAGCTGCGACAAGACCCACACCTGTCCTCCATGTCCT |
| | (cah-LS) (G1m3) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTCGCGAACCTCAAGTCTGTACACTGCCT |
| | | CCTAGCCGCGAGGAAATGACCAAGAACCAGGTGTCCCTGAGCTGC |
| | | GCCGTGAAGGGCTTTTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGGTGTCCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1291) |
| | Hinge-CH2 (GAIE)-CH3 | GAACCCAAGAGCTGCGACAAGACCCACACCTGTCCTCCATGTCCT |
| | (cak-LS) (G1m3) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTAGGGAACCCCAGGTTTACACCCTGCCT |
| | | CCATGCCGCGAGGAAATGACCAAGAACCAGGTGTCCCTGTGGTGC |
| | | CTGGTCAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1292) |
| | Hinge-CH2-CH3 (cah-LS) | GAACCCAAGAGCTGCGACAAGACCCACACCTGTCCTCCATGTCCT |
| | (G1m17) | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTCGCGAACCTCAAGTCTGTACACTGCCTC |
| | | CTAGCCGCGAGGAAATGACCAAGAACCAGGTGTCCCTGAGCTGCG |
| | | CCGTGAAGGGCTTTTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGGTGTCCAAGCTGACAGTGG |
| | | ACAAGAGCAGATGGCAGCAGGGCAACGTGTTCAGCTGCAGCGTG |
| | | CTGCATGAAGCCCTGCACAGCCACTACACCCAGAAGTCCCTGTCT |
| | | CTGAGCCCCGGCAAGTGATAA (SEQ ID NO: 1293) |
| | Hinge-CH2 (GAALIE)-CH3 | GAACCCAAGAGCTGCGACAAGACCCACACCTGTCCTCCATGTCCT |
| | (cah-LS) (G1m17) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTCTGCCTGAGGAAAAGACCATCAGCA |
| | | AGGCCAAGGGCCAGCCTCGCGAACCTCAAGTCTGTACACTGCCT |
| | | CCTAGCCGCGAGGAAATGACCAAGAACCAGGTGTCCCTGAGCTGC |
| | | GCCGTGAAGGGCTTTTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGGTGTCCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1294) |
| | Hinge-CH2 (GAIE)-CH3 | GAACCCAAGAGCTGCGACAAGACCCACACCTGTCCTCCATGTCCT |
| | (cah-LS) (G1m17) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTCGCGAACCTCAAGTCTGTACACTGCCT |
| | | CCTAGCCGCGAGGAAATGACCAAGAACCAGGTGTCCCTGAGCTGC |
| | | GCCGTGAAGGGCTTTTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGGTGTCCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1295) |
| | Hinge-CH2-CH3 (cak-LS) | GAACCCAAGAGCTGCGACAAGACCCACACCTGTCCTCCATGTCCT |
| | (G1m17) | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACCCTGCCTC |
| | | CATGCCGCGAGGAAATGACCAAGAACCAGGTGTCCCTGTGGTGCC |
| | | TGGTCAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACAGTGG |
| | | ACAAGAGCAGATGGCAGCAGGGCAACGTGTTCAGCTGCAGCGTG |
| | | CTGCATGAAGCCCTGCACAGCCACTACACCCAGAAGTCCCTGTCT |
| | | CTGAGCCCCGGCAAGTGATAA (SEQ ID NO: 1296) |
| | Hinge-CH2 (GAALIE)-CH3 | GAACCCAAGAGCTGCGACAAGACCCACACCTGTCCTCCATGTCCT |
| | (cak-LS) (G1m17) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTCTGCCTGAGGAAAAGACCATCAGCA |
| | | AGGCCAAGGGCCAGCCTAGGGAACCCCAGGTTTACACCCTGCCT |
| | | CCATGCCGCGAGGAAATGACCAAGAACCAGGTGTCCCTGTGGTGC |
| | | CTGGTCAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1297) |
| | Hinge-CH2 (GAIE)-CH3 | GAACCCAAGAGCTGCGACAAGACCCACACCTGTCCTCCATGTCCT |
| | (cak-LS) (G1m17) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTAGGGAACCCCAGGTTTACACCCTGCCT |
| | | CCATGCCGCGAGGAAATGACCAAGAACCAGGTGTCCCTGTGGTGC |
| | | CTGGTCAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1298) |
| | Hinge-CH2-CH3 (cah-LS) | GAACCCAAGAGCTGCGACAAGACCCACACCTGTCCTCCATGTCCT |
| | (G1m17,1) | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTCGCGAACCTCAAGTCTGTACACTGCCTC |
| | | CTAGCCGCGACGAACTGACCAAGAACCAGGTGTCCCTGAGCTGCG |
| | | CCGTGAAGGGCTTTTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGGTGTCCAAGCTGACAGTGG |
| | | ACAAGAGCAGATGGCAGCAGGGCAACGTGTTCAGCTGCAGCGTG |
| | | CTGCATGAAGCCCTGCACAGCCACTACACCCAGAAGTCCCTGTCT |
| | | CTGAGCCCCGGCAAGTGATAA (SEQ ID NO: 1299) |
| | Hinge-CH2 (GAALIE)-CH3 | GAACCCAAGAGCTGCGACAAGACCCACACCTGTCCTCCATGTCCT |
| | (cah-LS) (G1m17,1) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTCTGCCTGAGGAAAAGACCATCAGCA |
| | | AGGCCAAGGGCCAGCCTCGCGAACCTCAAGTCTGTACACTGCCT |
| | | CCTAGCCGCGACGAACTGACCAAGAACCAGGTGTCCCTGAGCTGC |
| | | GCCGTGAAGGGCTTTTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGGTGTCCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1300) |
| | Hinge-CH2 (GAIE)-CH3 | GAACCCAAGAGCTGCGACAAGACCCACACCTGTCCTCCATGTCCT |
| | (cah-LS) (G1m17,1) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTCGCGAACCTCAAGTCTGTACACTGCCT |
| | | CCTAGCCGCGACGAACTGACCAAGAACCAGGTGTCCCTGAGCTGC |
| | | GCCGTGAAGGGCTTTTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGGTGTCCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1301) |
| | Hinge-CH2-CH3 (cak-LS) | GAACCCAAGAGCTGCGACAAGACCCACACCTGTCCTCCATGTCCT |
| | (G1m17,1) | GCTCCAGAACTGCTCGGCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | AGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACAGTGCTGCACCA |
| | | GGATTGGCTGAACGGCAAAGAGTACAAGTGCAAGGTGTCC |
| | | AACAAGGCCCTGCCTGCTCCTATCGAGAAAACCATCAGCAA |
| | | GGCCAAGGGCCAGCCTAGGGAACCCCAGGTTTACACCCTGCCTC |
| | | CATGCCGCGACGAACTGACCAAGAACCAGGTGTCCCTGTGGTGCC |
| | | TGGTCAAGGGCTTCTACCCTTCCGATATCGCCGTGGAATGGGAGA |
| | | GCAATGGCCAGCCTGAGAACAACTACAAGACAACCCCTCCTGTGC |
| | | TGGACAGCGACGGCTCATTCTTCCTGTACAGCAAGCTGACAGTGG |
| | | ACAAGAGCAGATGGCAGCAGGGCAACGTGTTCAGCTGCAGCGTG |
| | | CTGCATGAAGCCCTGCACAGCCACTACACCCAGAAGTCCCTGTCT |
| | | CTGAGCCCCGGCAAGTGATAA (SEQ ID NO: 1302) |
| | Hinge-CH2 (GAALIE)-CH3 | GAACCCAAGAGCTGCGACAAGACCCACACCTGTCCTCCATGTCCT |
| | (cak-LS) (G1m17,1) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTCTGCCTGAGGAAAAGACCATCAGCA |
| | | AGGCCAAGGGCCAGCCTAGGGAACCCCAGGTTTACACCCTGCCT |
| | | CCATGCCGCGACGAACTGACCAAGAACCAGGTGTCCCTGTGGTGC |
| | | CTGGTCAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1303) |
| | Hinge-CH2 (GAIE)-CH3 | GAACCCAAGAGCTGCGACAAGACCCACACCTGTCCTCCATGTCCT |
| | (cak-LS) (G1m17,1) | GCTCCAGAACTGCTGGCCGGACCTTCCGTGTTCCTGTTTCC |
| | | TCCAAAGCCTAAGGACACCCTGATGATCAGCAGAACCCCTG |
| | | AAGTGACCTGCGTGGTGGTGGATGTGTCTCACGAGGACCC |
| | | TGAAGTGAAGTTCAATTGGTACGTGGACGGCGTGGAAGTG |
| | | CACAACGCCAAGACCAAGCCTAGAGAGGAACAGTACAACA |
| | | GCACCTACAGAGTGGTGTCCGTGCTGACCGTGCTGCACCA |
| | | GGATTGGCTGAACGGCAAAGAGTACAAGTGCAAGGTGTCC |
| | | AACAAGGCCCTGCCTGCCCCTGAGGAAAAGACCATCAGCA |
| | | AGGCCAAGGGCCAGCCTAGGGAACCCCAGGTTTACACCCTGCCT |
| | | CCATGCCGCGACGAACTGACCAAGAACCAGGTGTCCCTGTGGTGC |
| | | CTGGTCAAGGGCTTCTACCCTTCCGATATCGCCGTGGAATGGGAG |
| | | AGCAATGGCCAGCCTGAGAACAACTACAAGACAACCCCTCCTGTG |
| | | CTGGACAGCGACGGCTCATTCTTCCTGTACAGCAAGCTGACAGTG |
| | | GACAAGAGCAGATGGCAGCAGGGCAACGTGTTCAGCTGCAGCGT |
| | | GCTGCATGAAGCCCTGCACAGCCACTACACCCAGAAGTCCCTGTC |
| | | TCTGAGCCCCGGCAAGTGATAA (SEQ ID NO: 1304) |

### Example 12: In Vivo Validation of Parental and Mutated PGDM1400 (IgG and scFv-Fc) RibobNAbs

Generation of polyribonucleotides encoding for PGDM1400 antibody agents as described herein allows the PGDM1400 antibody agents to be delivered in combination with other polyribonucleotides that encode additional anti-HIV antibody agents. Use of the technologies and approaches described herein also allows for simultaneous production of different antibody agents from polyribonucleotides. The formats of antibody agents have been designed to minimize or eliminate the risk of immunoglobulin chain mispairing. Being able to combine multiple antibody agent formulations as described herein (e.g., including a PGDM1400 antibody agent) allows for the development of a composition (e.g., a pharmaceutical composition) that delivers multiple antibody agents together so that they can bind different epitopes of the HIV virus, thereby minimizing viral escape through mutations.

The present Example shows that polyribonucleotides encoding PGDM1400 antibody agents can induce production of PGDM1400 L/S scFv-Fc. Furthermore, the present Example shows that polyribonucleotides encoding PGDM1400 antibody agents can drive a high antibody titer in the serum of test subjects over a period of time. The example shows a direct comparison of the PK profile of PGDM1400 L/S scFv-Fc with PGDM1400 L/S IgG. A mutation (“L/S”) was introduced in the CH₃ domain of certain antibody agents to improve binding to the FcRn receptor and thereby improve the terminal kinetic and increase the half-life of the antibody agent (see, for example, Zalevsky et al., 2010, Nature biotechnology). In this Example, the pharmacokinetic properties of PGDM1400 (polyribonucleotides as shown in SEQ ID NOs: 1306 and 1312 (HC and LC, respectively)), PGDM1400 L/S IgG (polyribonucleotides as shown in SEQ ID NOs: 1309 and 1312 (HC and LC, respectively)), and PGDM1400 L/S scFv-Fc agents (polyribonucleotides as shown in SEQ ID NO: 1351 (“VL-LL5-VH”)) were evaluated to assess the mutation's effect on such antibody qualities. NSG hFcRn TG32 mice were used, where the murine FcRn receptor has been exchanged to the human version.

The concentration of the PGDM1400 IgG antibody and the L/S mutated PGDM1400 IgG and scFv-Fc antibody was analyzed in serum of NSG hFcRn Tg32 mice.

NSG hFcRn TG32 mice were injected intravenously with LNP-encapsulated polyribonucleotides encoding PGDM1400 L/S scFv-Fc (1 Chain), PGDM1400 IgG and PGDM1400 L/S IgG antibody agents (1.5:1 HC/LC) in 150 μL of PBS. Blood was sampled from the mice at various time points, and serum was analyzed for the abundance of the RibobNAbs over a time period of 33 days with Gyros ELISA, as described herein.

### Results

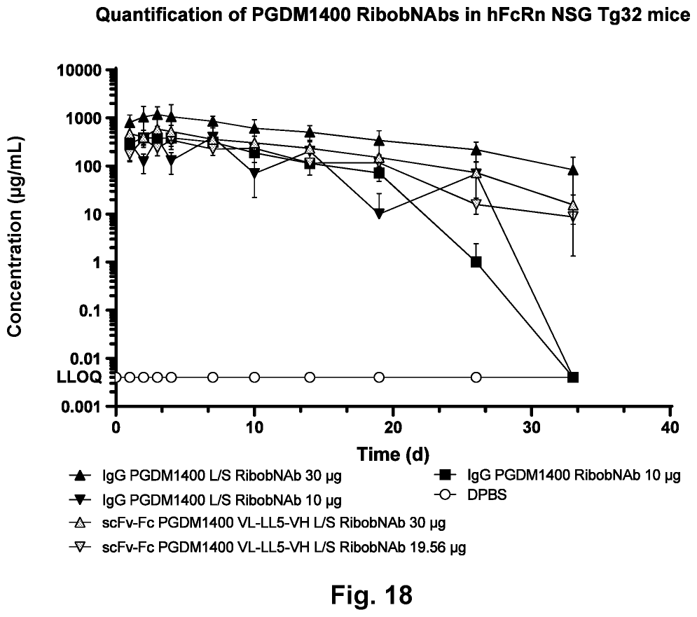

The results are presented in FIG. 18 in log scale. The 1-18 scFv-Fc and IgG variants were all successfully produced in vivo. Both the 1-18 L/S scFv-Fc and 1-18 L/S IgG antibody agents were expressed in vivo in a dose-dependent manner. The scFv-Fc variant has a lower Cmax compared to the IgG variants (~100 μg/ml vs ~1000 μg/ml). Furthermore, the 1-18 L/S IgG (10 μg Dose) antibody agent displayed improved pharmacokinetics as compared to the 1-18 IgG (10 μg Dose) antibody agent, confirming that the L/S mutation can indeed extend antibody half-life.

### EQUIVALENTS

Those skilled in the art will recognize, or be able to ascertain using no more than routine experimentation, many equivalents to the specific embodiments of technologies described herein. The scope of the present disclosure is not intended to be limited to the above Description, but rather is as set forth in the following claims.

Lengthy table referenced here
US20260217799A1-20260730-T00001
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260217799A1). An electronic copy of the table will also be available from the USPTO upon request and payment of the fee set forth in 37 CFR 1.19(b)(3).

1. A polyribonucleotide encoding an immunoglobulin chain of an antibody agent, wherein the immunoglobulin chain comprises a single chain fragment variable (scFv), and the scFv comprises a heavy chain variable (VH) domain, a linker, and a light chain variable (VL) domain,
wherein the VH domain comprises an HCDR1, an HCDR2, and an HCDR3, wherein the HCDR1, HCDR2, and HCDR3 comprise an amino acid sequence according to SEQ ID NO: 18, 21, and 24, respectively, and
wherein the VH domain comprises a first mutation at residue 3 and a second mutation at residue 5 relative to the amino acid sequence according to SEQ ID NO: 1494; and
wherein the VL domain comprises an LCDR1, an LCDR2, and an LCDR3, wherein the LCDR1, LCDR2, and LCDR3 comprise an amino acid sequence according to SEQ ID NO: 27, 30, and 33, respectively.

2. The polyribonucleotide of claim 1, wherein the first mutation comprises a substitution mutation that results in a positively charged amino acid being present at residue 3.

3. The polyribonucleotide of claim 2, wherein the positively charged amino acid comprises an amino acid selected from Lys (K), Arg (R), or His (H).

4. The polyribonucleotide of claim 2 or 3, wherein the positively charged amino acid comprises a His (H) residue.

5. The polyribonucleotide of any one of claims 1-4, wherein the second mutation comprises a substitution mutation that results in a polar amino acid being present at residue 5.

6. The polyribonucleotide of claim 5, wherein the polar amino acid comprises an amino acid selected from Ser (S), Thr (T), Tyr (Y), Asn (N), or Gln (Q).

7. The polyribonucleotide of claim 5 or 6, wherein the polar amino acid comprises a Thr (T) residue.

8. The polyribonucleotide of any one of claims 1-7, wherein the VH domain comprises an amino acid sequence that is at least 85% identical to the amino acid sequence according to SEQ ID NO: 36.

9. The polyribonucleotide of any one of claims 1-8, wherein the VH domain comprises an amino acid sequence that is at least 90% identical to the amino acid sequence according to SEQ ID NO: 36.

10. The polyribonucleotide of any one of claims 1-9, wherein the VH domain comprises an amino acid sequence SEQ ID NO: 1494 with substitution mutations Q3H and V5T.

11. The polyribonucleotide of any one of claims 1-10, wherein the VH domain comprises an amino acid sequence according to SEQ ID NO: 36.

12. The polyribonucleotide of any one of claims 1-11, wherein the VL domain comprises an amino acid sequence that is at least 85% identical to the amino acid sequence according to SEQ ID NO: 43.

13. The polyribonucleotide of any one of claims 1-12, wherein the VL domain comprises an amino acid sequence that is at least 90% identical to the amino acid sequence according to SEQ ID NO: 43.

14. The polyribonucleotide of any one of claims 1-13, wherein the VL domain comprises an amino acid sequence according to SEQ ID NO: 43.

15. The polyribonucleotide of any one of claims 1-14, wherein the scFv comprises, in order:
(i) the VH domain,
(ii) the linker, and
(iii) the VL domain.

16. The polyribonucleotide of any one of claims 1-14, wherein the scFv comprises, in order:
(i) the VL domain,
(ii) the linker, and
(iii) the VH domain.

17. The polyribonucleotide of any one of claims 1-16, wherein the linker comprises an amino acid sequence according to SEQ ID NO: 48.

18. The polyribonucleotide of any one of claims 1-16, wherein the linker comprises an amino acid sequence according to SEQ ID NO: 59.

19. The polyribonucleotide of any one of claims 1-18, wherein the immunoglobulin chain further comprises a second linker following the scFv domain.

20. The polyribonucleotide of claim 19, wherein the scFv and the second linker comprises or consists of an amino acid sequence according to SEQ ID NO: 64, 67, 70, or 73.

21. The polyribonucleotide of any one of claims 1-20, wherein the immunoglobulin chain comprises a hinge domain following the second linker.

22. The polyribonucleotide of claim 21, wherein the hinge domain comprises or consists of an amino acid sequence according to SEQ ID NO: 167.

23. The polyribonucleotide of any one of claims 1-9, wherein the immunoglobulin chain comprises one or more constant domains, and wherein the scFv is operably linked to the one or more constant domains.

24. The polyribonucleotide of claim 10, wherein the hinge domain is between the scFv and the one or more constant domains.

25. The polyribonucleotide of claim 10 or claim 11, wherein the one or more constant domains comprise a CH₃ domain.

26. The polyribonucleotide of claim 25, wherein the CH₃ domain comprises a G1m17,1 or a G1m3 allotype.

27. The polyribonucleotide of claim 26, wherein the CH₃ domain comprises one or more substitution mutations, wherein the one or more substitution mutations comprise or consist of M88L, N94S, or a combination thereof, and wherein the substitution mutation positions are relative to the amino acid sequence according to SEQ ID NO: 1495.

28. The polyribonucleotide of claim 27, wherein the CH₃ domain comprises substitution mutations at residue 16 and residue 18 relative to the amino acid sequence according to SEQ ID NO: 1495.

29. The polyribonucleotide of claim 28, wherein the CH₃ domain comprises substitution mutations D16E and L18M relative to the amino acid sequence according to SEQ ID NO: 1495.

30. The polyribonucleotide of any one of claims 25-29, wherein the CH₃ domain comprises or consists of an amino acid sequence according to SEQ ID NO: 116.

31. The polyribonucleotide of claim 1, wherein the immunoglobulin chain comprises or consists of a sequence according to SEQ ID NO: 1343.

32. The polyribonucleotide of claim 1, wherein the immunoglobulin chain comprises or consists of a sequence according to SEQ ID NO: 1346.

33. The polyribonucleotide of claim 1, wherein the immunoglobulin chain comprises or consists of a sequence according to SEQ ID NO: 1349.

34. The polyribonucleotide of claim 1, wherein the immunoglobulin chain comprises or consists of a sequence according to SEQ ID NO: 1352.

35. The polyribonucleotide of any one of claims 1-34, wherein the polyribonucleotide comprises a ribonucleic acid sequence encoding a secretion signal.

36. The polyribonucleotide of claim 35, wherein the secretion signal comprises a ribonucleic acid sequence that is at least 90% identical to SEQ ID NO: 4 or 8.

37. The polyribonucleotide of claim 36, wherein the secretion signal comprises a ribonucleic acid sequence comprises SEQ ID NO: 4 or 8.

38. The polyribonucleotide of any one of claims 1-37, wherein the polyribonucleotide comprises one or more non-coding sequence elements.

39. The polyribonucleotide of claim 38, wherein the one or more non-coding sequence elements enhances RNA stability and/or translation efficiency.

40. The polyribonucleotide of claim 38 or 39, wherein the one or more non-coding sequence elements comprise a 3′ untranslated region (UTR), a 5′ UTR, a 5′-cap, a polyadenine (polyA) tail, or any combination thereof.

41. The polyribonucleotide of claim 40, wherein the polyA tail is or comprises a modified polyA sequence, preferably an interrupted polyA tail.

42. The polyribonucleotide of claim 41, wherein the polyA tail comprises or consists of a sequence that is at least 90% identical to SEQ ID NO: 16.

43. The polyribonucleotide of any one of claims 40-42, wherein the 3′ UTR comprises or consists of a nucleic acid sequence that is at least 90% identical to SEQ ID NO: 14.

44. The polyribonucleotide of any one of claims 40-43, wherein the 5′ UTR comprises or consists of a nucleic acid sequence that is at least 90% identical to SEQ ID NO: 10.

45. The polyribonucleotide of any one of claims 40-44, wherein the 5′-cap is (m₂^{7,3′}-O)Gppp(m²′°)ApG.

46. The polyribonucleotide of any one of claims 1-45, wherein the polyribonucleotide comprises one or more modified ribonucleotides.

47. The polyribonucleotide of claim 46, wherein the one or more modified ribonucleotides comprise pseudouridine.

48. The polyribonucleotide of any one of claims 1-47, wherein the polyribonucleotide comprises or consists of a ribonucleic acid sequence according to SEQ ID NO: 1000.

49. The polyribonucleotide of any one of claims 1-47, wherein the polyribonucleotide comprises or consists of a ribonucleic acid sequence according to SEQ ID NO: 1048.

50. The polyribonucleotide of any one of claims 1-47, wherein the polyribonucleotide comprises or consists of a ribonucleic acid sequence according to SEQ ID NO: 1096.

51. The polyribonucleotide of any one of claims 1-47, wherein the polyribonucleotide comprises or consists of a ribonucleic acid sequence according to SEQ ID NO: 1144.

52. A polyribonucleotide comprising or consisting of a ribonucleic acid sequence according to any one of SEQ ID NOs: 1000, 1048, 1096, or 1044.

53. A composition comprising one or more polyribonucleotides of any one of claims 1-52.

54. The composition of claim 53, wherein the composition further comprises lipid nanoparticles, polyplexes (PLX), lipidated polyplexes (LPLX), or liposomes,
wherein the one or more polyribonucleotides are fully or partially encapsulated within the lipid nanoparticles, polyplexes (PLX), lipidated polyplexes (LPLX), or liposomes.

55. The composition of claim 53 or claim 54, wherein the composition further comprises lipid nanoparticles, wherein the one or more polyribonucleotides are encapsulated within the lipid nanoparticles.

56. The composition of claim 54 or claim 55, wherein the lipid nanoparticles are cationic lipid nanoparticles.

57. A pharmaceutical composition comprising the composition of any one of claims 53-56 and at least one pharmaceutically acceptable excipient.

58. The pharmaceutical composition of claim 57 for use in the treatment and/or prevention of HIV comprising administering the pharmaceutical composition to a subject.

59. A method comprising administering the pharmaceutical composition of claim 57 or claim 58 to a subject.

60. The method of claim 59 or the pharmaceutical composition for use according to claim 58, wherein administering the pharmaceutical composition to the subject results in expression of the antibody agent in the subject.

61. The method of claim 59 or 60 or the pharmaceutical composition for use according to claim 58, wherein the antibody agent is expressed in the subject at a titer of:
(a) at least 1 μg/ml in plasma; or
(b) at least 1 μg/ml in serum.

62. The method of claim 61, wherein the antibody agent is expressed in the subject at a titer of:
(a) at least 10 μg/ml in plasma; or
(b) at least 10 μg/ml in serum.

63. The method of claim 62, wherein the antibody agent is detectable in serum of a subject for period of time of at least 5 days, at least 10 days, at least 15 days, at least 20 days, at least 25 days, or at least 30 days.

64. The method of claim 62 or claim 63, wherein the antibody agent is detectable in serum of a subject for a period of time of at least 30 days.

65. The method of any one of claims 59-64 or the pharmaceutical composition for use according to claim 58, wherein the antibody agent is capable of neutralizing one or more HIV strains when tested in the TZM-bl cell pseudovirus neutralization assay at antibody agent concentrations up to 25 μg/ml.

66. The method of any one of claims 59-65 or the pharmaceutical composition for use according to claim 58, wherein the antibody agent delivered as a polyribonucleotide has higher neutralization activity of one or more HIV strains compared to an equivalent amount of parental control antibody delivered as a polyribonucleotide, wherein the parental control antibody is an IgG antibody comprising the same VH and VL domain as the antibody agent.

67. The method of claim 65 or claim 66, wherein the one of more HIV strains comprises one or more HIV strains selected from ZM53M.PB12, Du156.12, Q769.d22, 0330.v4.c3, R2184.c04, 89-F1_2_25, CAP204_2_00_F6_6, Ce1176_A3, 6980.v0.c31, PVO.4, CAP45, CNE8, T250-4, 3103.v3.c10 and C1080_c3.

68. The method of any one of claims 59-67 or the pharmaceutical composition for use according to claim 58, wherein the subject has or is at risk of developing an HIV infection.

69. The method of any one of claims 59-68, wherein the method is a method of treating and/or preventing an HIV infection.

70. Use of the composition of any one of claims 53-56 or the pharmaceutical composition of claim 57 or claim 58 for the treatment and/or prevention of HIV in a subject.

71. A method of producing an antibody agent comprising administering to cells the composition of any one of claims 51-54 or the pharmaceutical composition of claim 57 or claim 58 so that the cells express and secrete the antibody agent.

72. The method of claim 71, wherein the cells are in a subject and the antibody agent is produced at a therapeutically relevant plasma concentration or a therapeutically relevant serum concentration.

73. The method of claim 72, wherein the therapeutically relevant plasma concentration or the therapeutically relevant serum concentration is at least 10 μg/ml.
